(19) United States
(12) Patent Application Publication
(10) Pub. No.: US 2024/0026350 A1
(43) Pub. Date: Jan. 25, 2024

(54) NUCLEIC ACID CONSTRUCTS COMPRISING GENE EDITING MULTI-SITES

(71) Applicant: IO BIOSCIENCES, INC., Chalfont, PA (US)

(72) Inventors: Sicco Hans POPMA, Chalfont, PA (US); Di ZHANG, Hillsborough, NJ (US)

(21) Appl. No.: 17/917,472

(22) PCT Filed: Apr. 7, 2021
(86) PCT No.: PCT/US2021/026240

§ 371 Date: Oct. 6, 2022

Related U.S. Application Data
(60) Provisional application No. 63/006,598, filed on Apr. 7, 2020.

(51) Int. Cl. C12N 15/11; C12N 15/90; C12N 9/22

(52) U.S. Cl. CPC C12N 15/11; C12N 15/907; C12N 9/22; C12N 2310/20

(57) ABSTRACT

Disclosed herein is a polynucleotide construct comprising one or more nuclease recognition sequences upstream and downstream of a Gene editing multi-site that comprises a plurality of recognition sequences for a site-specific recombinase or a nuclease. The plurality of recognition sequences facilitate insertion of one or more exogenous donor genes into the host cell.

### CROSS-REFERENCE

This application claims the benefit of U.S. Provisional Application No. 63/006,598, filed Apr. 7, 2020; which application is incorporated herein by reference in its entirety.

### BACKGROUND OF THE DISCLOSURE

Cell therapies enter a new era with the advent of widely available and constantly improving gene modification techniques. Gene modification of cells allows for genetic properties to be deleted, corrected or added in a transient or permanent fashion. For example, the addition of chimeric antigen receptors to patient's white blood cells has led to personalized cell therapies that specifically kill targeted tumor cells in the field of immune oncology. Several clinical proofs of concept studies have now shown promising results for this therapeutic approach. This information can now be used to create cell therapies that adhere to more classic pharmaceutical and biotechnology drug development and commercial models allowing for maximum patient access, give healthcare providers options for treatment, and provide commercial value to the developer. These personalized clinical studies show feasibility of the concept, but face significant scalability and commercial challenges before it can become widely available to all patients in need. There remains a need to provide an avenue to translate the proof of concept studies to a more widely available system, for use in a broader spectrum of patients or against a broader spectrum of conditions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

### SUMMARY OF THE DISCLOSURE

In one aspect provided herein is a gene editing multi-site (GEMS) construct for insertion into an insertion site in a genome of a host cell, wherein said GEMS construct comprises: a GEMS sequence comprising a plurality of first recognition sequences for a site specific recombinase, wherein at least one of the plurality of first recognition sequences can undergo a site specific recombination with a second recognition sequence of the site specific recombinase, when contacted with the site specific recombinase.

In some embodiments, each of the plurality of first recognition sequences can undergo the site specific recombination with the second recognition sequence of the site specific recombinase, when contacted with the site specific recombinase. In some embodiments, the plurality of first recognition sequences comprise at least 3, 4, 5, 6, 7, 8, 9, 10, 11, 12, 13, 14, 15, 16, 17, 18, 19, 20, 25, or more first recognition sequences. In some embodiments, the site specific recombinase is a serine recombinase, or a tyrosine recombinase. In some embodiments, the serine recombinase is a serine integrase. In some embodiments, the serine recombinase is a Bxb1 integrase, a phiBT1, a R4 integrase, a TP901 integrase, gamma-delta resolvase, Tn3 resolvase, a phiC31 integrase, γδ resolvase, Gin invertase. In some embodiments, the tyrosine recombinase is a tyrosine integrase.

In some embodiments, the tyrosine recombinase is a Cre recombinase, a Flp-recombinase, a XerC recombinase, a λ integrase, a HK022 integrase, a P22 integrase, a HP1 integrase, a L5 integrase.

In some embodiments, the plurality of first recognition sequence is an att site. In some embodiments, the plurality of first recognition sequence is a bacterial genomic recombination site (attB) or a phage genomic recombination site (attP). In some embodiments, the second recognition sequence is an att site that is selected from is a bacterial genomic recombination site (attB) or a phage genomic recombination site (attP). In some embodiments, the first recognition sequence is an attB site, and the second recognition sequence is an attp site. In some embodiments, the first recognition sequence is an attP site, and the second recognition sequence is an attB site. In some embodiments, at least 2, 3, 4, 5, 6, 7, 8, 9, 10, 11, 12, 13, 14, 15, 16, 17, 18, 19, 20, 25, or more of the plurality of first recognition sequences is an attB site. In some embodiments, at least 2, 3, 4, 5, 6, 7, 8, 9, 10, 11, 12, 13, 14, 15, 16, 17, 18, 19, 20, 25, or more of the plurality of first recognition sequences is an attP site. In some embodiments, each of the plurality of the first recognition sequences is the same. In some embodiments, each of the plurality of first recognition sequences is an attP site, or an attB site. In some embodiments, at least one of said plurality of first recognition sequences is heterologous to said genome. In some embodiments, each of said plurality of nuclease recognition sequences is heterologous to said genome.

In some embodiments, the second recognition sequence is heterologous to said genome. In some embodiments, the GEMS sequence is heterologous to said genome. In some embodiments, each of said plurality of first recognition sequences is non-coding. In some embodiments, the GEMS sequence is non-coding. In some embodiments, each of the plurality of the first recognition sequence comprise at least about 10 to about 500 nucleotides. In some embodiments, each of the plurality of the first recognition sequence comprise at least about 20 to about 300 nucleotides. In some embodiments, each of the plurality of the first recognition sequence comprise at least about 30 to about 200 nucleotides. In some embodiments, each of the plurality of the first recognition sequence comprise at least about 40 to about 100 nucleotides. In some embodiments, said GEMS sequence further comprises one or more polynucleotide spacers separating said plurality of first recognition sequences.

In some embodiments, each of said one or more polynucleotide spacers comprise at least about 2 to about 10,000 nucleotides. In some embodiments, each of said one or more polynucleotide spacers comprise at least about 25 to about 100 nucleotides. In some embodiments, each of said one or more polynucleotide spacers comprise each about 25 to about 50 nucleotides. In some embodiments, each of said one or more polynucleotide spacers comprises a unique sequence. In some embodiments, at least one of said plurality of first recognition sequences comprises a sequence set forth in SEQ ID NO: 106, SEQ ID NO: 107, or reverse complements thereof. In some embodiments, each of said plurality of first recognition sequences comprises a sequence set forth in SEQ ID NOs: 106, SEQ ID NO: 107, or reverse complements thereof. In some embodiments, said GEMS sequence comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 105.

In some embodiments, said GEMS sequence comprises the sequence set forth in SEQ ID NO: 105. In some embodiments, the GEMS construct of any one of embodiments above, further comprises a first flanking insertion sequence homologous to a first genome sequence upstream of said insertion site, said first flanking insertion sequence located upstream of said GEMS sequence, and a second flanking insertion sequence homologous to a second genome sequence downstream of said insertion site, said second flanking insertion sequence located downstream of said GEMS sequence. In some embodiments, said first flanking insertion sequence, said second flanking insertion sequence, or both comprise at least 12 nucleotides.

In some embodiments, said first flanking insertion sequence, said second flanking insertion sequence, or both comprise at least 18 nucleotides. In some embodiments, said first flanking insertion sequence, said second flanking insertion sequence, or both comprise at least 50 nucleotides. In some embodiments, said first flanking insertion sequence, said second flanking insertion sequence, or both comprise at least 100 nucleotides.

In some embodiments, said first flanking insertion sequence, said second flanking insertion sequence, or both comprise at least 500 nucleotides. In some embodiments, said insertion site is a safe harbor site of said genome. In some embodiments, said safe harbor site is an adeno-associated virus site 1 (AAVs1) site, a Rosa26 site, a C-C motif receptor 5 (CCR5) site, or a Hipp11 (H11) site. In some embodiments, the GEMS construct further comprises a first nuclease recognition sequence upstream of said GEMS sequence. In some embodiments, further comprises a second nuclease recognition sequence downstream of said GEMS sequence. In some embodiments, said first and/or said second nuclease recognition sequence is recognized by a nuclease that is a zinc finger nuclease, a transcription activator-like effector nuclease, a meganuclease, a Cas protein, or a Cpf 1 protein.

In some embodiments, said nuclease is said meganuclease. In some embodiments, said meganuclease is an I-SceI meganuclease. In some embodiments, said first nuclease recognition sequence is upstream of said first flanking insertion sequence and/or said second nuclease recognition sequence for said nuclease is downstream of said second flanking insertion sequence. In some embodiments, said GEMS construct further comprises a reporter gene. In some embodiments, said reporter gene encodes a fluorescent protein. In some embodiments, said fluorescent protein is green fluorescent protein (GFP). In some embodiments, said reporter gene is regulated by an inducible promoter. In some embodiments, said inducible promoter is induced by doxycycline, isopropyl-β-thiogalactopyranoside (IPTG), galactose, a divalent cation, lactose, arabinose, xylose, N-acyl homoserine lactone, tetracycline, a steroid, a metal, an alcohol, heat, light or a combination thereof.

In one aspect provided herein is a donor vector for site specific integration of an exogenous polynucleotide into a genome of a host cell, wherein the genome of the host cell comprises a first recognition sequence, the donor vector comprising: (a) the exogenous polynucleotide; and (b) a second recognition sequence for a site specific recombinase, wherein the second recognition sequence can undergo a site specific recombination with the first recognition sequence of the site specific recombinase, when contacted with the site specific recombinase; and (c) a nucleic acid sequence encoding a modified selectable marker polypeptide, wherein the modified selectable marker polypeptide exhibits a reduced activity relative to a corresponding WT selectable marker polypeptide.

In some embodiments, the nucleic acid sequence encoding the modified selectable marker polypeptide is an antibiotic resistance gene, and wherein the reduced activity comprises reduced resistance to an antibiotic relative to the corresponding WT selectable marker polypeptide. In some embodiments, the antibiotic is selected from a group consisting of puromycin, hygromycin, blasticidin, and neomycin. In some embodiments, the modified selectable marker polypeptide exhibits a reduced activity by at least 2%, 5%, 10%, 15%. 20%, 25%, 30%, 35%, 40%, 45%, 50% or more relative to the corresponding WT selectable marker protein. In some embodiments, the modified selectable marker polypeptide comprises a modification selected from an amino acid insertion, an amino acid substitution, or an amino acid deletion relative to the corresponding WT selectable marker polypeptide. In some embodiments, the amino acid substitution is a non-conservative amino acid substitution.

In some embodiments, the nucleic acid sequence encoding a modified selectable marker polypeptide comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 84. In some embodiments, the modified selectable marker polypeptide is a modified neomycin phosphotransferase. In some embodiments, the modified neomycin phosphotransferase comprises a D227V amino acid substitution relative to the corresponding WT neomycin phosphotransferase. In some embodiments, the donor vector further comprises a reporter gene. In some embodiments, the reporter gene encodes a bioluminescent protein, a chromogenic protein, or a fluorescent protein. In some embodiments, the reporter gene encodes the fluorescent protein that is a green fluorescent protein (GFP) or a variant thereof.

In some embodiments, the donor vector comprises a promoter operably linked to the exogenous polynucleotide, and/or the nucleic acid sequence encoding a modified selectable marker polypeptide. In some embodiments, the promoter is an inducible promoter is induced by doxycycline, isopropyl-β-thiogalactopyranoside (IPTG), galactose, a divalent cation, lactose, arabinose, xylose, N-acyl homoserine lactone, tetracycline, a steroid, a metal, an alcohol, or a combination thereof. In some embodiments, said inducible promoter is induced by heat or light. In some embodiments, the promoter is a CMV promoter, or a Chinese hamster EF1-a (CHEF1a) promoter.

In some embodiments, the exogenous polynucleotide encodes a therapeutic polypeptide. In some embodiments, said exogenous polynucleotide comprises a first polynucleotide, and a second polynucleotide connected by a linker polynucleotide. In some embodiments, the first polynucleotide encodes a heavy chain polypeptide of an antibody, or a functional fragment thereof. In some embodiments, the second polynucleotide encodes a light chain polypeptide of an antibody, or a functional fragment thereof. In some embodiments, the first polynucleotide that encodes a heavy chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 86, or SEQ ID NO: 110. In some embodiments, the second polynucleotide that encodes a light chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 85, or SEQ ID NO: 109.

In some embodiments, the first polynucleotide that encodes a heavy chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 86, and the second polynucleotide that encodes a light chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 85. In some embodiments, the first polynucleotide that encodes a heavy chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 110, and the second polynucleotide that encodes a light chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 109. In some embodiments, the antibody, or a fragment thereof is a PD-L1 binding antibody or a fragment thereof. In some embodiments, the antibody, or a fragment thereof is a VEGF binding antibody or a fragment thereof.

In some embodiments, the linker polynucleotide encodes a rigid linker, a flexible linker, a cleavable linker, a self-cleavable linker, a peptide linker, a linker cleavable via ribosome skipping, or any combination thereof. In some embodiments, the self-cleavable linker comprises a 2A sequence e.g., a P2A, T2A, E2A, or a F2A sequence, or a furin recognition sequence. In some embodiments, the therapeutic polypeptide is an antibody or a functional fragment thereof. In some embodiments, said therapeutic polypeptide comprises a chimeric antigen receptor (CAR), a T-cell receptor (TCR), a B-cell receptor (BCR), an αβ receptor, a γδ T-receptor, dopamine, insulin, proinsulin, or a portion thereof, or a combination thereof. In some embodiments, said therapeutic polypeptide comprises a CD19 CAR or a portion thereof. In some embodiments, the donor vector further comprises a nucleic acid sequence encoding for the site specific recombinase. In some embodiments, the site specific recombinase is a serine recombinase or a tyrosine recombinase.

In one aspect provided herein is a genetically engineered cell comprising a gene editing multi-site (GEMS) sequence in said cell's genome, said GEMS sequence comprising a plurality of first recognition sequences for a site specific recombinase, wherein at least one of the plurality of first recognition sequence can undergo a site specific recombination with a second recognition sequence of the site specific recombinase, when contacted with the site specific recombinase, and wherein each of the plurality of first recognition sequence is heterologous to the genome. In some embodiments, the plurality of first recognition sequence comprises at least 3, 4, 5, 6, 7, 8, 9, 10, 11, 12, 13, 14, 15, 16, 17, 18, 19, 20, or more first recognition sequences. In some embodiments, the site specific recombinase is a serine recombinase, or a tyrosine recombinase. In some embodiments, the serine recombinase is a serine integrase.

In some embodiments, the serine recombinase is a Bxb1 integrase, a phiBT1, a R4 integrase, a TP901 integrase, gamma-delta resolvase, Tn3 resolvase, a phiC31 integrase, γδ resolvase, Gin invertase. In some embodiments, the tyrosine recombinase is a tyrosine integrase. In some embodiments, the plurality of first recognition sequence is an att site. In some embodiments, the plurality of first recognition sequence is a bacterial genomic recombination site (attB) or a phage genomic recombination site (attP). In some embodiments, the second recognition sequence is an att site selected from is a bacterial genomic recombination site (attB) or a phage genomic recombination site (attP). In some embodiments, the first recognition sequence is an attB site and the second recognition sequence is an attp site.

In some embodiments, the first recognition sequence is an attP site and the second recognition sequence is an attB site. In some embodiments, at least 2, 3, 4, 5, 6, 7, 8, 9, 10, 11, 12, 13, 14, 15, 16, 17, 18, 19, 20, 25, or more of the plurality of first recognition sequences is an attB site. In some embodiments, at least 2, 3, 4, 5, 6, 7, 8, 9, 10, 11, 12, 13, 14, 15, 16, 17, 18, 19, 20, 25, or more of the plurality of first recognition sequences is an attP site. In some embodiments, each of the plurality of the first recognition sequence is the same. In some embodiments, each of the plurality of first recognition sequence is an attP site or an attB site. In some embodiments, the second recognition sequence is heterologous to said genome. In some embodiments, the GEMS sequence is heterologous to said genome. In some embodiments, each of said plurality of first recognition sequence is non-coding. In some embodiments, the GEMS sequence is non-coding.

In some embodiments, each of the plurality of the first recognition sequence comprise at least about 10 to about 500 nucleotides. In some embodiments, each of the plurality of the first recognition sequence comprise at least about 20 to about 300 nucleotides. In some embodiments, each of the plurality of the first recognition sequence comprise at least about 30 to about 200 nucleotides. In some embodiments, each of the plurality of the first recognition sequence comprise at least about 40 to about 100 nucleotides. In some embodiments, said GEMS sequence further comprises one or more polynucleotide spacers separating said plurality of first recognition sequences. In some embodiments, each of said one or more polynucleotide spacers comprise at least about 2 to about 10,000 nucleotides. In some embodiments, each of said one or more polynucleotide spacers comprise at least about 25 to about 100 nucleotides. In some embodiments, each of said one or more polynucleotide spacers comprise at least about 25 to about 50 nucleotides.

In some embodiments, each of said one or more polynucleotide spacers comprises a unique sequence. In some embodiments, at least one of said plurality of first recognition sequences comprises a sequence set forth in SEQ ID NOs: 106, SEQ ID NO: 107, or reverse complements thereof. In some embodiments, each of said plurality of first recognition sequences is selected from a sequence set forth in SEQ ID NOs: 106, SEQ ID NO: 107, or reverse complements thereof. In some embodiments, said GEMS sequence comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 105. In some embodiments, said GEMS sequence is SEQ ID NO: 105. In some embodiments, said GEMS sequence is inserted in a safe harbor site of said cell's genome. In some embodiments, said safe harbor site is an adeno-associated virus site 1 (AAVs1) site, a Rosa26 site, C-C motif receptor 5 (CCR5) site, or a Hipp11 (H11) site.

In some embodiments, said genetically engineered cell is a mammalian cell. In some embodiments, the genetically engineered cell further comprises an exogenous polynucleotide encoding a therapeutic polypeptide inserted within or adjacent to the GEMS sequence. In some embodiments, said exogenous polynucleotide comprises a first polynucleotide, and a second polynucleotide connected by a linker polynucleotide.

In some embodiments, the first polynucleotide encodes a heavy chain polypeptide of an antibody, or a functional fragment thereof. In some embodiments, the second polynucleotide encodes a light chain polypeptide of an antibody, or a functional fragment thereof.

In some embodiments, the first polynucleotide that encodes a heavy chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 86, or SEQ ID NO: 110. In some embodiments, the second polynucleotide that encodes a light chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 85, or SEQ ID NO: 109. In some embodiments, the first polynucleotide that encodes a heavy chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 86, and the second polynucleotide that encodes a light chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 85. In some embodiments, the first polynucleotide that encodes a heavy chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 110, and the second polynucleotide that encodes a light chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 109.

In some embodiments, the antibody, or a fragment thereof is a PD-L1 binding antibody or a fragment thereof. In some embodiments, the antibody, or a fragment thereof is a VEGF binding antibody or a fragment thereof.

In some embodiments, the linker polynucleotide encodes a rigid linker, a flexible linker, a cleavable linker, a self-cleavable linker, a peptide linker, a linker cleavable via ribosome skipping, or any combination thereof. In some embodiments, the self-cleavable linker comprises a 2A sequence e.g., a P2A, T2A, E2A, or a F2A sequence, or a furin recognition sequence. In some embodiments, the therapeutic polypeptide is an antibody or a functional fragment thereof. In some embodiments, the exogenous polynucleotide further comprises a nucleic acid sequence encoding a modified selectable marker polypeptide, wherein the modified selectable marker polypeptide exhibits a reduced activity relative to a corresponding WT selectable marker polypeptide.

In some embodiments, the genetically engineered cell is a mammalian cell. In some embodiments, the genetically engineered cell is a CHO cell. In some embodiments, the genetically engineered cell is a stem cell. In some embodiments, the genetically engineered cell is a plant cell. In one aspect provided herein is a method of producing a cell comprising a gene editing multi-site (GEMS) sequence, the method comprising: introducing into the cell said GEMS construct of any one of aspects above. In some embodiments, said cell is a eukaryotic cell. In some embodiments, said cell is a mammalian cell. In some embodiments, said cell is a CHO cell. In some embodiments, said cell is a human cell. In some embodiments, said cell is a stem cell or a non-stem cell. In some embodiments, said stem cell is an adult stem cell, a somatic stem cell, a non-embryonic stem cell, an embryonic stem cell, a hematopoietic stem cell, a pluripotent stem cell, or a trophoblast stem cell. In some embodiments, said stem cell is a mammalian trophoblast stem cell. In some embodiments, said stem cell is a human trophoblast stem cell. In some embodiments, said non-stem cell is a T-cell. In some embodiments, said T-cell is an αβ T-cell, an NK T-cell, a γδ T-cell, a regulatory T-cell, a T helper cell, or a cytotoxic T-cell.

Provided herein is a genetically engineered cell generated by the method of any one of aspects above.

Provided herein is a method of producing a genetically engineered cell, the method comprising: (a) introducing into a host cell: (i) the GEMS construct of any one of aspects above, (ii) a donor vector comprising an exogenous polynucleotide, and a second recognition site for the site specific recombinase, wherein the at least one of the plurality of first recognition sequences can undergo a site specific recombination with the second recognition sequence of the site specific recombinase, when contacted with the site specific recombinase, and (iii) the site specific recombinase, or a vector comprising a nucleic acid sequence encoding the site specific recombinase; and (b) culturing the cell from step (a) under conditions permissive for the site specific recombination between the at least one of the plurality of first recognition sequences and the second recognition sequence, when contacted with the site specific recombinase, wherein the site specific recombination results in site specific insertion of the exogenous polynucleotide within the at least one of the plurality of first recognition sequence, thereby generating the genetically engineered cell.

In some embodiments, the donor vector is one that is described in one of aspects above. In some embodiments, the culturing of step (b) further comprises culturing under selective conditions that require expression of the modified selectable marker polypeptide. In some embodiments, the method further comprises selecting a cultured cell from step (b) that expresses the selectable marker polypeptide.

Provided herein is a method comprising; (a) providing a genetically engineered cell of any one of aspects above, (b) introducing into the genetically engineered cell: (i) a donor vector comprising an exogenous polynucleotide, and a second recognition site for a site specific recombinase, wherein at least one of the plurality of first recognition sequence can undergo a site specific recombination with a first recognition sequence of the site specific recombinase, when contacted with the site specific recombinase, and

(iii) the site specific recombinase or a vector comprising a nucleic acid sequence encoding the site specific recombinase; and (c) culturing the genetically engineered cell from step (b) under conditions permissive for the site specific recombination between the at least one of the plurality of first recognition sequences and the second recognition sequence, when contacted with the site specific recombinase, wherein the site specific recombination results in site specific insertion of the exogenous polynucleotide within the at least one of the plurality of first recognition sequence.

Provided herein is a method for generating a genetically engineered cell comprising multiple copies of an exogenous polynucleotide into a genome of the cell, comprising: (a) providing a genetically engineered cell of any one of aspects above; (b) introducing into the genetically engineered cell, (i) a plurality of the donor vectors of any one of aspects above, and (ii) the site specific recombinase, or a vector comprising a nucleic acid sequence encoding said site specific recombinase; (c) culturing the cell from step (b); and (d) selecting a cell cultured from step (b) that expresses the selectable marker polypeptide, wherein the culturing of step (c) comprises culturing under conditions permissive for the site specific recombination between the second recognition sequence of each of the plurality of donor vectors and a selected first recognition sequence merfrom the plurality of first recognition sequences, when contacted with the site specific recombinase, and wherein the culturing of step (c) comprises culturing under selective conditions that require expression of the modified selectable marker polypeptide, to thereby generate a cell comprising multiple copies of the exogenous polynucleotide.

Provided herein is a kit comprising: (a) said genetically engineered cell of any one of aspects above; or (b) a host cell; and the GEMS construct of any one aspects above. In some embodiments, the kit of (a) and the kit of (b) further comprises the donor vector of any one of aspects above.

Provided herein is a gene editing multi-site (GEMS) construct for insertion into an insertion site in a genome of a Chinese hamster ovary (CHO) cell, wherein said GEMS construct comprises: a GEMS sequence comprising a plurality of nuclease recognition sequences, wherein at least one of the plurality of nuclease recognition sequences is heterologous to the genome of the CHO cell.

In some embodiments, each of the plurality of the nuclease recognition sequences is heterologous to the genome. In some embodiments, the GEMS sequence is heterologous to the genome. In some embodiments, the GEMS sequence is non-coding. In some embodiments, said plurality of nuclease recognition sequences comprises a recognition sequence for a zinc finger nuclease, a transcription activator-like effector nuclease, a meganuclease, a Cas protein, a Cpf 1 protein, or a combination thereof. In some embodiments, one or more nuclease recognition sequences of said plurality of nuclease recognition sequences comprise a recognition sequence for a Cas protein or a Cpf1 protein which further comprises a target sequence and a protospacer adjacent motif (PAM) sequence or reverse complements thereof. In some embodiments, said plurality of nuclease recognition sequences comprises at least 3, 4, 5, 6, 7, 8, 9, 10, 11, 12, 13, 14, 15, 16, 17, 18, 19, 20, 25, or more unique nuclease recognition sequences.

In some embodiments, at least one of said plurality of nuclease recognition sequences is selected from the group consisting of sequences SEQ ID NOs: 89, 91, 93, 95, 97, 99, 101, 103, and reverse complements thereof. In some embodiments, each of said plurality of nuclease recognition sequences is individually selected from the group consisting of SEQ ID NOs: 89, 91, 93, 95, 97, 99, 101, 103, and reverse complements thereof. In some embodiments, said plurality of nuclease recognition sequences comprises at least 3, 4, 5, 6, 7, 8, 9, 10, 11, 12, 13, 14, 15, 16, 17, 18, 19, 20, 25, or more unique target sequences. In some embodiments, said plurality of nuclease recognition sequences comprises at least 3, 4, 5, 6, 7, 8, 9, 10, 11, 12, 13, 14, 15, 16, 17, 18, 19, 20, 25, or more unique PAM sequences. In some embodiments, at least one target sequence in said plurality of nuclease recognition sequences is heterologous to said genome. In some embodiments, each target sequence of said plurality of nuclease recognition sequences is heterologous to said genome. In some embodiments, at least one target sequence in said plurality of nuclease recognition sequences is from about 17 to about 24 nucleotides in length. In some embodiments, each target sequence in said plurality of nuclease recognition sequences is from about 17 to about 24 nucleotides in length. In some embodiments, at least one target sequence in said plurality of nuclease recognition sequences is 20 nucleotides in length. In some embodiments, each target sequence in said plurality of nuclease recognition sequences is 20 nucleotides in length. In some embodiments, at least one target sequence in said plurality of nuclease recognition sequences is GC-rich.

In some embodiments, at least one target sequence in said plurality of nuclease recognition sequences comprises from about 40% to about 80% G and C nucleotides. In some embodiments, at least one target sequence in said plurality of nuclease recognition sequences comprises less than 40% G and C nucleotides. In some embodiments, at least one target sequence in said plurality of nuclease recognition sequences comprises more than 80% G and C nucleotides. In some embodiments, at least one target sequence in said plurality of nuclease recognition sequences is AT-rich. In some embodiments, at least one target sequence in said plurality of nuclease recognition sequences comprises from about 40% to about 80% A and T nucleotides. In some embodiments, at least one target sequence in said plurality of nuclease recognition sequences comprises less than 40% A and T nucleotides. In some embodiments, at least one target sequence in said plurality of nuclease recognition sequences comprises more than 80% A and T nucleotides. In some embodiments, at least one of said plurality of nuclease recognition sequences is a recognition sequence for said Cas protein.

In some embodiments, each of said plurality of nuclease recognition sequences is a recognition sequence for said Cas protein. In some embodiments, said Cas protein comprises Cas1, Cas1B, Cas2, Cas3, Cas4, Cas5, Cas5d, Cas5t, Cas5h, Cas5a, Cas6, Cas7, Cas8, Cas9, Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. In some embodiments, wherein at least one of said plurality of nuclease recognition sequences is a Cas9 recognition sequence. In some embodiments, each of said plurality of nuclease recognition sequences is a Cas9 recognition sequence. In some embodiments, at least one of said plurality of nuclease recognition sequences is a Cpf1 recognition sequence. In some embodiments, each of said plurality of nuclease recognition sequences is a Cpf1 recognition sequence.

In some embodiments, each PAM sequence in said plurality of nuclease recognition sequences is independently selected from the group consisting of: CC, NG, YG, NGG, NAA, NAT, NAG, NAC, NTA, NTT, NTG, NTC, NGA, NGT, NGC, NCA, NCT, NCG, NCC, NRG, TGG, TGA, TCG, TCC, TCT, GGG, GAA, GAC, GTG, GAG, CAG, CAA, CAT, CCA, CCN, CTN, CGT, CGC, TAA, TAC, TAG, TGG, TTG, TCN, CTA, CTG, CTC, TTC, AAA, AAG, AGA, AGC, AAC, AAT, ATA, ATC, ATG, ATT, AWG, AGG, GTG, TTN, YTN, TTTV, TYCV, TATV, NGAN, NGNG, NGAG, NGCG, NGGNG, NGRRT, NGRRN, NNGRRT, NNAAAAN, NNNNGATT, NAAAAC, NNAAAAAW, NNAGAA, NNNNACA, GNNNCNNA, NNNNGATT, NNAGAAW, NNGRR, TGGAGAAT, AAAAW, GCAAA, and TGAAA. In some embodiments, each PAM sequence in said plurality of nuclease recognition sequences is unique. In some embodiments, said GEMS sequence further comprises one or more polynucleotide spacers separating said plurality of nuclease recognition sequences.

In some embodiments, said one or more polynucleotide spacers comprise, individually, from about 2 to about 10,000 nucleotides. In some embodiments, said one or more polynucleotide spacers comprise, individually, from about 25 to about 50 nucleotides. In some embodiments, each of said one or more polynucleotide spacers comprises a unique sequence. In some embodiments, said GEMS sequence comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to the sequence set forth in SEQ ID NO: 1, or SEQ ID NO: 3. In some embodiments, said GEMS sequence comprises the sequence set forth in SEQ ID NO: 1, or SEQ ID NO: 3. In some embodiments, said GEMS sequence is SEQ ID NO: 1, or SEQ ID NO: 3. In some embodiments, the GEMS construct of any one of aspects above, further comprises: a first flanking insertion sequence homologous to a first genome sequence upstream of said insertion site, said first flanking insertion sequence located upstream of said GEMS sequence; and a second flanking insertion sequence homologous to a second genome sequence downstream of said insertion site, said second flanking insertion sequence located downstream of said GEMS sequence.

In some embodiments, said first flanking insertion sequence, said second flanking insertion sequence, or both comprise at least 12 nucleotides. In some embodiments, said first flanking insertion sequence, said second flanking insertion sequence, or both comprise at least 18 nucleotides. In some embodiments, said first flanking insertion sequence, said second flanking insertion sequence, or both comprise at least 50 nucleotides. In some embodiments, said first flanking insertion sequence, said second flanking insertion sequence, or both comprise at least 100 nucleotides. In some embodiments, said first flanking insertion sequence, said second flanking insertion sequence, or both comprise at least 500 nucleotides. In some embodiments, said insertion sequence is in a safe harbor site of said genome. In some embodiments, said safe harbor site is an adeno-associated virus site 1 (AAVs1) site, a Rosa26 site, a C-C motif receptor 5 (CCR5) site, or a Hipp11 (H11) site

In some embodiments, the GEMS construct further comprises a first recognition sequence for a nuclease upstream of said GEMS sequence. In some embodiments, the GEMS construct further comprises a second recognition sequence for said nuclease downstream of said GEMS sequence. In some embodiments, said nuclease is a zinc finger nuclease, a transcription activator-like effector nuclease, a meganuclease, a Cas protein, or a Cpf 1 protein. In some embodiments, said nuclease is said meganuclease. In some embodiments, said meganuclease is an I-SceI meganuclease. In some embodiments, said first recognition sequence for said nuclease is upstream of said first flanking insertion sequence. In some embodiments, said second recognition sequence for said nuclease is downstream of said second flanking insertion sequence. In some embodiments, said GEMS construct further comprises a reporter gene. In some embodiments, said reporter gene encodes a fluorescent protein. In some embodiments, said fluorescent protein is green fluorescent protein (GFP). In some embodiments, said reporter gene is regulated by an inducible promoter. In some embodiments, said inducible promoter is induced by doxycycline, isopropyl-P-thiogalactopyranoside (IPTG), galactose, a divalent cation, lactose, arabinose, xylose, N-acyl homoserine lactone, tetracycline, a steroid, a metal, an alcohol, or a combination thereof. In some embodiments, said inducible promoter is induced by heat or light.

Provided herein is a method of producing a CHO cell comprising a gene editing multi-site (GEMS), the method comprising: introducing into a CHO cell said GEMS construct of any one of aspects above.

Provided herein is a genetically engineered cell comprising a gene editing multi-site (GEMS) sequence in said cell's genome, said GEMS sequence comprising a plurality of nuclease recognition sequences, wherein the genetically engineered cell is a CHO cell.

In some embodiments, said plurality of nuclease recognition sequences comprises at least 3, 4, 6, 7, 8, 9, 10, 11, 12, 13, 14, 15, 16, 17, 18, 19, 20, 25, or more nuclease recognition sequences. In some embodiments, said plurality of nuclease recognition sequences comprises a recognition sequence for a zinc finger nuclease, a transcription activator-like effector nuclease, a meganuclease, a Cas protein, a Cpf 1 protein, or a combination thereof. In some embodiments, one or more nuclease recognition sequences of said plurality of nuclease recognition sequences comprise a recognition sequence for a Cas protein or a Cpf1 protein, which further comprises a target sequence and a protospacer adjacent motif (PAM) sequence or reverse complements thereof. In some embodiments, said plurality of nuclease recognition sequences comprises at least 3, 4, 5, 6, 7, 8, 9, 10, 11, 12, 13, 14, 15, 16, 17, 18, 19, 20, 25, or more unique nuclease recognition sequences. In some embodiments, at least one of said plurality of nuclease recognition sequences is heterologous to said cell's genome. In some embodiments, each of said plurality of nuclease recognition sequences is heterologous to said cell's genome. In some embodiments, at least one of said plurality of nuclease recognition sequences is selected from the group consisting of sequences SEQ ID NOs: 89, 91, 93, 95, 97, 99, 101, 103, and reverse complements thereof. In some embodiments, each of said plurality of nuclease recognition sequences is individually selected from the group consisting of SEQ ID NOs: 89, 91, 93, 95, 97, 99, 101, 103, and reverse complements thereof.

In some embodiments, said plurality of nuclease recognition sequences comprises at least 3, 4, 5, 6, 7, 8, 9, 10, 11, 12, 13, 14, 15, 16, 17, 18, 19, 20, 25, or more unique target sequences. In some embodiments, said plurality of nuclease recognition sequences comprises at least 3, 4, 5, 6, 7, 8, 9, 10, 11, 12, 13, 14, 15, 16, 17, 18, 19, 20, 25, or more unique PAM sequences. In some embodiments, at least one target sequence in said plurality of nuclease recognition sequences is heterologous to said cell's genome. In some embodiments, each target sequence of said plurality of nuclease recognition sequences is heterologous to said cell's genome.

In some embodiments, at least one target sequence in said plurality of nuclease recognition sequences is from about 17 to about 24 nucleotides in length. In some embodiments, each target sequence in said plurality of nuclease recognition sequences is from about 17 to about 24 nucleotides in length. In some embodiments, at least one target sequence in said plurality of nuclease recognition sequences is 20 nucleotides in length. In some embodiments, each target sequence in said plurality of nuclease recognition sequences is 20 nucleotides in length. In some embodiments, at least one target sequence in said plurality of nuclease recognition sequences is GC-rich. In some embodiments, at least one target sequence in said plurality of nuclease recognition sequences comprises from about 40% to about 80% G and C nucleotides. In some embodiments, at least one target sequence in said plurality of nuclease recognition sequences comprises less than 40% G and C nucleotides. In some embodiments, at least one target sequence in said plurality of nuclease recognition sequences comprises more than 80% G and C nucleotides.

In some embodiments, at least one target sequence in said plurality of nuclease recognition sequences is AT-rich. In some embodiments, at least one target sequence in said plurality of nuclease recognition sequences comprises from about 40% to about 80% A and T nucleotides. In some embodiments, at least one target sequence in said plurality of nuclease recognition sequences comprises less than 40% A and T nucleotides.

In some embodiments, at least one target sequence in said plurality of nuclease recognition sequences comprises more than 80% A and T nucleotides. In some embodiments, at least one of said plurality of nuclease recognition sequences is a recognition sequence for a Cas protein. In some embodiments, each of said plurality of nuclease recognition sequences is a recognition sequence for a Cas protein. In some embodiments, said Cas protein comprises Cas1, Cas1B, Cas2, Cas3, Cas4, Cas5, Cas5d, Cas5t, Cas5h, Cas5a, Cash, Cas7, Cas8, Cas9, Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof.

In some embodiments, at least one of said plurality of nuclease recognition sequences is a Cas9 recognition sequence. In some embodiments, each of said plurality of nuclease recognition sequences is a Cas9 recognition sequence. In some embodiments, at least one of said plurality of nuclease recognition sequences is a Cpf1 recognition sequence. In some embodiments, each of said plurality of nuclease recognition sequences is a Cpf1 recognition sequence. In some embodiments, at least one of said plurality of nuclease recognition sequences is an Argonaute recognition sequence. In some embodiments, each PAM sequence in said plurality of nuclease recognition sequences is independently selected from the group consisting of: CC, NG, YG, NGG, NAA, NAT, NAG, NAC, NTA, NTT, NTG, NTC, NGA, NGT, NGC, NCA, NCT, NCG, NCC, NRG, TGG, TGA, TCG, TCC, TCT, GGG, GAA, GAC, GTG, GAG, CAG, CAA, CAT, CCA, CCN, CTN, CGT, CGC, TAA, TAC, TAG, TGG, TTG, TCN, CTA, CTG, CTC, TTC, AAA, AAG, AGA, AGC, AAC, AAT, ATA, ATC, ATG, ATT, AWG, AGG, GTG, TTN, YTN, TTTV, TYCV, TATV, NGAN, NGNG, NGAG, NGCG, NGGNG, NGRRT, NGRRN, NNGRRT, NNAAAAN, NNNNGATT, NAAAAC, NNAAAAAW, NNAGAA, NNNNACA, GNNNCNNA, NNNNGATT, NNAGAAW, NNGRR, TGGAGAAT, AAAAW, GCAAA, and TGAAA.

In some embodiments, each PAM sequence in said plurality of nuclease recognition sequences is unique. In some embodiments, said GEMS sequence further comprises one or more polynucleotide spacers separating said plurality of nuclease recognition sequences. In some embodiments, said one or more polynucleotide spacers comprise, individually, from about 2 to about 10,000 nucleotides. In some embodiments, said one or more polynucleotide spacers comprise, individually, from about 25 to about 50 nucleotides. In some embodiments, each of said one or more polynucleotide spacers comprises a unique sequence. In some embodiments, said GEMS sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1, or SEQ ID NO: 3. In some embodiments, said GEMS sequence comprises SEQ ID NO: 1, or SEQ ID NO: 3.

In some embodiments, said GEMS sequence is SEQ ID NO: 1, or SEQ ID NO: 3. In some embodiments, said GEMS sequence is inserted in a safe harbor site of said cell's genome. In some embodiments, said safe harbor site is an adeno-associated virus site 1 (AAVs1) site, a Rosa26 site, a CCR5 site, or a Hipp11 (H11) site.

In some embodiments, said cell further comprises a donor nucleic acid sequence inserted within or adjacent to said GEMS sequence. In some embodiments, said donor nucleic acid sequence encodes a therapeutic polypeptide. In some embodiments, said therapeutic protein comprises a chimeric antigen receptor (CAR), a T-cell receptor (TCR), a B-cell receptor (BCR), an αβ receptor, a γδ T-receptor, dopamine, insulin, proinsulin, an antibody, a functional fragment thereof, or a combination thereof. In some embodiments, said therapeutic polypeptide comprises a CD19 CAR or a portion thereof. In some embodiments, the cell further comprises a nucleic acid sequence coding for a suicide gene, wherein the suicide gene encodes an apoptosis inducing molecule. In some embodiments, the apoptosis inducing molecule is fused to an inducer ligand binding domain.

In some embodiments, the nucleic acid sequence encoding an apoptosis inducing molecule is operably linked to a nucleic acid sequence encoding a regulatory element. In some embodiments, the regulatory element is a promoter. In some embodiments, the promoter is an inducible promoter. In some embodiments, the promoter is selected from a group consisting of cyclooxygenase promoter, a tumor necrosis factor promoter, an interleukin regulated promoter, alcohol-regulated promoter, steroid regulated promoter, dexamethasone regulated promoter, tetracycline regulated promoter, metal regulated promoter, light regulated promoter, and temperature regulated promoter. In some embodiments, the apoptosis inducing molecule is a caspase, a protease, or a prodrug activating enzyme. In some embodiments, the apoptosis inducing molecule is Caspase-1, Caspase-2, Caspase-3, Caspase-4, Caspase-5, Caspase-6, Caspase-7, Caspase-8, Caspase-9, Caspase-10, Granzyme A, Granzyme B, viral thymidine kinase, Cytosine deaminase, Fas ligand, TRAIL, or APO3L.

In some embodiments, the genetically engineered cell further comprises an exogenous polynucleotide encoding a therapeutic polypeptide inserted within or adjacent to the GEMS sequence. In some embodiments, said exogenous polynucleotide comprises a first polynucleotide, and a second polynucleotide connected by a linker polynucleotide. In some embodiments, the first polynucleotide encodes a heavy chain polypeptide of an antibody, or a functional fragment thereof. In some embodiments, the second polynucleotide encodes a light chain polypeptide of an antibody, or a functional fragment thereof. In some embodiments, the first polynucleotide that encodes a heavy chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 86, or SEQ ID NO: 110.

In some embodiments, the second polynucleotide that encodes a light chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 85, or SEQ ID NO: 109. In some embodiments, the first polynucleotide that encodes a heavy chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 86, and the second polynucleotide that encodes a light chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 85.

In some embodiments, the first polynucleotide that encodes a heavy chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 110, and the second polynucleotide that encodes a light chain polypeptide of an antibody or a fragment thereof comprises a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to sequence set forth in SEQ ID NO: 109. In some embodiments, the antibody, or a fragment thereof is a PD-L1 binding antibody or a fragment thereof. In some embodiments, the antibody, or a fragment thereof is a VEGF binding antibody or a fragment thereof.

Provided herein is an engineered nucleic acid vector comprising in 5′ to 3′ order: a donor nuclease recognition sequence selected from the group consisting of said plurality of nuclease recognition sequences from said GEMS sequence of said genetically engineered cell of any one of aspects above, or its reverse complement; a first donor flanking sequence homologous to a genomic sequence upstream of said selected nuclease recognition sequence; a second donor flanking sequence homologous to a genomic sequence downstream of said selected nuclease recognition sequence; and a copy of said donor nuclease recognition sequence or a reverse complement thereof. In some embodiments, said first donor flanking sequence and said second donor flanking sequence do not comprise said selected nuclease recognition sequence. In some embodiments, said first donor flanking sequence and said second donor flanking sequence comprise, individually, from about 10 to about 1,000 nucleotides. In some embodiments, said first donor flanking sequence and said second donor flanking sequence comprise, individually, from about 100 to about 750 nucleotides. In some embodiments, said first donor flanking sequence and said second donor flanking sequence comprise, individually, from about 150 to about 600 nucleotides. In some embodiments, said genomic sequence upstream of said selected nuclease recognition sequence is immediately adjacent to said selected nuclease recognition sequence.

In some embodiments, said genomic sequence downstream of said selected nuclease recognition sequence is immediately adjacent to said selected nuclease recognition sequence. In some embodiments, said vector further comprises a donor insertion site in between said first donor flanking sequence and said second donor flanking sequence. In some embodiments, said donor insertion site comprises a restriction enzyme site, a recognition sequence for a Cas protein, or a combination thereof.

Provided herein is a kit comprising: said genetically engineered cell of any one of aspects above; and said engineered nucleic acid vector described above.

Provided herein is a method comprising: providing said genetically engineered cell comprising a GEMS sequence of any one of aspects above; introducing into said genetically engineered cell a nucleic acid vector comprising, in 5′ to 3′ order: a first donor flanking sequence homologous to a genomic sequence upstream of a selected nuclease recognition sequence from said plurality of nuclease recognition sequences in said GEMS sequence, said donor nucleic acid sequence, and a second donor flanking sequence homologous to a genomic sequence downstream of said selected nuclease recognition sequence; introducing into said genetically engineered cell a guide polynucleotide; and introducing into said genetically engineered cell a nuclease that recognizes said selected nuclease recognition sequence when bound to said guide polynucleotide.

In some embodiments, said nucleic acid vector further comprises said selected nuclease recognition sequence or a reverse complement thereof upstream of said first donor flanking sequence. In some embodiments, said nucleic acid vector further comprises said selected nuclease recognition sequence or a reverse complement thereof downstream of said second donor flanking sequence. In some embodiments, said first donor flanking sequence and said second donor flanking sequence do not comprise said selected nuclease recognition sequence. In some embodiments, said first donor flanking sequence and said second donor flanking sequence comprise, individually, from about 10 to about 1,000 nucleotides. In some embodiments, said first donor flanking sequence and said second donor flanking sequence comprise, individually, from about 100 to about 750 nucleotides. In some embodiments, said first donor flanking sequence and said second donor flanking sequence comprise, individually, from about 150 to about 600 nucleotides. In some embodiments, said genomic sequence upstream of said selected nuclease recognition sequence is immediately adjacent to said selected nuclease recognition sequence. In some embodiments, said genomic sequence downstream of said selected nuclease recognition sequence is immediately adjacent to said selected nuclease recognition sequence.

In some embodiments, said donor nucleic acid sequence encodes a therapeutic polypeptide. In some embodiments, said therapeutic polypeptide comprises a chimeric antigen receptor (CAR), a T-cell receptor (TCR), a B-cell receptor (BCR), an αβ receptor, a γδ T-receptor, dopamine, insulin, proinsulin, an antibody, or a functional fragment thereof, or a combination thereof. In some embodiments, said therapeutic protein comprises a CD19 CAR or a portion thereof.

### BRIEF DESCRIPTION OF THE DRAWINGS

The features of the present disclosure are set forth with particularity in the appended claims. A better understanding of the features and advantages of the present will be obtained by reference to the following detailed description that sets forth illustrative embodiments, in which the principles of the disclosure are utilized, and the accompanying drawings of which:

FIG. 1 shows a representation of a gene editing multi-site (GEMS) sequences with multiple nuclease (CRISPR/Cas9) recognition sequence (GEMS-Cas9sites). The GEMS sequence has Rosa26 homology arms that are at 5′ and 3′ flanking the multiple nuclease recognition sequences. The GEMS as shown include protospacer adjacent motif (PAM) compatible with different crRNA as a part of the guide RNA, and targeting sequence for nuclease Cas9. The GEMS sequence is a non-coding sequence. The GEMS sequence is inserted in a genome of a cell, for example by CRISPR/Cas modification in the Rosa26 homology flanking arms.

FIGS. 2A-2B show a representation of a gene editing multi-site (GEMS) sequences with multiple recombinase(Bxb1) recognition sequence. The GEMS sequence has Rosa26 homology arms that are at 5′ and 3′ flanking the multiple recombinase recognition sequences. The GEMS sequence is a non-coding sequence. The GEMS sequence is inserted in a genome of a cell, for example by CRISPR/Cas modification in the Rosa26 homology flanking arms. FIG. 2A shows the GEMS include multiple recognition sequence for a recombinase; attP. FIG. 2B shows the GEMS include multiple recognition sequence for a recombinase; attB.

FIG. 3 shows representation of insertion of a donor plasmid with GFP expression cassette, bxb1 recognition sequence (attB) and gene encoding modified selectable marker polypeptide neomycin transferase with reduced activity (Top panel). The bottom panel shows a site specific recombination between attB (Bxb1 recognition sequence) on the donor plasmid and a BxB1 recognition sequence (attP) within the GEMS sequence in the genome of a cell, resulting in site specific integration of the GFP expression cassette and gene encoding selectable marker.

FIG. 4 shows representation of insertion of a donor plasmid with an antibody (mAb) expression cassette, bxb1 recognition sequence (attB) and gene encoding modified selectable marker polypeptide neomycin transferase with reduced activity (top panel). The bottom panel shows a site specific recombination between attB (Bxb1 recognition sequence) on the donor plasmid and a BxB1 recognition sequence (attP) within the GEMS sequence in the genome of a cell, resulting in site specific integration of the antibody expression cassette and gene encoding selectable marker. The antibody expression cassette include a nucleic acid sequence encoding a heavy chain or a fragment thereof of an antibody and nucleic acid sequence encoding a light chain or a fragment thereof of an antibody linked by a linker polynucleotide.

FIG. 5A shows representation of site-specific integration of multiple copies of a gene expression cassette, e.g., multiple copies of a GFP expression cassette in a GEMS sequence inserted within a genome of a cell, by introducing multiple donor plasmids in a cell with a GEMS sequence. Each donor plasmid comprises a copy of the gene expression cassette, e.g., GFP expression cassette. Site specific recombination occurs between recombinase recognition sequence, attB on each of multiple donor plasmid with a GFP expression cassette, and a selected recombinase recognition sequence, attP from the multiple attP sequence within the GEMS sequence. The site-specific recombination occurs in presence of a recombinase (e.g., Bxb1) and results in site-specific integration of multiple copies of GFP expression cassette.

FIG. 5B shows representation of site-specific integration of multiple copies of an antibody (mAb) expression cassette in a GEMS sequence inserted within a genome of a cell, by introducing multiple donor plasmids in a cell with a GEMS sequence. Each donor plasmid comprises a copy of the antibody expression cassette. Site specific recombination occurs between recombinase recognition sequence, attB on each of multiple donor plasmid with an antibody (mAb) expression cassette, and a selected recombinase recognition sequence, attP from the multiple attP sequence within the GEMS sequence. The site-specific recombination occurs in presence of a recombinase (e.g., Bxb1) and results in site-specific integration of multiple copies of an antibody (mAb) expression cassette. The antibody expression cassette includes a nucleic acid sequence encoding a heavy chain or a fragment thereof of an antibody and nucleic acid sequence encoding a light chain or a fragment thereof of an antibody linked by a linker polynucleotide.

FIG. 6 shows maps of GEMS plasmid; CHO_Rosa26_GEMS_Cas9 sites with multiple nuclease recognition sequences.

FIG. 7A shows the schematic illustration of a GEMS sequence i.e., GEMS_Cas9 integrated into Rosa26 loci via Cas9-mediated homology recombination on both 5′ homology arm (5-HA) and 3′ homology arm (3-HA). The spanning region of a plurality of nuclease recognition sequences, i.e., GEMS_Cas9-1, and GEMS_Cas9-2 is also shown along with the 5′ junction and 3′-junction for site-specific integration verification by PCR. FIG. 7B shows DNA gel electrophoresis of PCR products for 5′ junction, GEMS_Cas9-1, GEMS_Cas9-2 and 3′-junction region for site-specific integration verification. FIG. 7C shows the nucleic acid sequence alignment between sequenced PCR products and reference sequences on both 5′ and 3′ junction site sequences between Rosa26 site and homology arm and between homology arm and GEMS_Cas9 targeting cassette.

FIG. 8A shows the schematic illustration of a GEMS sequence with a plurality of nuclease recognition sequences, i.e., 8 sites of GEMS_Cas9sites, and the location for two PCR products (2088 bp and 1958 bp in size) covering different sets of nuclease recognition sites for nuclease assay. FIG. 8B shows cutting efficiency of the designed sgRNAs in the in vitro nuclease assay. Six designed sgRNA were tested in the in vitro assay for their ability to cut the nuclease recognition sites, i.e., GEMS_Cas9sites sequence. Cas9 nuclease can specifically and completely cut nuclease recognition sites 1, 2, 4, 5, 6 and site 7 of GEMS sequence in the presence of corresponding site-specific sgRNA.

FIG. 9A shows the schematic illustration of a GEMS sequence with a plurality of nuclease recognition sequences i.e., the 8 sites of GEMS_Cas9sites and the engineering of a transgene expressing CD19-CAR in site 7. FIG. 9B shows the expression of CD19 CAR on the cell surface of two representative monoclonal cell lines (H8 and H10) with CD19 CAR integrated in site 7 of GEMS_Cas9sites of CHO-K1 cells by immunostaining with CD19 Fc fusion protein (left panels) and with anti-CD3zeta antibody (middle panels) and overlapping images (right panels). The expression of CD19-CAR were detected in the majority of the cells.

FIG. 10A shows the schematic illustration of a transgene expressing CD19-CAR integrated into site 7 of GEMS_Cas9sites via Cas9-mediated homology recombination on both 5′ homology arm (5-HA) and 3′ homology arm (3-HA). Also shows the spanning region of CD19-CAR transgene, 5′ junction and 3′-junction for site-specific integration verification by PCR. FIG. 10B shows DNA gel electrophoresis of PCR products for 5′ junction, CD19-CAR transgene, and 3′-junction region for verification of site-specific integration of CD19-CAR on two representative monoclonal cell lines (H4 and H8). FIG. 10C shows the nucleic acid sequence alignment between sequenced PCR products and reference sequences on both 5′ and 3′ junction site sequences between GEMS and homology arm and between homology arm and CD19-CAR expression cassette.

FIG. 11A shows the schematic illustration of a GEMS sequence with plurality of nuclease recognition sequences in i.e., 8 sites of GEMS_Cas9sites and the engineering of a transgene expressing GFP in site 2 and a transgene expression CD19-CAR already engineered in site 7 of GEMS_Cas9sites. FIG. 11B shows the expression of GFP (left panels) and CD19 CAR on the cell surface (middle panels) of three representative monoclonal cell lines (K1, K2 and K8) with GFP in site 2 and CD19 CAR integrated in site 7 of GEMS_Cas9sites of CHO-K1 cells. The expression of CD19-CAR was immunostained by anti-CD3zeta antibody. The expression of both GFP and CD19-CAR transgenes were detected and overlapped in the cells (right panels).

FIG. 12A shows the schematic illustration of transgene expressing GFP integrated into site 2 of GEMS_Cas9sites via Cas9-mediated homology recombination on both 5′ homology arm (5-HA) and 3′ homology arm (3-HA). Also shows the spanning region of GFP transgene, 5′ junction and 3′-junction for site-specific integration verification by PCR. FIG. 12B shows DNA gel electrophoresis of PCR products for 5′ junction, GFP transgene and 3′-junction region for site-specific integration verification on two representative monoclonal cell lines (K2 and K4). FIG. 12C shows the nucleic acid sequence alignment between sequenced PCR products and reference sequences on both 5′ and 3′ junction site sequences between GEMS and homology arm and between homology arm and GFP transgene.

FIG. 13A shows the schematic illustration of a GEMS sequence with a plurality of nuclease recognition sequences, i.e., 8 sites of GEMS_Cas9sites and engineering a transgene expressing a PD-L1 antibody in site 4, a transgene expressing GFP engineered in site 2, and a transgene expressing CD19-CAR engineered in site 7 of GEMS_Cas9sites. FIG. 13B shows the SDS-PAGE gel resolving the heavy chain and light chain of PD-L1 antibody expressed and purified from two representative monoclonal cell lines (R-B and R-F) and control antibody. FIG. 13C shows the expression of GFP (left panels) and CD19 CAR on the cell surface (middle panels) of two representative monoclonal cell lines (R-C and R-F) with GFP transgene in site 2 and CD19 CAR transgene integrated in site 7 of GEMS_Cas9sites of CHO-K1 cells. The expression of CD19-CAR was immunostained by anti-CD3zeta antibody. The expression of GFP and CD19-CAR were detected and overlapped in the cells (right panels).

FIG. 14A shows the schematic illustration of transgene expressing PD-L1 antibody integrated into site 4 of GEMS_Cas9sites via Cas9-mediated homology recombination on both 5′ homology arm (5-HA) and 3′ homology arm (3-HA). Also shows the spanning region of PD-L1 antibody transgene, 5′ junction and 3′-junction for site-specific integration verification by PCR. FIG. 14B shows DNA gel electrophoresis of PCR products for PD-L1 antibody, 5′ junction and 3′-junction region for site-specific integration verification on four representative monoclonal cell lines (R-B, R-C, R-D and R-F). FIG. 14C shows the nucleic acid sequence alignment between sequenced PCR products and reference sequences on both 5′ and 3′ junction site sequences between GEMS and homology arm and between homology arm and PD-L1 mAb transgene.

FIG. 15 shows maps of GEMS plasmid; CHO_Rosa26_GEMS_Bxb1sites with multiple recombinase recognition sequences.

FIG. 16A shows the schematic illustration of a GEMS sequence with a plurality of recognition sequences for a site-specific recombinase, i.e., GEMS_Bxb1 integrated into Rosa26 loci via Cas9-mediated homology recombination on both 5′ homology arm (5-HA) and 3′ homology arm (3-HA). Also shows the spanning region of GEMS_Bxb1, 5′ junction and 3′-junction for site-specific integration verification by PCR. FIG. 16B shows DNA gel electrophoresis of PCR products for 5′ junction, 3′-junction and GEMS_Bxb1 region for site-specific integration verification. FIG. 16C shows the nucleic acid sequence alignment between sequenced PCR products and reference sequences on both 5′ and 3′ junction site sequences between Rosa26 site and homology arm and between homology arm and GEMS_Bxb1 targeting cassette.

FIG. 17 shows phase contrast image and GFP expression image of cells from two representative candidate cell clones with GFP transgene engineered into GEMS_Bxb1sites.

### DEFINITIONS

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

As used herein the term “consisting essentially of” refers to those elements required for a given embodiment. The term permits the presence of elements that do not materially affect the basic and novel or functional characteristic(s) of that embodiment of the invention.

The term “about” in relation to a reference numerical value and its grammatical equivalents as used herein can include the numerical value itself and a range of values plus or minus 10% from that numerical value.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “multiple gene editing site(s)” and “gene editing multi-site(s) (GEMS)” are used interchangeably herein. A GEMS construct can comprise a multiple gene editing site or a gene editing multi-site. The multi-gene editing site comprises a plurality of recognition sequence for a nuclease (e.g., TALEN or a Cas9 nuclease) or a site-specific recombinase (e.g., serine recombinase such as Bxb1). A GEMS construct can comprise primary endonuclease recognition sites and a multiple gene editing site or a gene editing multi-site. In some embodiments, one or more of the primary endonuclease recognition sites are positioned upstream of the multiple gene editing site, and one or more of the primary endonuclease recognition sites are positioned downstream of the multiple gene editing site. A GEMS construct can comprise flanking insertion sequences, wherein each of said flanking insertion sequences are homologous to a genome sequence at said insertion site; and a GEMS sequence adjacent to said flanking insertion sequences, wherein said GEMS sequence comprises a plurality of nuclease recognition sequences, wherein each of said plurality of nuclease recognition sequences comprises a target sequence and a protospacer adjacent motif (PAM) sequence, wherein said target sequence binds a guide polynucleotide following insertion of said GEMS construct at said insertion site. In an embodiment, the GEMS construct can further comprise a polynucleotide spacer which separates at least one recognition sequence from an adjacent recognition sequence, wherein the recognition sequence is a recognition sequence for a site-specific recombinase (e.g., BxB1), or wherein the recognition sequence is a recognition sequence for a nuclease (e.g., TALEN or a Cas9 nuclease). In some embodiment, the GEMS construct comprises a pair of homology arms which flank the GEMS sequence. In some embodiments, at least one homology arm of the pair of homology arms comprises a homology arm sequence that is homologous to a sequence of a safe harbor site of a host cell genome. In an embodiment, the plurality of nuclease recognition sequences is a plurality of editing sites (e.g., a plurality of PAMs), which each comprise a secondary endonuclease recognition site. The primary endonuclease recognition sites (e.g., insertion site) upstream and downstream of the multiple gene editing site facilitate insertion of the GEMS into the genome of a host cell. Thus, the GEMS constructs can be used, for example, to transfect a host cell and, once in the host cell, the upstream and downstream primary endonuclease recognition sites facilitate insertion of the multiple gene editing site into a chromosome. Once the multiple gene editing site is inserted into a chromosome, the host cell can be further modified with donor nucleic acid sequences or donor genes or portions thereof that are inserted into one or more of the editing sites of the multiple gene editing site. In some embodiments, insertion of the multiple gene editing site into a chromosome is stable integration into the chromosome.

“site-specific recombinase” or “recombinase” as used herein refers to an enzyme that recognizes and binds specific nucleic acid sequence(s) or “recognition sequence for a site specific recombinase”. In some embodiments, a site-specific recombinase recognizes and binds two recognition sequences; a first recognition sequence for the site-specific recombinase and a second recognition sequence for the site-specific recombinase, and mediates or catalyzes a site-specific recombination between the two recognition sequences. Thus, as used herein, a recombinase is “specific for” a recognition sequence when the recombinase can recognize and bind the recognition sequence and can mediate site-specific recombination between two recognition sequences. In some embodiments, the site specific recombination between the two recognition sequences for the recombinase results in the excision, integration, inversion, or exchange of DNA fragments between the recognition sequences for the recombinase. In some embodiments, a first recognition sequence and a second recognition sequence for the recombinase reside on separate DNA molecules. A first recognition sequence and a second recognition sequences for a site-specific recombinase are not necessarily identical.

“recognition sequence for a site-specific recombinase” or “recognition site for a site-specific recombinase” is a specific nucleic acid sequence which is recognized and bound to by a site-specific recombinase. In some embodiments, a site-specific recombinase recognizes and binds two recognition sequences; a first recognition sequence for the site-specific recombinase and a second recognition sequence for the site-specific recombinase, and mediates or catalyzes a site-specific recombination between the two recognition sequences. Thus, as used herein, a recombinase is “specific for” a recognition sequence when the recombinase can recognize and bind the recognition sequence and can mediate site-specific recombination between two recognition sequences.

As used herein, “site-specific recombination” refers to site-specific recombination that is effected between the nucleic acid sequence of two recognition sequences for a site-specific recombinase (e.g., a first recognition sequence and a second recognition sequence on a single nucleic acid molecule or between two different molecules) that requires the presence of the site specific recombinase.

“Heterologous” DNA in a host cell, in the present context refers to exogenous DNA not originating from the cell.

The term “genome” includes chromosomal as well as mitochondrial, chloroplast and viral DNA or RNA.

By “integration” or “insertion” it is meant that a polynucleotide of interest (e.g., a GEMS sequence or an exogenous polynucleotide encoding a desired product (e.g., a recombinant protein or a therapeutic polypeptide) is stably inserted into the cellular genome, i.e., covalently linked to the nucleic acid sequence within the cell's chromosomal DNA. By “targeted integration” or “site-specific integration” it is meant that the polynucleotide of interest is inserted into the cell's chromosomal or mitochondrial DNA at a specific site, or “integration site”.

As used herein, “selective conditions” means conditions that require expression of a selectable marker for survival of the cell.

The term “flanking insertion sequence” refers to a nucleotide sequences homologous to a genome sequence at the insertion site; wherein the GEMS sequence adjacent to the flanking insertion sequences is inserted at the insertion site. The flanking insertion sequences can comprise a pair of flanking insertion sequences, and said pair of flanking insertion sequences flank said GEMS sequence. In some cases, at least one flanking insertion sequence of said pair of flanking insertion sequences can comprise an insertion sequence that is homologous to a sequence of a safe harbor site (e.g., AAVs1, Rosa26, CCR5) of said genome. In some cases, the flanking insertion sequence is recognized by meganuclease, zinc finger nuclease, TALEN, CRISPR/Cas9, CRISPR/Cpf1, and/or Argonaut.

The term “host cell” refers to a cell comprising and capable of integrating one or more GEMS construct into its genome. The GEMS construct provided herein can be inserted into any suitable host cell. In some cases, the GEMS construct is integrated into a safe harbor site (e.g., Rosa26, AAVS1, CCR5, Hipp11 (H11) site). In some cases, the host cell is a mammalian cell. In some cases, the host cell is a rodent cell, e.g., Chinese hamster ovary (CHO) cell. In some cases, the host cell is a Chinese hamster ovary (CHO) cell. In some cases, the host cell is a stem cell. The host cell can be a prokaryotic or eukaryotic cell. Insertion of the construct can proceed according to any technique suitable in the art. For example, transfection, lipofection, or temporary membrane disruption such as electroporation or deformation can be used to insert the construct into the host cell. Viral vectors or non-viral vectors can be used to deliver the construct in some aspects. In an embodiment, the host cell can be competent for any endonuclease described herein. In an embodiment, the host cell can be competent for any site-specific recombinase described herein. Competency permits integration of the multiple gene editing site into the host cell genome. The host cell can be a primary isolate, obtained from a subject and optionally modified as necessary to make the cell competent for any required endonuclease. In some aspects, the host cell is a cell line. In some aspects, the host cell is a primary isolate or progeny thereof. In some aspects, the host cell is a stem cell. The stem cell can be an embryonic stem cell, a non-embryonic stem cell or an adult stem cell. The stem cell is preferably pluripotent, and not yet differentiated or begun a differentiation process. In some aspects, the host cell is a fully differentiated cell. When the host cell, transfected with the GEMS construct, divides, the multiple gene editing site of the construct can be integrated with the host cell genome such that progeny of the host cell can carry the multiple gene editing site. A host cell comprising an integrated multiple gene editing site can be cultured and expanded in order to increase the number of cells available for receiving donor gene sequences. Stable integration ensures subsequent generations of cells can have the multiple gene editing sites.

The term “donor nucleic acid sequence(s)”, “donor gene(s)” or “donor gene(s) of interest” or “exogenous polynucleotide” or “transgene” are used interchangeably herein, and refer to a nucleic acid sequence(s) or gene(s) inserted into the host cell genome at the multiple gene editing site. Donor nucleic acid sequences can be DNA. Donor nucleic acid sequences can be provided on an additional plasmid or other suitable vector that is inserted into the host cell. Transfection, lipofection, or temporary membrane disruption such as electroporation or deformation can be used to insert the vector comprising the donor nucleic acid sequence into the host cell. The donor nucleic acid sequences can be exogenous genes, or portions thereof, including engineered genes. The donor nucleic acid sequences can encode any protein or portion thereof that the user desires that the host cell express. The donor nucleic acid sequences (including genes) can further comprise a reporter gene, which can be used to confirm expression. The expression product of the reporter gene can be substantially inert such that its expression along with the donor gene of interest does not interfere with the intended activity of the donor gene expression product, or otherwise interfere with other natural processes in the cell, or otherwise cause deleterious effects in the cell. The donor nucleic acid sequence can also comprise regulatory elements that permit controlled expression of the donor gene. For example, the donor nucleic acid sequence can comprise a repressor operon or inducible operon. The expression of the donor nucleic acid sequence can thus be under regulatory control such that the gene is only expressed under controlled conditions. In some aspects, the donor nucleic acid sequence includes no regulatory elements, such that the donor gene is effectively constitutively expressed. In some embodiments, the donor nucleic acid sequence encoding is the green fluorescent protein (GFP) under a tetracycline (Tet)-inducible promoter.

An exogenous polynucleotide of protein X can refer to an exogenous polynucleotide comprising a nucleotide sequence that encodes protein X. As used herein, in some cases, an exogenous polynucleotide that encodes protein X can be an exogenous polynucleotide encoding 100% or about 100% of the amino acid sequence of protein X. In some cases, an exogenous polynucleotide that encodes protein X can encode the full or partial amino sequence of protein X. For example, the exogenous polynucleotide can encode at least or at least about 99%, 95%, 90%, 80%, 70%, 60%, 50%, 40%, 30%, 20%, 10%, or 5%, e.g., from or from about 99% to 90%; 90% to 80%; 80% to 70%; 70% to 60%; or 60% to 50%; of the amino acid sequence of protein X. Expression of an exogenous polynucleotide can ultimately result in a functional protein, e.g., a partially or fully functional protein. An exogenous polynucleotide can also encode an RNA (e.g., mRNA, shRNA, siRNA, or microRNA). In some cases, where an exogenous polynucleotide encodes for an mRNA, this can in turn be translated into a polypeptide (e.g., a protein). Therefore, it is contemplated that an exogenous polynucleotide can encode for protein. An exogenous polynucleotide can, in some instances, encode a protein or a portion of a protein. Additionally, a protein can have one or more mutations (e.g., deletion, insertion, amino acid replacement, or rearrangement) compared to a wild-type polypeptide. In some cases, an exogenous polynucleotide encodes a therapeutic protein. In some cases, an exogenous polynucleotide encodes an antibody or a fragment thereof. A protein can be a natural polypeptide or an artificial polypeptide (e.g., a recombinant polypeptide). In some cases the exogenous polynucleotide encodes a heavy chain polypeptide of an antibody or a fragment thereof. In some cases the exogenous polynucleotide encodes a light chain polypeptide of an antibody or a fragment thereof. An exogenous polynucleotide can encode a fusion protein formed by two or more polypeptides. In some cases, an exogenous polynucleotide comprises a first polynucleotide, and a second polynucleotide connected via linker polynucleotide. In some cases, the first polynucleotide encodes a heavy chain, or a light chain polypeptide of an antibody or a fragment thereof. In some cases, the second polynucleotide encodes a heavy chain, or a light chain polypeptide of an antibody or a fragment thereof. The compositions (e.g, GEMS constructs, or genetically engineered cells), and methods of the present disclosure is not limited to a particular antibody, and can be employed for expression of any antibody known or unknown in the art. Accordingly, the present disclosure contemplates any commercial antibody known in the art. For example, non limiting examples of a PD-L1 binding antibody include Atezolizumab, Avelumab, and Durvalumab. For example, non-limiting examples of a VEGF binding antibody include axitinib, bevacizumab, cabozantinib, lapatinib, lenvatinib, pazopanib, ponatinib, ramucirumab, ranibizumab, regorafenib, sorafenib, sunitinib, and vandetanib.

In some embodiments, the donor nucleic acid encodes a CAR construct (e.g., CD19 CAR). In some embodiments, the donor nucleic acid sequences comprise a nucleotide sequence of SEQ ID NO: In some embodiments, the donor nucleic acid sequences comprise a nucleotide sequence of SEQ ID NO: 21. In some embodiments, the donor nucleic acid sequences comprise a nucleotide sequence of SEQ ID NO: 22. In some embodiments, the donor nucleic acid sequences comprise a nucleotide sequence of SEQ ID NO: 23. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 21. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 22. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 23. In some embodiments, the exogenous polynucleotide encodes an antibody or a fragment thereof. In some embodiments, the antibody is a PD-L1 binding antibody. In some embodiments, the antibody is a VEGF binding antibody. In some embodiments, the exogenous polynucleotide encodes a heavy chain polypeptide of an antibody, or a fragment thereof. In some embodiments, the exogenous polynucleotide encodes a light chain of an antibody, or a fragment thereof. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 85. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 86. In some embodiments, the exogenous polynucleotide comprises a nucleic acid sequence comprising a first polynucleotide that encodes a light chain polypeptide of an antibody, or a fragment thereof. In some embodiments, the exogenous polynucleotide comprises a nucleic acid sequence comprising a second polynucleotide that encodes a heavy chain, or a fragment thereof of an antibody or a fragment thereof. In some embodiments, the first polynucleotide that encodes a light chain polypeptide or a fragment thereof comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 85. In some embodiments, the first polynucleotide that encodes a heavy chain polypeptide or a fragment thereof comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 86. In some embodiments, the first polynucleotide and the second polynucleotide are linked by a linker polynucleotide. In some embodiments, a linker polynucleotide comprises a sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 87 or SEQ ID NO: 88. In some embodiments, the exogenous polynucleotide further comprises a nucleic acid sequence encoding for a selectable marker polypeptide. In some embodiments, the nucleic acid sequence encoding a selectable marker polypeptide is a selectable marker gene. In some embodiments, the exogenous polynucleotide comprises a nucleic acid sequence encoding a modified selectable marker polypeptide. In some embodiments, a selectable marker polypeptide confers resistance to an antibiotic. In some embodiments, the antibiotic is puromycin, hygromycin, blasticidin, and neomycin. In some embodiments, the modified selectable marker polypeptide exhibits reduced activity by at least 2%, 5%, 10%, 15%. 20%, 25%, 30%, 35%, 40%, 45%, 50% or more relative to the corresponding WT selectable marker protein. In some embodiments, the reduced the modified selectable marker polypeptide is a modified neomycin phosphotransferase. In some embodiments, the modified neomycin phosphotransferase comprises a D227V amino acid substitution relative to the corresponding WT neomycin phosphotransferase. In some embodiments, a nucleic acid sequence encoding a modified selectable marker polypeptide comprises at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 84.

The term “isolated” and its grammatical equivalents as used herein refer to the removal of a nucleic acid from its natural environment. The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” It is to be understood, however, that nucleic acids and proteins can be formulated with diluents or adjuvants and still for practical purposes be isolated. For example, nucleic acids typically are mixed with an acceptable carrier or diluent when used for introduction into cells. The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

“Polynucleotide(s)”, “oligonucleotide(s)”, “nucleic acid(s)”, “nucleotide(s)”, “polynucleic acid(s)”, or any grammatical equivalent as used herein refers to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein can be introduced into a cell by, for example, transfection, transformation, or transduction.

“Transfection,” “transformation,” or “transduction” as used herein refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage, viral, or non-viral vectors can be introduced into host cells, after growth of infectious particles in suitable packaging cells, many of which are commercially available. In some embodiments, lipofection, nucleofection, or temporary membrane disruption (e.g., electroporation or deformation) can be used to introduce one or more exogenous polynucleotides into the host cell.

A “safe harbor” region or “safe harbor” site is a portion of the chromosome where one or more donor genes, including transgenes, can integrate, with substantially predictable expression and function, but without inducing adverse effects on the host cell or organism, including but not limited to, without perturbing endogenous gene activity or promoting cancer or other deleterious condition. See, Sadelain M et al. (2012) Nat. Rev. Cancer 12:51-58. In an embodiment, the safe harbor site is the adeno-associated virus site 1 (AAVS1), a naturally occurring site of integration of AAV virus on chromosome 19. In an embodiment, the safe harbor site is the chemokine (C-C motif) receptor 5 (CCR5) gene, a chemokine receptor gene known as an HIV-1 coreceptor. In an embodiment, the safe harbor site is the human ortholog of the mouse Rosa26 locus, a locus extensively validated in the murine setting for the insertion of ubiquitously expressed transgenes. By way of example, in humans, there is a safe harbor locus on chromosome 19 (PPP1R12C) that is known as AAVS1. In mice, the Rosa26 locus is known as a safe harbor locus. The human AAVS1 site is particularly useful for receiving transgenes in embryonic stem cells and for pluripotent stem cells. In some embodiments, a safe harbor region is a safe harbor region in the genome of a CHO cell. In some embodiments, a safe harbor region is a Hipp11 (H11) locus.

“Polypeptide”, “peptide” and their grammatical equivalents as used herein refer to a polymer of amino acid residues. A “mature protein” is a protein which is full-length and which, optionally, includes glycosylation or other modifications typical for the protein in a given cellular environment. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The terms “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence can be compared to a reference sequence of the same number of contiguous positions after the two sequences are aligned optimally. Methods of alignment of sequences for comparison are well-known in the art. Optimal alignment of sequences for comparison can be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein are at least 80%, 85%, 90%, 98% 99% or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 90% sequence identity or more, at least 95%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

“CD19”, cluster of differentiation 19 or B-lymphocyte antigen CD19, is a protein that in human is encoded by the CD19 gene. The CD19 gene encodes a cell surface molecule that assembles with the antigen receptor of B lymphocytes in order to decrease the threshold for antigen receptor-dependent stimulation. CD19 is expressed on follicular dendritic cells and B cells. In fact, it is present on B cells from earliest recognizable B-lineage cells during development to B-cell blasts but is lost on maturation to plasma cells. It primarily acts as a B cell co-receptor in conjunction with CD21 and CD81. Upon activation, the cytoplasmic tail of CD19 becomes phosphorylated, which leads to binding by Src-family kinases and recruitment of PI-3 kinase. As on T cells, several surface molecules form the antigen receptor and form a complex on B lymphocytes. The (almost) B cell-specific CD19 phosphoglycoprotein is one of these molecules. The others are CD21 and CD81. These surface immunoglobulin (sIg)-associated molecules facilitate signal transduction. On B cells, anti-immunoglobulin antibody mimicking exogenous antigen causes CD19 to bind to sIg and internalize with it. The reverse process has not been demonstrated, suggesting that formation of this receptor complex is antigen-induced. This molecular association has been confirmed by chemical studies.

An “expression vector” or “vector” is any genetic element, e.g., a plasmid, chromosome, virus, transposon, behaving either as an autonomous unit of polynucleotide replication within a cell. (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, transposons, bacteriophages and cosmids. Vectors can contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors can be capable of directly expressing nucleic acid sequence products encoded therein without ligation or integration of the vector into host cell DNA sequences. In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11:1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP. Vector also can comprise a selectable marker gene.

The term “selectable marker gene” as used herein refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150:1 (1981); Santerre et al., Gene, 30: 147 (1984); Kent et al., Science, 237: 901-903 (1987); Wigler et al., Cell, 11: 223 (1977); Szybalska & Szybalski, Proc. Natl. Acad. Sci. USA, 48: 2026 (1962); Lowy et al., Cell, 22: 817 (1980); and U.S. Pat. Nos. 5,122,464 and 5,770,359.

The term “coding sequence” as used herein refers to a segment of a polynucleotide that codes for protein. The region or sequence is bounded nearer the 5′ end by a start codon and nearer the 3′ end with a stop codon. Coding sequences can also be referred to as open reading frames.

The term “operably linked” as used herein refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence Enhancers and silencers can be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

The term “induce”, “induction” and its grammatical equivalents as used herein refer to an increase in nucleic acid sequence transcription, promoter activity and/or expression brought about by a transcriptional regulator, relative to some basal level of transcription.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences Enhancers can be located upstream, within, or downstream of coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

The term “promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off with an inducer at certain stages of development of an organism or in a particular tissue. Non-limiting examples of inducible promoters include alcohol-regulated promoters, tetracycline-regulated promoters, steroid-regulated promoters, metal-regulated promoters, pathogenesis-regulated promoters, temperature-regulated promoters and light-regulated promoters, isopropyl-P-thiogalactopyranoside (IPTG) inducible promoter.

As used herein, the term “guide RNA” and its grammatical equivalents can refer to an RNA which can be specific for a target DNA and can form a complex with Cas protein. An RNA/Cas complex can assist in “guiding” Cas protein to a target DNA.

The term “protospacer adjacent motif (PAM)” or PAM-like motif refers to a 2-6 base pair DNA sequence immediately following the DNA sequence targeted by Cas protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence varies by the species of the bacteria from which the Cas protein is derived. For example, the PAM sequence for Cas9 from Streptococcus pyogenes is NGG (N could be any of A, T, C or G). For another example, the PAM sequence for Neisseria meningitides is NNNNGATT. The PAM sequence for Streptococcus thermophilus is NNAGGAA. The PAM sequence for Treponema denticola is NAAAAC.

“T cell” or “T lymphocyte” as used herein is a type of lymphocyte that plays a central role in cell-mediated immunity. They can be distinguished from other lymphocytes, such as B cells and natural killer cells (NK cells), by the presence of a T-cell receptor (TCR) on the cell surface.

“T helper cells” (T H cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}9, T_{H}17, T_{H}22 or T_{FH} (T follicular helper cells), which secrete different cytokines to facilitate different types of immune responses. Signaling from the APCs directs T cells into particular subtypes.

“Cytotoxic T cells” (TC cells, or CTLs) or “cytotoxic T lymphocytes” destroy virus-infected cells and tumor cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Through IL-10, adenosine, and other molecules secreted by regulatory T cells, the CD8+ cells can be inactivated to an anergic state, which prevents autoimmune diseases.

“Memory T cells” are a subset of antigen-specific T cells that persist long-term after an infection has resolved. They quickly expand to large numbers of effector T cells upon re-exposure to their cognate antigen, thus providing the immune system with memory against past infections. Memory T cells comprise three subtypes: central memory T cells (T_{CM} cells) and two types of effector memory T cells (T_{EM} cells and T_{EMRA} cells). Memory cells can be either CD4+ or CD8+. Memory T cells typically express the cell surface proteins CD45RO, CD45RA and/or CCR7.

“Regulatory T cells” (Treg cells), formerly known as suppressor T cells, play a role in the maintenance of immunological tolerance. Their major role is to shut down T cell-mediated immunity toward the end of an immune reaction and to suppress autoreactive T cells that escaped the process of negative selection in the thymus.

“Natural killer cells” or “NK cells” are a type of cytotoxic lymphocyte critical to the innate immune system. The role NK cells play is analogous to that of cytotoxic T cells in the vertebrate adaptive immune response. NK cells provide rapid responses to viral-infected cells, acting at around 3 days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells. NK cells (belonging to the group of innate lymphoid cells) are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting interferon gamma. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice.

“Natural killer T cells” (NKT cells—not to be confused with natural killer cells of the innate immune system) bridge the adaptive immune system with the innate immune system. Unlike conventional T cells that recognize peptide antigens presented by major histocompatibility complex (MHC) molecules, NKT cells recognize glycolipid antigen presented by a molecule called CD1d. Once activated, these cells can perform functions ascribed to both T helper (T_{H}) and cytotoxic T (TC) cells (i.e., cytokine production and release of cytolytic/cell killing molecules). They are also able to recognize and eliminate some tumor cells and cells infected with herpes viruses.

“Adoptive T cell transfer” refers to the isolation and ex vivo expansion of tumor specific T cells to achieve greater number of T cells than what can be obtained by vaccination alone or the patient's natural tumor response. The tumor specific T cells are then infused into patients with cancer in an attempt to give their immune system the ability to overwhelm remaining tumor via T cells which can attack and kill cancer. There are many forms of adoptive T cell therapy being used for cancer treatment; culturing tumor infiltrating lymphocytes or TIL, isolating and expanding one particular T cell or clone, and even using T cells that have been engineered to potently recognize and attack tumors.

The term “antibody” as used herein includes IgG (including IgG1, IgG2, IgG3, and IgG4), IgA (including IgA1 and IgA2), IgD, IgE, or IgM, and IgY. The term includes whole antibodies, including “antigen-binding fragments” or “functional fragment thereof”, or “fragment of an antibody”, “antibody fragment”, “functional fragment of an antibody” and other interchangeable terms for similar binding fragments. The term further includes single-chain whole antibodies. Antigen-binding fragments include, but are not limited to, Fab, Fab′ and F(ab′)2, Fd (consisting of VH and CH1), single-chain variable fragment (scFv), single-chain antibodies, disulfide-linked variable fragment (dsFv) and fragments comprising either a VL or VH domain. An antibody includes, for example, monoclonal antibodies, chimeric antibodies, humanized antibodies, human antibodies, recombinant antibodies, chemically engineered antibodies, deimmunized antibodies, affinity-matured antibodies, multispecific antibodies (for example, bispecific antibodies and polyreactive antibodies), heteroconjugate antibodies, antibody fragments, and combinations thereof (e.g., a monoclonal antibody that is also deimmunized, a humanized antibody that is also deimmunized, etc.).

An antibody can be, for example, murine, chimeric, humanized, heteroconjugate, bispecific, diabody, triabody, or tetrabody. The antigen binding fragment can include, for example, Fab′, F(ab′)2, Fab, Fv, rlgG, scFv, hcAbs (heavy chain antibodies), a single domain antibody, VHH, VNAR, sdAbs, or nanobody.

The antibodies can be from any animal origin. Antigen-binding fragments, including single-chain antibodies, can comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies can be monoclonal, polyclonal, chimeric, humanized, and human monoclonal and polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding domain of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

“Antibody like molecules” can be for example proteins that are members of the Ig-superfamily which are able to selectively bind a partner. MHC molecules and T cell receptors are such molecules. In one embodiment, the antibody-like molecule is an TCR. In one embodiment, the TCR has been modified to increase its MHC binding affinity.

The terms “fragment of an antibody,” “antibody fragment,” “functional fragment of an antibody,” “antigen-binding domain” or its grammatical equivalents are used interchangeably herein to mean one or more fragments or portions of an antibody that retain the ability to specifically bind to an antigen (see, generally, Holliger et al., Nat. Biotech., 23(9):1126-1129 (2005)). The antibody fragment desirably comprises, for example, one or more CDRs, the variable region (or portions thereof), the constant region (or portions thereof), or combinations thereof. Non-limiting examples of antibody fragments include (i) a Fab fragment, which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites.

“Epitope”, “antigenic determinant”, “antigen recognition moiety”, “antigen recognition domain”, and their grammatical equivalents refer to a molecule or portion of an antigen to which specifically e.g., an antibody or a receptor binds. In one embodiment, the antigen recognition moiety is in an antibody, antibody like molecule or fragment thereof and the antigen is a tumor antigen.

As used herein, a “target binding antibody” refers to an antibody that comprises an antigen binding domain from an antibody or from a non-antibody that can bind to an antigen. Accordingly, as used herein, a “PD-L1 binding antibody” refers to an antibody that comprises an antigen binding domain from an antibody or from a non-antibody that can bind a PD-L1 polypeptide. As used herein, a “VEGF binding antibody” refers to an antibody that comprises an antigen binding domain from an antibody or from a non-antibody that can bind a PD-L1 polypeptide. The term “PD-L1” or “Programmed death-ligand 1” as used herein, refers, to any native or variant (whether native or synthetic) PD-L1. The term “VEGF” or “Vascular endothelial growth factor” as used herein, refers, to any native or variant (whether native or synthetic) PD-L1. In some embodiments, the VEGF is a VEGF-A. The term “native sequence” specifically encompasses naturally occurring truncated or secreted forms (e.g., an extracellular domain sequence), naturally occurring variant forms (e.g., alternatively spliced forms) and naturally-occurring allelic variants. The polypeptide and nucleic acid sequences of PD-L1 and VEGF are known in the art and publically available e.g., from the NCBI website. In some embodiments, the PD-L1 polypeptide includes or is derived from human PD-L1 having the amino acid sequence set forth at NCBI reference sequence number: NP_054862.1, which is incorporated herein in its entirety. In some embodiments, the VEGF polypeptide includes or is derived from human VEGF having the amino acid sequence set forth at NCBI reference sequence number: NP_001020537.2, which is incorporated herein in its entirety.

A “functional variant” of a protein used herein refers to a polypeptide, or a protein having substantial or significant sequence identity or similarity to the reference polypeptide, and retains the biological activity of the reference polypeptide of which it is a variant. In some embodiments, a functional variant, for example, comprises the amino acid sequence of the reference protein with at least or about 1, 2, 3, 4, 5, 6, 7, 8, 9, 10, 11, 12, 13, 14, 15, 16, 17, 18, 19, or 20 conservative amino acid substitutions. Functional variants encompass, for example, those variants of the CAR described herein (the parent CAR) that retain the ability to recognize target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 70% identical, about 75% identical, about 80% identical, about 85% identical, about 90% identical, about 95% identical, or about 99% identical to the nucleic acid sequence encoding the parent CAR.

The term “functional portion,” when used in reference to a CAR, refers to any part or fragment of a CAR described herein, which part or fragment retains the biological activity of the CAR of which it is a part (the parent CAR). In reference to a nucleic acid sequence encoding the parent CAR, a nucleic acid sequence encoding a functional portion of the CAR can encode a protein comprising, for example, about 10%, 25%, 30%, 50%, 68%, 80%, 90%, 95%, or more, of the parent CAR.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained. Alternatively or additionally, the functional variants can comprise the amino acid sequence of the reference protein with at least one non-conservative amino acid substitution.

The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the parent CAR.

“Proliferative disease” as referred to herein means a unifying concept that excessive proliferation of cells and turnover of cellular matrix contribute significantly to the pathogenesis of several diseases, including cancer is presented.

“Patient” or “subject” as used herein refers to a mammalian subject diagnosed with or suspected of having or developing a proliferative disorder such as cancer. In some embodiments, the term “patient” refers to a mammalian subject with a higher than average likelihood of developing a proliferative disorder such as cancer. Exemplary patients can be humans, non-human primates, cats, dogs, pigs, cattle, cats, horses, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with or suspected of having a disease or disorder, for instance, but not restricted to a proliferative disorder such as cancer, an infectious disease, an autoimmune disease, an inflammatory disease. In some cases, a cancer is a solid tumor or a hematologic malignancy. In some instances, the cancer is a solid tumor. In other instances, the cancer is a hematologic malignancy. In some cases, the cancer is a metastatic cancer. In some cases, the cancer is a relapsed or refractory cancer. In some instances, the cancer is a solid tumor. Exemplary solid tumors include, but are not limited to, anal cancer; appendix cancer; bile duct cancer (i.e., cholangiocarcinoma); bladder cancer; brain tumor; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments leukemia can be, for instance, acute lymphoblastic leukemia (ALL), acute myeloid leukemia (AML), chronic lymphocytic leukemia (CLL) and chronic myeloid leukemia (CML).

“Administering” is referred to herein as providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise a target cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the term “treatment”, “treating”, or its grammatical equivalents refers to obtaining a desired pharmacologic and/or physiologic effect. In embodiments, the effect is therapeutic, i.e., the effect partially or completely cures a disease and/or adverse symptom attributable to the disease. To this end, the inventive method comprises administering a therapeutically effective amount of the composition comprising the host cells expressing the inventive nucleic acid sequence, or a vector comprising the inventive nucleic acid sequences.

The term “therapeutically effective amount”, therapeutic amount″, “immunologically effective amount”, “anti-tumor effective amount”, “tumor-inhibiting effective amount” or its grammatical equivalents refers to an amount effective, at dosages and for periods of time necessary, to achieve a desired therapeutic result. The therapeutically effective amount can vary according to factors such as the disease state, age, sex, and weight of the individual, and the ability of a composition described herein to elicit a desired response in one or more subjects. The precise amount of the compositions of the present disclosure to be administered can be determined by a physician with consideration of individual differences in age, weight, tumor size, extent of infection or metastasis, and condition of the patient (subject).

Alternatively, the pharmacologic and/or physiologic effect of administration of one or more compositions described herein to a patient or a subject of can be “prophylactic,” i.e., the effect completely or partially prevents a disease or symptom thereof. A “prophylactically effective amount” refers to an amount effective, at dosages and for periods of time necessary, to achieve a desired prophylactic result (e.g., prevention of disease onset).

Some numerical values disclosed throughout are referred to as, for example, “X is at least or at least about 100; or 200 [or any numerical number].” This numerical value includes the number itself and all of the following:
i) X is at least 100;
ii) X is at least 200;
iii) X is at least about 100; and
iv) X is at least about 200.

All these different combinations are contemplated by the numerical values disclosed throughout. All disclosed numerical values should be interpreted in this manner, whether it refers to an administration of a therapeutic agent or referring to days, months, years, weight, dosage amounts, etc., unless otherwise specifically indicated to the contrary.

The ranges disclosed throughout are sometimes referred to as, for example, “X is administered on or on about day 1 to 2; or 2 to 3 [or any numerical range].” This range includes the numbers themselves (e.g., the endpoints of the range) and all of the following:
i) X being administered on between day 1 and day 2;
ii) X being administered on between day 2 and day 3;
iii) X being administered on between about day 1 and day 2;
iv) X being administered on between about day 2 and day 3;
v) X being administered on between day 1 and about day 2;
vi) X being administered on between day 2 and about day 3;
vii) X being administered on between about day 1 and about day 2; and
viii) X being administered on between about day 2 and about day 3.

All these different combinations are contemplated by the ranges disclosed throughout. All disclosed ranges should be interpreted in this manner, whether it refers to an administration of a therapeutic agent or referring to days, months, years, weight, dosage amounts, etc., unless otherwise specifically indicated to the contrary.

### Gene Editing Multi-Sites (Gems)

Gene modified cell therapies are rapidly moving through clinical development and are the new drug frontier. However, these therapies are individualized solutions and therefore lack economy of scale and have limited patient access. These challenges offer the opportunity to create solutions that can support the economy of scale and make the therapy available to all patients in need. One solution can be to create “off the shelf” products. These products are derived from a donor and then expanded to be used in many recipients. Off the shelf products need to overcome some challenge to become of therapeutic and commercial value. Such challenge include overcoming rejection and sensitization; improve reliability of the gene modifications to reduce safety risks and cost; expanding therapeutic cell to high numbers (˜10⁹ cells, or more, per treatment); increasing dose to donor ratios (doses generated per donor) which will decrease development and manufacturing cost.

Provided herein is a nucleic acid construct comprising a multiple gene editing site or a gene editing multi-sites (GEMS) for facilitating gene editing and genetic engineering. The construct comprises DNA, and can be in the form of a plasmid. The term “multiple gene editing sites” and “gene editing multi-sites” are used interchangeably herein. The GEMS system can offer significant benefits, such as plug and play system to reduce development cost; exact known location of gene insert which enhances safety; standard tools to insert any gene construct allowing customization; and a possibility to be introduced in any source cell type preferably a self-renewing source. In some embodiments, the GEMS construct comprises eukaryotic nucleotides. In some embodiments, the GEMS construct comprises a GEMS sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. In some embodiments, the GEMS construct comprises a nucleotide sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, SEQ ID NO: 3 and/or SEQ ID NO: 105.

In some embodiments, the GEMS construct comprises a plurality of a first recognition sequences for a site-specific recombinase. In some embodiments, each of the first recognition sequence can undergo a site-specific recombination with a second recognition sequence for the site specific recombinase, when contact with the site-specific recombinase. In some embodiments, the site specific recombinase is a serine recombinase or a tyrosine recombinase. In some embodiments, the site specific recombinase is a serine integrase. In some embodiments, the site specific recombinase is a tyrosine recombinase. In some embodiments, the site-specific recombinase is a Bxb1 recombinase. In some embodiments, the site-specific recombinase comprises a sequence that is at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 108.

The plurality of first recognition sequences for a site-specific recombinase can comprise at least 3, 4, 5, 6, 7, 8, 9, 10, 11, 12, 13, 14, 15, 16, 17, 18, 19, 20, 25, or more recognition sequences. In some embodiments, the plurality of first recognition sequences can be unique nuclease recognition sequences. In some embodiments, at least one of the plurality of first recognition sequences can be heterologous to the genome. In some embodiments, each of said plurality of first recognition sequences can be heterologous to the genome. In some embodiments, the plurality of first recognition sequences comprises an att site. In some embodiments, the plurality of first recognition sequences comprises an attB site, and the second recognition sequence for the site-specific recombinase comprises an attP site. In some embodiments, the plurality of first recognition sequences comprises an attP site, and the second recognition sequence for the site-specific recombinase comprises an attB site. In some embodiments, at least 2, 3, 4, 5, 6, 7, 8, 9, 10, 11, 12, 13, 14, 15, 16, 17, 18, 19, 20, 25, or more of the plurality of first recognition sequences is an attB site. In some embodiments, at least 2, 3, 4, 5, 6, 7, 8, 9, 10, 11, 12, 13, 14, 15, 16, 17, 18, 19, 20, 25, or more of the plurality of first recognition sequences is an attP site. In some embodiments, at least one of the plurality of first recognition sequences for a site specific recombinase can be selected, for example, from SEQ ID NOs: 106,107, or reverse complements thereof. In some embodiments, each of the plurality of nuclease recognition sequences can be selected from SEQ ID NOs: 106,107, or reverse complements thereof.

In some embodiments, the GEMS construct comprises a GEMS sequence comprising a plurality of nuclease recognition sequences, wherein each of the plurality of nuclease recognition sequences comprises a target sequence and a protospacer adjacent motif (PAM), or reverse complement thereof. The plurality of nuclease recognition sequences can comprise at least 3, 4, 5, 6, 7, 8, 9, 10, 11, 12, 13, 14, 15, 16, 17, 18, 19, 20, 25, or more nuclease recognition sequences. In some embodiments, the plurality of nuclease recognition sequences can be unique nuclease recognition sequences. In some embodiments, at least one of the plurality of nuclease recognition sequences can be heterologous to the genome. In some embodiments, each of said plurality of nuclease recognition sequences can be heterologous to the genome. In some embodiments, at least one of the plurality of nuclease recognition sequences can be selected, for example, from SEQ ID NOs: 89, 91, 93, 95, 97, 99, 101, 103, or reverse complements thereof. In some embodiments, each of the plurality of nuclease recognition sequences can be selected from SEQ ID NOs: 89, 91, 93, 95, 97, 99, 101, 103, or reverse complements thereof.

In some embodiments, the plurality of nuclease recognition sequences can comprise, at least 3, 4, 5, 6, 7, 8, 9, 10, 11, 12, 13, 14, 15, 16, 17, 18, 19, 20, 25, or more target sequences. In some embodiments, the target sequences of each of the plurality of nuclease recognition sequences can be the same, although in other embodiments, the target sequences of each of the plurality of nuclease recognition sequences can be unique. In some embodiments, at least one target sequence in the plurality of nuclease recognition sequence can be heterologous to the genome. In other embodiments, each target sequence of the plurality of nuclease recognition sequences can be heterologous to the genome. The target sequence can be from about 10 to about 30 nucleotides in length, from about 15 to about 25 nucleotides in length, and from about 17 to about 24 nucleotides in length. In some aspects, the target sequence is about 20 nucleotides in length. In some embodiments, the target sequence can be GC-rich, such that at least about 40% of the target sequence is made up of G or C nucleotides. The GC content of the target sequence can from about 40% to about 80%, though GC content of less than about 40% or greater than about 80% can be used. In some embodiments, the target sequence can be AT-rich, such that at least about 40% of the target sequence is made up of A or T nucleotides. The AT content of the target sequence can from about 40% to about 80%, though AT content of less than about 40% or greater than about 80% can be used.

Methods described herein can take advantage of a CRISPR/Cas system. For example, double-strand breaks (DSBs) can be generated using a CRISPR/Cas system, e.g., a type II CRISPR/Cas system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at a target site sequence which hybridize to nucleotides of a gRNA sequence and that have a protospacer-adjacent motif (PAM) following the nucleotides of a target sequence. Accordingly, the plurality of nuclease recognition sequences of the construct disclosed herein is a recognition sequence for a Cas protein. The Cas protein can be, for example, Cas1, Cas1B, Cas2, Cas3, Cas4, Cas5, Cas5d, Cas5t, Cas5h, Cas5a, Cash, Cas7, Cas8, Cas9, Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. The PAM sequences of the Cas proteins are well known in the art. Non limiting examples of PAM sequences include CC, NG, YG, NGG, NAA, NAT, NAG, NAC, NTA, NTT, NTG, NTC, NGA, NGT, NGC, NCA, NCT, NCG, NCC, NRG, TGG, TGA, TCG, TCC, TCT, GGG, GAA, GAC, GTG, GAG, CAG, CAA, CAT, CCA, CCN, CTN, CGT, CGC, TAA, TAC, TAG, TGG, TTG, TCN, CTA, CTG, CTC, TTC, AAA, AAG, AGA, AGC, AAC, AAT, ATA, ATC, ATG, ATT, AWG, AGG, GTG, TTN, YTN, TTTV, TYCV, TATV, NGAN, NGNG, NGAG, NGCG, NGGNG, NGRRT, NGRRN, NNGRRT, NNAAAAN, NNNNGATT, NAAAAC, NNAAAAAW, NNAGAA, NNNNACA, GNNNCNNA, NNNNGATT, NNAGAAW, NNGRR, NNNNNNN, TGGAGAAT, AAAAW, GCAAA, and TGAAA. In some embodiments, each PAM sequence of the plurality of nuclease recognition sequences can be unique.

In some embodiments, each of the plurality of nuclease recognition sequences can be contiguous with other nuclease recognition sequences but each nuclease recognition sequence can be separated from an adjacent sequence by a polynucleotide spacer. The polynucleotide spacer can comprise any suitable number of nucleotides. The spacer length can be from about 2 nucleotides (base pairs in a double stranded construct) to about 10,000 or more nucleotides. In some embodiments, the space length is about 2 to about 5 nucleotides, from about 5 to about 10 nucleotides, from about 10 to about 20 nucleotides, from about 20 to about 30 nucleotides, from about 30 to about 40 nucleotides, from about 40 to about 50 nucleotides, from about 50 to about 100 nucleotides, from about 100 to about 200 nucleotides, from about 200 to about 300 nucleotides, from about 300 to about 400 nucleotides, from about 400 to about 500 nucleotides, from about 500 to about 1,000 nucleotides, from about 1,000 to about 2,000 nucleotides, from about 2,000 to about 5,000 nucleotides, or from about 5,000 to about 10,000 nucleotides. In some aspects, the spacer length is from about 5 to about 1000 nucleotides, from about 10 to about 100 nucleotides, or from about 25 to about 50 nucleotides.

In some embodiments, the GEMS construct further comprises a first flanking insertion sequence homologous to a first genome sequence upstream of the insertion site, where the first flanking insertion sequence is located upstream of the GEMS sequence; and a second flanking insertion sequence homologous to a second genome sequence downstream of the insertion site, where the second flanking insertion sequence is located downstream of the GEMS sequence. In some cases, at least the first flanking insertion sequence, the second flanking insertion sequence or both can comprise at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 100 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, or at least 1,000 nucleotides. In some embodiments, at least the first flanking insertion sequence, the second flanking insertion sequence or both comprises a sequence homologous to a sequence of a safe harbor site (e.g., Rosa26, AAVS1, CCR5) of a host cell genome. In some embodiments, the first flanking insertion sequence can be Rosa26 5′ homology arm sequence comprising a nucleotide sequence of SEQ ID NO: 7. In some embodiments, the second flanking insertion sequence can be Rosa26 3′ homology arm sequence comprising a nucleotide sequence of SEQ ID NO: 8.

In some embodiments, the insertion site can be a safe harbor site. The GEMS construct can be targeted to and can stably integrate into a safe harbor site (e.g., Rosa26, AAVS1, CCR5) of a chromosome. A “safe harbor” site is a portion of the chromosome where one or more donor genes, including transgenes, can integrate, with substantially predictable expression and function, but without inducing adverse effects on the host cell or organism, including but not limited to, without perturbing endogenous gene activity or promoting cancer or other deleterious condition. See, Sadelain M et al. (2012) Nat. Rev. Cancer 12:51-58. By way of example, in humans, there is a safe harbor locus on chromosome 19 (PPP1R12C) that is known as AAVS1. In mice, the Rosa26 locus is known as a safe harbor locus. The human AAVS1 site can be particularly useful for receiving transgenes in embryonic stem cells and for pluripotent stem cells. The human AAVS1 site is preferred for use in accordance with some aspects of the construct. In some embodiments, the first flanking insertion sequence can be Rosa26 homology arm sequence comprising a nucleotide sequence of SEQ ID NO: 7. In some embodiments, the second flanking insertion sequence can be Rosa26 3′ homology arm sequence comprising a nucleotide sequence of SEQ ID NO: 8. In some embodiments, Rosa26 CRISPR targeting sequence comprises a nucleotide sequence of SEQ ID NO: 9. In some embodiments, Rosa26 CRISPR gRNA sequence comprises a nucleotide sequence of SEQ ID NO: 10.

To insert the GEMS construct into the safe harbor locus (e.g., Rosa26, AAVS1, CCR5), endonuclease activity in the cell can be used. In some embodiments, the GEMS construct comprises a first meganuclease recognition sequence upstream of the GEMS sequence. In some embodiments, the GEMS construct can further comprise a second meganuclease recognition sequence downstream of the GEMS sequence. The first meganuclease recognition sequence can be upstream of the first flanking insertion sequence. The second meganuclease sequence can be downstream of the second flanking insertion sequence. The first meganuclease recognition sequence, the second meganuclease recognition sequence, or both can comprise an I-SceI meganuclease recognition sequence. In some embodiments, an I-SceI meganuclease recognition sequence comprises a nucleic acid sequence that is at least 50%, 60%, 70%, 80%, 90%, 95% or more identical to sequence set forth in SEQ ID NO: 2. The meganuclease recognition sequence allow the GEMS construct to be cleaved by a meganuclease in the cell in order to generate a donor sequence comprising the GEMS sequence. This donor sequence comprising GEMS sequence can then be inserted into a safe harbor locus. A compatible meganuclease recognizes the recognition sequence, and cleaves the construct accordingly. In some embodiments, the meganuclease recognition sequences are in common with meganuclease recognition sequences present at the safe harbor locus. In this way, the meganuclease can cleave the safe harbor locus, allowing insertion of the free (cleaved from the construct) GEMS sequence into the cleaved safe harbor locus. This insertion can proceed via homologous or non-homologous end joining (NHEJ) in the cell. Thus, the meganuclease recognition sequences can be tailored to nucleases that produce compatible ends at the site of the double stranded breaks in the construct DNA and in the safe harbor locus.

The meganuclease recognition sequences upstream and downstream of the GEMS sequence facilitate insertion of the GEMS sequence into the genome of a host cell. Thus, the constructs can be used, for example, to transfect a recipient cell and, once in the recipient cell, the upstream and downstream meganuclease recognition sequences facilitate insertion of the GEMS sequence into a chromosome. Once the GEMS sequence is inserted into a chromosome, the cell can be further modified with donor genes or portions thereof that are inserted into one or more of the plurality of nuclease recognition site in the GEMS sequence. In some embodiments, insertion of the GEMS sequence into a chromosome (e.g., safe harbor sequence of a genome) is stable integration into the chromosome.

In some embodiments, the GEMS sequence is introduced in the cells by random integration. Cells can be further screened for insertion in a select safe harbor site, for example, by amplification of genomic DNA region within a safe harbor site. Cells showing integration of GEMS sequence within a safe harbor site can be used to further engineer a transgene within the GEMS sequence inserted in the safe harbor site.

In some embodiments, the GEMS construct can further comprise a reporter gene such as a gene coding for a fluorescent protein (e.g., green fluorescent protein). The expression of the reporter gene can be regulated by an inducible promoter. Inducible promoter can be induced, for example, by doxycycline, isopropyl-P-thiogalactopyranoside (IPTG), galactose, a divalent cation, lactose, arabinose, xylose, N-acyl homoserine lactone, tetracycline, a steroid, a metal, an alcohol, or a combination thereof. The methods described herein allows a DNA construct (e.g., GEMS construct, a gene of interest) entry into a host cell by e.g., calcium phosphate/DNA co-precipitation, microinjection of DNA into a nucleus, electroporation, bacterial protoplast fusion with intact cells, transfection, lipofection, infection, particle bombardment, sperm mediated gene transfer, or any other technique known by one skilled in the art.

### Site Specific Modification

Inserting one or more GEMS constructs disclosed herein can be site-specific. For example, one or more transgenes can be inserted adjacent to Rosa26, AAVS1, CCR5, Hipp11 (H11). In some embodiments, the GEMS sequence adjacent to the flanking insertion sequences is inserted at the insertion site. The flanking insertion sequences can comprise a pair of flanking insertion sequences, and said pair of flanking insertion sequences flank said GEMS sequence. In some cases, at least one flanking insertion sequence of said pair of flanking insertion sequences can comprise an insertion sequence that is homologous to a sequence of a safe harbor site (e.g., AAVs1, Rosa26, CCR5, Hipp11 (H11)) of said genome. In some cases, the flanking insertion sequence is recognized by meganuclease, zinc finger nuclease, TALEN, CRISPR/Cas9, CRISPR/Cpf1, and/or Argonaut. In some cases, the flanking sequence has a length of about 14 to 40 nucleotides. In some cases, the flanking sequence has a length of about 18 to 36 nucleotides. In some cases, the flanking sequence has a length of about 28 to 40 nucleotides. In some cases, the flanking sequence has a length of about 19 to 22 nucleotides. In some cases, the flanking sequence has a length of at least 18 nucleotides. In some cases, the flanking sequence has a length of at least 50 nucleotides. In some cases, the flanking sequence has a length of at least 100 nucleotides. In some cases, the flanking sequence has a length of at least 500 nucleotides. In some embodiments, the first flanking insertion sequence can be Rosa26 5′ homology arm sequence comprising a nucleotide sequence of SEQ ID NO: 7. In some embodiments, the second flanking insertion sequence can be Rosa26 3′ homology arm sequence comprising a nucleotide sequence of SEQ ID NO: 8. In some embodiments, Rosa26 CRISPR targeting sequence comprises a nucleotide sequence of SEQ ID NO: 9. In some embodiments, Rosa26 CRISPR gRNA sequence comprises a nucleotide sequence of SEQ ID NO: 10.

Modification of a targeted locus of a cell can be produced by introducing DNA into cells, where the DNA has homology to the target locus. DNA can include a marker gene, allowing for selection of cells comprising the integrated construct. Homologous DNA in a target vector can recombine with a chromosomal DNA at a target locus. The DNA construct to be inserted can be flanked on both sides by homologous DNA sequences, a 3′ recombination arm, and a 5′ recombination arm. In some embodiments, the GEMS construct comprises a GEMS sequence of SEQ ID NO: 1, or SEQ ID NO: 3. In some embodiments, the GEMS construct comprises a GEMS sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. In some embodiments, the GEMS construct comprises a nucleotide sequence of SEQ ID NO: 1, SEQ ID NO: 3, and/or SEQ ID NO: 105.

A variety of enzymes can catalyze insertion of foreign DNA into a host genome. For example, site-specific recombinases can be clustered into two protein families with distinct biochemical properties, namely tyrosine recombinases (in which DNA is covalently attached to a tyrosine residue) and serine recombinases (where covalent attachment occurs at a serine residue). In some cases, recombinases can comprise Cre, fC31 integrase (a serine recombinase derived from Streptomyces phage fC31), or bacteriophage derived site-specific recombinases (including Flp, lambda integrase, bacteriophage HK022 recombinase, bacteriophage R4 integrase and phage TP901-1 integrase).

Cre/lox recombination is a tyrosine family site-specific recombinase technology, used to carry out deletions, insertions, translocations and inversions at specific sites in the DNA of cells. It allows the DNA modification to be targeted to a specific cell type or be triggered by a specific external stimulus. It can be implemented both in eukaryotic and prokaryotic systems. The Cre/lox system consists of a single enzyme, Cre recombinase, that recombines a pair of short target sequences called the Lox sequences. This system can be implemented without inserting any extra supporting proteins or sequences. The Cre enzyme and the original Lox site called the LoxP sequence are derived from bacteriophage P1. Placing Lox sequences appropriately allows genes to be activated, repressed, or exchanged for other genes. At a DNA level many types of manipulations can be carried out. The activity of the Cre enzyme can be controlled so that it is expressed in a particular cell type or triggered by an external stimulus like a chemical signal or a heat shock.

Flp/FRT recombination is a site-directed recombination technology used to manipulate an organism's DNA under controlled conditions in vivo. It is analogous to Cre/lox recombination but involves the recombination of sequences between short flippase recognition target (FRT) sites by the recombinase flippase(Flp) derived from the 2 μm plasmid of baker's yeast Saccharomyces cerevisiae. The Flp protein is a tyrosine family site-specific recombinase. This family of recombinases performs its function via a type IB topoisomerase mechanism causing the recombination of two separate strands of DNA. Recombination is carried out by a repeated two-step process. The initial step causes the creation of a Holliday junction intermediate. The second step promotes the resulting recombination of the two complementary strands.

The CRISPR/Cas system can be used to perform site specific insertion. For example, a nick on an insertion site in the genome can be made by CRISPR/Cas to facilitate the insertion of a transgene at the insertion site.

Certain aspects disclosed herein can utilize vectors. Any plasmids and vectors can be used as long as they are replicable and viable in a selected host. Vectors known in the art and those commercially available (and variants or derivatives thereof) can be engineered to include one or more recombination sites for use in the methods. Vectors that can be used include, but not limited to, bacterial expression vectors (such as pBs, pQE-9 (Qiagen), phagescript, PsiX174, pBluescript SK, pB5KS, pNH8a, pNH16a, pNH18a, pNH46a (Stratagene), pTrc99A, pKK223-3, pKK233-3, pDR540, pRIT5 (Pharmacia), and variants or derivatives thereof), eukaryotic expression vectors (such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, pYACneo (Clontech), pSVK3, pSVL, pMSG, pCH110, pKK232-8 (Pharmacia, Inc.), p3′SS, pXT1, pSG5, pPbac, pMbac, pMClneo, pOG44 (Stratagene, Inc.), pYES2, pAC360, pBlueBa-cHis A, B, and C, pVL1392, pBlueBac111, pCDM8, pcDNA1, pZeoSV, pcDNA3, pREP4, pCEP4, pEBVHis (Invitrogen, Corp.), pWLneo, pSv2cat, pOG44, pXT1, pSG (Stratagene) pSVK3, pBPv, pMSG, pSVL (Pharmiacia), and variants or derivatives thereof), and any other plasmids and vectors replicable and viable in the host cell.

Vectors known in the art and those commercially available (and variants or derivatives thereof) can in accordance with the present disclosure be engineered to include one or more recombination sites for use in the methods of the present disclosure. These vectors can be used to express a gene, e.g., a transgene, or portion of a gene of interest. A gene of portion or a gene can be inserted by using known methods, such as restriction enzyme-based techniques.

One or more recombinases can be introduced into a host cell before, concurrently with, or after the introduction of a target vector (e.g., a GEMS vector). The recombinase can be directly introduced into a cell as a protein, for example, using liposomes, coated particles, or microinjection. Alternately, a polynucleotide, either DNA or messenger RNA, encoding the recombinase can be introduced into the cell using a suitable expression vector. The targeting vector components can be useful in the construction of expression cassettes containing sequences encoding a recombinase of interest. However, expression of the recombinase can be regulated in other ways, for example, by placing the expression of the recombinase under the control of a regulatable promoter (i.e., a promoter whose expression can be selectively induced or repressed).

Recombinases for use in the practice of the present disclosure can be produced recombinantly or purified as previously described. Polypeptides having the desired recombinase activity can be purified to a desired degree of purity by methods known in the art of protein ammonium sulfate precipitation, purification, including, but not limited to, size fractionation, affinity chromatography, HPLC, ion exchange chromatography, heparin agarose affinity chromatography (e.g., Thorpe & Smith, Proc. Nat. Acad. Sci. 95:5505-5510, 1998.).

In one embodiment, the recombinases can be introduced into the eukaryotic cells that contain the recombination attachment sites at which recombination is desired by any suitable method. Methods of introducing functional proteins, e.g., by microinjection or other methods, into cells are well known in the art. Introduction of purified recombinase protein ensures a transient presence of the protein and its function, which is often a preferred embodiment. Alternatively, a gene encoding the recombinase can be included in an expression vector used to transform the cell, in which the recombinase-encoding polynucleotide is operably linked to a promoter which mediates expression of the polynucleotide in the eukaryotic cell. The recombinase polypeptide can also be introduced into the eukaryotic cell by messenger RNA that encodes the recombinase polypeptide. It is generally preferred that the recombinase be present for only such time as is necessary for insertion of the nucleic acid fragments into the genome being modified. Thus, the lack of permanence associated with most expression vectors is not expected to be detrimental. One can introduce the recombinase gene into the cell before, after, or simultaneously with, the introduction of the exogenous polynucleotide of interest. In one embodiment, the recombinase gene is present within the vector that carries the polynucleotide that is to be inserted; the recombinase gene can even be included within the polynucleotide. In other embodiments, the recombinase gene is introduced into a transgenic eukaryotic organism. Transgenic cells or animals can be made that express a recombinase constitutively or under cell-specific, tissue-specific, developmental-specific, organelle-specific, or small molecule-inducible or repressible promoters. The recombinases can be also expressed as a fusion protein with other peptides, proteins, nuclear localizing signal peptides, signal peptides, or organelle-specific signal peptides (e.g., mitochondrial or chloroplast transit peptides to facilitate recombination in mitochondria or chloroplast).

For example, a recombinase can be from the Integrase or Resolvase families. The Integrase family of recombinases has over one hundred members and includes, for example, FLP, Cre, and lambda integrase. The Integrase family, also referred to as the tyrosine family or the lambda integrase family, uses the catalytic tyrosine's hydroxyl group for a nucleophilic attack on the phosphodiester bond of the DNA. Typically, members of the tyrosine family initially nick the DNA, which later forms a double strand break. Examples of tyrosine family integrases include Cre, FLP, SSV1, and lambda (λ) integrase. In the resolvase family, also known as the serine recombinase family, a conserved serine residue forms a covalent link to the DNA target site (Grindley, et al., (2006) Ann Rev Biochem 16:16).

In one embodiment, the recombinase is an isolated polynucleotide sequence comprising a nucleic acid sequence that encodes a recombinase selecting from the group consisting of a SPβc2 recombinase, a SF370.1 recombinase, a Bxb1 recombinase, an A118 recombinase and a ΦRv1 recombinase. Examples of serine recombinases are described in detail in U.S. Pat. No. 9,034,652, hereby incorporated by reference in its entirety.

In one embodiment, a method for site-specific insertion of an exogenous polynucleotide in a genome of a cell comprises introducing a GEMS construct comprising a GEMS sequence comprising a plurality of first recognition sequences for a site-specific recombinase to generate a genetically engineered cell comprising a GEMS sequence inserted in its genome. In some embodiments, the method further comprises providing a nucleic acid sequence comprising a second recognition sequence for the site specific recombinase (e.g., Bxb1) and an exogenous polynucleotide; contacting the first and second recognition sequences with the site-specific recombinase polypeptide, resulting in a site-specific recombination between at least one of the plurality of first recognition sequence and the second recognition sequence. In some embodiments, the site specific recombination results in site-specific insertion of the exogenous polynucleotide within or adjacent to the GEMS sequence inserted within the genome of the cell. In some embodiments, the first recognition sequence is attP or attB. In some embodiments, the second recognition sequence is attB or attP. In some embodiments, the site specific recombinase is selected from the group consisting of a Listeria monocytogenes phage recombinase, a Streptococcus pyogenes phage recombinase, a Bacillus subtilis phage recombinase, a Mycobacterium tuberculosis phage recombinase and a Mycobacterium smegmatis phage recombinase. In some embodiments, the first recognition sequence is attB, and the second recognition sequence is attP. In some embodiments, the first recognition sequence is attP, and the second recognition sequence is attB. In an embodiment the recombinase is selected from the group consisting of an A118 recombinase, a SF370.1 recombinase, a SPβc2 recombinase, a ΦRy1 recombinase, and a Bxb1 recombinase. In one embodiment the recombination results in integration.

Further embodiments provide for the introduction of a site-specific recombinase into a cell whose genome is to be modified. One embodiment relates to a method for inserting multiple copies of an exogenous polynucleotide in a eukaryotic cell comprises providing a eukaryotic cell that comprises a plurality first recognition sequence for a site specific recombinase inserted in an insertion site in its genome. In some embodiments, the method further comprises providing the cell comprising a plurality of first recognition sequence for a recombinase with a plurality of a donor vector comprising a second recognition sequence for the site-specific recombinase and an exogenous polynucleotide, wherein the exogenous polynucleotide comprises a nucleic acid sequence encoding a modified selectable marker polypeptide; introducing in the cell the site specific recombinase polypeptide or a nucleic acid sequence encoding the site specific recombinase polypeptide, resulting in site specific recombination between the second recognition sequence of each of the plurality of donor vector and a select first recognition sequence from the plurality of first recognition sequences, wherein the site-specific recombination results in integration of multiple copies of the exogenous polypeptide in the genome of the cell.

In some embodiments, the first recognition sequence is attP or attB. In some embodiments, the second recognition sequence is attB or attP. In some embodiments, the site specific recombinase is selected from the group consisting of a Listeria monocytogenes phage recombinase, a Streptococcus pyogenes phage recombinase, a Bacillus subtilis phage recombinase, a Mycobacterium tuberculosis phage recombinase and a Mycobacterium smegmatis phage recombinase. In some embodiments, the first recognition sequence is attB, and the second recognition sequence is attP. In some embodiments, the first recognition sequence is attP, and the second recognition sequence is attB. In an embodiment the recombinase is selected from the group consisting of an A118 recombinase, a SF370.1 recombinase, a SPβc2 recombinase, a ΦRy1 recombinase, and a Bxb1 recombinase. In one embodiment the recombination results in integration.

### Site-Specific Recombinases:

Recombinases can be classified into two distinct families: serine recombinases (e.g., resolvases, integrases and invertases) and tyrosine recombinases (e.g., integrases), based on distinct biochemical properties. Serine recombinases and tyrosine recombinases are further divided into bidirectional recombinases and unidirectional recombinases. Bi-directional recombinase performs reversible site-specific recombination, and are therefore can also be called as reversible recombinase. Unidirectional recombinase performs irreversible recombination, and therefore can also be called irreversible recombinase. Accordingly, in some embodiments, a recombinase is a bi-directional recombinase, or a unidirectional recombinase. In some embodiments, a serine recombinase is a bidirectional serine recombinase or a unidirectional serine recombinase. In some embodiments, a tyrosine recombinase is a bi-directional tyrosine recombinase or a unidirectional serine recombinase.

Examples of bidirectional serine recombinases include, without limitation, β-six, CinH, ParA and γδ; and examples of unidirectional serine recombinases include, without limitation, Bxb1, φC31, TP901, TG1, φBT1, R4, φRV1, φFC1, MR11, A118, U153 and gp29. The serine recombinase family also has two distinct members with the division being based on size of the enzyme. The small serine recombinase sub-family contains β-six (Diaz et al. 2001), γδ-res (Schwikardi and Droge 2000), CinH-RS2 (Kholodii 2001; Thomson and Ow 2006) and ParA-MRS (Gerlitz et al. 1990; Thomson et al. 2009), where β, γδ, CinH and ParA are small serine recombinases, and six,res, RS2 and MRS are the respective DNA recognition sequences. While recombination mediated by these small serine recombinases (a.k.a. resolvases) utilizes identical recognition sites only intra-molecular excision events are observed. Therefore, an excision event mediated by the small serine recombinases is considered irreversible. The large serine recombinase sub-family is represented by phiC31 (Thomason et al. 2001; Rubtsova et al. 2008), TP901-1 (Stoll et al. 2002), R4 (Olivares et al. 2001) and Bxb1 (Kim et al. 2003; Keravala et al. 2006; Thomson and Ow 2006). These enzymes act on two recognition sequences that differ in sequence, typically known as recognition sites attB and attP, to yield hybrid product sites known as attL and attR. Excision, inversion or integration reactions can occur, but because the recognition site sequences of attB and attP are changed to attL and attR, the reverse reaction cannot occur. Therefore, phiC31, TP901-1, R4, and Bxb1 are considered examples of irreversible serine recombinase. A reversal of the reaction is only possible though the addition of a second protein, the corresponding excisionase (Thorpe et al. 2000; Ghosh et al. 2006).

Examples of bidirectional tyrosine recombinases include, without limitation, Cre, FLP, and R; and unidirectional tyrosine recombinases include, without limitation, Lambda, HK101, HK022 and pSAM2. The serine and tyrosine recombinase names stem from the conserved nucleophilic amino acid residue that the recombinase uses to attack the DNA and which becomes covalently linked to the DNA during strand exchange. The first and best-characterized group has members that include the Cre-lox (Sauer and Henderson 1990), FLP-FRT (Golic and Lindquist 1989) and R-RS (Onouchi et al. 1991) systems where Cre, FLP and R are bidirectional tyrosine recombinases and lox, FRT, and RS are the respective identical DNA recognition sequences (i.e. recognition sequences the enzymes recognize to perform site-specific recombination). Within this bidirectional tyrosine sub-family, the recombinase-mediated genetic cross-over occurs between the two identical recognition sequences. Because of the identical nature of the recognition sequences the recombination reaction is fully reversible, although intra-molecular recombination (excision) is highly favored over inter-molecular reactions (integration). The unidirectional tyrosine recombinase has non-identical recognition sites typically known as attB (attachment site bacteria) and attP (attachment site phage) and performs irreversible recombination in the absence of a helper protein, termed an excisionase. Nonlimiting examples of unidirectional tyrosine recombinases include HK022 (Kolot et al. 1999; Gottfried et al. 2005) and a modified form of (Christ and Droge 2002).

The outcome of site-specific recombination depends, in part, on the location and orientation of two DNA recognition sequences that are to be recombined, when contacted with the recombinase. Recombinases can also be classified as irreversible or reversible. As used herein, an “irreversible recombinase” refers to a recombinase that can catalyze recombination between two complementary recombination sequences (e.g., a first recognition sequence and a second recognition sequence, but cannot catalyze recombination between the hybrid sites that are formed by this recombination without the assistance of an additional factor. Thus, an “irreversible recognition site” refers to a recognition sequence for a recombinase that can serve as the first of two DNA recognition sequences for an irreversible recombinase and that is modified to a hybrid recognition site following recombination at that site. A “complementary irreversible recognition site” refers to a recombinase recognition site that can serve as the second of two DNA recognition sequences for an irreversible recombinase and that is modified to a hybrid recombination site following homologous recombination at that site. For example, attB and attP, described below, are the irreversible recombination sites for Bxb1 and phiC31 recombinases—attB is the complementary irreversible recombination site of attP, and vice versa. Recently, it was shown that the attB/attP sites can be mutated to create orthogonal B/P pairs that only interact with each other but not the other mutants. This allows a single recombinase to control the excision or integration or inversion of multiple orthogonal B/P pairs.

The phiC31 (φC31) integrase, for example, catalyzes only the attB×attP reaction in the absence of an additional factor not found in eukaryotic cells. The recombinase cannot mediate recombination between the attL and attR hybrid recombination sites that are formed upon recombination between attB and attP. Because recombinases such as the phiC31 integrase cannot alone catalyze the reverse reaction, the phiC31 attB×attP recombination is stable. Irreversible recombinases, and nucleic acids that encode the irreversible recombinases, are described in the art and can be obtained using routine methods. Examples of irreversible recombinases include, without limitation, phiC31 (φC31) recombinase, coliphage P4 recombinase, coliphage lambda integrase, Listeria A118 phage recombinase, and actinophage R4 Sre recombinase, HK101, HK022, pSAM2, Bxb1, TP901, TG1, φBT1, φRV1, φFC1, MR11, U153 and gp29.

Conversely, a “reversible recombinase” refers to a recombinase that can catalyze recombination between two complementary recombinase recognition sites and, without the assistance of an additional factor, can catalyze recombination between the sites that are formed by the initial recombination event, thereby reversing it. The product-sites generated by recombination are themselves substrates for subsequent recombination. Examples of reversible recombinase systems include, without limitation, the Cre-lox and the Flp-frt systems, R, β-six, CinH, ParA and γδ.

The recombinases provided herein are not meant to be exclusive examples of recombinases that can be used in embodiments of the invention. Any recombinase known to those skilled in the art, new orthogonal recombinases or designed synthetic recombinases with defined DNA specificities can be useful in the embodiments of the disclosure.

In some embodiments, the recombinase is a serine recombinase. In some embodiments, the recombinase is a tyrosine recombinase. In some embodiments, the serine recombinase is a serine integrase. In some embodiments, the serine recombinase is a Bxb1 integrase, a phiBT1, a R4 integrase, a TP901 integrase, gamma-delta resolvase, Tn3 resolvase, a phiC31 integrase, γδ resolvase, Gin invertase. In some embodiments, the tyrosine recombinase is a tyrosine integrase. In some embodiments, the tyrosine recombinase is a Cre recombinase, a Flp-recombinase, a XerC recombinase, a integrase, a HK022 integrase, a P22 integrase, a HP1 integrase, a L5 integrase.

In some embodiments, the recombinase is Bxb1 recombinase. In some embodiments, a nucleic acid sequence encoding a recombinase comprises a sequence that is at least 45%, 50%, 55%, 60%, 65%, 70%, 75%, 80%, 85%, 90%, 95%, 97%, 98%, 99% or 100% identical to SEQ ID NO: 108.

### Introducing Site-Specific Recombinase

In some embodiments, the methods of the disclosure comprise introducing a site-specific recombinase polypeptide or a vector comprising a nucleic acid sequence encoding the site-specific recombinase polypeptide into a host cell. Methods of introducing functional proteins into cells are well known in the art. Introduction of purified site-specific recombinase polypeptide ensures a transient presence of the polypeptide and its function. Alternatively, a nucleic acid sequence encoding the site-specific recombinase can be included in an expression vector used to transform the cell.

In some embodiments, the site-specific recombinases used in the practice of the methods of present disclosure can be introduced into a cell (e.g., a genetically engineered cell comprising a GEMS sequence) before, concurrently with, or after the introduction of a donor vector comprising an exogenous polynucleotide. In some embodiments, a donor vector comprises an exogenous polynucleotide and a second recognition site for the site-specific recombinase. In some embodiments, a donor vector comprises an exogenous polynucleotide and a second recognition site for the site-specific recombinase. In some embodiments, the site-specific recombinases used in the practice of the methods of present disclosure can be introduced into a cell (e.g., a genetically engineered cell comprising a GEMS sequence) before, concurrently with, or after the introduction of a GEMS construct. The recombinase can be directly introduced into a cell as a polypeptide, for example, using liposomes, coated particles, or microinjection. Alternately, a nucleic acid sequence encoding the recombinase can be introduced into the cell using a suitable expression vector. The donor vector components described herein are useful in the construction of vector comprising nucleic acid sequences encoding a recombinase of interest. Expression of a recombinase is typically desired to be transient. Accordingly, vectors providing transient expression of a recombinase are preferred in the practice of the present invention. However, expression of the recombinase can be regulated in other ways, for example, by placing the expression of the recombinase under the control of a regulatable promoter (i.e., a promoter whose expression can be selectively induced or repressed). In some embodiments, a recombinase can be introduced into a cell that comprises at least a first recognition sequence for the recombinase at which a site-specific recombination is desired by any suitable method. In some embodiments, a recombinase can be introduced into a cell that comprises a first recognition sequence and a second recognition sequence between which a site-specific recombination is desired upon contact by the recombinase. Methods of introducing functional proteins, e.g., by microinjection or other methods, into cells are well known in the art. Introduction of purified recombinase protein ensures a transient presence of the protein and its function, which is preferred in some embodiments. Alternatively, a nucleic acid sequence encoding a recombinase can be included in an expression vector used to transform the cell, in which the nucleic acid sequence encoding the recombinase is operably linked to a promoter which mediates expression of the nucleic acid sequence in the cell. The recombinase polypeptide can also be introduced into the eukaryotic cell by messenger RNA that encodes the recombinase polypeptide.

It is generally preferred that the recombinase be present for only such time as is necessary for insertion of an exogenous polynucleotide of interest into the genome being modified. Thus, the lack of permanence associated with most expression vectors is not expected to be detrimental. In some embodiments, a nucleic acid sequence encoding a recombinase is present within the vector (e.g., donor vector) that carries the exogenous polynucleotide that is to be inserted. In some embodiments, a nucleic acid sequence encoding a recombinase can be included within the exogenous polynucleotide. In other embodiments, a nucleic acid sequence encoding a recombinase is introduced into a transgenic organism, e.g., a transgenic plant, animal, fungus, or the like, which is then crossed with an organism that contains the corresponding recognition sequences for the recombinase. Transgenic cells or animals can be made that express a recombinase constitutively or under cell-specific, tissue-specific, developmental-specific, organelle-specific, or small molecule-inducible or repressible promoters. In some embodiments, a recombinase can be expressed as a fusion protein with other peptides, proteins, nuclear localizing signal peptides, signal peptides, or organelle-specific signal peptides (e.g., mitochondrial or chloroplast transit peptides to facilitate recombination in mitochondria or chloroplast). In some embodiments, a nucleic acid encoding a recombinase comprises a sequence that is at least 45%, 50%, 55%, 60%, 65%, 70%, 75%, 80%, 85%, 90%, 95%, 97%, 98%, 99% or 100% identical to SEQ ID NO: 108.

Sequences encoding recombinases useful in the practice of the present invention are known and include, but are not limited to, the following: Cre—Sternberg, et al., J. Mol. Biol. 187:197-212; φC31—Kuhstoss and Rao, J. Mol. Biol. 222:897-908, 1991; TP901-1—Christiansen, et al., J. Bact. 178:5164-5173, 1996; R4—Matsuura, et al., J. Bact. 178:3374-3376, 1996, Bxb1—Russell JP et al. Biotechniques. 2006 April; 40(4):460, 462, 464. In some embodiments, recombinases for use in the methods of the present disclosure can be produced recombinantly or purified as previously described. Polypeptides having the desired recombinase activity can be purified to a desired degree of purity by methods known in the art of protein ammonium sulfate precipitation, purification, including, but not limited to, size fractionation, affinity chromatography, HPLC, ion exchange chromatography, heparin agarose affinity chromatography (e.g., Thorpe & Smith, Proc. Nat. Acad. Sci. 95:5505-5510, 1998.).

### Recombinase Recognition Sites

Non-limiting examples of recognition sequence for a site-specific recombinase include lox sites, att sites, dif sites and frt sites. Typically, two different recognition sequences (termed “complementary sites”); a first recognition sequence and a second recognition sequence, are involved in a site-specific recombination event. Recognition sequences for a given recombinase are well known in the art. For example, six, res, RS2 and MRS are the respective DNA recognition sequences for serine recombinases, β, γδ, CinH and ParA respectively. Two recognition sequences that differ in sequence, typically known as recognition sites attB and attP, are known recognition sequences for the serine recombinase sub-family represented by phiC31, TP901-1, R4 and Bxb1. The sequence of attB for different recombinases is different. The sequence of attP for different recombinases is different. In some embodiments, the sequence of attB and attP for a given recombinase is different. The recombinases recognizes and binds specific attB and attP sequence and mediates a site specific recombination between these recognition sequences to yield hybrid product sites known as attL and attR. Additional examples of recognition sequences include lox, FRT, and RS that are recognition sequences for Cre, FLP and R respectively. Recognition sites for HK022 and a modified form of known as attB (attachment site bacteria) and attP (attachment site phage). As it related to the instant disclosure, a plurality of one recognition sequence, for example, a plurality of first recognition sequence for a given site-specific recombinase is present, in the GEMS sequence disclosed herein and another, for example, the second recognition sequence for the same site-specific recombinase is present on the donor vector comprising an exogenous polynucleotide that is to be integrated within or adjacent to the GEMS sequence upon site-specific recombination between at least one of the plurality of first recognition sequence provided in the GEMS sequence and second recognition sequence provided in the donor vector. The terms “attB” and “attP,” which refer to attachment sites or recognition site for recombination, originally from a bacterial target and a phage donor, respectively, are used herein although recombination sites for particular recombinase can have different names. Upon site-specific recombination between a attB and a attP site, and concomitant integration of an exogenous polynucleotide at a target recognition sequence, the recombination sites that flank the integrated exogenous polynucleotide are referred to as “attL” and “attR.” In general, the recombinases contemplated in the disclosure involve two recognition sites, one that is positioned in the integration site (the site into which a nucleic acid is to be integrated), such as a GEMS sequence inserted into a genome of a cell and another adjacent a nucleic acid of interest to be introduced into the integration site (e.g., a donor vector). AttB and attP sites are general names for the recombination site pairs (e.g., a first recognition sequence and a second recognition sequence) that are recognized by and recombined by recombinases, such as, phiC31 and Bx1. In general, the sequence of attB and attP sites are different from each other and further the sequences of attB and attP sites pairs that recognized and recombined by different recombinases (e.g., integrates) are different. The length of the recognition sequence can vary, and includes, for example, sequences that are at least 10, 12, 14, 16, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more nucleotides in length.

For example, a first recognition sequence for a site-specific recombinase can be provided in the genome of a host cell as a GEMS sequence comprising a plurality of first recognition sequences, and a second recognition sequence for the site-specific recombinase can be present adjacent to the exogenous polynucleotide sequence of interest (e.g., on a donor vector). In one aspect provided herein is a GEMS construct comprising a plurality of first recognition sequences for a site specific recombinase. In one aspect provided herein is a donor vector comprising an exogenous polynucleotide and a second recognition site for the site specific recombinase. In some embodiments, the first recognition sequence is an attB site and the second recognition sequence is an attP site. In some embodiments, the first recognition sequence is an attB site and the second recognition sequence is an attP site. In some embodiments, at least 2, 3, 4, 5, 6, 7, 8, 9, 10, 11, 12, 13, 14, 15, 16, 17, 18, 19, 20, 25, or more of the plurality of first recognition sequences is an attB site. In some embodiments, at least 2, 3, 4, 5, 6, 7, 8, 9, 11, 12, 13, 14, 15, 16, 17, 18, 19, 20, 25, or more of the plurality of first recognition sequences is an attP site. In some embodiments, each of the plurality of recognition sequences for a site-specific recombinase is the same. In some embodiments, each of the plurality of first recognition sequences is an attP site, or an attB site. In some embodiments, each of the plurality of first recognition sequences is heterologous to the genome of a cell in which it is to be inserted or is inserted (e.g., a genetically engineered cell comprising a GEMS sequence comprising a plurality of first recognition sequences). In some embodiments, at least one of the plurality of first recognition sequences is heterologous to the genome of a cell in which it is to be inserted or is inserted (e.g., a genetically engineered cell comprising a GEMS sequence comprising a plurality of first recognition sequences). In some embodiments, the second recognition sequence is heterologous to the genome of a cell in which it is introduced for recombination with a first recognition sequence of the plurality of first recognition sequences. In some embodiments, each of the plurality of first recognition sequences is an attP site, or an attB site. In some embodiments, a second recognition sequence is an attP site, or an attB site. In some embodiments, a nucleic acid sequence encoding an attP site is at least 45%, 50%, 55%, 60%, 65%, 70%, 75%, 80%, 85%, 90%, 95%, 97%, 98%, 99% or 100% identical to SEQ ID NO: 106. In some embodiments, a nucleic acid sequence encoding an attP site is at least 45%, 50%, 55%, 60%, 65%, 70%, 75%, 80%, 85%, 90%, 95%, 97%, 98%, 99% or 100% identical to SEQ ID NO: 107. In some embodiments at least 1, 2, 3, 4, 5, 6, 7, 8, 9, 10, 11, 12, 13, 14, 15, 16, 17, 18, 19, 20, 25, or more first recognition sequence of the plurality of first recognition sequences for a site-specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 106 or 107. In some embodiments, each of the first recognition sequences of the plurality of first recognition sequences for a site-specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 106 or 107. In some embodiments, a second recognition sequence comprises a nucleic acid sequence that is at least 45%, 50%, 55%, 60%, 65%, 70%, 75%, 80%, 85%, 90%, 95%, 97%, 98%, 99% or 100% identical to SEQ ID NO: 106 or 107. In some embodiments, at least one of the first recognition sequences of the plurality of the first recognition sequences for a site-specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 106, and the second recognition sequence for a site specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 107. In some embodiments, at each of the plurality of the first recognition sequences for a site-specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 106, and the second recognition sequence for a site specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 107. In some embodiments, at least one of the first recognition sequences of the plurality of the first recognition sequences for a site-specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 107, and the second recognition sequence for a site specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 106. In some embodiments, at each of the plurality of the first recognition sequences for a site-specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 107, and the second recognition sequence for a site specific recombinase comprises a nucleic acid sequence that is at least 45%, 50%, 55%, 60%, 65%, 70%, 75%, 80%, 85%, 90%, 95%, 97%, 98%, 99% or 100% identical to SEQ ID NO: 106.

In an embodiment, the GEMS construct comprises a plurality of first recognition sequences for a site-specific recombinase. In some embodiments, each of the plurality of first recognition sequences for a site-specific recombinase can undergo site-specific recombination with a second recognition sequence of the same site specific recombinase (e.g., Bxb1). The term “recognition site(s) and “recognition sequence(s)” are used interchangeably herein. In an embodiment, the GEMS construct can further comprise a polynucleotide spacer or a plurality of polynucleotide spacers which separates at least one of the first recognition sequences from the plurality of first recognition sequences for a site-specific recombinase from an adjacent first recognition sequence. In an embodiment, the GEMS construct can further comprise a polynucleotide spacer or a plurality of polynucleotide spacers which separates at least one of the nuclease recognition sequences from the plurality of nuclease recognition sequences for a site-specific recombinase from an adjacent nuclease recognition sequence. The polynucleotide spacer can be about 2 to about 10,000 nucleotides in length. The polynucleotide spacer can be about 25 to about 50 nucleotides in length. The polynucleotide spacer can be about 2 nucleotides, about 5 nucleotides, about nucleotides, about 15 nucleotides, about 20 nucleotides, about 25 nucleotides, about 30 nucleotides, about 35 nucleotides, about 40 nucleotides, about 45 nucleotides, about 50 nucleotides, about 60 nucleotides, about 70 nucleotides, about 80 nucleotides, about 90 nucleotides, about 100 nucleotides, about 1,000 nucleotides, about 2,000 nucleotides, about 3,000 nucleotides, about 4,000 nucleotides, about nucleotides, about 6,000 nucleotides, about 7,000 nucleotides, about 8,000 nucleotides, about 9,000 nucleotides, and about 10,000 nucleotides in length. In some cases, a first polynucleotide spacer separating a first recognition sequence from an adjacent first recognition sequence is the same sequence as a second polynucleotide spacer separating the first recognition sequence from another adjacent first recognition sequence of the plurality of first recognition sequence for a site-specific recombinase. In some cases, a first polynucleotide spacer separating a first recognition sequence from an adjacent first recognition sequence has a different sequence than a second polynucleotide spacer separating the first recognition sequence from another adjacent first recognition sequence of the plurality of first recognition sequence for a site-specific recombinase.

In an embodiment, the GEMS construct comprises a plurality of first recognition sequences for a site-specific recombinase that allow for insertion of one or more donor nucleic acid sequences or one or more of exogenous polynucleotides into the chromosome at e.g., the safe harbor region via the GEMS sequence. In some embodiments, the one or more exogenous polynucleotide can comprise a gene, or a portion thereof, encoding any polypeptide of interest or portion thereof. The gene can encode, for example, a therapeutic polypeptide, or an immune protein, or a signal protein, or any other protein that the practitioner intends to be expressed in the host cell. In some embodiments, the therapeutic polypeptide is an antibody or a fragment thereof. In some embodiments, the therapeutic protein is a CD19 CAR. In some embodiments, the GEMS construct comprises a GEMS sequence of SEQ ID NO: 1, or SEQ ID NO: 3. In some embodiments, the GEMS construct comprises a GEMS sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. In some embodiments, the plurality of first recognition sequences can comprise at least 3, 4, 5, 6, 7, 8, 9, 10, 11, 12, 13, 14, 15, 16, 17, 18, 19, 25, or more first recognition sequences. In some embodiments, the plurality of first recognition sequences can be unique first recognition sequences. In some embodiments, at least one of the plurality of first recognition sequences can be heterologous to the genome. In some embodiments, each of said plurality of first recognition sequences can be heterologous to the genome. In some embodiments, at least one of the plurality of first recognition sequences can be selected, for example, from SEQ ID NOs: 106, 107, or reverse complements thereof. In some embodiments, each of the plurality of nuclease recognition sequences can be selected from SEQ ID NOs: 106, 107, or reverse complements thereof.

In some embodiments, the plurality of first recognition sequences can comprise, at least 3, 4, 5, 6, 7, 8, 9, 10, 11, 12, 13, 14, 15, 16, 17, 18, 19, 20, 25, or more attB sites. In some embodiments, the plurality of first recognition sequences can comprise, at least 3, 4, 5, 6, 7, 8, 9, 10, 11, 12, 13, 14, 15, 16, 17, 18, 19, 20, 25, or more attP sites. In some embodiments, at least one of the plurality of first recognition sequence can be heterologous to the genome. In other embodiments, each of the plurality of first recognition sequences can be heterologous to the genome. The first recognition sequence can be from about 10 to about 500 nucleotides in length, from about 20 to about 300 nucleotides in length, from about 30 to about 200 nucleotides in length, and from about 40 to about 100 nucleotides in length.

In some embodiments, the GEMS construct comprises a first flanking insertion, a second flanking insertion sequence or both that is homologous to a sequence of a safe harbor site (e.g., Rosa26, AAVS1, CCR5, Hipp11 (H11) site) of a host cell genome. In some embodiments, the first flanking insertion sequence can be Rosa26 5′ homology arm sequence comprising a nucleotide sequence of SEQ ID NO: 7. In some embodiments, the second flanking insertion sequence can be Rosa26 3′ homology arm sequence comprising a nucleotide sequence of SEQ ID NO: 8. In some embodiments, Rosa26 CRISPR targeting sequence comprises a nucleotide sequence of SEQ ID NO: 9. In some embodiments, Rosa26 CRISPR gRNA sequence comprises a nucleotide sequence of SEQ ID NO: 10.

The number of first recognition sequences in the GEMS construct can vary. In an embodiment, the GEMS construct comprises a plurality of first recognition sequences. In an embodiment, the plurality of first recognition sequences is a plurality of serine recombinase recognition sequences and/or tyrosine recombinase recognition sequences. In an embodiment, the plurality of first recognition sequences is a plurality of serine integrase recognition sequences and/or tyrosine integrase recognition sequences. In an embodiment, the plurality of first recognition sequences is a plurality of BxB1 recombinase recognition sequences. The GEMS construct can comprise at least two first recognition sequences. The GEMS construct can comprise at least three first recognition sequences. The GEMS construct can comprise at least four first recognition sequences. The GEMS construct can comprise at least five first recognition sequences. The GEMS construct can comprise at least six first recognition sequences. The GEMS construct can comprise at least seven first recognition sequences. The GEMS construct can comprise at least eight first recognition sequences. The GEMS construct can comprise at least nine first recognition sequences. The GEMS construct can comprise at least ten first recognition sequences. The GEMS construct can comprise more than ten first recognition sequences. The GEMS construct can comprise more than fifteen first recognition sequences. The GEMS construct can comprise more than twenty first recognition sequences. The GEMS construct can comprise a plurality of first recognition sequences, wherein each of first recognition sequences are different from each other. In some embodiments, the GEMS construct comprises a GEMS sequence of SEQ ID NO: 1, or SEQ ID NO: 3. In some embodiments, the GEMS construct comprises a GEMS sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. In some embodiments, the GEMS construct comprises a nucleotide sequence of SEQ ID NO: 1, and/or SEQ ID NO: 105. In some embodiments, the GEMS construct comprises a homology arm sequence that is homologous to a sequence of a safe harbor site (e.g., Rosa26, AAVS1, CCR5) of a host cell genome. In some embodiments, the Rosa26 5′ homology arm sequence comprises a nucleotide sequence of SEQ ID NO: 7. In some embodiments, the Rosa26 3′ homology arm sequence comprises a nucleotide sequence of SEQ ID NO: 8.

### Methods of Present Disclosure:

In some embodiments, provided herein is a GEMS construct comprising a GEMS sequence comprising a plurality of first recognition sequences for a site-specific recombinase. In some embodiments, provided herein is a method of producing a genetically engineered cell comprising a GEMS sequence, comprising introducing into a host cell, a GEMS construct provided herein. In some embodiments, provided herein is a genetically engineered cell comprising a GEMS sequence comprising a plurality of first recognition sequences for a site-specific recombinase. In some embodiments, at least one of the plurality of first recognition sequences can undergo a site-specific recombination with a second recognition sequence for the site-specific recombinase, when contacted with the site-specific recombinase. In some embodiments, each of the plurality of first recognition sequences can undergo a site-specific recombination with a second recognition sequence for the site-specific recombinase, when contacted with the site-specific recombinase. Therefore, in some embodiments, the methods of the present disclosure involve a site specific recombination between a first recognition sequence and a second recognition sequence of a site specific recombinase, upon contact with the site-specific recombinase. The recombinase then mediates a site specific recombination between the first recognition sequence and the second recognition sequence. As it relates to the present disclosure, in some embodiments, the first recognition sequence is at least one of a plurality of first recognition sequences of the GEMS sequence provided herein. In some embodiments, the second recognition sequence is provided on a donor vector comprising an exogenous polynucleotide that is to be integrated within or adjacent to the GEMS sequence by a site specific recombination. Accordingly, the site-specific recombination between the first and second recognition sequence results in integration of an exogenous polynucleotide within or adjacent to a GEMS sequence comprising a plurality of first recognition sequences. Thus, one can obtain integration of a donor vector that contains one recombination site (e.g., a second recognition sequence) into a genetically engineered cell's genome that includes the corresponding recombination site i.e., a genetically engineered cell comprising a GEMS sequence comprising a plurality of first recognition sequence. In some embodiments, the integration is stable. Such methods are useful, for example, for obtaining stable integration into the eukaryotic chromosome of a transgene that is present on the donor vector.

Targeted integration of transgenes into predefined genetic loci is a desirable goal for many applications. In some embodiments, provided herein is a method for site-specific integration of an exogenous polynucleotide into a genome of a host cell. First, a GEMS construct comprising a GEMS sequence comprising a plurality of first recognition sequences for a site-specific recombinase is inserted at a genomic site, either at a random or at a predetermined location (e.g., a safe harbor site). This step allows generation of a genetically engineered cell comprising a GEMS sequence comprising the plurality of first recognition sequences inserted at a genomic site, either at a random or at a predetermined location (e.g., a safe harbor site). Subsequently, the cells are transfected with a donor vector carrying the exogenous polynucleotide of interest and the second recombination site and a source for recombinase (expression plasmid, RNA, protein, or virus expressing recombinase). Recombination between the first and second recognition sequences leads to integration of exogenous polynucleotide of interest.

Provided herein is a method for producing a genetically engineered cell. In some embodiments, the method for producing a genetically engineered cell comprises introducing into a host cell, a GEMS construct comprising a plurality of first recognition sequences for a site-specific recombinase disclosed herein, wherein at least one of plurality of first recognition sequences can undergo a site-specific recombination with a second recognition sequence for the site-specific recombinase, when contacted with the site-specific recombinase. In some embodiments, the methods of producing a genetically engineered cell further comprises introducing into a host cell, a donor vector comprising a desired exogenous polynucleotide to be integrated into the genome of the host cell and a second recognition sequence for the site specific recombinase. In some embodiments, the methods of producing a genetically engineered cell comprises introducing into a genetically engineered cell comprising a GEMS sequence comprising a plurality of first recognition sequences for a site-specific recombinase, a donor vector comprising a desired exogenous polynucleotide to be integrated into the genome of the host cell and a second recognition sequence for the site specific recombinase. In some embodiments, the methods of producing a genetically engineered cell further comprises introducing into the host cell or a genetically engineered cell comprising a GEMS sequence comprising a plurality of first recognition sequences, a site specific recombinase or a vector comprising a nucleic acid sequence encoding the site specific recombinase. In some embodiments, introduction of the site-specific recombinase results in a site-specific recombination between at least one of the plurality of first recognition sequences and the second recognition sequence, when contacted with the site specific recombinase. In some embodiments, the site specific recombination results in site-specific insertion of the exogenous polynucleotide, within or adjacent to the GEMS sequence, for example, within the at least one of the plurality of first recognition sequence that undergoes site specific recombination with the second recognition sequence. Accordingly, in some embodiments, the methods for producing a genetically engineered cell further comprises culturing host cell under conditions permissive for the site specific recombination. In some embodiments, the methods of generating a genetically engineered cell further comprises culturing under selective conditions that require expression of a selectable marker polypeptide (e.g., a modified selectable marker polypeptide). In some embodiments, the methods of generating a genetically engineered cell further comprises selecting a cell cultured under selective conditions that expresses a selectable marker polypeptide (e.g., a modified selectable marker polypeptide).

The methods of the disclosure are also useful for obtaining translocations of chromosomes. For example, in these embodiments, a plurality of first recognition sequence for a site-specific recombinase is inserted within one chromosome (e.g., within a safe harbor locus) and a second recognition sequence for the site specific recombinase that can serve as a substrate for site-specific recombination with at least one of the plurality of first recognition sequence is placed on a second chromosome. Upon contacting a first recognition sequence from the plurality of first recognition sequence and the second recognition sequence with a recombinase, site-specific recombination occurs that results in swapping of the two chromosome arms. For example, one can construct two strains of an organism, a first strain which includes the plurality of a first recognition sequence in its chromosome and a second strain that contains the second recognition sequence and a polynucleotide of interest (e.g., an exogenous polynucleotide). The two strains are then crossed, to obtain a progeny strain that comprises the chromosome of the first strain with the exogenous polynucleotide. Upon contacting the attachment sites with the recombinase, chromosome arm swapping occurs.

### Site-Specific Integration of an Exogenous Polynucleotide:

In one aspect, the present disclosure relates to a method for a site-specific recombination comprising: providing a host cell with a. a GEMS construct comprising a plurality of first recognition sequences for a recombinase; and b. a donor vector comprising an exogenous polynucleotide of interest and a second recognition sequence for a recombinase, or providing a genetically engineered cell comprising a GEMS sequence comprising a plurality of first recognition sequences for a recombinase inserted into its genome; with a donor vector comprising an exogenous polynucleotide of interest and a second recognition sequence for the recombinase. In some embodiments of the method, at least one of the plurality of first recognition sequences can undergo a site specific recombination with the second recognition sequence of the site specific recombinase, when contacted with the site specific recombinase. In some embodiments, the method further comprises introducing a source recombinase (e.g., a recombinase polypeptide or a vector comprising a nucleic acid sequence encoding a recombinase) resulting in site specific recombination between at least one of the plurality of first recognition sequences and the second recognition sequence, wherein the recombinase polypeptide can mediate the site-specific recombination between at least one of the plurality of first recognition sequences and the second recognition sequence. In some embodiments, the site specific recombination results in site-specific integration of the exogenous polynucleotide within or adjected to the GEMS sequence comprising the plurality of first recognition sequences for a recombinase, for example within or adjacent to the at least one of the plurality of first recognition sequences for a recombinase. Accordingly, in some embodiments, provided herein is a method for site-specific integration of an exogenous polynucleotide.

The present disclosure further comprises methods for obtaining a genetically engineered cell (e.g., a genetically engineered cell comprising a stably integrated exogenous polynucleotide sequence), the method comprising: providing a host cell with a. a GEMS construct comprising a plurality of first recognition sequences for a recombinase, and b. a donor vector comprising an exogenous polynucleotide of interest and a second recognition sequence for a recombinase; or providing a genetically engineered cell comprising a GEMS sequence comprising a plurality of a first recognition sequence for a recombinase inserted into its genome; with a donor vector comprising an exogenous polynucleotide of interest and a second recognition sequence for a recombinase. In some embodiments of the method, at least one of the plurality of first recognition sequences can undergo a site specific recombination with the second recognition sequence of the site specific recombinase, when contacted with the site specific recombinase. In some embodiments, the method further comprises introducing a source recombinase (e.g., a recombinase polypeptide or a vector comprising a nucleic acid sequence encoding a recombinase) resulting in site specific recombination between at least one of the plurality of first recognition sequences and the second recognition sequence, wherein the recombinase polypeptide can mediate the site-specific recombination between at least one of the plurality of first recognition sequences and the second recognition sequence. In some embodiments, the site specific recombination results in stable integration of the exogenous polynucleotide within or adjected to the GEMS sequence comprising the plurality of first recognition sequences, for example within or adjacent to the at least one of the plurality of first recognition sequences.

### Methods for Simultaneous Insertion of Plurality of Exogenous Polynucleotide

Provided herein is a method for simultaneously inserting a plurality of exogenous polynucleotides in a genome of a host cell, the method comprising; (a) providing a genetically engineered cell comprising a GEMS sequence comprising a plurality of first recognition sequence for a site specific recombinase inserted in its genome, (b) introducing into the genetically engineered cell (i) a plurality of a donor vector, wherein each donor vector comprises a second recognition sequence for the site specific recombinase, an exogenous polynucleotide and a nucleic acid sequence encoding a modified selectable marker polypeptide and (ii) the site specific recombinase or a vector comprising a nucleic acid sequence encoding the recombinase, (c) culturing said genetically engineered cell from step (b), and selecting a cell cultured in step (c) that expresses the modified selectable marker polypeptide, wherein the culturing of step (c) comprises culturing under conditions permissive for the site specific recombination between the second recognition sequence of each of the plurality of donor vectors and a selected first recognition sequence from the plurality of first recognition sequences, when contacted with the site specific recombinase, and wherein the culturing of step (c) comprises culturing under selective conditions that require expression of the modified selectable marker polypeptide, to thereby result in simultaneous insertion of a plurality of exogenous polynucleotides in the genome of the cell. The method results in multiple site-specific recombinations between the plurality of first recognition sequences and the second recognition sequence on the plurality of donor vector. Accordingly, in one aspect provided herein is a method for obtaining multiple site-specific recombinations in a cell. By multiple is meant at least 2, 3, 4, 5, 6, 7, 8, 9, 10, 11, 12, 13, 14, 15, 16, 27, 18, 19, 20, 22, 24, 25, 30, 35, 40, 45, 50 or more site-specific recombinations. It is understood that the number of recombinations is limited only by the number of first recognition sequences in the plurality of first recognition sequences provided an excess of donor vector is provided to the cell. In some embodiments, the plurality of exogenous polynucleotides comprises at least 2, 3, 4, 5, 6, 7, 8, 9, 10, 11, 12, 13, 14, 15, 16, 27, 18, 19, 20, 22, 24, 25, 30, 35, 40, 45, 50 or more exogenous polynucleotide. In some embodiments, at least 2, 3, 4, 5, 6, 7, 8, 9, 10, 11, or more exogenous polynucleotides in the plurality of polynucleotides encode a same product (e.g., a recombinant protein, a therapeutic polypeptide). In some embodiments, each of the exogenous polynucleotide in the plurality of exogenous polynucleotide encode the same product (e.g., a recombinant protein, a therapeutic polypeptide). By plurality of donor vector is meant at least 2, 3, 4, 5, 6, 7, 8, 9, 10, 11, 12, 13, 14, 15, 16, 27, 18, 19, 20, 22, 24, 25, 30, 35, 40, 45, 50 or more copies of donor vectors. In some embodiments, at least 2, 3, 4, 5, 6, 7, 8, 9, 10, 11, or more copies of donor vector comprising an exogenous polynucleotide that encode a same product (e.g., a recombinant protein, a therapeutic polypeptide). In some embodiments, each of the donor vector of the plurality of donor vector comprise an exogenous polynucleotide that encode the same product (e.g., a recombinant protein, a therapeutic polypeptide). In some embodiments, each exogenous polynucleotide is inserted in the genome of the cell simultaneously, for example by a single introduction of the plurality of donor vector and the recombinase.

As used herein, the term “simultaneous,” when used with respect to multiple integration, encompasses a period of time beginning at the point at which a recombinase or a vector comprising a nucleic acid sequence encoding a recombinase, and a plurality of donor vector comprising an exogenous polynucleotide to be integrated into a cell's genome is introduced into a cell (e.g., introduced into a genetically engineered cell comprising a GEMS sequence), and ending at the point at which the cell, or clonal populations thereof, is selected for successful integration of the exogenous polynucleotide from the plurality of donor vectors within or adjacent a GEMs sequence. In some embodiments, the period of time encompassed by “simultaneous” is at least the amount of time required for the recombinase to bind the first recognition sequence and the second recognition sequence and mediate or catalyze a site specific recombination between the first and second recognition sequences. In some embodiments, the period of time encompassed by “simultaneous” is at least 6, 12, 24, 36, 48, 60, 72, 96 or more than 96 hours, beginning at the point at which a recombinase or a vector comprising a nucleic acid sequence encoding a recombinase, and a plurality of donor vector comprising an exogenous polynucleotide to be integrated into a cell's genome is introduced into a cell (e.g., introduced into a genetically engineered cell comprising a GEMS sequence). In another aspect, a method for simultaneously inserting a plurality of exogenous polynucleotides in a host cell comprises; (a) providing a host cell, i. a GEMS construct comprising a plurality of first recognition sequences for a site-specific recombinase, ii. a plurality of a donor vector, wherein each donor vector comprises a second recognition sequence for the site specific recombinase, an exogenous polynucleotide and a nucleic acid sequence encoding a modified selectable marker polypeptide and iii. the site specific recombinase or a vector comprising a nucleic acid sequence encoding the recombinase, (b) culturing said host cell from step (a), and (c) selecting a cell cultured in step (b) that expresses the modified selectable marker polypeptide, wherein the culturing of step (b) comprises culturing under conditions permissive for the site specific recombination between the second recognition sequence of each of the plurality of donor vectors and a selected first recognition sequence from the plurality of first recognition sequences, when contacted with the site specific recombinase, and wherein the culturing of step (b) comprises culturing under selective conditions that require expression of the modified selectable marker polypeptide, to thereby result in simultaneous insertion of a plurality of exogenous polynucleotides in the genome of the cell.

Provided herein is a method for generating a genetically engineered cell comprising multiple copies of an exogenous polynucleotide inserted in its genome, the method comprising (a) providing a genetically engineered cell comprising a GEMS sequence comprising a plurality of first recognition sequence for a site specific recombinase inserted in its genome, (b) introducing into the genetically engineered cell (i) a plurality of a donor vector comprising a second recognition sequence for the recombinase disclosed herein, an exogenous polynucleotide and a nucleic acid sequence encoding a modified selectable marker polypeptide and (ii) the site specific recombinase or a vector comprising a nucleic acid sequence encoding the recombinase, (c) culturing said genetically engineered cell from step (b), and selecting a cell cultured in step (c) that expresses the modified selectable marker polypeptide, wherein the culturing of step (c) comprises culturing under conditions permissive for the site specific recombination between the second recognition sequence of each of the plurality of donor vectors and a selected first recognition sequence from the plurality of first recognition sequences, when contacted with the site specific recombinase, and wherein the culturing of step (c) comprises culturing under selective conditions that require expression of the modified selectable marker polypeptide, to thereby generate a genetically engineered cell comprising multiple copies of an exogenous polynucleotide inserted in its genome, for e.g., within or adjacent to the GEMS sequence. In some embodiments, the plurality of exogenous polynucleotides comprises at least 2, 3, 4, 5, 6, 7, 8, 9, 10, 11, 12, 13, 14, 15, 16, 27, 18, 19, 20, 22, 24, 25, 30, 35, 40, 45, 50 or more exogenous polynucleotide. In some embodiments, at least 2, 3, 4, 5, 6, 7, 8, 9, 10, 11, or more exogenous polynucleotides in the plurality of polynucleotides encode a same product (e.g., a recombinant protein, a therapeutic polypeptide). In some embodiments, each of the exogenous polynucleotide in the plurality of exogenous polynucleotide encode the same product (e.g., a recombinant protein, a therapeutic polypeptide). By plurality of donor vector is meant at least 2, 3, 4, 5, 6, 7, 8, 9, 10, 11, 12, 13, 14, 15, 16, 27, 18, 19, 20, 22, 24, 25, 30, 35, 40, 45, 50 or more copies of donor vectors. In some embodiments, at least 2, 3, 4, 5, 6, 7, 8, 9, 10, 11, or more copies of donor vector comprising an exogenous polynucleotide that encode a same product (e.g., a recombinant protein, a therapeutic polypeptide). In some embodiments, each of the donor vector of the plurality of donor vector comprise an exogenous polynucleotide that encode the same product (e.g., a recombinant protein, a therapeutic polypeptide). In another aspect provided herein is, a method for generating a genetically engineered cell comprising multiple copies of an exogenous polynucleotide inserted in its genome; (a) providing a host cell, i. a GEMS construct comprising a plurality of first recognition sequences for a site-specific recombinase, ii. a plurality of a donor vector, wherein each donor vector comprises a second recognition sequence for the site specific recombinase, an exogenous polynucleotide and a nucleic acid sequence encoding a modified selectable marker polypeptide and iii. the site specific recombinase or a vector comprising a nucleic acid sequence encoding the recombinase, (b) culturing said host cell from step (a), and (c) selecting a cell cultured in step (b) that expresses the modified selectable marker polypeptide, wherein the culturing of step (b) comprises culturing under conditions permissive for the site specific recombination between the second recognition sequence of each of the plurality of donor vectors and a selected first recognition sequence from the plurality of first recognition sequences, when contacted with the site specific recombinase, and wherein the culturing of step (b) comprises culturing under selective conditions that require expression of the modified selectable marker polypeptide, to thereby result in simultaneous insertion of a plurality of exogenous polynucleotides in the genome of the cell.

The copy number of a vector, an expression cassette, an amplification unit, a gene or indeed any defined exogenous polynucleotide is the number of identical copies that are present in a host cell at any time. By multiple copies is meant that an exogenous polynucleotide can be present in 2, 3, 4, 5, 6, 7, 8, 9, 10, 11, 12, 13, 14, 15, 16, 27, 18, 19, 20, 22, 24, 25, 30, 35, 40, 45, 50 or more copies in the genome. An autonomously replicating vector can be present in one, or several hundred copies per host cell. For autonomous replication, the vector can further comprise an origin of replication enabling the vector to replicate autonomously in the host cell in question. Examples of bacterial origins of replication are the origins of replication of plasmids pBR322, pUC19, pACYC177, pACYC184, pUBHO, pE194, pTA1060, and pAMI31.

### Methods of Introduction

Nucleic acids of the disclosure, for example, a construct (e.g., a GEMS construct), a vector (e.g., a donor vector disclosed herein), nucleic acid sequence (e.g., nucleic acid sequence encoding a recombinase) or a polynucleotide can be introduced in a cell by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267: 963-967; Wu and Wu, 1988, J. Biol. Chem. 263: 14621-14624; and Hartmut et al., Canadian Patent Application No. 2,012,311, filed Mar. 15, 1990). For the past decade, there has been increasing use of liposomes for encapsulation and transfection of nucleic acids in vitro. Synthetic cationic lipids can be used to prepare liposomes for in vivo transfection (Feigner et al., 1987, Proc. Natl. Acad. Sci. U.S.A. 84: 7413; Mackey, et al., 1988, Proc. Natl. Acad. Sci. U.S.A. 85:8027-8031; and Ulmer et al., 1993, Science 259: 1745-1748). The use of cationic lipids can promote encapsulation of negatively charged nucleic acids, and also promote fusion with negatively charged cell membranes (Feigner and Ringold, 1989, Science 337:387-388). Particularly useful lipid compounds and compositions for transfer of nucleic acids are described in International Patent Publications W095/18863 and W096/17823, and in U.S. Pat. No. 5,459,127. The use of lipofection to introduce exogenous genes into the specific organs in vivo has certain practical advantages.

Molecular targeting of liposomes carrying nucleic acids of the disclosure to specific cells can be beneficial. In some embodiments, directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules could be coupled to liposomes chemically. Other molecules are also useful for facilitating transfection of a nucleic acid in vivo, such as a cationic oligopeptide (e.g., W095/21931), peptides derived from DNA binding proteins (e.g., W096/25508), or a cationic polymer (e.g., W095/21931). It is also possible to introduce a vector in vivo as a naked DNA plasmid (see U.S. Pat. Nos. 5,693,622, 5,589,466 and 5,580,859). Receptor-mediated DNA delivery approaches can also be used (Curiel et al., 1992, Hum. Gene Ther. 3: 147-154; and Wu and Wu, 1987, J. Biol. Chem. 262: 4429-4432).

The term “transfection” means the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell. “Transformation” refers to the transfer of a nucleic acid fragment into the genome of a host organism, resulting in genetically stable inheritance.

Suitable means for introducing the nucleic acids of the disclosure in a cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, nucleic acids of the disclosure, for example, a construct (e.g., a GEMS construct), a vector (e.g., a donor vector disclosed herein), nucleic acid sequence (e.g., nucleic acid sequence encoding a recombinase) or a polynucleotide can be introduced in a cell by nucleofection. In another embodiment, nucleic acids of the disclosure can be introduced into the cell by microinjection. For example, by microinjection into the nucleus or the cytoplasm of a cell (e.g., host cell). Alternatively, by microinjection into a pronucleus of a one cell embryo.

In embodiments, different nucleic acids of the disclosure can be introduced simultaneously or sequentially. For example, one or more of nucleic acids selected from a GEMS construct disclosed herein, a donor vector disclosed herein and a recombinase or a vector comprising a nucleic acid sequence encoding the recombinase, can be introduced simultaneously or sequentially. In some embodiments, the nucleic acid sequence encoding a recombinase is provided on the donor vector comprising an exogenous polynucleotide and a second recognition sequence for a recombinase.

### Cell Culture Conditions

In some embodiments, the methods of the present disclosure comprise culturing cells under conditions permissive for a site specific recombination between the at least one of the plurality of first recognition sequences for a site-specific recombinase and the second recognition sequence for the site-specific recombinase, when contacted with the site specific recombinase. In some embodiments, the methods of the present disclosure comprise culturing cells under selective conditions that require expression of a selectable marker polypeptide (e.g., a modified selectable marker polypeptide). In general, the cell will be maintained under conditions appropriate for the particular cell. Suitable cell culture conditions are well known in the art and are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the type of cell to be cultured. For example, cells can be suspended in any appropriate nutrient medium, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture can contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Conditions that promote the survival of cells are typically permissive of nonhomologous end joining and homologous recombination, and site-specific recombination. Routine optimization can be used, in all cases, to determine the best techniques for a particular cell type. In some embodiments, culturing can be carried out for at least 5 min, 10 min, 15 min, min, 30 min, 45 min, 1 hour, 2, hours, 5 hours, 10 hours, 12 hours, 15 hours, 20 hours, 24 hours, 48 hours or more. In some embodiments, culturing can be carried out for at least 1 day, 2 days, 3 days, 4 days, 5 days, 10 days, 15 days, 20 days, 25 days, 1 month, or more. In some embodiments, at the end of culturing period the culture medium can be replaced with fresh medium and cells can be cultured further.

In some embodiments, the methods of the present disclosure comprise culturing cells under selective conditions. Any appropriate selective conditions that require expression of corresponding selectable marker polypeptide (e.g., a WT selectable marker polypeptide or a modified selectable marker polypeptide) can be employed to select cells, e.g., cells that have been targeted by nucleic acid compositions of the subject application (e.g., constructs and vectors disclosed herein). Where the selectable marker polypeptide is one that induces resistance to an antibiotic, the selective conditions can comprise for example, culturing cells in presence of the antibiotic. In some embodiments, the methods disclosed herein comprise selecting cells expressing a selectable marker polypeptide. In some embodiments, the selection can be positive selection; that is, the genetically engineered cells are isolated from a population, e.g. to create an enriched population of genetically engineered cells comprising the genetic modification. In other instances, the selection can be negative selection; that is, the population is isolated away from the genetically engineered cells, e.g. to create an enriched population of cells that do not comprise the genetic modification. Any convenient selectable marker polypeptide can be employed, for example, a drug selectable marker polypeptide, e.g. a marker polypeptide that prevents cell death in the presence of a drug, a marker polypeptide that promotes cell death in the presence of a drug, an imaging marker, etc.; an imaging marker polypeptide that can be selected for using imaging technology, e.g. fluorescence activated cell sorting; a polypeptide or peptide that may be selected for using affinity separation techniques, e.g. fluorescence activated cell sorting, magnetic separation, affinity chromatography, “panning” with an affinity reagent attached to a solid matrix, etc.; and the like. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell composition that are highly enriched in genetically engineered cells are achieved in this manner. By “highly enriched”, it is meant that the genetically engineered cells will be 70% or more, 75% or more, 80% or more, 85% or more, 90% or more of the cell composition, for example, about 95% or more, or 98% or more of the cell composition. In other words, the composition may be a substantially pure composition of genetically engineered cells.

Genetically engineered cells produced by the methods described herein can be used immediately. Alternatively, the genetically engineered cells can be frozen or lyophilized at liquid nitrogen temperatures and stored for long periods of time, being thawed and capable of being reused. In such cases, the genetically engineered cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The genetically engineered cells can be cultured in vitro under various culture conditions. The genetically engineered cells can be expanded in culture, i.e. grown under conditions that promote their proliferation. Culture medium can be liquid or semi-solid, e.g. containing agar, methylcellulose, etc. The genetically engineered cells can be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Cells that have been genetically engineered can be transplanted to a human subject for purposes such as gene therapy, e.g. to treat a disease, or as an antiviral, antipathogenic, or anticancer therapeutic. The subject may be a neonate, a juvenile, or an adult. The genetically engineered cells of the present invention can be formulated into cell compositions that are pharmaceutical compositions that include a pharmaceutically acceptable carrier. Examples of pharmaceutically acceptable carriers include saline, buffers, diluents, fillers, salts, stabilizers, solubilizers, cell culture medium, and other materials which are well known in the art. In some embodiments, the formulations are free of detectable DMSO (dimethyl sulfoxide).

### Constructs and Vectors

The constructs (e.g., GEMS construct) and vector (e.g., donor vector) disclosed herein can comprise additional nucleic acid fragments such as control sequences, nucleic acid sequences encoding a selectable marker polypeptide, a reporter gene encoding a reporter polypeptide and the like as discussed below.

In certain cases, the reporter gene encodes a “reporter polypeptide”. A reporter polypeptide refers to a protein that can be used to measure expression of exogenous polypeptide of interest typically fused in frame with the reporter protein. A reporter polypeptide generally produces a measurable signal such as fluorescence, luminescence or color and can be used to locate and, optionally, visualize cells, e.g. cells that have been targeted by nucleic acid compositions (constructs and vectors) of the subject application. In some embodiments, a reporter gene encodes a bioluminescent protein, a chromogenic protein or a fluorescent protein. The presence of a reporter polypeptide (e.g., a bioluminescent protein, a chromogenic protein or a fluorescent protein) in a cell or organism is readily observed. For example, fluorescent proteins (e.g., GFP) cause a cell to fluoresce when excited with light of a particular wavelength, luciferases cause a cell to catalyze a reaction that produces light, and enzymes such as β-galactosidase convert a substrate to a colored product. Reporter polypeptide for use in accordance with the disclosure include any reporter polypeptide, e.g., a bioluminescent protein, a chromogenic protein, a fluorescent protein described herein or known to one of ordinary skill in the art.

There are several different ways to measure or quantify a reporter polypeptide depending on the particular reporter polypeptide and what kind of characterization data is desired. In some embodiments, microscopy can be a useful technique for obtaining both spatial and temporal information on reporter activity, particularly at the single cell level. In some embodiments, flow cytometers can be used for measuring the distribution in reporter activity across a large population of cells. In some embodiments, plate readers may be used for taking population average measurements of many different samples over time. In some embodiments, instruments that combine such various functions, may be used, such as multiplex plate readers designed for flow cytometers, and combination microscopy and flow cytometric instruments.

Fluorescence from florescence proteins can be readily quantified using a microscope, plate reader or flow cytometer equipped to excite the fluorescent protein with the appropriate wavelength of light. Several different fluorescent proteins are available, thus multiple gene expression measurements can be made in parallel. Examples of reporter genes encoding fluorescent proteins that can be used in accordance with the disclosure include, without limitation, those proteins provided in U.S. Patent Application No. 2012/0003630 (see Table 59), incorporated herein by reference. In some embodiments, a reporter gene encodes a fluorescent protein. In some embodiments, the fluorescent protein is a green fluorescent protein, a red fluorescent protein, a yellow fluorescent protein, a blue fluorescent protein, a cyan fluorescent protein, or an orange fluorescent protein.

Luciferases can also be used for visualizing or quantifying a signal from a bioluminescent protein for detecting and measuring expression levels of polypeptide of interest (e.g., encoded by an exogenous polynucleotide) fused in frame with a bioluminescent protein, as cells tend to have little to no background luminescence in the absence of a luciferase. Luminescence can be readily quantified using a plate reader or luminescence counter. Examples of reporter genes encoding a bioluminescent protein (e.g., luciferases) that can be used in accordance with the disclosure include, without limitation, dmMyD88-linker-Rluc, dmMyD88-linker-Rluc-linker-PEST191, and firefly luciferase (from Photinus pyralis).

Enzymes that produce colored substrates (“colorimetric enzymes” or chromogenic proteins) can also be used as reporter polypeptide. Enzymatic products can be quantified using spectrophotometers or other instruments that can take absorbance measurements including plate readers. Like luciferases, enzymes such as β-galactosidase can be used for detecting and measuring expression of levels of a polypeptide of interest (e.g., encoded by an exogenous polynucleotide) fused in frame with a bioluminescent protein. Examples of reporter genes encoding colorimetric enzymes or chromogenic proteins that can be used in accordance with the disclosure include, without limitation, lacZ alpha fragment, lacZ (encoding beta-galactosidase, full-length), and xylE. A chromogenic protein can require addition of a substrate for detection, e.g., horseradish peroxidase (HRP), β-galactosidase, and the like. A bioluminescent protein can require addition of substrate such as luciferin. Alternatively, a fluorescent protein can provide a detectable signal that does not require the addition of a substrate for detection, e.g. a fluorophore or chromophore dye, e.g. Alexa Fluor 488® or Alexa Fluor 647®, or a protein that comprises a fluorophore or chromophore, e.g. a fluorescent protein. As used herein, a fluorescent protein (FP) refers to a protein that possesses the ability to fluoresce (i.e., to absorb energy at one wavelength and emit it at another wavelength). For example, a green fluorescent protein (GFP) refers to a polypeptide that has a peak in the emission spectrum at 510 nm or about 510 nm. A variety of FPs that emit at various wavelengths are known in the art. FPs of interest include, but are not limited to, a green fluorescent protein (GFP), yellow fluorescent protein (YFP), orange fluorescent protein (OFP), cyan fluorescent protein (CFP), blue fluorescent protein (BFP), red fluorescent protein (RFP), far-red fluorescent protein, or near-infrared fluorescent protein and variants thereof.

A nucleic acid sequence encoding a selectable marker polypeptide or selectable marker, for use in the present invention encodes a protein that can be used for selection of genetically engineered cells, e.g. because upon expression of the protein in the genetically engineered cell, it provides a growth advantage to the cell expressing the selectable marker polypeptide, as compared to a corresponding cell that does not. In some embodiments, a nucleic acid sequence encoding a selectable marker polypeptide provides resistance to a selection agent (e.g. an antibiotic) upon expression of the encoded selectable marker polypeptide in a cell (e.g., a genetically engineered cell), which selection agent causes lethality and/or growth inhibition of a cell not expressing the selectable marker polypeptide. The selectable marker according to the invention must thus be functional in a cell of interest to be genetically engineered, and hence being capable of being selected for. Any selectable marker polypeptide fulfilling this criterion can in principle be used according to the present invention. Such selectable markers are well known in the art and routinely used when cell clones are to be obtained, and several examples are provided herein.

For convenience and as generally accepted by the skilled person, in many publications as well as herein, often a nucleic acid sequence encoding a selectable marker polypeptide that causes resistance to a selection agent is referred to as the ‘selectable agent (resistance) gene’ or ‘selection agent (resistance) polypeptide’, respectively, although the official names may be different, e.g. a nucleic acid sequence encoding a selectable marker polypeptide conferring resistance to neomycin (as well as to G418 and kanamycin) is often referred to as neomycin (resistance) (or neor) gene, while the official name is aminoglycoside 3′-phosphotransferase gene, and the encoded selectable marker polypeptide is neomycin (resistance) polypeptide, while the official name is aminoglycoside 3′-phosphotransferase, APH 3′ II. Nucleic acid sequences of several resistance genes encoding a selectable marker polypeptide are known in the art and can be used in the present disclosure.

In some embodiments, the selectable marker polypeptide provides resistance against lethal or growth-inhibitory effects of a selection agent selected from the group consisting of the bleomycin family of antibiotics, puromycin, blasticidin, hygromycin, an aminoglycoside antibiotic, neomycin, hygromycin, ampicillin, kanamycin, methotrexate, and methionine sulphoximine.

A nucleic acid sequence a selectable marker providing resistance to bleomycin family of antibiotics is e.g., a nucleic acid sequence comprising a “ble” gene, including but not limited to Sh ble, Tn5 ble and Sable. In general the gene products encoded by the ble genes confer to their host resistance to the copper-chelating glycopeptide antibiotics of the bleomycin family, which are DNA-cleaving glycopeptides. Examples of antibiotics of the bleomycin family for use as selection agents in accordance with the present invention include but are not limited to bleomycin, phleomycin, tallysomycin, pepleomycin and Zeocin™. Other examples of selectable markers which can be used in the invention are selectable markers which can be used as auxotrophic (metabolic) selection markers and include e.g. a cystathionine gamma-lyase gene, a DHFR gene and a glutamine synthetase (GS) gene. A potential advantage of the use of these types of metabolic enzymes as selectable marker polypeptides is that they can be used to keep the cells under continuous selection, which can be advantageous under certain circumstances.

A “selectable marker polypeptide” means a polypeptide that upon expression in a cell induces a selective advantage or disadvantage to said cell. For example, a common selectable marker polypeptide is one that induces resistance to a particular antibiotic or a chemical. A selectable marker polypeptide can be encoded by an antibiotic or chemical resistance gene, that is able to be selected for based upon the selectable marker polypeptide's activity, i.e., induction resistance to an antibiotic, resistance to a herbicide, colorimetric signal, fluorescent signal, bioluminescent signal, and/or enzymatic activity. Non-limiting example of a selectable marker polypeptide include a polypeptide that is encoded by an antibiotic resistance gene, a herbicide resistance gene, an enzyme, a calorimetric polypeptide, a bioluminescent polypeptide, a fluorescent polypeptide, a chromogenic polypeptide, and the like, wherein the effect is used to track the inheritance of an exogenous polynucleotide of interest and/or to identify a cell or organism that has inherited the exogenous polynucleotide of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. By a “selection marker polypeptide” or “selectable marker polypeptide” it is meant an agent that can be used to select cells, e.g., cells that have been targeted by nucleic acid compositions of the subject application (e.g., constructs and vectors disclosed herein).

### Modified Selectable Marker Polypeptide:

In some embodiments, a modified selectable marker polypeptide, for example, mutants or derivatives of a selectable marker polypeptide are suitably used according to the disclosure, and are therefore included within the scope of the term ‘selectable marker polypeptide’, as long as the modified selectable marker polypeptide is still functional. As used herein a “modified selectable marker polypeptide” is a selectable marker polypeptide comprising one or more modifications that may or may not result in reduced activity (e.g., providing resistance to a selection agent) relative to a corresponding unmodified WT selectable marker polypeptide. In some embodiments, a construct (e.g., GEMS construct), or a vector (e.g., a donor vector) of the present disclosure comprises a nucleic acid sequence encoding a modified selectable marker polypeptide. In some embodiments, a modified selectable marker polypeptide comprises reduced activity compared to its wild-type counterpart (e.g., corresponding WT selectable marker polypeptide). In some embodiments, using a modified selectable marker polypeptide allows a further level of control in fine tuning of the stringency of selection of genetically engineered cells of the invention. In some embodiments, a nucleic acid sequence encoding a modified selectable marker polypeptide encodes a selectable marker polypeptide comprising one or more mutations that reduce the activity of the selectable marker polypeptide compared to its wild-type counterpart (e.g., corresponding WT selectable marker polypeptide). In some embodiments, a modified selectable marker polypeptide exhibits reduced activity by at least 2%, 5%, 10%, 15%. 20%, 25%, 30%, 35%, 40%, 45%, 50% or more relative to the corresponding WT selectable marker protein. In some embodiments, a modified selectable marker polypeptide comprises one or more modifications selected from an amino acid substitution, an amino acid insertion, an amino acid deletion, or a combination thereof. In some embodiments, the amino acid substitution can be a conservative or a non-conservative substitution. In some embodiments, the nucleic acid sequence encoding a modified selectable marker polypeptide comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 84. In some embodiments, a modified selectable marker polypeptide is a modified neomycin resistance polypeptide i.e., modified neomycin phosphotransferase. In some embodiments, a modified selectable marker polypeptide comprises a D227V amino acid substitution relative to the corresponding WT neomycin phosphotransferase. As non-limiting examples for a modified selectable marker polypeptide, proline at position 9 in the zeocin resistance polypeptide can be mutated, e.g. to Thr or Phe (see e.g. example 14 of WO 2006/048459, incorporated by reference herein), and for the neomycin resistance polypeptide, amino acid residue 182 or 261 or both may further be mutated (see e.g. WO 01/32901). In some embodiments, a modified selectable marker polypeptide with reduced activity is selected from the group consisting of: a) a zeocin resistance polypeptide wherein proline at position 9 is changed into a different amino acid; b) a zeocin resistance polypeptide wherein valine at position 10 is changed into a different amino acid; c) a zeocin resistance polypeptide wherein threonine at position 12 is changed into a different amino acid; d) a zeocin resistance polypeptide wherein arginine at position 14 is changed into a different amino acid; e) a zeocin resistance polypeptide wherein glutamic acid at position 21 is changed into a different amino acid; f) a zeocin resistance polypeptide wherein phenylalanine at position 22 is changed into a different amino acid; g) a zeocin resistance polypeptide wherein aspartic acid at position 25 is changed into a different amino acid; h) a zeocin resistance polypeptide wherein glycine at position 28 is changed into a different amino acid; i) a zeocin resistance polypeptide wherein phenylalanine at position 33 is changed into a different amino acid j) a zeocin resistance polypeptide wherein glycine at position 35 is changed into a different amino acid; k) a zeocin resistance polypeptide wherein glutamic acid at position 73 is changed into a different amino acid; 1) a zeocin resistance polypeptide wherein alanine at position 76 is changed into a different amino acid; m) a zeocin resistance polypeptide wherein valine at position 82 is changed into a different amino acid; n) a zeocin resistance polypeptide wherein aspartic acid at position 88 is changed into a different amino acid; o) a zeocin resistance polypeptide wherein methionine at position 94 is changed into a different amino acid; and, p) a neomycin resistance polypeptide wherein at least one of amino acid residue 182 and 261 is changed into a different amino acid. In some embodiments a modified selectable marker polypeptide comprises one or more silent mutation relative to a corresponding WT selectable marker polypeptide that do not alter the encoded protein because of the redundancy of the genetic code. Further mutations that lead to conservative amino acid mutations or to other mutations are also encompassed, as long as the encoded modified selectable marker polypeptide still has activity, which may or may not be lower than that of a corresponding wild-type selectable marker polypeptide. In some embodiments, a modified selectable marker polypeptide comprises an amino acid sequence that is at least 60%, at least 70%, at least 80%, at least 90%, or at least 95% identical to a corresponding WT selectable marker polypeptide. Testing for activity of the selectable marker proteins can be done by routine methods.

A variety of vectors (e.g., donor vectors) are suitable for use in the practice of the present disclosure both for prokaryotic expression and eukaryotic expression. In some embodiments, the constructs and vectors of the present disclosure can comprise one or more of the following features: a promoter, promoter-enhancer sequences, a selection marker sequence, an origin of replication, an inducible element sequence, an epitope-tag sequence, and the like.

Promoter and promoter-enhancer sequences are DNA sequences to which RNA polymerase binds and initiates transcription. The promoter determines the polarity of the transcript by specifying which strand will be transcribed. Bacterial promoters consist of consensus sequences, −35 and −10 nucleotides relative to the transcriptional start, which are bound by a specific sigma factor and RNA

polymerase. Eukaryotic promoters are more complex. Most promoters utilized in expression vectors are transcribed by RNA polymerase II. General transcription factors (GTFS) first bind specific sequences near the start and then recruit the binding of RNA polymerase IL In addition to these minimal promoter elements, small sequence elements are recognized specifically by modular DNA-binding/trans-activating proteins (e.g. AP-1, SP-1) that regulate the activity of a given promoter. Viral promoters serve the same function as bacterial or eukaryotic promoters and either provide a specific RNA polymerase in trans (bacteriophage T7) or recruit cellular factors and RNA polymerase (SV40, RSV, CMV). Viral promoters can be preferred as they are generally particularly strong promoters. Promoters can be, furthermore, either constitutive or regulatable (i.e., inducible or repressible). Inducible elements are DNA sequence elements which act in conjunction with promoters and bind either repressors (e.g. lacO/LAC Iq repressor system in E. coli) or inducers (e.g.gall/GAL4 inducer system in yeast). In either case, transcription is virtually “shut off” until the promoter is repressed or induced, at which point transcription is “turned-on.” Examples of constitutive promoters include the int promoter of bacteriophage X, the bla promoter of the 13-lactamase gene sequence of pBR322, the CAT promoter of the chloramphenicol acetyl transferase gene sequence of pPR325, and the like. Examples of inducible prokaryotic promoters include the major right and left promoters of bacteriophage (PL and PR), the trp, reca, lacZ, AraC and gal promoters of E. coli, the α-amylase (Ulmanen Ett at., J. Bacteriol. 162:176-182, 1985) and the sigma-28-specific promoters of B. subtilis (Gilman et al., Gene sequence 32:11-20(1984)), the promoters of the bacteriophages of Bacillus (Gryczan, In: The Molecular Biology of the Bacilli, Academic Press, Inc., NY (1982)), Streptomyces promoters (Ward et at., Mol. Gen. Genet. 203:468-478, 1986), and the like. Exemplary prokaryotic promoters are reviewed by Glick (J. had. Microtiot. 1:277-282, 1987); Cenatiempo (Biochimie 68: 505-516, 1986); and Gottesman (Ann. Rev. Genet. 18:415-442, 1984). For example, the exogenous polynucleotide (e.g., encoding a therapeutic polypeptide of interest) can be operably linked to a suitable promoter control sequence for expression in a eukaryotic cell. The promoter control sequence can be constitutive or regulated (i.e., inducible or tissue-specific). Suitable constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (EDI)-alpha promoter, ubiquitin promoters, actin promoters, tubulin promoters, immunoglobulin promoters, fragments thereof, or combinations of any of the foregoing. Non-limiting examples of suitable inducible promoter control sequences include those regulated by antibiotics (e.g., tetracycline-inducible promoters), and those regulated by metal ions (e.g., metallothionein-1 promoters), steroid hormones, small molecules (e.g., alcohol-regulated promoters), heat shock, and the like. Non-limiting examples of tissue specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphs1 promoter, OG-2 promoter, SP-B promoter, SYN1 promoter, and WASP promoter. The promoter sequence can be wild type or it can be modified for more efficient or efficacious expression. Other control elements that may be present include additional transcription regulatory and control elements (i.e., partial promoters, promoter traps, start codons, enhancers, introns, insulators, polyA signals, termination signal sequences, and other expression elements) can also be present.

Non-limiting example of eukaryotic promoters include, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-5 95S, 1984), the CMV promoter, the EF-1 promoter, ecdysone-responsive promoter(s), tetracycline-responsive promoter, and the like. Exemplary promoters for use in the present invention are selected such that they are functional in cell type (and/or animal or plant) into which they are being introduced.

A further element useful in a vector (e.g., donor vector) or a construct) e.g., a GEMS construct is an origin of replication. Replication origins are unique DNA segments that contain multiple short repeated sequences that are recognized by multimeric origin-binding proteins and that play a key role in assembling DNA replication enzymes at the origin site. Suitable origins of replication for use in expression vectors employed herein include E. coli oriC, colE1 plasmid origin, 211 and ARS (both useful in yeast systems), sf1, SV40, EBV oriP (useful in mammalian systems), and the like.

Epitope tags are short peptide sequences that are recognized by epitope specific antibodies.

A fusion protein comprising a recombinant protein and an epitope tag can be simply and easily purified using an antibody bound to a chromatography resin. The presence of the epitope tag furthermore allows the recombinant protein to be detected in subsequent assays, such as Western blots, without having to produce an antibody specific for the recombinant protein itself. Examples of commonly used epitope tags include V5, glutathione-S-transferase (GST), hemaglutinin (HA), the peptide Phe-His-His-Thr-Thr, chitin binding domain, and the like.

A further useful element in a vector or construct is a multiple cloning site or polylinker. Synthetic DNA encoding a series of restriction endonuclease recognition sites is inserted into a plasmid vector, for example, downstream of the promoter element. These sites are engineered for convenient cloning of DNA into the vector at a specific position.

The foregoing elements can be combined to produce vectors or constructs suitable for use in the methods of the invention. Those of skill in the art would be able to select and combine the elements suitable for use in their particular system in view of the teachings of the present specification. Suitable prokaryotic vectors include plasmids such as those capable of replication in E. coli (for example, pBR322, Co1E1, pSC101, PACYC 184, itVX, PRSET, pBAD (Invitrogen, Carlsbad, Calif.) and the like). Such plasmids are disclosed by Sambrook (cf. “Molecular Cloning: A Laboratory Manual,” second edition, edited by Sambrook, Fritsch, & Maniatis, Cold Spring Harbor Laboratory, (1989)). Bacillus plasmids include pC194, pC221, pT127, and the like, and are disclosed by Gryczan (In: The Molecular Biology of the Bacilli, Academic Press, NY (1982), pp. 307-329). Suitable Streptomyces plasmids include pli101 (Kendall et al., J. Bacteriol. 169:4177-4183, 1987), and Streptonzyces bacteriophages such as (I)C31 (Chater et al., In: Sixth International Symposium on Actinomycetales Biology, Akademiai Kaido, Budapest, Hungary (1986), pp. 45-54). Pseudomonas plasmids are reviewed by John et al. (Rev. Infect. Dis. 8:693-704, 1986), and Izaki (Jpn. J. Bacteriol. 33:729-742, 1978).

Suitable eukaryotic plasmids include, for example, BPV, EBV, vaccinia, SV40, 2-micron circle, pcDNA3.1, pcDNA3.1/GS, pDual, pYES2/GS, pMT, p IND, pIND(Sp1), pVgRXR (Invitrogen), and the like, or their derivatives. Such plasmids are well known in the art (Botstein et al., Miami Wntr. SyTnp. 19:265-274, 1982; Broach, In: The Molecular Biology of the Yeast Saccharomyces: Life Cycle and Inheritance“, Cold Spring Harbor Laboratory, Cold Spring Harbor, N.Y., p. 445-470, 1981; Broach, Cell 28:203-204, 1982; Dilon et at., J. Clin. Hematol. Onco1.10: 39-48, 1980; Maniatis, In: Cell Biology: A Comprehensive Treatise, Vol. 3, Gene Sequence Expression, Academic Press, NY, pp. 563-608, 1980. The targeting cassettes described herein can be constructed utilizing methodologies known in the art of molecular biology (see, for example, Ausubel or Maniatis) in view of the teachings of the specification. In some embodiments, a donor vector can be assembled by inserting, into a suitable vector backbone, a second recognition sequence for a recombinase, polynucleotides encoding sequences of interest operably linked to a promoter of interest; and, optionally a sequence encoding a selectable marker polypeptide (e.g., a modified selectable marker polypeptide).

A preferred method of obtaining polynucleotides, including suitable regulatory sequences (e.g., promoters) is PCR. General procedures for PCR are taught in MacPherson et al., PCR: A PRACTICAL APPROACH, (IRL Press at Oxford University Press, (1991)). PCR conditions for each application reaction may be empirically determined. A number of parameters influence the success of a reaction. Among these parameters are annealing temperature and time, extension time, Mg2+ and ATP concentration, pH, and the relative concentration of primers, templates and deoxyribonucleotides. After amplification, the resulting fragments can be detected by agarose gel electrophoresis followed by visualization with ethidium bromide staining and ultraviolet illumination.

### Methods of Using Genetically Engineered Cell Comprising a GEMS Sequence

In one aspect provided herein are genetically engineered cell comprising a GEMS sequence (e.g., comprising a plurality of first recognition sequences for a recombinase, or a plurality of nuclease recognition sequences) inserted in its genome. The genetically engineered cell comprising a GEMS sequence inserted in its genome can be used for the production of a desired product encoded by an exogenous polynucleotide such as a recombinant protein, a biopharmaceutical protein, a therapeutic protein or a therapeutic polypeptide. Specifically, the plurality of recognition sequence(s) in the GEMS sequences (e.g., a plurality of first recognition sequence for a recombinase or a plurality of nuclease recognition sequence) can be targeted by a recombinase and/or a nuclease) for integration of an exogenous polynucleotide encoding the protein of interest (e.g., therapeutic polypeptide). There are several advantages to using the methods and cells described herein containing GEMS sequence that can be retargeted for the expression of a protein of interest (e.g., a recombinant protein or a therapeutic polypeptide). First, one can increase the expression of a desired protein by increasing the efficiency of the targeted integration (incorporation of the desired genetic material) by choosing a stable genomic locus or loci to insert the GEMS sequence (for subsequent retargeting). Use of a highly efficient targeting endonuclease or recombinase to integrate a desired exogenous polynucleotide into a known, stable location in the genome results not only in the efficient integration of the exogenous polynucleotide, but also the continued, stable expression of a protein encoded by the exogenous polynucleotide following integration.

Consequently, this leads to increased cell line stability and decreased clone-to-clone and molecule-to molecule (desired protein of interest) heterogeneity, resulting in overall decreased cell line development times and increased protein expression. Furthermore, using the methods described herein, it is possible to generate genetically engineered cells comprising multiple copies of an exogenous polynucleotide encoding the same desired protein (e.g., a recombinant protein or a therapeutic polypeptide) or integration of more than one different exogenous polynucleotide encoding different protein of interest, thereby providing maximal flexibility as to the protein production that can be achieved. In addition, the inclusion of optional sequences, such as selectable marker polypeptide, reporter sequences, and/or regulatory control element sequences allows one to further customize the bioproduction capability.

Thus, in a further aspect, the genetically engineered cells described herein comprising a GEMS sequence comprising a plurality of a first recognition sequence inserted in its genome, can be reengineered to express an exogenous polynucleotide encoding a polypeptide of interest, e.g., for the production of a therapeutic polypeptide of interest, the method comprising: (a) introducing into the genetically engineered cell: (i) a donor vector comprising an exogenous polynucleotide and the second recognition site for the site specific recombinase, wherein at least one of the plurality of first recognition sequence can undergo a site specific recombination with the second recognition sequence of the site specific recombinase, when contacted with the site specific recombinase, and (ii) the site specific recombinase or a vector comprising a nucleic acid sequence encoding the site specific recombinase; and (b) culturing the genetically engineered cell from step (a) under conditions permissive for the site specific recombination between the at least one of the plurality of first recognition sequences and the second recognition sequence, when contacted with the site specific recombinase, wherein the site specific recombination results in site specific insertion of the exogenous polynucleotide within the at least one of the plurality of first recognition sequence. The protein of interest (e.g., a recombinant protein or a therapeutic polypeptide) encoded by the exogenous polynucleotide can be expressed from the genetically engineered cells using standard protein expression procedures and protocols. Steps (a)(i) and (a)(ii) can be performed simultaneously or sequentially; that is, the donor comprising an exogenous polynucleotide and the second recognition site for the site specific recombinase, and the site specific recombinase or a vector comprising a nucleic acid sequence encoding the site specific recombinase can be administered to the cell at the same time or can be administered in separate steps.

In yet another aspect, the genetically engineered cells described herein comprising a GEMS sequence comprising a plurality of first recognition sequence for a site-specific recombinase inserted into its genome, can be used to generate a genetically engineered cell comprising multiple copies of an exogenous polynucleotide, for e.g., for increased production of a protein of interest (e.g., a recombinant protein or a therapeutic polypeptide), the method comprises; (a) providing a genetically engineered cell comprising a GEMS sequence comprising a plurality of first recognition sequence for a site-specific recombinase; (b) introducing into the cell (i) a plurality of donor vector comprising an exogenous polynucleotide and a second recognition sequence for the site-specific recombinase and nucleic acid sequence encoding a modified selectable marker polypeptide, and (ii) the site specific recombinase, or a vector comprising a nucleic acid sequence encoding said site specific recombinase; (c) culturing the cell under conditions permissive for the site specific recombination between the second recognition sequence of each of the plurality of donor vectors and a selected first recognition sequence from the plurality of first recognition sequences, when contacted with the site specific recombinase, and (d) selecting a cell cultured from step (b) that expresses the selectable marker polypeptide. In some embodiments, the genetically engineered cells comprising a GEMS sequence comprising a plurality of first recognition sequence for a site-specific recombinase inserted into its genome, can be further genetically engineered to generate therapeutic cells, (for example, genetically engineered cells comprising an exogenous polynucleotide chimeric antigen receptor (CAR), a T-cell receptor (TCR), a B-cell receptor (BCR), an αβ receptor, a γδ T-receptor, dopamine, insulin, proinsulin, or a portion thereof).

### Nuclease Recognition Sites

In an embodiment, the GEMS construct comprises a plurality of nuclease recognition sequences, wherein each of the plurality of nuclease recognition sequences comprises a target sequence and a protospacer adjacent motif (PAM) sequence or reverse complements thereof. The target sequence binds to a guide polynucleotide (e.g., gRNA) following insertion of the GEMS construct at the insertion site. In an embodiment, the nuclease is an endonuclease. The term “nuclease recognition site(s) and “nuclease recognition sequence(s)” are used interchangeably herein.

In an embodiment, the GEMS construct can further comprise a polynucleotide spacer or a plurality of polynucleotide spacers which separates at least one of the first recognition sequences from the plurality of first recognition sequences for a site-specific recombinase from an adjacent first recognition sequence. In an embodiment, the GEMS construct can further comprise a polynucleotide spacer or a plurality of polynucleotide spacers which separates at least one of the nuclease recognition sequences from the plurality of nuclease recognition sequences for a site-specific recombinase from an adjacent nuclease recognition sequence. The polynucleotide spacer can be about 2 to about 10,000 nucleotides in length. The polynucleotide spacer can be about 25 to about 50 nucleotides in length. The polynucleotide spacer can be about 2 nucleotides, about 5 nucleotides, about 10 nucleotides, about 15 nucleotides, about 20 nucleotides, about 25 nucleotides, about 30 nucleotides, about 35 nucleotides, about 40 nucleotides, about 45 nucleotides, about 50 nucleotides, about 60 nucleotides, about 70 nucleotides, about 80 nucleotides, about 90 nucleotides, about 100 nucleotides, about 1,000 nucleotides, about 2,000 nucleotides, about 3,000 nucleotides, about 4,000 nucleotides, about 5,000 nucleotides, about 6,000 nucleotides, about 7,000 nucleotides, about 8,000 nucleotides, about 9,000 nucleotides, and about 10,000 nucleotides in length. In some cases, a first polynucleotide spacer separating a nuclease recognition sequence from an adjacent nuclease recognition sequence is the same sequence as a second polynucleotide spacer separating the nuclease recognition sequence from another adjacent nuclease recognition sequence. In some cases, a first polynucleotide spacer separating a nuclease recognition sequence from an adjacent nuclease recognition sequence has a different sequence than a second polynucleotide spacer separating the nuclease recognition sequence from another adjacent nuclease recognition sequence.

In an embodiment, the GEMS construct comprises a GEMS sequence that comprises a plurality of nuclease recognition sequences that allow for insertion of one or more donor nucleic acid sequences into the chromosome at e.g., the safe harbor region via the GEMS sequence. In some embodiments, the one or more donor nucleic acid sequences can comprise a gene, or a portion thereof, encoding any polypeptide of interest or portion thereof. The gene can encode, for example, a therapeutic protein, or an immune protein, or a signal protein, or any other protein that the practitioner intends to be expressed in the host cell. In some embodiments, the therapeutic protein is a CD19 CAR. In some embodiments, the GEMS construct comprises a GEMS sequence of SEQ ID NO: 1, or SEQ ID NO: 3. In some embodiments, the GEMS construct comprises a GEMS sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. The plurality of nuclease recognition sequences can comprise at least 3, 4, 5, 6, 7, 8, 9, 10, 11, 12, 13, 14, 15, 16, 17, 18, 19, 20, 25, or more nuclease recognition sequences. In some embodiments, the plurality of nuclease recognition sequences can be unique nuclease recognition sequences. In some embodiments, at least one of the plurality of nuclease recognition sequences can be heterologous to the genome. In some embodiments, each of said plurality of nuclease recognition sequences can be heterologous to the genome. In some embodiments, at least one of the plurality of nuclease recognition sequences can be selected, for example, from SEQ ID NOs: 89, 91, 93, 97, 99, 101, 103, or reverse complements thereof. In some embodiments, each of the plurality of nuclease recognition sequences can be selected from SEQ ID NOs: 89, 91, 93, 95, 97, 99, 101, 103, or reverse complements thereof.

In some embodiments, the plurality of nuclease recognition sequences can comprise, at least 3, 4, 5, 6, 7, 8, 9, 10, 11, 12, 13, 14, 15, 16, 17, 18, 19, 20, 25, or more target sequences. In some embodiments, the target sequences of each of the plurality of nuclease recognition sequences can be the same, although in other embodiments, the target sequences of each of the plurality of nuclease recognition sequences can be unique. In some embodiments, at least one target sequence in the plurality of nuclease recognition sequence can be heterologous to the genome. In other embodiments, each target sequence of the plurality of nuclease recognition sequences can be heterologous to the genome. The target sequence can be from about 10 to about 30 nucleotides in length, from about 15 to about 25 nucleotides in length, and from about 17 to about 24 nucleotides in length. In some aspects, the target sequence is about 20 nucleotides in length. In some embodiments, the target sequence can be GC-rich, such that at least about 40% of the target sequence is made up of G or C nucleotides. The GC content of the target sequence can from about 40% to about 80%, though GC content of less than about 40% or greater than about 80% can be used. In some embodiments, the target sequence can be AT-rich, such that at least about 40% of the target sequence is made up of A or T nucleotides. The AT content of the target sequence can from about 40% to about 80%, though AT content of less than about 40% or greater than about 80% can be used.

In some embodiments, the GEMS construct comprises a first flanking insertion, a second flanking insertion sequence or both that is homologous to a sequence of a safe harbor site (e.g., Rosa26, AAVS1, CCR5) of a host cell genome. In some embodiments, the first flanking insertion sequence can be Rosa26 5′ homology arm sequence comprising a nucleotide sequence of SEQ ID NO: 7. In some embodiments, the second flanking insertion sequence can be Rosa26 3′ homology arm sequence comprising a nucleotide sequence of SEQ ID NO: 8. In some embodiments, Rosa26 CRISPR targeting sequence comprises a nucleotide sequence of SEQ ID NO: 9. In some embodiments, Rosa26 CRISPR gRNA sequence comprises a nucleotide sequence of SEQ ID NO: 10. The number of nuclease recognition sequences in the GEMS construct can vary. In an embodiment, the GEMS construct comprises a plurality of nuclease recognition sites. In an embodiment, the plurality of nuclease recognition sites is a plurality of Cas nuclease recognition sequences. The GEMS construct can comprise at least two nuclease recognition sites. The GEMS construct can comprise at least three nuclease recognition sequences. The GEMS construct can comprise at least four nuclease recognition sequences. The GEMS construct can comprise at least five nuclease recognition sequences. The GEMS construct can comprise at least six nuclease recognition sequences. The GEMS construct can comprise at least seven nuclease recognition sequences. The GEMS construct can comprise at least eight nuclease recognition sequences. The GEMS construct can comprise at least nine nuclease recognition sequences. The GEMS construct can comprise at least ten nuclease recognition sequences. The GEMS construct can comprise more than ten nuclease recognition sequences. The GEMS construct can comprise more than fifteen nuclease recognition sequences. The GEMS construct can comprise more than twenty nuclease recognition sequences. The GEMS construct can comprise a first nuclease recognition sequence that is different from a sequence of a second nuclease recognition sequence. The GEMS construct can comprise a plurality of nuclease recognition sequences, wherein each of nuclease recognition sequences are different from each other. In some embodiments, the GEMS construct comprises a GEMS sequence of SEQ ID NO: 1, or SEQ ID NO: 3. In some embodiments, the GEMS construct comprises a GEMS sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. In some embodiments, the GEMS construct comprises a nucleotide sequence of SEQ ID NO: 1, SEQ ID NO: 3, and/or SEQ ID NO: 105. In some embodiments, the GEMS construct comprises a homology arm sequence that is homologous to a sequence of a safe harbor site (e.g., Rosa26, AAVS1, CCR5) of a host cell genome. In some embodiments, the Rosa26 5′ homology arm sequence comprises a nucleotide sequence of SEQ ID NO: 7. In some embodiments, the Rosa26 3′ homology arm sequence comprises a nucleotide sequence of SEQ ID NO: 8.

The plurality of nuclease recognition sites can comprise a plurality of recognition sequences for a zinc finger nuclease (ZFN), a transcription activator-like effector nuclease (TALEN), a clustered regularly interspaced short palindromic repeats (CRISPR) associated nuclease (Cas), an Argonaute protein taken from Pyrococcus furiosus (PfAgo), or a combination thereof. For example, a GEMS sequence can comprise a plurality of different secondary nuclease recognition sites, which can differ in the type of nuclease that recognizes the site (e.g., ZFN, TALEN, or Cas), and which can differ among the recognition site sequences themselves. There are numerous recognition sequences for each type of nuclease, such that the multiple gene editing site can comprise different recognition sequences for the same type of endonuclease.

In some embodiments, one or more primary nuclease recognition sequences in GEMS construct can comprise a zinc finger nuclease (ZFN) recognition sequence, a transcription activator-like effector nuclease (TALEN) recognition sequence, a clustered regularly interspaced short palindromic repeats (CRISPR) associated nuclease, or a meganuclease recognition sequence. ZFNs and TALENs can be fused to the Fok1 endonuclease.

A ZFN generally comprises a zinc finger DNA binding protein and a DNA-cleavage domain. As used herein, a “zinc finger DNA binding protein” or “zinc finger DNA binding domain” is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein (ZFP). Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data.

As used herein, the term “transcription activator-like effector nuclease” or “TAL effector nuclease” or “TALEN” refers to a class of artificial restriction endonucleases that are generated by fusing a TAL effector DNA binding domain to a DNA cleavage domain. In some embodiments, the TALEN is a monomeric TALEN that can cleave double stranded DNA without assistance from another TALEN. The term “TALEN” is also used to refer to one or both members of a pair of TALENs that are engineered to work together to cleave DNA at the same site. TALENs that work together can be referred to as a left-TALEN and a right-TALEN, which references the handedness of DNA.

Meganuclease refers to a double-stranded endonuclease having a large oligonucleotide recognition site, e.g., DNA sequences of at least 12 base pairs (bp) or from 12 bp to 40 bp. The meganuclease can also be referred to as rare-cutting or very rare-cutting endonuclease. The meganuclease of the present disclosure can be monomeric or dimeric. The meganuclease can include any natural meganuclease such as a homing endonuclease, but can also include any artificial or man-made meganuclease endowed with high specificity, either derived from homing endonucleases of group I introns and inteins, or other proteins such as zinc finger proteins or group II intron proteins, or compounds such as nucleic acid fused with chemical compounds.

In some embodiments, the meganuclease can be one of four separated families on the basis of well conserved amino acids motifs, namely the LAGLIDADG family, the G1Y-YIG family, the His-Cys box family, and the HNH family (Chevalier et al., 2001, N. A. R, 29, 3757-3774). According to one embodiment, the meganuclease is a I-Dmo I, PI-Sce I, I-SceI, PI-Pfu I, I-Cre I, I-Ppo I, or a hybrid homing endonuclease I-Dmo I/I-Cre I called E-Dre I (Chevalier et al., 2001, Nat Struct Biol, 8, 312-316). In some cases, the meganuclease is the I-SceI meganuclease, which recognizes the nucleic acid sequence TAGGGATAACAGGGTAAT (SEQ ID NO: 2). In some cases, the GEMS construct comprises the I-SceI meganuclease recognition sequence (primary endonuclease recognition sequence) upstream, downstream, or both upstream and downstream of the multiple gene editing site.

In some embodiments, a host cell to which the GEMS construct is transfected is preferably competent for the endonuclease (expresses the endonuclease) such as meganuclease that recognizes the meganuclease recognition sequence. For competency, the cell can be a cell that naturally expresses the particular endonuclease that recognizes the primary recognition sequences of the construct, or the cell can be separately transfected with a gene encoding the endonuclease such that the cell expresses an exogenous endonuclease. For example, where the GEMS construct includes a ZFN recognition sequence as the primary endonuclease recognition sequence, the cell can be competent for a zinc finger nuclease, which serves as the primary endonuclease to cleave the construct for insertion of the multiple gene editing site into the chromosome. For example, where the GEMS construct includes a TALEN recognition sequence as the primary endonuclease recognition sequence, the cell can be competent for a transcription activator-like effector nuclease, which serves as the primary endonuclease to cleave the construct for insertion of the multiple gene editing site into the chromosome. For example, where the GEMS construct includes a meganuclease recognition sequence as the primary endonuclease recognition sequence, the cell can be competent for a meganuclease which serves as the primary endonuclease to cleave the construct for insertion of the GEMS sequence into the chromosome. For example, where the GEMS construct comprises the I-SceI meganuclease recognition sequence as the primary endonuclease recognition sequence, the cell to which the construct is transfected can be a I-SceI meganuclease-competent cell, and the I-SceI meganuclease serves as the primary endonuclease, which serves as the primary endonuclease to cleave the construct for insertion of the multiple gene editing site into the chromosome. In some embodiments, the GEMS construct comprises a first meganuclease recognition sequences upstream of the GEMS sequence. In some embodiments, the GEMS construct can further comprise a second meganuclease recognition sequence downstream of the GEMS sequence. The first meganuclease recognition sequence can be upstream of the first flanking insertion sequence. The second meganuclease sequence can be downstream of the second flanking insertion sequence. The second meganuclease recognition sequence can be downstream of the second flanking insertion sequence. The first meganuclease recognition sequence, the second meganuclease recognition sequence, or both can comprise an I-SceI meganuclease recognition sequence. The meganuclease recognition sequence allows the GEMS construct to be cleaved by a meganuclease in the cell in order to generate a donor sequence comprising GEMS sequence.

### CRISPR/Cas9 System

Clustered Regularly Interspaced Short Palindromic Repeats (CRISPR) is a family of DNA sequences in bacteria. The sequences contain snippets of DNA from viruses that have attacked the bacterium. These snippets are used by the bacterium to detect and destroy DNA from similar viruses during subsequent attacks. These sequences play a key role in a bacterial defense system, and form the basis of a technology known as CRISPR/Cas9 that effectively and specifically changes genes within organisms.

Methods described herein can take advantage of a CRISPR/Cas system. For example, double-strand breaks (DSBs) can be generated using a CRISPR/Cas system, e.g., a type II CRISPR/Cas system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of a target sequence.

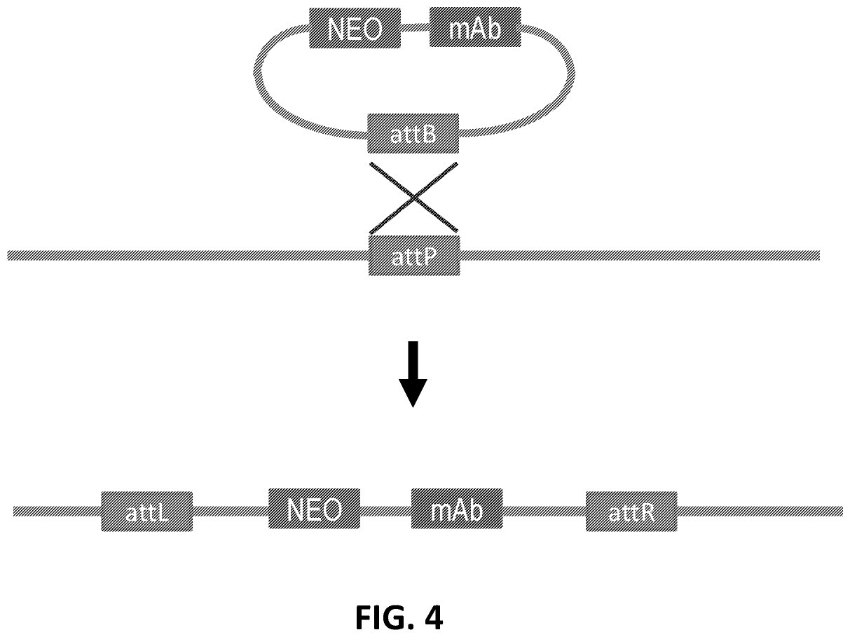

In some embodiments, the target sequence of each secondary endonuclease recognition site in the multiple gene editing site can be the same, although in some aspects, the target sequence of each secondary endonuclease recognition site can be different from other target sequences in the multiple gene editing site. The target sequence can be from about 10 to about 30 nucleotides in length, from about 15 to about 25 nucleotides in length, and from about 17 to about 24 nucleotides in length (FIGS. 4-6). In some aspects, the target sequence is about 20 nucleotides in length.

In some embodiments, the target sequence can be GC-rich, such that at least about 40% of the target sequence is made up of G or C nucleotides. The GC content of the target sequence can from about 40% to about 80%, though GC content of less than about 40% or greater than about 80% can be used. In some embodiments, the target sequence can be AT-rich, such that at least about 40% of the target sequence is made up of A or T nucleotides. The AT content of the target sequence can from about 40% to about 80%, though AT content of less than about 40% or greater than about 80% can be used.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cash, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used. Cas9 can refer to a polypeptide with at least or at least about 50%, 60%, 70%, 80%, 90%, 91%, 92%, 93%, 94%, 95%, 96%, 97%, 98%, 99%, or 100% sequence identity and/or sequence homology to a wild type exemplary Cas9 polypeptide (e.g., Cas9 from S. pyogenes). Cas9 can refer to a polypeptide with at most or at most about 50%, 60%, 70%, 80%, 90%, 91%, 92%, 93%, 94%, 95%, 96%, 97%, 98%, 99%, or 100% sequence identity and/or sequence homology to a wild type exemplary Cas9 polypeptide (e.g., from S. pyogenes). Cas9 can refer to the wild type or a modified form of the Cas9 protein that can comprise an amino acid change such as a deletion, insertion, substitution, variant, mutation, fusion, chimera, or any combination thereof.

In some embodiments, the methods described herein can utilize an engineered CRISPR system. The Engineered CRISPR system contains two components: a guide RNA (gRNA or sgRNA) or a guide polynucleotide; and a CRISPR-associated endonuclease (Cas protein). The gRNA is a short synthetic RNA composed of a scaffold sequence necessary for Cas-binding and a user-defined ˜20 nucleotide spacer that defines the genomic target to be modified. Thus, a skilled artisan can change the genomic target of the CRISPR specificity is partially determined by how specific the gRNA targeting sequence is for the genomic target compared to the rest of the genome. In some embodiments, the sgRNA is any one of sequences in SEQ ID NOs: 24-32 (Table 6). In some embodiments, the guide RNA is selected from Table 7. In some embodiments, the guide RNA targets a site in the GEMS sequence selected from SEQ ID NO: 1, or SEQ ID NO: 3. In some embodiment, the guide RNA comprises a sequence selected from SEQ ID NOs: 90, 92, 94, 96, 98, 100, 102, or 104. In some embodiments, Rosa26 CRISPR targeting sequence comprises a nucleotide sequence of SEQ ID NO: 9. In some embodiments, Rosa26 CRISPR gRNA sequence comprises a nucleotide sequence of SEQ ID NO: 10. In some embodiments, GEMS sequence targeting sequence comprises a nucleotide sequence of SEQ ID NO: 89, 91, 93, 95, 97, 99, 101 or 103. In some embodiments, GEMS sequence guide RNA sequence comprises a nucleotide sequence of SEQ ID NO: 90, 92, 94, 96, 98, 100, 102, or 104. In some embodiments, the GEMS sequence targeting sequence comprises a nucleotide sequence selected from Table 7.

The Cas9 nuclease has two functional endonuclease domains: RuvC and HNH. Cas9 undergoes a second conformational change upon target binding that positions the nuclease domains to cleave opposite strands of the target DNA. The end result of Cas9-mediated DNA cleavage is a double-strand break (DSB) within the target DNA (˜3-4 nucleotides upstream of the PAM sequence). The resulting DSB is then repaired by one of two general repair pathways: (1) the efficient but error-prone non-homologous end joining (NHEJ) pathway; or (2) the less efficient but high-fidelity homology directed repair (HDR) pathway.

The “efficiency” of non-homologous end joining (NHEJ) and/or homology directed repair (HDR) can be calculated by any convenient method. For example, in some cases, efficiency can be expressed in terms of percentage of successful HDR. For example, a surveyor nuclease assay can be used can be used to generate cleavage products and the ratio of products to substrate can be used to calculate the percentage. For example, a surveyor nuclease enzyme can be used that directly cleaves DNA containing a newly integrated restriction sequence as the result of successful HDR. More cleaved substrate indicates a greater percent HDR (a greater efficiency of HDR). As an illustrative example, a fraction (percentage) of HDR can be calculated using the following equation [(cleavage products)/(substrate plus cleavage products)] (e.g., b+c/a+b+c), where “a” is the band intensity of DNA substrate and “b” and “c” are the cleavage products.

In some cases, efficiency can be expressed in terms of percentage of successful NHEJ. For example, a T7 endonuclease I assay can be used to generate cleavage products and the ratio of products to substrate can be used to calculate the percentage NHEJ. T7 endonuclease I cleaves mismatched heteroduplex DNA which arises from hybridization of wild-type and mutant DNA strands (NHEJ generates small random insertions or deletions (indels) at the site of the original break). More cleavage indicates a greater percent NHEJ (a greater efficiency of NHEJ). As an illustrative example, a fraction (percentage) of NHEJ can be calculated using the following equation: (1-(1-(b+c/a+b+c)).sup.½).times.100, where “a” is the band intensity of DNA substrate and “b” and “c” are the cleavage products (Ran et. al., Cell. 2013 Sep. 12; 154(6):1380-9).

The NHEJ repair pathway is the most active repair mechanism, and it frequently causes small nucleotide insertions or deletions (indels) at the DSB site. The randomness of NHEJ-mediated DSB repair has important practical implications, because a population of cells expressing Cas9 and a gRNA or a guide polynucleotide can result in a diverse array of mutations. In most cases, NHEJ gives rise to small indels in the target DNA that result in amino acid deletions, insertions, or frameshift mutations leading to premature stop codons within the open reading frame (ORF) of the targeted gene. The ideal end result is a loss-of-function mutation within the targeted gene.

While NHEJ-mediated DSB repair often disrupts the open reading frame of the gene, homology directed repair (HDR) can be used to generate specific nucleotide changes ranging from a single nucleotide change to large insertions like the addition of a fluorophore or tag.

In order to utilize HDR for gene editing, a DNA repair template containing the desired sequence can be delivered into the cell type of interest with the gRNA(s) and Cas9 or Cas9 nickase. The repair template can contain the desired edit as well as additional homologous sequence immediately upstream and downstream of the target (termed left & right homology arms). The length of each homology arm can be dependent on the size of the change being introduced, with larger insertions requiring longer homology arms. The repair template can be a single-stranded oligonucleotide, double-stranded oligonucleotide, or a double-stranded DNA plasmid. The efficiency of HDR is generally low (<10% of modified alleles) even in cells that express Cas9, gRNA and an exogenous repair template. The efficiency of HDR can be enhanced by synchronizing the cells, since HDR takes place during the S and G2 phases of the cell cycle. Chemically or genetically inhibiting genes involved in NHEJ can also increase HDR frequency.

In some embodiments, Cas9 is a modified Cas9. A given gRNA targeting sequence can have additional sites throughout the genome where partial homology exists. These sites are called off-targets and need to be considered when designing a gRNA. In some embodiments, Rosa26 CRISPR targeting sequence comprises a nucleotide sequence of SEQ ID NO: 9. In some embodiments, GEMS sequence targeting sequence comprises a nucleotide sequence of SEQ ID NO: 89, 91, 93, 95, 97, 99, 101 or 103. In some embodiments, GEMS sequence guide RNA sequence comprises a nucleotide sequence of SEQ ID NO: 90, 92, 94, 96, 98, 100, 102, or 104. In some embodiments, the GEMS sequence targeting sequence is selected from Table 7. In addition to optimizing gRNA design, CRISPR specificity can also be increased through modifications to Cas9. Cas9 generates double-strand breaks (DSBs) through the combined activity of two nuclease domains, RuvC and HNH. Cas9 nickase, a D10A mutant of SpCas9, retains one nuclease domain and generates a DNA nick rather than a DSB. Thus, two nickases targeting opposite DNA strands are required to generate a DSB within the target DNA (often referred to as a double nick or dual nickase CRISPR system). This requirement dramatically increases target specificity, since it is unlikely that two off-target nicks can be generated within close enough proximity to cause a DSB. The nickase system can also be combined with HDR-mediated gene editing for specific gene edits.

In some embodiments, the modified Cas9 is a high fidelity Cas9 enzyme. In some embodiments, the high fidelity Cas9 enzyme is SpCas9(K855A), eSpCas9(1.1), SpCas9-HF1, or hyper accurate Cas9 variant (HypaCas9). The modified Cas9 eSpCas9(1.1) contains alanine substitutions that weaken the interactions between the HNH/RuvC groove and the non-target DNA strand, preventing strand separation and cutting at off-target sites. Similarly, SpCas9-HF1 lowers off-target editing through alanine substitutions that disrupt Cas9's interactions with the DNA phosphate backbone. HypaCas9 contains mutations (SpCas9 N692A/M694A/Q695A/H698A) in the REC3 domain that increase Cas9 proofreading and target discrimination. All three high fidelity enzymes generate less off-target editing than wildtype Cas9.

In some cases, Cas9 is a variant Cas9 protein. A variant Cas9 polypeptide has an amino acid sequence that is different by one amino acid (e.g., has a deletion, insertion, substitution, fusion) when compared to the amino acid sequence of a wild type Cas9 protein. In some instances, the variant Cas9 polypeptide has an amino acid change (e.g., deletion, insertion, or substitution) that reduces the nuclease activity of the Cas9 polypeptide. For example, in some instances, the variant Cas9 polypeptide has less than 50%, less than 40%, less than 30%, less than 20%, less than 10%, less than 5%, or less than 1% of the nuclease activity of the corresponding wild-type Cas9 protein. In some cases, the variant Cas9 protein has no substantial nuclease activity. When a subject Cas9 protein is a variant Cas9 protein that has no substantial nuclease activity, it can be referred to as “dCas9.”

In some cases, a variant Cas9 protein has reduced nuclease activity. For example, a variant Cas9 protein exhibits less than about 20%, less than about 15%, less than about 10%, less than about 5%, less than about 1%, or less than about 0.1%, of the endonuclease activity of a wild-type Cas9 protein, e.g., a wild-type Cas9 protein.

In some cases, a variant Cas9 protein can cleave the complementary strand of a guide target sequence but has reduced ability to cleave the non-complementary strand of a double stranded guide target sequence. For example, the variant Cas9 protein can have a mutation (amino acid substitution) that reduces the function of the RuvC domain. As a non-limiting example, in some embodiments, a variant Cas9 protein has a D10A (aspartate to alanine at amino acid position 10) and can therefore cleave the complementary strand of a double stranded guide target sequence but has reduced ability to cleave the non-complementary strand of a double stranded guide target sequence (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21).

In some cases, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a target sequence (e.g., a single stranded target sequence) but retains the ability to bind a target sequence (e.g., a single stranded target sequence).

In some cases, a variant Cas9 protein has a reduced ability to cleave both the complementary and the non-complementary strands of a double stranded target DNA. As a non-limiting example, in some cases, the variant Cas9 protein harbors both the D10A and the H840A mutations such that the polypeptide has a reduced ability to cleave both the complementary and the non-complementary strands of a double stranded target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors W476A and W1126A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors H840A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors H840A, D10A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors D10A, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

In some cases, when a variant Cas9 protein harbors W476A and W1126A mutations or when the variant Cas9 protein harbors P475A, W476A, N477A, D1125A, W1126A, and D1127A mutations, the variant Cas9 protein does not bind efficiently to a PAM sequence. Thus, in some such cases, when such a variant Cas9 protein is used in a method of binding, the method need not include a PAM-mer. In other words, in some cases, when such a variant Cas9 protein is used in a method of binding, the method can include a guide RNA, but the method can be performed in the absence of a PAM-mer (and the specificity of binding is therefore provided by the targeting segment of the guide RNA).

Other residues can be mutated to achieve the above effects (i.e. inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a variant Cas9 protein that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 protein can still bind to target DNA in a site-specific manner (because it is still guided to a target DNA sequence by a guide RNA) as long as it retains the ability to interact with the guide RNA.

Alternatives to S. pyogenes Cas9 can include RNA-guided endonucleases from the Cpf1 family that display cleavage activity in mammalian cells. CRISPR from Prevotella and Francisella 1 (CRISPR/Cpf1) is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. Unlike Cas9 nucleases, the result of Cpf1-mediated DNA cleavage is a double-strand break with a short 3′ overhang. Cpf1's staggered cleavage pattern can open up the possibility of directional gene transfer, analogous to traditional restriction enzyme cloning, which can increase the efficiency of gene editing. Like the Cas9 variants and orthologues described above, Cpf1 can also expand the number of sites that can be targeted by CRISPR to AT-rich regions or AT-rich genomes that lack the NGG PAM sites favored by SpCas9. The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alpha-helical recognition lobe of Cas9. Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Functional Cpf1 doesn't need the trans-activating CRISPR RNA (tracrRNA), therefore, only CRISPR (crRNA) is required. This benefits genome editing because Cpf1 is not only smaller than Cas9, but also it has a smaller sgRNA molecule (proximately half as many nucleotides as Cas9). The Cpf1-crRNA complex cleaves target DNA or RNA by identification of a protospacer adjacent motif 5′-YTN-3′ in contrast to the G-rich PAM targeted by Cas9. After identification of PAM, Cpf1 introduces a sticky-end-like DNA double-stranded break of 4 or 5 nucleotides overhang.

### Protospacer Adjacent Motif

The protospacer adjacent motif (PAM) or PAM-like motif refers to a 2-6 base pair DNA sequence immediately following the DNA sequence targeted by the Cas9 nuclease in the CRISPR bacterial adaptive immune system. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence is essential for target binding, but the exact sequence depends on a type of Cas protein. Non-limiting examples of Cas proteins include Cas1, Cas1B, Cas2, Cas3, Cas4, Cas5, Cas5d, Cas5t, Cas5h, Cas5a, Cash, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof.

In an embodiment, the GEMS sequence comprises a plurality of nuclease recognition sites for the CRISPR-associated endonuclease Cas9. In an embodiment, each nuclease recognition site is specific to a Cas9 enzyme from a different species of bacteria. A Cas9 nuclease recognition site can comprises a targeting sequence coupled to a nucleotide protospacer adjacent motif (PAM) sequence. In some embodiments, Rosa26 CRISPR targeting sequence comprises a nucleotide sequence of SEQ ID NO: 9. In some embodiments, the GEMS sequence targeting sequence can comprise a sequence selected from SEQ ID NOs: 89, 91, 93, 95, 97, 99, 101, 103 or reverse complements thereof. In some embodiments, the guide RNA targets a site in the GEMS sequence selected from SEQ ID NO: 1, or SEQ ID NO: 3. In some embodiment, the guide RNA comprises a sequence selected from SEQ ID NOs: 90, 92, 94, 96, 98, 100, 102, or 104. Different bacteria species encode different Cas9 nuclease proteins, which recognize different PAM sequences. Thus, to facilitate Cas9-facilitated insertion of donor genes into the multiple gene editing site, the multiple gene editing site can comprise a plurality of secondary endonuclease recognition sites for Cas9 that each comprise a target sequence coupled to a PAM sequence.

Each Cas9 nuclease target sequence can be coupled to a PAM sequence. Among the Cas9 nuclease recognition sites in the multiple gene editing site, each PAM sequence can be different from the other PAM sequences (e.g., variable PAM region and constant crRNA region), even if the target sequence is the same among the Cas9 nuclease recognition sites. In some cases, each PAM sequence can be the same as the other PAM sequences, though in such cases, the target sequence can be different among the Cas9 nuclease recognition sites (e.g., constant PAM region and variable crRNA region).

The PAM sequence can be any PAM sequence known in the art. Suitable PAM sequences include, but are not limited to, CC, NG, YG, NGG, NAA, NAT, NAG, NAC, NTA, NTT, NTG, NTC, NGA, NGT, NGC, NCA, NCT, NCG, NCC, NRG, TGG, TGA, TCG, TCC, TCT, GGG, GAA, GAC, GTG, GAG, CAG, CAA, CAT, CCA, CCN, CTN, CGT, CGC, TAA, TAC, TAG, TGG, TTG, TCN, CTA, CTG, CTC, TTC, AAA, AAG, AGA, AGC, AAC, AAT, ATA, ATC, ATG, ATT, AWG, AGG, GTG, TTN, YTN, TTTV, TYCV, TATV, NGAN, NGNG, NGAG, NGCG, AAAAW, GCAAA, TGAAA, NGGNG, NGRRT, NGRRN, NNGRRT, NNAAAAN, NNNNGATT, NAAAAC, NNAAAAAW, NNAGAA, NNNNACA, GNNNCNNA, NNNNGATT, NNAGAAW, NNGRR, and TGGAGAAT, and any variation thereof. Different PAM sequences recognized by different Cas9 enzyme species are listed in Tables 1-2.

TABLE 1
Cas Enzyme and PAM Sequences

| Species of Cas enzyme | PAM Sequence |
|---|---|
| Streptococus pyogenes (Sp); | 3′NGG |
| SpCas9 | |
| SpCas9 D1135E variant | 3′NGG (reduced |
| | NAG binding) |
| SpCas9 VRER variant | 3′NGCG |
| (D1135V, G1218R, | |
| R1335E, T1337R) | |
| SpCas9 EQR variant | 3′NGAG |
| (D1135E, R1335Q, T1337R) | |
| SpCas9 VQR variant | 3′NGAN or NGNG |
| (D1335V, R1335Q, T1337R) | |
| Staphylococcus aures (Sa); | 3′NNGRRT or |
| SaCas9 | NNGRR(N) |
| Acidaminococcus sp. | 5′TTTV |
| (AsCpf1) and | |
| Lachnospiraceae bacterium | |
| (LbCpf1) | |
| AsCpf1 RR variant | 5′TYCV |
| LbCpf1 RR variant | 5′TYCV |
| AsCpfl RVR variant | 5′TATV |
| Neisseria meningitides (Nm) | 3′NNNNGATT |
| Streptococcus thermophiles | 3′NNAGAAW |
| (St) | |
| Treponema denticola (Td) | 3′NAAAAC |
| Additional Cas9 species | PAM sequence may not |
| | be characterized |

* Y is a pyrimidine; N is any nucleotide base; W is A or T.

TABLE 2
Variable PAMs

| 5′ to 3′ Strand | | 3′ to 5′ Strand | |
|---|---|---|---|
| NGRRT | Staphylococcus aures (Sa); | NGAG (Tgag) | Staphylococcus pyogenes v1 |
| (CgAAt) | Neisseria meningitis | | EQR variant (Spv1) |
| NGGNG | Streptococcus thermophiles A | NGCG (cgcg) | Staphylococcus pyogenes |
| (CggAg) | (St-A) (CRISPR3) | | VRER variant (Svrer) |
| NAAAAC | Treponema denticola (Td) | NNNNGATT | Neiseria Meningitis (Mn) |
| (Gaaaac) | | (CTAGgatt) | |
| GCAAA | Streptococcus thermophiles B (St | NNAGAAW | Staphylococcus Thermophiles |
| | LMG18311) | (GCagaaT) | (St) |
| TGGAGAAT | | TAA | Haloferax valcanii |
| GNNNCNNA | Pasteurella multocida (Pm) | AAAAW | Staphylococcus thermophiles B |
| (gAGAcGAa) | | (aaaaT) | (StB) |
| TGAAA | Lactobacillus casei (Lc) | NNAAAAAW | |
| | | (CGaaaaaT) | |

In some embodiments, the PAM sequence can be on the sense strand or the antisense strand. The PAM sequence can be oriented in any direction. For example, the Cas9 nuclease recognition sites (the secondary endonuclease recognition sites) in the multiple gene editing site, which comprises a target sequence and a PAM sequence, can be on either or both of the sense strand or antisense strand of the construct, and can be oriented in any direction. In an embodiment, the gene editing site crRNA sequence can be 5′-NNNNNNNNNNNNNNNNNNNN-gRNA-3′ (Table 3). In an embodiment, the gene editing site crRNA sequence can be 3′-gRNA-NNNNNNNNNNNNNNNNNNNN-5′ (Table 4).

TABLE 3
GEMS Editing Site crRNA Sequences
(PAM on 5′ to 3′ strand; sense,
non-template strand)

| SEQ ID | |
|---|---|
| NO | Sequences |
| 33 | UGAAUUAGAUUUGCGUUACU |
| 34 | UCACAAUCACUCAAGAAGCA |
| 35 | CUUUAGACACAGUAAGACAA |
| 36 | CCCGCAAUAGAGAGCUUUGA |
| 37 | GAACGUATCUGCAUGUCUAG |
| 38 | CAUGCCUUUAGAAUUCAGUA |
| 39 | UGUGUUAGCGCGCUGAUCUG |
| 40 | UACGAAGUCGAGAUAAAAUG |
| 41 | GCAUAACCAGUACGCAAGAU |
| 42 | UUUUGCUACAUCUUGUAAUA |
| 43 | AUUAUAAUAUUCAGUAGAAA |
| 44 | CAGCTACGAGUCACGAUGUA |
| 45 | CAAUGACAAUAGCGAUAACG |
| 46 | GUUACGUUCGCGAAGCGUUG |
| 47 | GCGUAACAACUUCUGAGUUG |

* 5′-NNNNNNNNNNNNNNNNNNNN-gRNA-3′

TABLE 4
GEMS Editing Site crRNA Sequences
(PAM on 3′ to 5′ strand;
anti-sense, template strand)

| SEQ ID | |
|---|---|
| NO | Sequences |
| 48 | AACAAUACAUACGUGUUCGU |
| 49 | UGCATCGCAAGCTCAUCGCG |
| 50 | AGCGUGUUCGUGUCAGAGCA |
| 51 | UCUACGAGACGCGCGACGUU |
| 52 | UACGAUAAAUAAUUGCGCAG |
| 53 | AAUUAAGAUUUCGUUAGCUU |
| 54 | AACAAUGUGCGCAUGACAUA |
| 55 | GACUGCGCAAUACGAUUUAG |
| 56 | GCAGUAACGUUCAUCUGCGC |
| 57 | AGCUAACGAAAGAGUAGCAU |
| 58 | UAGACGCUCGCUAAAUCUUU |
| 59 | UCGCACUGUCGAGCUAUCAC |
| 60 | GACUAGCGUCACGUAAGAGU |
| 61 | AGCUAGCAUGUAUCUAGGAC |
| 62 | UGCGCGUGCGUCGACAUAUU |

* 3′-gRNA-NNNNNNNNNNNNNNNNNNNN-5′

TABLE 5
GEMS 2.0 Editing Site crRNA Sequences

| SEQ ID | |
|---|---|
| NO | Sequences |
| 63 | AUCCGUAUUCCGACGUACGA |
| 64 | CGUACUGUGAUACACGCGAC |
| 65 | GGCGCUCCGAUAAAUCGCUA |
| 66 | AUUACCGAUACGAUACGAAC |
| 67 | ACGGACGCGCAACCGUCGUC |
| 68 | UAAUCGGUUGCGCCGCUCGG |
| 69 | UUAUUUACCCCGCGCGAGGU |
| 70 | GUUGUAUCGUACGUCGGUCU |
| 71 | AGUAUUCGAGUACGCGUCGA |
| 72 | GUAUUCGAGUACGCGUCGAU |
| 73 | GCGUGCGAUCGUACCGUGUA |
| 74 | CGCAUGGCAAUCUACGCGCG |
| 75 | GUGAACCGACCCGGUCGAUC |
| 76 | UUCUUCGAUACGGUACGAAU |
| 77 | UUUAUAUGGGACGCGUACGC |
| 78 | AGAGUGGCCGCGAUAAUCGA |
| 79 | UAAUCCUCGCGGUAACCGGU |
| 80 | AGAGUGGGCGCGAAUAUCGU |

TABLE 6
Cutting Efficiencies of Tested sgRNAs

| SEQ ID | | | % |
|---|---|---|---|
| NO | sgRNA | sgRNA sequences | Cutting |
| 24 | CCT-16 | TGCTTGTGCATACATAACAA | 18.8 |
| 25 | CCT-04 | CCCGCAATAGAGAGCTTTGA | 15.3 |
| 26 | CCT-19 | TTGCAGCGCGCAGAGCATCT | 13.6 |
| 27 | CCT-10 | TTTTGCTACATCTTGTAATA | 12.0 |
| 28 | CCT-22 | ATACAGTACGCGTGTAACAA | 10.5 |
| 29 | CCT-25 | TACGATGAGAAAGCAATCGA | 9.1 |
| 30 | CCT-13 | CAATGACAATAGCGATAACG | 6.2 |
| 31 | CCT-01 | TGAATTAGATTTGCGTTACT | 0 |
| 32 | CCT-07 | TGTGTTAGCGCGCTGATCTG | 0 |

In an embodiment, S. pyogenes Cas9 (SpCas9) can be used as a CRISPR endonuclease for genome engineering. However, others can be used. In some cases, a different endonuclease can be used to target certain genomic targets. In some cases, synthetic SpCas9-derived variants with non-NGG PAM sequences can be used. Additionally, other Cas9 orthologues from various species have been identified and these “non-SpCas9s” can bind a variety of PAM sequences that can also be useful for the present disclosure. For example, the relatively large size of SpCas9 (approximately 4 kb coding sequence) can lead to plasmids carrying the SpCas9 cDNA that cannot be efficiently expressed in a cell. Conversely, the coding sequence for Staphylococcus aureus Cas9 (SaCas9) is approximately 1 kilo base shorter than SpCas9, possibly allowing it to be efficiently expressed in a cell. Similar to SpCas9, the SaCas9 endonuclease is capable of modifying target genes in mammalian cells in vitro and in mice in vivo. In some cases, a Cas protein can target a different PAM sequence. In some cases, a target gene can be adjacent to a Cas9 PAM, 5′-NGG, for example. In other cases, other Cas9 orthologs can have different PAM requirements. For example, other PAMs such as those of S. thermophilus (5′-NNAGAA for CRISPR1 and 5′-NGGNG for CRISPR3) and Neisseria meningiditis (5′-NNNNGATT) can also be found adjacent to a target gene.

A transgene of the present disclosure can be inserted adjacent to any PAM sequence from any Cas, or Cas derivative, protein. In some cases, a PAM can be found every, or about every, 8 to 12 base pairs in the GEMS construct. A PAM can be found every 1 to 15 base-pairs in in the GEMS construct. A PAM can also be found every 5 to 20 base-pairs in in the GEMS construct. In some cases, a PAM can be found every 5, 6, 7, 8, 9, 10, 11, 12, 13, 14, 15, 16, 17, 18, 19, 20, or more base-pairs in the GEMS construct. In an embodiment, a PAM can be found at or between every 5-10, 10-15, 15-20, 20-25, 25-30, 35-40, 40-45, 45-50, 50-55, 55-60, 60-65, 65-70, 70-75, 75-80, 80-85, 85-90, 90-95, or 95-100 base pairs in the GEMS construct. In an embodiment, a PAM can be found at or between more than 100 base pairs, more than 200 base pairs, more than 300 base pairs, more than 400 base pairs, or more than 500 base pairs in the GEMS construct. In some embodiments, the GEMS construct comprises a GEMS sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a homology arm sequence that is homologous to a sequence of a safe harbor site (e.g., Rosa26, AAVS1, CCR5, Hipp11 (H11)) of a host cell genome. In some embodiments, the Rosa26 5′ homology arm sequence comprises a nucleotide sequence of SEQ ID NO: 7. In some embodiments, the Rosa26 3′ homology arm sequence comprises a nucleotide sequence of SEQ ID NO: 8.

In some embodiments, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some cases, an adjacent cut can be or can be about 3 base pairs upstream of a PAM. In some cases, an adjacent cut can be or can be about 10 base pairs upstream of a PAM. In some cases, an adjacent cut can be or can be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs.

In an embodiment, the GEMS construct comprises a plurality of the secondary endonuclease recognition site. In an embodiment, the plurality of the secondary endonuclease recognition site is a plurality of PAM. Each PAM in the plurality of PAM can be in any orientation (5′ or 3′). The number of PAM sequences in the GEMS construct can vary. In an embodiment, the GEMS construct comprises a plurality of PAM. In an embodiment, the GEMS construct can comprise one or more PAM. In an embodiment, the GEMS construct can comprise two or more PAM. In an embodiment, the GEMS construct can comprise three or more PAM. In an embodiment, the GEMS construct can comprise four or more PAM. In an embodiment, the GEMS construct can comprise five or more PAM. In an embodiment, the GEMS construct can comprise six or more PAM. In an embodiment, the GEMS construct can comprise seven or more PAM. In an embodiment, the GEMS construct can comprise eight or more PAM. In an embodiment, the GEMS construct can comprise nine or more PAM. In an embodiment, the GEMS construct can comprise ten or more PAM. In an embodiment, the GEMS construct can comprise eleven or more PAM. In an embodiment, the GEMS construct can comprise twelve or more PAM. In an embodiment, the GEMS construct can comprise thirteen or more PAM. In an embodiment, the GEMS construct can comprise fourteen or more PAM. In an embodiment, the GEMS construct can comprise fifteen or more PAM. In an embodiment, the GEMS construct can comprise sixteen or more PAM. In an embodiment, the GEMS construct can comprise seventeen or more PAM. In an embodiment, the GEMS construct can comprise eighteen or more PAM. In an embodiment, the GEMS construct can comprise nineteen or more PAM. In an embodiment, the GEMS construct can comprise twenty or more PAM. In an embodiment, the GEMS construct can comprise thirty or more PAM. In an embodiment, the GEMS construct can comprise forty or more PAM.

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the amino-terminus, about or more than about 1, 2, 3, 4, 5, 6, 7, 8, 9, 10 NLSs at or near the carboxy-terminus, or any combination of these (e.g., one or more NLS at the amino-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise about 6 NLSs. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

### Guide Polynucleotides

As used herein, the term “guide polynucleotide(s)” refer to a polynucleotide which can be specific for a target sequence and can form a complex with Cas protein. In an embodiment, the guide polynucleotide is a guide RNA. As used herein, the term “guide RNA (gRNA)” and its grammatical equivalents can refer to an RNA which can be specific for a target DNA and can form a complex with Cas protein. An RNA/Cas complex can assist in “guiding” Cas protein to a target DNA.

A method disclosed herein also can comprise introducing into a host cell at least one guide RNA or guide polynucleotide, e.g., DNA encoding at least one guide RNA. A guide RNA or a guide polynucleotide can interact with a RNA-guided endonuclease to direct the endonuclease to a specific target site, at which site the 5′ end of the guide RNA base pairs with a specific protospacer sequence in a chromosomal sequence.

A guide RNA or a guide polynucleotide can comprise two RNAs, e.g., CRISPR RNA (crRNA) and transactivating crRNA (tracrRNA). A guide RNA or a guide polynucleotide can sometimes comprise a single-chain RNA, or single guide RNA (sgRNA) formed by fusion of a portion (e.g., a functional portion) of crRNA and tracrRNA. A guide RNA or a guide polynucleotide can also be a dual RNA comprising a crRNA and a tracrRNA. Furthermore, a crRNA can hybridize with a target DNA. In an embodiment, a guide RNA can be a fixed guide RNA with PAM variants. For example, the GEMS construct can be designed to comprise a crRNA sequence of 5′-CUUACUACAUGUGCGUGUUC-(gRNA)-3′, wherein PAM can be on sense, non-template strand. For example, the GEMS construct can be designed to comprise a crRNA sequence of 3′-(gRNA)AAAUGAGCAGCAUACUAACA-5′, wherein PAM can be on anti-sense, template strand.

In some embodiments, Rosa26 CRISPR targeting sequence comprises a nucleotide sequence of SEQ ID NO: 9. In some embodiments, Rosa26 CRISPR gRNA sequence comprises a nucleotide sequence of SEQ ID NO: 10. In some embodiments, the GEMS sequence targeting sequence can comprise a sequence selected from SEQ ID NOs: 89, 91, 93, 95, 97, 99, 101, 103, or reverse complements thereof. In some embodiments, the guide RNA targets a site in the GEMS sequence selected from SEQ ID NO: 1. In some embodiments, the guide RNA targets a site in the GEMS sequence selected from SEQ ID NO: 3. In some embodiment, the guide RNA comprises a sequence selected from SEQ ID NOs: 90, 92, 94, 96, 98, 100, 102, or 104.

As discussed above, a guide RNA or a guide polynucleotide can be an expression product. For example, a DNA that encodes a guide RNA can be a vector comprising a sequence coding for the guide RNA. A guide RNA or a guide polynucleotide can be transferred into a cell by transfecting the cell with an isolated guide RNA or plasmid DNA comprising a sequence coding for the guide RNA and a promoter. A guide RNA or a guide polynucleotide can also be transferred into a cell in other way, such as using virus-mediated gene delivery.

A guide RNA or a guide polynucleotide can be isolated. For example, a guide RNA can be transfected in the form of an isolated RNA into a cell or organism. A guide RNA can be prepared by in vitro transcription using any in vitro transcription system known in the art. A guide RNA can be transferred to a cell in the form of isolated RNA rather than in the form of plasmid comprising encoding sequence for a guide RNA.

A guide RNA or a guide polynucleotide can comprise three regions: a first region at the 5′ end that can be complementary to a target site in a chromosomal sequence, a second internal region that can form a stem loop structure, and a third 3′ region that can be single-stranded. A first region of each guide RNA can also be different such that each guide RNA guides a fusion protein to a specific target site. Further, second and third regions of each guide RNA can be identical in all guide RNAs.

A first region of a guide RNA or a guide polynucleotide can be complementary to sequence at a target site in a chromosomal sequence such that the first region of the guide RNA can base pair with the target site. In some cases, a first region of a guide RNA can comprise from or from about 10 nucleotides to 25 nucleotides (i.e., from 10 nucleotides to nucleotides; or from about 10 nucleotides to about 25 nucleotides; or from 10 nucleotides to about 25 nucleotides; or from about 10 nucleotides to 25 nucleotides) or more. For example, a region of base pairing between a first region of a guide RNA and a target site in a chromosomal sequence can be or can be about 10, 11, 12, 13, 14, 15, 16, 17, 18, 19, 20, 22, 23, 24, 25, or more nucleotides in length. Sometimes, a first region of a guide RNA can be or can be about 19, 20, or 21 nucleotides in length.

A guide RNA or a guide polynucleotide can also comprise a second region that forms a secondary structure. For example, a secondary structure formed by a guide RNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A guide RNA or a guide polynucleotide can also comprise a third region at the 3′ end that can be essentially single-stranded. For example, a third region is sometimes not complementarity to any chromosomal sequence in a cell of interest and is sometimes not complementarity to the rest of a guide RNA. Further, the length of a third region can vary. A third region can be more than or more than about 4 nucleotides in length. For example, the length of a third region can range from or from about 5 to 60 nucleotides in length.

A guide RNA or a guide polynucleotide can target any exon or intron of a gene target. In some cases, a guide can target exon 1 or 2 of a gene, in other cases; a guide can target exon 3 or 4 of a gene. A composition can comprise multiple guide RNAs that all target the same exon or in some cases, multiple guide RNAs that can target different exons. An exon and an intron of a gene can be targeted.

A guide RNA or a guide polynucleotide can target a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30, or anywhere between 1-100 nucleotides in length. A target nucleic acid can be at most or at most about 5, 10, 15, 16, 17, 18, 19, 20, 21, 22, 23, 24, 25, 30, 40, 50, or anywhere between 1-100 nucleotides in length. A target nucleic acid sequence can be or can be about 20 bases immediately 5′ of the first nucleotide of the PAM. A guide RNA can target a nucleic acid sequence. A target nucleic acid can be at least or at least about 1-10, 1-20, 1-30, 1-40, 1-50, 1-60, 1-70, 1-80, 1-90, or 1-100 nucleotides.

A guide polynucleotide, for example, a guide RNA, can refer to a nucleic acid that can hybridize to another nucleic acid, for example, the target nucleic acid or protospacer in a genome of a cell. A guide polynucleotide can be RNA. A guide polynucleotide can be DNA. The guide polynucleotide can be programmed or designed to bind to a sequence of nucleic acid site-specifically. A guide polynucleotide can comprise a polynucleotide chain and can be called a single guide polynucleotide. A guide polynucleotide can comprise two polynucleotide chains and can be called a double guide polynucleotide. A guide RNA can be introduced into a cell or embryo as an RNA molecule. For example, a RNA molecule can be transcribed in vitro and/or can be chemically synthesized. An RNA can be transcribed from a synthetic DNA molecule, e.g., a gBlocks® gene fragment. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III). Plasmid vectors that can be used to express guide RNA include, but are not limited to, px330 vectors and px333 vectors. In some cases, a plasmid vector (e.g., px333 vector) can comprise at least two guide RNA-encoding DNA sequences.

A DNA sequence encoding a guide RNA or a guide polynucleotide can also be part of a vector. Further, a vector can comprise additional expression control sequences (e.g., enhancer sequences, Kozak sequences, polyadenylation sequences, transcriptional termination sequences, etc.), selectable marker sequences (e.g., GFP or antibiotic resistance genes such as puromycin), origins of replication, and the like. A DNA molecule encoding a guide RNA can also be linear. A DNA molecule encoding a guide RNA or a guide polynucleotide can also be circular.

When DNA sequences encoding an RNA-guided endonuclease and a guide RNA are introduced into a cell, each DNA sequence can be part of a separate molecule (e.g., one vector containing an RNA-guided endonuclease coding sequence and a second vector containing a guide RNA coding sequence) or both can be part of a same molecule (e.g., one vector containing coding (and regulatory) sequence for both an RNA-guided endonuclease and a guide RNA).

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

In some cases, a gRNA or a guide polynucleotide can comprise modifications. A modification can be made at any location of a gRNA or a guide polynucleotide. More than one modification can be made to a single gRNA or a guide polynucleotide. A gRNA or a guide polynucleotide can undergo quality control after a modification. In some cases, quality control can include PAGE, HPLC, MS, or any combination thereof.

A modification of a gRNA or a guide polynucleotide can be a substitution, insertion, deletion, chemical modification, physical modification, stabilization, purification, or any combination thereof.

A gRNA or a guide polynucleotide can also be modified by 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-0-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, or any combination thereof.

In some cases, a modification is permanent. In other cases, a modification is transient. In some cases, multiple modifications are made to a gRNA or a guide polynucleotide. A gRNA or a guide polynucleotide modification can alter physio-chemical properties of a nucleotide, such as their conformation, polarity, hydrophobicity, chemical reactivity, base-pairing interactions, or any combination thereof.

A modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a gRNA or a guide polynucleotide. A modification can also enhance biological activity. In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Promoter

“Promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. Yet other promoters are tissue specific or activated promoters, including but not limited to T-cell specific promoters.

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Non-limiting exemplary promoters include the simian virus 40 (SV40) early promoter, mouse mammary tumor virus long terminal repeat (LTR) promoter, human immunodeficiency virus (HIV) long terminal repeat (LTR) promoter, adenovirus major late promoter (Ad MLP), a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), mouse mammary tumor virus (MMTV), moloney murine leukemia virus (MoMuLV) promoter, an avian leukemia virus promoter, an Epstein-Barr virus immediate early promoter, an actin promoter, a myosin promoter, an elongation factor-1, promoter, an hemoglobin promoter, a creatine kinase promoter, and an Ovian leukemia virus promoter. U6 promoters are useful for expression non-coding RNAs (e.g., targeter-RNAs, activator-RNAs, single guide RNAs) in eukaryotic cells.

The present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired, or turning off the expression when expression is not desired.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Examples of inducible promoters are alcohol-regulated promoters, tetracycline-regulated promoters, steroid-regulated promoters, metal-regulated promoters, pathogenesis-regulated promoters, temperature-regulated promoters and light-regulated promoters. An inducible promoter allows control of the expression using one or more chemical, biological, and/or environmental inducers. Non-limiting exemplary inducers include doxycycline, isopropyl-P-thiogalactopyranoside (IPTG), galactose, a divalent cation, lactose, arabinose, xylose, N-acyl homoserine lactone, tetracycline, a steroid, a metal, an alcohol, heat, or light.

Examples of inducible promoters include, but are not limited to T7 RNA polymerase promoter, T3 RNA polymerase promoter, Isopropyl-beta-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, and the like. Inducible promoters can therefore be regulated by molecules including, but not limited to, doxycycline; RNA polymerase, e.g., T7 RNA polymerase; an estrogen receptor; an estrogen receptor fusion; and the like.

An inducible promoter utilizes a ligand for dose-regulated control of expression of said at least two genes. In some cases, a ligand can be selected from a group consisting of ecdysteroid, 9-cis-retinoic acid, synthetic analogs of retinoic acid, N,N′-diacylhydrazines, oxadiazolines, dibenzoylalkyl cyanohydrazines, N-alkyl-N,N′-diaroylhydrazines, N-acyl-N-alkylcarbonylhydrazines, N-aroyl-N-alkyl-N′-aroylhydrazines, arnidoketones, 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, RG-115819 (3,5-Dimethyl-benzoic acid N-(1-ethyl-2,2-dimethyl-propyl)-N′-(2-methyl-3-methoxy-benzoyl)-hydrazide-), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide), and any combination thereof.

Expression control sequences can also be used in constructs. For example, an expression control sequence can comprise a constitutive promoter, which is expressed in a wide variety of cell types. For example, among suitable strong constitutive promoters and/or enhancers are expression control sequences from DNA viruses (e.g., SV40, polyoma virus, adenoviruses, adeno-associated virus, pox viruses, CMV, HSV, etc.) or from retroviral LTRs. Tissue-specific promoters can also be used and can be used to direct expression to specific cell lineages.

In some embodiments, the promoter is an inducible promoter. In some embodiments, the promoter is a non-inducible promoter. In some cases, the promoter can be a tissue-specific promoter. Herein “tissue-specific” refers to regulated expression of a gene in a subset of tissues or cell types. In some cases, a tissue-specific promoter can be regulated spatially such that the promoter drives expression only in certain tissues or cell types of an organism. In other cases, a tissue-specific promoter can be regulated temporally such that the promoter drives expression in a cell type or tissue differently across time, including during development of an organism. In some cases, a tissue-specific promoter is regulated both spatially and temporally. In certain embodiments, a tissue-specific promoter is activated in certain cell types either constitutively or intermittently at particular times or stages of the cell type. For example, a tissue-specific promoter can be a promoter that is activated when a specific cell such as a T cell or a NK cell is activated. T cells can be activated in a variety of ways, for example, when presented with peptide antigens by MHC class II molecules or when an engineered T cells comprising an antigen binding polypeptide engages with an antigen. In one instance, such an engineered T cell or NK cell expresses a chimeric antigen receptor (CAR) or T-cell receptor (TCR).

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters can also be referred to as enhancers, transcriptional control elements, control sequences, etc. Any convenient spatially restricted promoter can be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) can depend on the organism. For example, various spatially restricted promoters are known for plants, flies, worms, mammals, mice, etc. Thus, a spatially restricted promoter can be used to regulate the expression of a nucleic acid encoding e.g., a reporter gene, a therapeutic protein, or a nuclease in a wide variety of different tissues and cell types, depending on the organism. Some spatially restricted promoters are also temporally restricted such that the promoter is in the “ON” state or “OFF” state during specific stages of embryonic development or during specific stages of a biological process.

For illustration purposes, non-limiting examples of spatially restricted promoters include neuron-specific promoters, adipocyte-specific promoters, cardiomyocyte-specific promoters, smooth muscle-specific promoters, or photoreceptor-specific promoters. Non-limiting examples of neuron-specific spatially restricted promoters include a neuron-specific enolase (NSE) promoter (e.g., EMBL HSENO2, X51956); an aromatic amino acid decarboxylase (AADC) promoter; a neurofilament promoter (e.g., GenBank HUMNFL, L04147); a synapsin promoter (e.g., GenBank HUMSYNIB, M55301); a thy-1 promoter (e.g., Chen et al. (1987) Cell 51:7-19; and Llewellyn, et al. (2010) Nat. Med. 16(10):1161-1166); a serotonin receptor promoter (e.g., GenBank S62283); a tyrosine hydroxylase promoter (T_{H}) (e.g., Oh et al. (2009) Gene Ther 16:437; Sasaoka et al. (1992) Mol. Brain Res. 16:274; Boundy et al. (1998 J. Neurosci. 18:9989; and Kaneda et al. (1991) Neuron 6:583-594); a GnRH promoter (e.g., Radovick et al. (1991) Proc. Natl. Acad. Sci. USA 88:3402-3406); an L7 promoter (e.g., Oberdick et al. (1990) Science 248:223-226); a DNMT promoter (e.g., Bartge et al. (1988 Proc. Natl. Acad. Sci. USA 85:3648-3652); an enkephalin promoter (e.g., Comb et al. (1988 EMBO J. 17:3793-3805); a myelin basic protein (MBP) promoter; a Ca2+-calmodulin-dependent protein kinase II-alpha (CamKII.alpha.) promoter (e.g., Mayford et al. (1996) Proc. Natl. Acad. Sci. USA 93:13250; and Casanova et al. (2001) Genesis 31:37); and a CMV enhancer/platelet-derived growth factor-0 promoter (e.g., Liu et al. (2004) Gene Therapy 11:52-60).

Non-limiting examples of adipocyte-specific spatially restricted promoters include aP2 gene promoter/enhancer, e.g., a region from −5.4 kb to +21 bp of a human aP2 gene (e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (e.g., Knight et al. (2003) Proc. Natl. Acad. Sci. USA 100:14725); a fatty acid translocase (FAT/CD36) promoter (e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (e.g., Mason et al. (1998 Endocrinol. 139:1013; and Chen et al. (1999) Biochem. Biophys. Res. Comm. 262:187); an adiponectin promoter (e.g., Kita et al. (2005) Biochem. Biophys. Res. Comm. 331:484; and Chakrabarti (2010) Endocrinol. 151:2408; an adipsin promoter (e.g., Platt et al. (1989) Proc. Natl. Acad. Sci. USA 86:7490); and a resistin promoter (e.g., Seo et al. (2003) Molec. Endocrinol. 17:1522).

Non-limiting examples of cardiomyocyte-specific spatially restricted promoters include control sequences derived from the following genes: myosin light chain-2, α-myosin heavy chain, AE3, cardiac troponin C, and cardiac actin (Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051).

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In an embodiment, the CMV promoter sequence comprises a nucleotide sequence of SEQ ID NO: 11 or SEQ ID NO: 82. In some embodiments, the CMV promoter comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 11 or SEQ ID NO: 82.

Another example of a suitable promoter is human elongation growth factor 1 alpha 1 (hEF1a1). In embodiments, the vector construct comprising the CARs and/or TCRs of the present disclosure comprises hEF1a1 functional variants. In an embodiment, the EF-1 alpha promoter sequence comprises a nucleotide sequence of SEQ ID NO: 18. In some embodiments, the EF-1 alpha promoter comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 18.

Another example of a suitable promoter is Chinese Hamster EF-1a (CHEF1-a). In embodiments, the vector construct comprising the CARS and/or TCRs of the present disclosure comprises CHEF1-a functional variants. In an embodiment, the CHEF1-a promoter sequence comprises a nucleotide sequence of SEQ ID NO: 83. In some embodiments, the CHEF1-a alpha promoter comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 83.

### Reporter System

In some aspects, the GEMS construct further comprises a reporter gene, which confirms that the GEMs sequence has been successfully inserted into the host cell genome. The reporter gene can encode a protein that does not does not interfere with insertion of donor genes, or interfere with other natural processes in the cell, or otherwise cause deleterious effects in the cell. The reporter gene can encode a detectable protein such as a fluorescent protein, including green fluorescent protein (GFP) (SEQ ID NO: 12) or related proteins such as yellow fluorescent protein, blue fluorescent protein, or red fluorescent protein. The reporter gene can be under control of an inducer (i.e., an inducible promoter). In an embodiment, the inducer is an alcohol, tetracycline, a steroid, a metal or isopropyl-β-thiogalactopyranoside (IPTG). In an embodiment, the inducer is heat or light. For example, the multiple gene editing site of the construct can comprise the gene encoding GFP as a reporter, with the GFP gene under a tetracycline (Tet) promoter, which inhibits the expression of the GFP protein until the cell is exposed to tetracycline. In an embodiment, the GFP sequence comprises a nucleotide sequence of SEQ ID NO: 12. In some embodiments, the GFP sequence comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 12.

In order to assess GEMS insertion and/or the expression of donor nucleotide sequences (e.g., CAR or portions thereof), the expression vector to be introduced into a cell can also contain either a selectable marker gene or a reporter gene or both to facilitate identification and selection of expressing cells from the population of cells sought to be transfected or infected through viral vectors. In some embodiments, the GEMS construct comprises a GEMS sequence of SEQ ID NO: 1. In some embodiments, the GEMS construct comprises a GEMS sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, and/or SEQ ID NO: 105. In some embodiments, the GEMS construct comprises a homology arm sequence that is homologous to a sequence of a safe harbor site (e.g., Rosa26, AAVS1, CCR5, Hipp11 (H11)) of a host cell genome. In some embodiments, the Rosa26 5′ homology arm sequence comprises a nucleotide sequence of SEQ ID NO: 7. In some embodiments, the Rosa26 3′ homology arm sequence comprises a nucleotide sequence of SEQ ID NO: 8.

In other aspects, the selectable marker can be carried on a separate piece of DNA and used in a co-transfection procedure. Both selectable markers and reporter genes can be flanked with appropriate regulatory sequences to enable expression in the host cells. In some embodiments, the selectable marker polypeptide is a WT selectable marker polypeptide. In some embodiments, the selectable marker polypeptide is a modified selectable marker polypeptide. In some embodiments, nucleic acid sequence encoding a selectable marker polypeptide include, for example, antibiotic-resistance genes, such as puromycin resistance gene (puro (SEQ ID NO: 13), neomycin resistance gene (neo) (SEQ ID NO: 84 or SEQ ID NO: 14), blasticidin resistance gene (bla) (SEQ ID NO: 19), hygromycin resistance gene (SEQ ID NO: 81) and ampicillin resistance gene and the like. In some embodiments, the nucleic acid sequence encoding a selectable marker polypeptide is a puromycin resistance gene sequence comprises a nucleotide sequence of SEQ ID NO: 13. In some embodiments, the nucleic acid sequence encoding a selectable marker polypeptide is a puromycin resistance gene sequence comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 13. In an embodiment, the nucleic acid sequence encoding a selectable marker polypeptide is a blasticidin resistance gene sequence comprises a nucleotide sequence of SEQ ID NO: 19. In some embodiments, the nucleic acid sequence encoding a selectable marker polypeptide is a hygromycin resistance gene sequence comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 81. In some embodiments, the nucleic acid sequence encoding a selectable marker polypeptide is a neomycin resistance gene sequence comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 14 or SEQ ID NO: 84. In some embodiments, nucleic acid sequence encoding a modified selectable marker protein comprises a nucleotide sequence of SEQ ID NO: 84. In some embodiments, nucleic acid sequence encoding a modified selectable marker protein comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 84. In some embodiments, a modified selectable marker polypeptide comprises a reduced activity relative to a corresponding WT selectable marker polypeptide. In some embodiments, a nucleic acid sequence encoding the WT selectable marker polypeptide comprises a sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 14.

Reporter genes can be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes can include genes encoding luciferase, beta-galactosidase, chloramphenicol acetyl transferase, secreted alkaline phosphatase, or the green fluorescent protein gene (e.g., Ui-Tei et al., FEBS Letters 479: 79-82 (2000)). Suitable expression systems are well known and can be prepared using known techniques or obtained commercially. In general, the construct with the minimal 5′ flanking region showing the highest level of expression of reporter gene is identified as the promoter. Such promoter regions can be linked to a reporter gene and used to evaluate agents for the ability to modulate promoter-driven transcription.

Regardless of the method used to introduce exogenous nucleic acids into the host, in order to confirm the presence of the recombinant DNA sequence in the host cell, a variety of assays can be performed. Such assays include, for example, molecular assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

### Host Cells

The GEMS construct provided herein can be inserted into any suitable host cell to generate a genetically engineered cell. The term “host cell” as used herein refers to an in vivo or in vitro eukaryotic cell (a cell from a unicellular or multicellular organism, e.g., a cell line) which can be, or has been, used as a recipient for the GEMS construct. Host cells or genetically engineered cells can further comprise donor nucleic acid sequences (e.g., encoding a therapeutic protein) as described herein inserted into the GEMS sequence. The term “host cell” includes the progeny of the original cell which has been targeted (e.g., transfected with a GEMS construct, a construct encoding a nuclease and/or a guide polynucleotide). It is understood that the progeny of a single cell is not necessarily be completely identical in morphology or in genomic or total DNA complement as the original parent, due to natural, accidental, or deliberate mutation. A host cell can be any eukaryotic cell (e.g., a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, or a human cell). Cells may be any suitable eukaryotic cell. In exemplary embodiments, the host cell is a Chinese Hamster Ovary (CHO) cell, such as cells from the CHO-K1 line or any other suitable cell line. While CHO cells may be the cell of choice, a variety of other cells may also be employed. In general, the cell will be a eukaryotic cell or a single cell eukaryotic organism. In some embodiments, the host cell is a mammalian cell.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines, in addition to CHO cells, include monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, human U2-OS osteosarcoma cells, human A549 cells, human K562 cells, human HEK293 cells, human HEK293T cells, human HCT116 cells, human MCF-7 cells, and TRI cells. For an extensive list of mammalian cell lines, those of ordinary skill in the art may refer to the American Type Culture Collection catalog (ATCC®, Manassas, Va.). In particular, cell lines useful in recombinant protein production and biopharmaceutical production can be used, for example, CHO cells, mouse myeloma cells (NS0), HEK293 and HEK293T.

Insertion of the GEMS construct can proceed according to any technique suitable in the art. For example, transfection, lipofection, or temporary membrane disruption such as electroporation or deformation can be used to insert the construct into the host cell. Viral vectors or non-viral vectors can be used to deliver the construct in some aspects. In some embodiments, the GEMS construct comprises a GEMS sequence of SEQ ID NO: 1, or SEQ ID NO: 3. In some embodiments, the GEMS construct comprises a GEMS sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, SEQ ID NO: 3, and/or SEQ ID NO: 105. In some embodiments, the GEMS construct comprises a first flanking insertion sequence, a second flanking insertion sequence, or both that is homologous to a sequence of a safe harbor site (e.g., Rosa26, AAVS1, CCR5, Hipp11 (H11)) of a host cell genome. In some embodiments, the first flanking insertion sequence can be Rosa26 5′ homology arm sequence comprising a nucleotide sequence of SEQ ID NO: 7. In some embodiments, the second flanking insertion sequence can be Rosa26 3′ homology arm sequence comprising a nucleotide sequence of SEQ ID NO: 8. In some embodiments, Rosa26 CRISPR targeting sequence comprises a nucleotide sequence of SEQ ID NO: 9. In some embodiments, Rosa26 CRISPR gRNA sequence comprises a nucleotide sequence of SEQ ID NO: 10.

In an embodiment, the host cell can be non-competent, and nucleases (e.g., endonucleases) can be transfected to the host cell. In an embodiment, the host cell can be competent for a nuclease, for example, a meganuclease, a Cas9 nuclease. Competency for the primary endonuclease permits integration of the multiple gene editing site into the host cell genome. The host cell can be a primary isolate, obtained from a subject and optionally modified as necessary to make the cell competent for either or both of the primary endonuclease and the secondary endonuclease.

In some aspects, the host cell is a cell line. In some aspects, the host cell is a primary isolate or progeny thereof. In some aspects, the host cell is a stem cell. The stem cell can be an embryonic stem cell or an adult cell. The stem cell is preferably pluripotent, and not yet differentiated or begun a differentiation process. In some aspects, the host cell is a fully differentiated cell. When the host cell, transfected with the construct, divides, a GEMS sequence can be integrated with the host cell genome such that progeny of the host cell can carry the GEMS. A host cell comprising an integrated GEMS sequence can be cultured and expanded in order to increase the number of cells available for receiving donor gene sequences. Stable integration ensures subsequent generations of cells can have the multiple gene editing sites.

The host cell can be further manipulated at locations outside of the multiple gene editing site. For example, the host cell can have one or more genes knocked out, or can have one or more genes knocked down with siRNA, shRNA, or other suitable nucleic acid for gene knock down. The host cell can also or alternatively have other genes edited or revised via any suitable editing technique. Such manipulations outside of the multiple gene editing site can, for example, permit the assessment of the effects of the donor nucleic acid sequence, or the protein it encodes, on the cell when other genes are knocked out, knocked down, or otherwise altered.

In some embodiments, the host cell manipulations outside of the multiple gene editing site, as well as manipulations by way of the addition of donor nucleic acid sequences, can favorably enhance the immunogenicity profile of the host cell. Thus, for example, via added donor nucleic acid sequences, the host cell can express one or more markers that impart compatibility with the immune system of the subject to which the host cell is administered in a therapeutic context.

Alternatively, via knockout or knockdown manipulations, the host cell can lack expression of one or more markers that would cause the cell to be recognized and destroyed by the immune system of the subject to which the host cell is administered in a therapeutic context.

In some embodiments, the host cell is a mammalian cell. In some embodiments, the host cell is a rodent cell. In some embodiments, the host cell is a hamster cell. In some embodiments, the host cell is a rodent ovary cell. In some embodiments, the host cell is a Chinese hamster ovary (CHO) cell.

In some embodiments, the host cell can be one or more cells from tissues or organs, the tissues or organs including brain, lung, liver, heart, spleen, pancreas, small intestine, large intestine, skeletal muscle, smooth muscle, skin, bones, adipose tissues, hairs, thyroid, trachea, gall bladder, kidney, ureter, bladder, aorta, vein, esophagus, diaphragm, stomach, rectum, adrenal glands, bronchi, ears, eyes, retina, genitals, hypothalamus, larynx, nose, tongue, spinal cord, or ureters, uterus, ovary and testis. For example, the host cell can be from brain, heart, liver, skin, intestine, lung, kidney, eye, small bowel, pancreas, or spleen.

In some embodiments, the host cell can be one or more of trichocytes, keratinocytes, gonadotropes, corticotropes, thyrotropes, somatotropes, lactotrophs, chromaffin cells, parafollicular cells, glomus cells melanocytes, nevus cells, Merkel cells, odontoblasts, cementoblasts corneal keratocytes, retina Muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, D cells, ECL cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic a cells, pancreatic β cells, pancreatic δ cells, pancreatic F cells (e.g., PP cells), pancreatic c cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, splenocytes (e.g., T lymphocytes, B lymphocytes, dendritic cells, microphages, leukocytes), trophoblast stem cells, or any combination thereof.

In some cases, the host cell is a T cell. In some cases, the T cell is an αβ T-cell, an NK T-cell, a γδ T-cell, a regulatory T-cell, a T helper cell, or a cytotoxic T-cell.

In some case, the host cell is a B cell. In some case, a B cell is a plasmablast, a plasma cell, a lymphoplasmacytoid cell, memory B cell, follicular B cell, marginal zone B cell, B-1 cell, naïve B cell, Regulatory B (Breg) cell

In one aspect provided herein is a genetically engineered cell, comprising a gene editing multi-site (GEMS) sequence in said cell's genome, said GEMS sequence comprising a plurality of nuclease recognition sequences, wherein each of said plurality of nuclease recognition sequences comprises a target sequence and a protospacer adjacent motif (PAM) sequence or reverse complements thereof. In some embodiments, the genetically engineered cell further comprises a donor nucleic acid sequence inserted within or adjacent to said GEMS sequence. In some embodiments, the donor nucleic acid encodes a donor nucleic acid sequence encodes a therapeutic protein. The therapeutic protein can comprise, for example, chimeric antigen receptor (CAR), a T-cell receptor (TCR), a B-cell receptor (BCR), an αβ receptor, a γδ T-receptor, or a combination thereof. The therapeutic protein can comprise, dopamine or a portion thereof, insulin, proinsulin, or a portion thereof.

In some embodiments, the genetically engineered cell can further comprise a genetic modification in order to reduce their immunogenicity. Accordingly, In some embodiments, the genetically engineered cell can further comprise a disruption in one or more genes encoding a human leucocyte antigen (HLA). The HLA can comprise, for example, HLA-A, HLA-B, HLA-C, HLA-E, HLA-F, HLA-G, HLA-DP, HLA-DQ, HLA-DR, or a combination thereof. In some embodiments, the genetically engineered cell can comprises a nucleic acid sequence coding for a suicide gene, wherein the suicide gene encodes an apoptosis inducing molecule. In some embodiments, the apoptosis inducing molecule is fused to an inducer ligand binding domain. The nucleic acid sequence encoding an apoptosis inducing molecule can be operably linked to a nucleic acid sequence encoding a regulatory element, for example a promoter. In some embodiments, the promoter can be inducible promoter. Examples of inducible promoters used for regulated gene expression are well known in the art. Non limiting examples include, cyclooxygenase promoter, a tumor necrosis factor promoter, an interleukin regulated promoter, alcohol-regulated promoter, steroid regulated promoter, dexamethasone regulated promoter, tetracycline regulated promoter, metal regulated promoter, light regulated promoter, and temperature regulated promoter, In some embodiments, the apoptosis inducing molecule encoded by the suicide gene can be a caspase, a protease, or a prodrug activating enzyme. Non-limiting examples of apoptosis inducing molecule include, Caspase-1, Caspase-2, Caspase-3, Caspase-4, Caspase-5, Caspase-6, Caspase-7, Caspase-8, Caspase-9, Caspase-10, Granzyme A, Granzyme B, viral thymidine kinase, Cytosine deaminase, Fas ligand, TRAIL, or APO3L.

### Stem Cells

In some cases, the host cell is a stem cell. In some cases, the host cell is an adult stem cell. In some cases, the host cell is an embryonic stem cell. In some cases, the host cell is a non-embryonic stem cell. In some cases, the host ells are derived from non-stem cells. In some cases, the host cells are derived from stem cells (e.g., embryonic stem cells, non-embryonic stem cells, pluripotent stem cells, placental stem cells, induced pluripotent stem cells, trophoblast stem cells etc.).

The term “stem cell” is used herein to refer to a cell (e.g., plant stem cell, vertebrate stem cell) that has the ability both to self-renew and to generate a differentiated cell type (Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and can or cannot retain the capacity to proliferate further. Stem cells can be characterized by both the presence of specific markers (e.g., proteins, RNAs, etc.) and the absence of specific markers. Stem cells can also be identified by functional assays both in vitro and in vivo, particularly assays relating to the ability of stem cells to give rise to multiple differentiated progeny. In an embodiment, the host cell is an adult stem cell, a somatic stem cell, a non-embryonic stem cell, an embryonic stem cell, hematopoietic stem cell, an include pluripotent stem cells, and a trophoblast stem cell.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs can be in the form of an established cell line, they can be obtained directly from primary embryonic tissue, or they can be derived from a somatic cell.

By “embryonic stem cell” (ESC) is meant a PSC that is isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells can be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. Nos. 7,029,913, 5,843,780, and 6,200,806, each of which is incorporated herein by its entirety. Methods for proliferating hESCs in the undifferentiated form are described in WO 99/20741, WO 01/51616, and WO 03/020920, each of which is incorporated herein by its entirety.

By “embryonic germ stem cell” (EGSC) or “embryonic germ cell” or “EG cell”, it is meant a PSC that is derived from germ cells and/or germ cell progenitors, e.g. primordial germ cells, i.e. those that can become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, each of which are incorporated herein by its entirety.

By “induced pluripotent stem cell” or “iPSC”, it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs can be found in, for example, U.S. Patent Publication Nos. US20090047263, US20090068742, US20090191159, US20090227032, US20090246875, and US20090304646, each of which are incorporated herein by its entirety. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell”, it is meant any cell in an organism that, in the absence of experimental manipulation, does not ordinarily give rise to all types of cells in an organism. In other words, somatic cells are cells that have differentiated sufficiently that they do not naturally generate cells of all three germ layers of the body, i.e. ectoderm, mesoderm and endoderm. For example, somatic cells can include both neurons and neural progenitors, the latter of which is able to naturally give rise to all or some cell types of the central nervous system but cannot give rise to cells of the mesoderm or endoderm lineages.

### Trophoblast Stem Cells

Trophoblast stem cells (TS cells) are precursors of differentiated placenta cells. In some instances, a TS cell is derived from a blastocyst polar trophectoderm (TE) or an extraembryonic ectoderm (ExE) cell. In some cases, TS is capable of indefinite proliferation in vitro in an undifferentiated state, and is capable of maintaining the potential multilineage differentiation capabilities in vitro. In some instances, a TS cell is a mammalian TS cell. Exemplary mammals include mouse, rat, rabbit, sheep, cow, cat, dog, monkey, ferret, bat, kangaroo, seals, dolphin, and human. In some embodiments, a TS cell is a human TS (hTS) cell.

In some instances, TS cells are obtained from fallopian tubes. Fallopian tubes are the site of fertilization and the common site of ectopic pregnancies, in which biological events such as the distinction between inner cell mass (ICM) and trophectoderm and the switch from totipotency to pluripotency with major epigenetic changes take place. In some instances, these observations provide support for fallopian tubes as a niche reservoir for harvesting blastocyst-associated stem cells at the preimplantation stage. Blastocyst is an early-stage preimplantation embryo, and comprises ICM which subsequently forms into the embryo, and an outer layer termed trophoblast which gives rise to the placenta.

In some embodiments, a TS cell is a stem cell used for generation of a progenitor cell such as for example a hepatocyte. In some embodiments, a TS cell is derived from ectopic pregnancy. In some embodiments, the TS cell is a human TS cell. In one embodiment, the human TS cell derived from ectopic pregnancies does not involve the destruction of a human embryo. In another embodiment, the human TS cell derived from ectopic pregnancies does not involve the destruction of a viable human embryo. In another embodiment, the human TS cell is derived from trophoblast tissue associated with non-viable ectopic pregnancies. In another embodiment, the ectopic pregnancy cannot be saved. In another embodiment, the ectopic pregnancy would not lead to a viable human embryo. In another embodiment, the ectopic pregnancy threatens the life of the mother. In another embodiment, the ectopic pregnancy is tubal, abdominal, ovarian or cervical.

During normal blastocyst development, ICM contact per se or its derived diffusible ‘inducer’ triggers a high rate of cell proliferation in the polar trophectoderm, leading to cell movement toward the mural region throughout the blastocyst stage and can continue even after the distinction of the trophectoderm from the ICM. The mural trophectoderm cells overlaying the ICM are able to retain a ‘cell memory’ of ICM. At the beginning of the implantation, the mural cells opposite the ICM cease division because of the mechanical constraints from the uterine endometrium. However, in an ectopic pregnancy in which the embryo is located within the fallopian tube, constraints do not exist in the fallopian tubes which result in continuing division of polar trophectoderm cells to form extraembryonic ectoderm (ExE) in the stagnated blastocyst. In some instances, the ExE-derived TS cells exist for up to 20 days in a proliferation state. As such, until clinical intervention occurs, the cellular processes can yield an indefinite number of hTS cells in the preimplantation embryos and such cells can retain cell memory from ICM.

In some instances, TS cells possess specific genes of ICM (e.g., OCT4, NANOG, SOX2, FGF4) and trophectoderm (e.g., CDX2, Fgfr-2, Eomes, BMP4), and express components of the three primary germ layers, mesoderm, ectoderm, and endoderm. In some instances, TS cells express embryonic stem (e.g., human embryonic stem) cell-related surface markers such as specific stage embryonic antigen (SSEA)-1, -3 and -4 and mesenchymal stem cell-related markers (e.g., CD44, CD90, CK7 and Vimentin). In other instances, hematopoietic stem cell markers (e.g., CD34, CD45, a6-integrin, E-cadherin, and L-selectin) are not expressed.

### Mammalian Trophoblast Stem Cells

In some embodiments, the host cell can be a mammalian trophoblast stem cell from rodents (e.g, mice, rats, guinea pigs, hamsters, squirrels), rabbits, cows, sheep, pigs, dogs, cats, monkeys, apes (e.g., chimpanzees, gorillas, orangutans), or humans. In one instance, a mammalian trophoblast stem cell herein is not from primates, e.g., monkeys, apes, humans. In another instance, a mammalian trophoblast stem cell herein is from primates, e.g., monkeys, apes, humans. In another instance, a mammalian trophoblast stem cell herein is human or humanized.

A mammalian trophoblast stem cell herein can be induced for differentiating into one or more kinds of differentiated cells prior to or after insertion of one or more GEMS constructs. In some embodiments, the GEMS construct comprises a GEMS sequence of SEQ ID NO: 1, or SEQ ID NO: 3. In some embodiments, the GEMS construct comprises a GEMS sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. In some embodiments, the GEMS construct comprises a nucleotide sequence of SEQ ID NO: 1, SEQ ID NO: 3 and/or SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, SEQ ID NO: 3, and/or SEQ ID NO: 105. In some embodiments, the GEMS construct comprises a first flanking insertion sequence, a second flanking insertion sequence, or both that is homologous to a sequence of a safe harbor site (e.g., Rosa26, AAVS1, CCR5, Hipp11 (H11)) of a host cell genome. In some embodiments, the first flanking insertion sequence can be Rosa26 homology arm sequence comprising a nucleotide sequence of SEQ ID NO: 7. In some embodiments, the second flanking insertion sequence can be Rosa26 3′ homology arm sequence comprising a nucleotide sequence of SEQ ID NO: 8. In some embodiments, Rosa26 CRISPR targeting sequence comprises a nucleotide sequence of SEQ ID NO: 9. In some embodiments, Rosa26 CRISPR gRNA sequence comprises a nucleotide sequence of SEQ ID NO: 10.

In one instance, the differentiated cell is a progenitor cell, e.g., a pancreatic progenitor cell. In one instance, the differentiated cell is a pluripotent stem cell. In one instance, the differentiated cell is an endodermal, mesodermal, or ectodermal progenitor cell. In one instance, the differentiated cell is a definitive endoderm progenitor cell. In one instance, the differentiated cell is a pancreatic endoderm progenitor cell. In one instance, the differentiated cell is a multipotent progenitor cell. In one instance, the differentiated cell is an oligopotent progenitor cell. In one instance, the differentiated cell is a monopotent, bipotent, or tripotent progenitor cell. In one instance, the differentiated cell is an endocrine, exocrine, or duct progenitor cell, e.g., an endocrine progenitor cell. In one instance, the differentiated cell is a beta-cell. In one instance, the differentiated cell is an insulin-producing cell. One or more differentiated cells can be used in any method disclosed herein.

In one aspect, provided herein are one or more differentiated cells comprising one or more GEMS constructs. In one instance, the isolated differentiated cell is a human cell. In one instance, the isolated differentiated cell has a normal karyotype. In one instance, the isolated differentiated cell has one or more immune-privileged characteristics, e.g., low or absence of CD33 expression and/or CD133 expression. One or more isolated differentiated cells disclosed herein can be used in any method disclosed herein.

In another aspect, provided herein is an isolated progenitor cell that expresses one or more transcription factors comprising Foxa2, Pdx1, Ngn3, Ptf1a, Nkx6.1, or any combination thereof. In one instance, the isolated progenitor cell expresses two, three, or four transcription factors of Foxa2, Pdx1, Ngn3, Ptf1a, Nkx6.1. In one instance, the isolated progenitor cell expresses Foxa2, Pdx1, Ngn3, Ptf1a, and Nkx6.1. In one instance, the isolated progenitor cell is an induced pluripotent stem cell. In one instance, the isolated progenitor cell is derived from a mammalian trophoblast stem cell, e.g., an hTS cell. In one instance, the isolated progenitor cell is a pancreatic progenitor cell. In one instance, the isolated progenitor cell is an endodermal, mesodermal, or ectodermal progenitor cell. In one instance, the isolated progenitor cell is a definitive endoderm progenitor cell. In one instance, the isolated progenitor cell is a pancreatic endoderm progenitor cell. In one instance, the isolated progenitor cell is a multipotent progenitor cell. In one instance, the isolated progenitor cell is an oligopotent progenitor cell. In one instance, the isolated progenitor cell is a monopotent, bipotent, or tripotent progenitor cell. In one instance, the isolated progenitor cell is an endocrine, exocrine, or duct progenitor cell, e.g., an endocrine progenitor cell. In one instance, the isolated progenitor cell is a beta-cell. In one instance, the isolated progenitor cell is an insulin-producing cell. In one instance, the isolated progenitor cell is from rodents (e.g, mice, rats, guinea pigs, hamsters, squirrels), rabbits, cows, sheep, pigs, dogs, cats, monkeys, apes (e.g., chimpanzees, gorillas, orangutans), or humans. In one instance, the isolated progenitor cell is a human cell. In one instance, the isolated progenitor cell has a normal karyotype. In one instance, the isolated progenitor cell has one or more immune-privileged characteristics, e.g., low or absence of CD33 expression and/or CD133 expression. An isolated progenitor cell disclosed herein can be used in any method disclosed herein.

In another aspect, provided herein is an isolated progenitor cell that expresses betatrophin, betatrophin mRNA, C-peptide, and insulin, wherein the isolated progenitor cell is differentiated from a mammalian trophoblast stem cell. In one instance, the isolated progenitor cell is from rodents (e.g, mice, rats, guinea pigs, hamsters, squirrels), rabbits, cows, sheep, pigs, dogs, cats, monkeys, apes (e.g., chimpanzees, gorillas, orangutans), or humans. In one instance, the isolated progenitor cell is a pancreatic progenitor cell. In one instance, the isolated progenitor cell is a human cell. In one instance, the isolated progenitor cell has a normal karyotype. In one instance, the isolated progenitor cell has one or more immune-privileged characteristics, e.g., low or absence of CD33 expression and/or CD133 expression. One or more isolated progenitor cells disclosed herein can be used in any method disclosed herein. In one instance, an isolated progenitor cell herein is an insulin-producing cell. One or more isolated progenitor cells herein can be used in any method disclosed herein. In one instance, a differentiated cell herein is an insulin-producing cell. In one instance, a differentiated cell herein is a neurotransmitter producing cell.

### Human Trophoblast Stem Cells

Human fallopian tubes are the site of fertilization and the common site of ectopic pregnancies in women, where several biological events take place such as the distinction between inner cell mass (ICM) and trophectoderm and the switch from totipotency to pluripotency with the major epigenetic changes. These observations provide support for fallopian tubes as a niche reservoir for harvesting blastocyst-associated stem cells at the preimplantation stage. Ectopic pregnancy accounts for 1 to 2% of all pregnancies in industrialized countries and are much higher in developing countries. Given the shortage in availability of human embryonic stem cells (hES cells) and fetal brain tissue, described herein is the use of human trophoblast stem cells (hTS cells) derived from ectopic pregnancy as a substitution for scarcely available hES cells for generation of progenitor cells.

In some embodiments, the hTS cells derived from ectopic pregnancies do not involve the destruction of a human embryo. In another instance, the hTS cells derived from ectopic pregnancies do not involve the destruction of a viable human embryo. In another instance, the hTS cells are derived from trophoblast tissue associated with non-viable ectopic pregnancies. In another instance, the ectopic pregnancy cannot be saved. In another instance, the ectopic pregnancy would not lead to a viable human embryo. In another instance, the ectopic pregnancy threatens the life of the mother. In another instance, the ectopic pregnancy is tubal, abdominal, ovarian or cervical.

In some embodiments, during blastocyst development, ICM contact per se or its derived diffusible ‘inducer’ triggers a high rate of cell proliferation in the polar trophectoderm, leading to cell movement toward the mural region throughout the blastocyst stage and can continue even after the distinction of the trophectoderm from the ICM. The mural trophectoderm cells overlaying the ICM are able to retain a ‘cell memory’ of ICM. Normally, at the beginning of implantation the mural cells opposite the ICM cease division because of the mechanical constraints from the uterine endometrium. However, no such constraints exist in the fallopian tubes, resulting in the continuing division of polar trophectoderm cells to form extraembryonic ectoderm (ExE) in the stagnated blastocyst of an ectopic pregnancy. In some embodiments, the ExE-derived TS cells exist for at least a 4-day window in a proliferation state, depending on the interplay of ICM-secreted fibroblast growth factor 4 (FGF4) and its receptor fibroblast growth factor receptor 2 (Fgfr2). In another instance, the ExE-derived TS cells exist for at least a 1-day, at least a 2-day, at least a 3-day, at least a 4-day, at least a 5-day, at least a 6-day, at least a 7-day, at least a 8-day, at least a 9-day, at least a 10-day, at least a 11-day, at least a 12-day, at least a 13-day, at least a 14-day, at least a 15-day, at least a 16-day, at least a 17-day, at least a 18-day, at least a 19-day, at least a 20-day window in a proliferation state. Until clinical intervention occurs, these cellular processes can yield an indefinite number of hTS cells in the preimplantation embryos; such cells retaining cell memory from ICM, reflected by the expression of ICM-related genes.

### Method of Differentiating Host Stem Cells

In an embodiment, the host stem cell can be differentiated prior to or after insertion of one or more GEMS constructs. In some embodiments, the GEMS construct comprises a GEMS sequence of SEQ ID NO: 1, or SEQ ID NO: 3. In some embodiments, the GEMS construct comprises a GEMS sequence of SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, or SEQ ID NO: 3. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 105. In some embodiments, the GEMS construct comprises a nucleotide sequence of SEQ ID NO: 1, SEQ ID NO: 3, and/or SEQ ID NO: 105. In some embodiments, the GEMS construct comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 1, SEQ ID NO: 3, and/or SEQ ID NO: 105. In some embodiments, the GEMS construct comprises a first flanking insertion sequence, a second flanking insertion sequence, or both that is homologous to a sequence of a safe harbor site (e.g., Rosa26, AAVS1, CCR5, Hipp11 (H11)) of a host cell genome. In some embodiments, the first flanking insertion sequence can be Rosa26 5′ homology arm sequence comprising a nucleotide sequence of SEQ ID NO: 7. In some embodiments, the second flanking insertion sequence can be Rosa26 3′ homology arm sequence comprising a nucleotide sequence of SEQ ID NO: 8. In some embodiments, Rosa26 CRISPR targeting sequence comprises a nucleotide sequence of SEQ ID NO: 9. In some embodiments, Rosa26 CRISPR gRNA sequence comprises a nucleotide sequence of SEQ ID NO: 10.

In one of many aspects, provided herein is a method of differentiating the host stem cell. In some embodiments, the host cell is a CHO cell. In some embodiments, the host cell is an eukaryotic cell. In some embodiments, the host cell is a mammalian cell. In an embodiment, the host stem cell is a mammalian trophoblast stem cell. In one instance, the mammalian trophoblast stem cell is a human trophoblast stem (hTS) cell. In one instance, the differentiated cell is a pluripotent stem cell. In one instance, the differentiated cell is a progenitor cell, e.g., a pancreatic progenitor cell. In one instance, the differentiated cell is an endodermal, mesodermal, or ectodermal progenitor cell, e.g., a definitive endoderm progenitor cell. In one instance, the differentiated cell is a pancreatic endoderm progenitor cell. In one instance, the differentiated cell is a multipotent progenitor cell. In one instance, the differentiated cell is an oligopotent progenitor cell. In one instance, the differentiated cell is a monopotent, bipotent, or tripotent progenitor cell. In one instance, the differentiated cell is an endocrine, exocrine, or duct progenitor cell, e.g., an endocrine progenitor cell. In one instance, the differentiated cell is a beta-cell. In one instance, the differentiated cell is an insulin-producing cell. One or more differentiated cells can be used in any method disclosed herein.

In some embodiments, the mammalian trophoblast stem cell herein is from rodents (e.g, mice, rats, guinea pigs, hamsters, squirrels), rabbits, cows, sheep, pigs, dogs, cats, monkeys, apes (e.g., chimpanzees, gorillas, orangutans), or humans.

In some embodiments, the method of differentiating the host stem cells activates miR-124. In one instance, the method of differentiating the host stem cells activates miR-124 spatiotemporarily, e.g., between about 1 hour to about 8 hours, at a definitive endoderm stage. In one instance, the method of differentiating the host stem cells elevates miR-124 expression. In one instance, the method of differentiating the host stem cells deactivates miR-124. In one instance, the method of differentiating the host stem cells decreases miR-124 expression. In one instance, the method of differentiating the host stem cells comprises contacting the mammalian trophoblast stem cell with one or more agents, e.g., proteins or steroid hormones. In one instance, the one or more agents comprise a growth factor, e.g., a fibroblast growth factor (FGF). In one instance, the FGF is one or more of FGF1, FGF2, FGF3, FGF4, FGF5, FGF6, FGF7, FGF8, FGF9, or FGF10. In one instance, the one or more agents comprise FGF2 (basic fibroblast growth factor, bFGF). In one instance, the method of differentiating the host stem cells comprises contacting the host stem cell with no more than about 200 ng/mL of FGF (e.g., bFGF), e.g., from 100 to 200 ng/mL. In one instance, the method of differentiating the host stem cells comprises contacting the host stem cell with no more than about 100 ng/mL of FGF (e.g., bFGF), e.g., from about to 1 ng/mL; or from about 1 to about 100 ng/mL of FGF (e.g., bFGF). In one instance, the concentration of FGF (e.g., bFGF) used herein is from about: 0.1-1, 1-10, 10-20, 20-30, 30-40, 40-50, 50-50-70, 80-90, or 90-100 ng/mL. In one instance, the concentration of FGF (e.g., bFGF) used herein is about: 0.1, 0.2, 0.4, 0.6, 0.8, 1, 2, 3, 4, 5, 6, 7, 8, 9, 10, 11, 12, 13, 14, 15, 16, 17, 18, 19, 20, 25, 30, 35, 45, 50, 60, 70, 80, or 90 ng/mL. In one instance, the one or more agents further comprise an antioxidant or reducing agent (e.g., 2-mercaptoethanol). In one instance, the one or more agents further comprise a vitamin (e.g., nicotinamide). In one instance, the method of differentiating host stem cell comprises contacting the mammalian trophoblast stem cell with FGF (e.g., bFGF), 2-mercaptoethanol, and nicotinamide. In one instance, the concentration of antioxidant/reducing agent (e.g., 2-mercaptoethanol) is no more than about 10 mmol/L, e.g., from about 0.1 to about 10 mmol/L. In one instance, the concentration of antioxidant/reducing agent (e.g., 2-mercaptoethanol) is from about: 0.1-1, 1-2, 2-3, 3-4, 4-5, 5-6, 6-7, 7-8, 8-9, or 9-10 mmol/L. In one instance, the concentration of antioxidant/reducing agent (e.g., 2-mercaptoethanol) is about: 0.2, 0.5, 1, 1.5, 2, 3, 4, 5, 6, 7, 8, or 9 mmol/L. In one instance, the concentration of antioxidant/reducing agent (e.g., 2-mercaptoethanol) is about 1 mmol/L. In one instance, the concentration of vitamin (e.g., nicotinamide) is no more than about 100 mmol/L, e.g., from about 1 to about 100 mmol/L. In one instance, the concentration of vitamin (e.g., nicotinamide) is from about: 1-10, 10-20, 20-30, 30-40, 40-50, 50-60, 50-70, 80-90, or 90-100 mmol/L. In one instance, the concentration of vitamin (e.g., nicotinamide) is about: 2, 4, 6, 8, 10, 12, 14, 16, 18, 30, 40, 50, 60, 70, 80, or 90 mmol/L. In one instance, the concentration of vitamin (e.g., nicotinamide) is about 10 mmol/L.

In one instance, the method of differentiating the host stem cells comprises contacting the host stem cell with one or more agents to regulate activity or expression level of cAMP Responsive Element Binding Protein 1 (CREB1). In one instance, the one or more agents regulate CREB1 phosphorylation. In one instance, the one or more agents comprise a vitamin metabolite, e.g., retinoic acid. In one instance, the one or more agents comprise a CREB1-binding protein. In one instance, the one or more agents regulate one or more factors comprising mix11, Cdx2, Oct4, Sox17, Foxa2, or GSK3β.

In one instance, the one or more agents comprise an exogenous miR-124 precursor or an exogenous anti-miR-124. In one instance, the host stem cell is transfected with the exogenous miR-124 precursor or the exogenous anti-miR-124. In one instance, cis-regulatory element (CRE) of TGACGTCA of promoters of the miR-124 is modulated. In some embodiments, the miR-124 is miR-124a, miR-124b, miR-124c, miR-124d, or miR-124e. In one instance, the miR-124 is miR-124a, e.g., Homo Sapiens miR-124a (hsa-miR-124a).

In one instance, the host stem cell differentiates into the differentiated cell within one day after the start of the differentiating. In some embodiments, induction of differentiation of the host stem cells comprises culturing an undifferentiated host stem cell in a medium comprising a growth factor (e.g., bFGF) under conditions (e.g., 12, 24, 48, 76, or 96 hours) sufficient to induce the differentiation. The medium can further comprise serum (e.g., FBS), carbohydrates (e.g., glucose), antioxidants/reducing agents (e.g., β-mercaptonethanol), and/or vitamins (e.g., nicotinamide). Yield of the differentiated cells is measured, e.g., insulin+/Ngn3+ cells or insulin+/glucagon+ cells as indicators for pancreatic progenitors. In one instance, FBS and insulin levels are positively correlated during FGF (e.g., bFGF) induction, e.g., as indicated by Western blot analysis.

In some embodiments, upon cell induction (e.g, by bFGF), a time-course analysis, e.g, for 4, 8, 16, 24, 32, 40, or 48 hours, can be conducted to monitor levels of transcription factors identifying the cascading stages of cell differentiation development. In some embodiments, declining Mix11 and high levels of T and Gsc can imply a transition from the host stem cells to mesendoderm. In some embodiments, dominant pluripotency transcription factors at each stage of differentiation include Cdx2 for mesendoderm, Oct4 or Nanog for DE, Cdx2 or Nanog for primitive gut endoderm, or Sox2 for pancreatic progenitors. In some embodiments, FGF (e.g., bFGF) induces multifaceted functions of miR-124a via upregulation of Oct4, Sox17, or Foxa2, but downregulation of Smad4 or Mix11 at the DE stage.

In some embodiments, during cell differentiation, levels of proteins or hormones characteristic to the target differentiated cells are also measured with a time-course analysis, e.g., for 4, 8, 16, 24, 32, or 48 hours. For example, betatrophin, C-peptide, and insulin are measured, e.g., with qPCR analysis, for pancreatic progenitor production.

In some embodiments, a growth factor is used to induce differentiation of the host stem cell. In one instance, the growth factor is FGF (e.g., bFGF), bone morphogenetic protein (BMP), or vascular endothelial growth factor (VEGF). In some embodiments, an effective amount of a growth factor is no more than about 100 ng/ml, e.g., about: 1, 2, 5, 10, 15, 20, 25, 30, 35, 40, 45, 50, 60, 70, 80, 90, or 100 ng/mL. In one instance, the host stem cell is a mammalian trophoblast stem cell. In one instance, the mammalian trophoblast stem cell is an hTS cell.

In some embodiments, a culture medium used to differentiate the host stem cell can further comprise an effective amount of a second agent that works synergistically with a first agent to induce differentiation into a mesendoderm direction. In some embodiments, the first and second agents are different growth factors. In some embodiments, the first agent is added to the culture medium before the second agent. In some embodiments, the second agent is added to the culture medium before the first agent. In one instance, the first agent is FGF (e.g., bFGF). In some embodiments, the second agent is BMP, e.g., BMP2, BMP7, or BMP4, added before or after the first agent. In some embodiments, an effective amount of a BMP is no more than about 100 ng/ml, e.g., about: 1, 2, 5, 10, 15, 20, 25, 30, 35, 40, 45, 50, 60, 70, 80, 90, or 100 ng/mL. In one instance, the host stem cell is a mammalian trophoblast stem cell. In one instance, the mammalian trophoblast stem cell is an hTS cell.

In some embodiments, a culture medium used to differentiate the host stem cell (e.g., a mammalian trophoblast stem cell) can comprise feeder cells. Feeder cells are cells of one type that are co-cultured with cells of another type, to provide an environment in which the cells of the second type can grow. In some embodiments, a culture medium used is free or essentially free of feeder cells. In some embodiments, a GSK-3 inhibitor is used to induce differentiation of the host stem cell.

### Method of Manufacturing Cells

Provided herein is a method of producing a cell (e.g., a genetically engineered cell) comprising: introducing into said cell a gene editing multi-site (GEMS) construct. In some embodiments, the GEMS construct comprises a GEMS sequence comprising a plurality of first recognition sequences for a site-specific recombinase, wherein the first recognition sequence can undergo a site-specific recombination with a second recognition sequence of a site-specific recombinase, when contacted with the site specific recombinase. In some embodiments, the GEMS construct comprises a GEMS sequence comprising a plurality of nuclease recognition sequences, wherein each of the plurality of nuclease recognition sequences comprises a target sequence and a protospacer adjacent motif (PAM), or reverse complement thereof.

In some embodiments, the method further comprises introducing into said host cell an endonuclease for mediating integration of said GEMS construct into said genome. In some embodiments, said nuclease is an endonuclease. In some embodiments, said endonuclease comprises a meganuclease, wherein said homology sequence of said homology arm comprises a consensus sequence of said meganuclease. In some embodiments, said meganuclease is I-SceI. In some embodiments, said endonuclease comprises a CRISPR-associated nuclease.

In some embodiments, the method further comprises introducing into said host cell a guide polynucleotide (e.g., a gRNA) for mediating integration of said GEMS construct into said genome. In some embodiments, said guide polynucleotide recognizes a sequence of said genome at said insertion site. In some embodiments, said insertion site is at a safe harbor site of the genome. In some embodiments, said safe harbor site comprises an AAVs1 site, a Rosa26 site, or a C-C motif receptor 5 (CCR5) site. In some embodiments, said GEMS construct element is integrated at said insertion site.

In some embodiments, the method further comprises introducing a donor vector comprising a donor nucleic acid sequences or an exogenous polynucleotide in said host cell for insertion within a GEMS sequence. In some embodiments, the donor nucleic acid sequence is inserted within a nuclease recognition sequence in the GEMS sequence. In some embodiments, the donor nucleic acid sequence is inserted such that it replaces a nuclease recognition sequence in the GEMS sequence. In some embodiments, at least 2, 3, 4, 5, 6, 7, 8, 9, 10, 11, 12, 13, 14, 15, 16, 17, 18, 19, 20, 25 or more donor nucleic acid sequences are introduced into the host cell.

Insertion of the donor nucleic acid can be mediated by a CRISPR-associated nuclease. In some embodiments, the method further comprises introducing said guide polynucleotide into said host cell. In some embodiments, said guide polynucleotide is a guide RNA. In some embodiments, the method further comprises introducing the CRISPR-associated nuclease into said host cell, wherein said nuclease when bound to said guide polynucleotide recognizes a nuclease recognition sequence of said plurality of nuclease recognition sequences.

In some embodiments, said donor nucleic acid sequence polynucleotide encodes a therapeutic protein. In some embodiments, said therapeutic protein comprises a chimeric antigen receptor (CAR). In some embodiments, said CAR is a CD19 CAR or a portion thereof. In some embodiments, said therapeutic protein comprises dopamine or a portion thereof. In some embodiments, said therapeutic protein comprises insulin, proinsulin, or a portion thereof.

In some embodiments, the donor nucleic acid sequences comprise a nucleotide sequence of SEQ ID NO: 20. In some embodiments, the donor nucleic acid sequences comprise a nucleotide sequence of SEQ ID NO: 21. In some embodiments, the donor nucleic acid sequences comprise a nucleotide sequence of SEQ ID NO: 22. In some embodiments, the donor nucleic acid sequences comprise a nucleotide sequence of SEQ ID NO: 23. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 20. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 21. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 22. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 23. In some embodiments, the exogenous polynucleotide encode an antibody or a fragment thereof. In some embodiments, the exogenous polynucleotide encode a heavy chain of an antibody or a fragment thereof. In some embodiments, the exogenous polynucleotide encode a light chain of an antibody or a fragment thereof. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 85. In some embodiments, the donor nucleic acid sequences comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 86. In some embodiments, the exogenous polynucleotide comprises a nucleic acid sequence comprising a first polynucleotide encoding a light chain polypeptide or a fragment thereof of an antibody or a fragment thereof. In some embodiments, the exogenous polynucleotide comprises a nucleic acid sequence comprising a second polynucleotide encoding a heavy chain polypeptide or a fragment thereof of an antibody or a fragment thereof. In some embodiments, the first polynucleotide encoding a light chain or a fragment thereof comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 85. In some embodiments, the first polynucleotide encoding a heavy chain or a fragment thereof comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 86. In some embodiments, the first polynucleotide and the second polynucleotide are linked by a linker polynucleotide. In some embodiments, a linker polynucleotide comprises a sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 87 or SEQ ID NO: 88.

In some embodiments, the method further comprise introducing into said cell comprising a GEMS sequence comprising a plurality of a first recognition sequence for a site-specific recombinase, a recombinase polypeptide or a nucleic acid sequence encoding said site-specific recombinase polypeptide.

In some embodiments, the method further comprises introducing into said cell comprising a GEMS sequence comprising a plurality of nuclease recognition sequences (i) a second guide polynucleotide, wherein said guide polynucleotide recognizes a second nuclease recognition sequence of said plurality of nuclease recognition sequences; (ii) a second nuclease, wherein said second nuclease recognizes said second nuclease recognition sequence when bound to said second guide polynucleotide; and (iii) a second donor nucleic acid sequence for integration within said second nuclease recognition sequence. In some embodiments, the method further comprises propagating said host cell.

Provided herein is a method of editing a genome comprising: obtaining a genetically engineered cell that comprises a gene editing multi-site (GEMS) construct inserted into a genome of a host cell at an insertion site, wherein said GEMS construct comprises a GEMS sequence comprising a plurality of nuclease recognition sequences, wherein each of the plurality of nuclease recognition sequences comprises a target sequence and a protospacer adjacent motif (PAM), or reverse complement thereof.

In some embodiments, said nuclease cleaves said GEMS sequence when bound to said guide polynucleotide to form a double-stranded break in said GEMS sequence. In some embodiments, the method further comprises introducing into said host cell a donor nucleic acid sequence, wherein said donor nucleic acid sequence is integrated into said GEMS sequence at said double-stranded break. In some embodiments, said donor nucleic acid sequence encodes a therapeutic protein. In some embodiments, said therapeutic protein comprises a chimeric antigen receptor (CAR). In some embodiments, said CAR is a CD19 CAR or a portion thereof. In some embodiments, said therapeutic protein comprises dopamine or a portion thereof. In some embodiments, said therapeutic protein comprises insulin, proinsulin, or a portion thereof.

In some embodiments, the method of editing a genome further comprises introducing into said host cell (i) a second guide polynucleotide, wherein said guide polynucleotide recognizes a second nuclease recognition sequence of said plurality of nuclease recognition sequences; (ii) a second nuclease, wherein said second nuclease recognizes said second nuclease recognition sequence when bound to said second guide polynucleotide; and (iii) a second donor nucleic acid sequence for integration within said second nuclease recognition sequence. In some embodiments, said host cell is a stem cell. In some embodiments, the method further comprises differentiating said stem cell into a T-cell. In some embodiments, said T-cell is selected from the group consisting of an αβ T-cell, an NK T-cell, a γδ T-cell, a regulatory T-cell, a T helper cell and a cytotoxic T-cell. In some embodiments, said differentiating occurs prior to said introducing said guide polynucleotide and said nuclease into said host cell. In some embodiments, said differentiating occurs after said introducing said guide polynucleotide and said nuclease into said host cell. In some embodiments, said insertion site is within a safe harbor site of said genome. In some embodiments, said safe harbor site comprises an AAVs1 site, a Rosa26 site, or a C-C motif receptor 5 (CCR5) site.

In some embodiments, said PAM sequence is selected from the group consisting of: CC, NG, YG, NGG, NAA, NAT, NAG, NAC, NTA, NTT, NTG, NTC, NGA, NGT, NGC, NCA, NCT, NCG, NCC, NRG, TGG, TGA, TCG, TCC, TCT, GGG, GAA, GAC, GTG, GAG, CAG, CAA, CAT, CCA, CCN, CTN, CGT, CGC, TAA, TAC, TAG, TGG, TTG, TCN, CTA, CTG, CTC, TTC, AAA, AAG, AGA, AGC, AAC, AAT, ATA, ATC, ATG, ATT, AWG, AGG, GTG, TTN, YTN, TTTV, TYCV, TATV, NGAN, NGNG, NGAG, NGCG, AAAAW, GCAAA, TGAAA, NGGNG, NGRRT, NGRRN, NNGRRT, NNAAAAN, NNNNGATT, NNAGAAW, NAAAAC, NNAAAAAW, NNAGAA, NAAAAC, NNNNACA, GNNNCNNA, NNNNGATT, NNAGAAW, NNGRR, and TGGAGAAT. In some embodiments, said nuclease is a CRISPR-associated nuclease. In some embodiments, said CRISPR-associated nuclease is a Cas9 enzyme.

In some embodiments, the genetically engineered cell can comprise an inhibition in expression of one or more genes related to eliciting an immune response in a host (e.g., MHC-class I genes, MHA-class II genes, genes encoding for one or more HLA, (32 microglobulin gene). Expression levels of genes can be reduced to various extents. For example, expression of one or more genes can be reduced by or by about 100%. In some cases, expression of one or more genes can be reduced by or by about 99%, 95%, 90%, 85%, 80%, 75%, 70%, 65%, 60%, 55%, or 50% of normal expression, e.g., compared to the expression of non-modified controls. In some cases, expression of one or more genes can be reduced by at least or to at least about 99% to 90%; 89% to 80%, 79% to 70%; 69% to 60%; 59% to 50% of normal expression, e.g., compared to the expression of non-modified controls. For example, expression of one or more genes can be reduced by at least or at least about 90% or by at least or at least about 90% to 99% of normal expression.

The methods described herein, can utilize techniques which can be used to allow a DNA or RNA construct entry into a host cell include, but are not limited to, calcium phosphate/DNA co-precipitation, microinjection of DNA into a nucleus, electroporation, bacterial protoplast fusion with intact cells, transfection, lipofection, infection, particle bombardment, sperm mediated gene transfer, or any other technique known by one skilled in the art.

Certain aspects disclosed herein can utilize vectors. Any plasmids and vectors can be used as long as they are replicable and viable in a selected host. Vectors known in the art and those commercially available (and variants or derivatives thereof) can be engineered to include one or more recombination sites for use in the methods. Vectors that can be used include, but not limited to eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3, pREP4, pCEP4, and pEBVHis (Invitrogen, Corp.), and variants or derivatives thereof.

### Gene Disruption

Gene disruption can be performed by any methods known in the art, for example, by knockout, knockdown, RNA interference, dominant negative, etc. Gene suppression can also be done in a number of ways. For example, gene expression can be reduced by knock out, altering a promoter of a gene, and/or by administering interfering RNAs (knockdown). If one or more genes are knocked down in a cell, the one or more genes can be reduced by administrating RNA interfering reagents, e.g., siRNA, shRNA, or microRNA. For example, a nucleic acid which can express shRNA can be stably transfected into a cell to knockdown expression. Furthermore, a nucleic acid which can express shRNA can be inserted into the genome of a cell, thus knocking down a gene with in a cell.

Disruption methods can also comprise overexpressing a dominant negative protein. This method can result in overall decreased function of a functional wild-type gene. Additionally, expressing a dominant negative gene can result in a phenotype that is similar to that of a knockout and/or knockdown.

In some cases a stop codon can be inserted or created (e.g., by nucleotide replacement), in one or more genes, which can result in a nonfunctional transcript or protein (sometimes referred to as knockout). For example, if a stop codon is created within the middle of one or more genes, the resulting transcription and/or protein can be truncated, and can be nonfunctional. However, in some cases, truncation can lead to an active (a partially or overly active) protein. In some cases, if a protein is overly active, this can result in a dominant negative protein, e.g., a mutant polypeptide that disrupts the activity of the wild-type protein.

This dominant negative protein can be expressed in a nucleic acid within the control of any promoter. For example, a promoter can be a ubiquitous promoter. A promoter can also be an inducible promoter, tissue specific promoter, and/or developmental specific promoter. The nucleic acid that codes for a dominant negative protein can then be inserted into a cell. Any known method can be used. For example, stable transfection can be used. Additionally, a nucleic acid that codes for a dominant negative protein can be inserted into a genome of a cell.

Gene disruption can be done using a CRISPR/Cas system. Methods to disrupt immunogenicity genes (e.g., MHA-Class I genes, MHC-class II genes, genes encoding HLA) using the CRISPR/Cas system are well known in the art. See for example, Hong et al, J. Immunother, 2017, the contents of which are incorporated herein by reference.

Expression can be measured by any known method, such as quantitative PCR (qPCR), including but not limited to PCR, real-time PCR (e.g., Sybr-green), and/or hot PCR. In some cases, expression of one or more genes can be measured by detecting the level of transcripts of the genes. For example, expression of one or more genes can be measured by Northern blotting, nuclease protection assays (e.g., RNase protection assays), reverse transcription PCR, quantitative PCR (e.g., real-time PCR such as real-time quantitative reverse transcription PCR), in situ hybridization (e.g., fluorescent in situ hybridization (FISH)), dot-blot analysis, differential display, serial analysis of gene expression, subtractive hybridization, microarrays, nanostring, and/or sequencing (e.g., next-generation sequencing). In some cases, expression of one or more genes can be measured by detecting the level of proteins encoded by the genes. For example, expression of one or more genes can be measured by protein immunostaining, protein immunoprecipitation, electrophoresis (e.g., SDS-PAGE), Western blotting, bicinchoninic acid assay, spectrophotometry, mass spectrometry, enzyme assays (e.g., enzyme-linked immunosorbent assays), immunohistochemistry, flow cytometry, and/or immunocytochemistry. Expression of one or more genes can also be measured by microscopy. The microscopy can be optical, electron, or scanning probe microscopy. Optical microscopy can comprise use of bright field, oblique illumination, cross-polarized light, dispersion staining, dark field, phase contrast, differential interference contrast, interference reflection microscopy, fluorescence (e.g., when particles, e.g., cells, are immunostained), confocal, single plane illumination microscopy, light sheet fluorescence microscopy, deconvolution, or serial time-encoded amplified microscopy. In some embodiments, the genetically engineered cells can further comprise disruption in one or more genes (e.g., genes encoding HLA) to reduce the immunogenicity of the cells.

In some embodiments, the genetically engineered cell further comprises a nucleic acid sequence coding for a suicide gene, wherein the suicide gene encodes an apoptosis inducing molecule. Nucleic acid encoding for suicide gene can be provided on an additional plasmid or other suitable vector that is inserted into the genetically engineered cell. The term “apoptosis” as used herein refers to the art recognized use of the term for an active process of programmed cell death characterized by morphological changes in the cell. Apoptosis is characterized by membrane blebbing and nuclear DNA fragmentation. As used herein, “suicide gene” is a nucleic acid coding for a product (e.g., an apoptosis inducing molecule), wherein the product causes cell death by itself or in the presence of other compounds. A representative example of such a suicide gene is one which codes for thymidine kinase of herpes simplex virus. Accordingly, in some embodiments, a suicide gene can be a gene coding for a prodrug-activating enzyme. Additional examples are thymidine kinase of varicella zoster virus and the bacterial gene cytosine deaminase which can convert 5-fluorocytosine to the highly toxic compound 5-fluorouracil. The expression of a suicide gene induces cell death. For, example when cells expressing thymidine kinase are contacted with ganciclovir, the thymidine kinase phosphorylates the nucleoside analog resulting in a form of the compound that can be further processed and incorporated into elongating DNA, leading to chain termination. Other genes encoding different enzymatic activities can be used as suicide genes. These include the E. coli purine nucleoside phosphorylase E gene, which generates toxic purines, and the bacterial cytosine deaminase gene which converts 5-fluorocytosine to 5-fluorouracil. Both of these genes function by the in situ conversion of a nucleoside analogue into a form that is incorporated into replicating DNA thereby interfering with the replication process. Other suicide genes can be employed include the E. coli nitroreductase gene (see Drabek, et al. Gene Therapy 4(2):93-100, 1997) that acts by converting the pro-drug CB 1954 into a cytotoxic DNA interstrand crosslinking agent and the hepatic cytochrome P450 2B1 (see Wei, et al. Human Gene Therapy 5(8):969-978, 1994) that acts by converting the anticancer drug cyclophospharmide into a toxic DNA-alkylating agent.

As used herein “prodrug” means any compound that can be converted to a toxic product, i.e. toxic to a genetically engineered cell of the present disclosure. The prodrug is converted to a toxic product by the gene product of the therapeutic nucleic acid sequence (suicide gene) in the vector useful in the method of the present invention. Representative examples of such a prodrug is ganciclovir which is converted in vivo to a toxic compound by HSV-thymidine kinase. The ganciclovir derivative subsequently is toxic to tumor cells. Other representative examples of prodrugs include acyclovir, FIAU [1-(2-deoxy-2-fluoro-β-D-arabinofuranosyl)-5-iodouracil], 6-methoxypurine arabinoside for VZV-TK, and 5-fluorocytosine for cytosine deambinase. Ganciclovir may be administered readily by a person having ordinary skill in this art. A person with ordinary skill would readily be able to determine the most appropriate dose and route for the administration of ganciclovir. Preferably, ganciclovir is administered in a dose of from about 1-20 mg/day/kg body weight. Preferably, acyclovir is administered in a dose of from about 1-100 mg/day/kg body weight and FIAU is administered in a dose of from about 1-50 mg/day/kg body weight.

An alternative approach to suicide genes involves expressing endogenous components of cellular apoptotic pathways. In some embodiments, the “apoptosis inducing molecule” can be a protein involved in the cellular apoptotic pathway. Non limiting examples include, members of the ICE/CED3 family of apoptosis inducing proteases (such as Caspase-1 (ICE), hICE, ICE-LAP45, Mch2 alpha), Caspase-2 (ICH1), Caspase-3 (CPP32, Yama, Apopain), Caspase-4 (TX, ICH2, ICE rel II), Caspase-5 (ICE rel III, TY), Caspase-6 (Mch-2), Caspase-7 (Mch-3, ICE-LAP3, CMH-1), Caspase-8 (MACH, FLICE, Mch-5), Caspase-9 (ICE-LAP6, Mch6) and Caspase-10 (Mch4)), members of the granzyme family (such as Granzyme A and Granzyme B), Fas ligand (FasL), and functional fragments, variants, and mixtures of any of these. Some embodiments employ Caspase 3, Caspase 4, Caspase 5, Granzyme B, and functional fragments, variants, and mixtures thereof. With the exception of FasL, these genes, when overexpressed following transfection, induce apoptosis in the transfected cells (Miura M., et al., (1993) Cell 75, 653-660; Chinnayan et al., (1995) Cell, 81, 505-512; Los, et al., (1995) Nature 375, 81; Muzio, et al., (1996) Cell 85, 817-827).

The term “caspase” as used herein refers to a cysteine protease that specifically cleaves proteins after Asp residues. Caspases exist as inactive proenzymes which undergo proteolytic processing at conserved aspartic residues to produce 2 subunits, large and small, that dimerize to form the active enzyme. This protein was shown to cleave and activate caspases 6, 7 and 9, and itself could be processed by caspases 8, 9 and 10. Caspases are initially expressed as zymogens, in which a large subunit is N-terminal to a small subunit. Caspases are generally activated by cleavage at internal Asp residues. Caspases are found in a myriad of organisms, including human, mouse, insect (e.g., Drosophila), and other invertebrates (e.g., C. elegans). The caspases include, but are not limited to, Caspase-1 (also known as “ICE”), Caspase-2 (also known as “ICH-1”), Caspase-3 (also known as “CPP32,” “Yama,” “apopain”), Caspase-4 (also known as “ICE.re111”; “TX,” “ICH-2”), Caspase-5 (also known as “ICE.re1111”; “TY”), Caspase-6 (also known as “Mch2”), Caspase-7 (also known as “Mch3,” “ICE-LAP3” “CMH-1”), Caspase-8 (also known as “FLICE;” “MACH;” “Mch5”), Caspase-9 (also known as “ICE-LAP6;” “Mch6”), Caspase-10 (also known as “Mch4,” “FLICE-2”). The term “apoptosis-inducing molecule” is also intended to include pro-forms of caspases, i.e., activatable intermediates in the apoptotic cascade. The caspases may be prepared inactive forms that require activation by an exogenous ligand which is an oligomerizing agent. The phrase “oligomerizing agent” as used herein refers to a ligand that facilitates the association of a number of components to form dimers, trimers, tetramers, or oligomers.

The oligomerizing agent can be used to associate like components, i.e., homodimerize. Alternatively, the oligomerizing agent can be used to associate different components, i.e, heterodimerize. The action of bringing the separate components together results in a triggering event that initiates cellular processes, such as apoptosis. For example, the oligomerizing agent can be a dimerizing agent such as AP20187 (Ariad), that facilitates the association of two caspases (e.g., caspase-3 and caspase 9), to trigger apoptosis in the cell. Accordingly, the oligomerizing agent provides an additional level of regulation in which apoptosis is activated when desired by administering the exogenous ligand which is an oligomerizing agent to the cell. Examples of ligands include, but are not limited to, AP20187 (Ariad), FK-509-type ligands, cyclosporin A-type ligands, tetracycline, steroid ligands, the tetracycline Tet-On/Tet-Off system, an ecdysone-dimerizer system, an antiprogestin-dimerizer system, and the courmarin-dimerizer system. In one embodiment, the oligomerizing agent is AP20187 (Ariad). Examples of specific dimerizing agents include, but are not limited to, FKBP:FK1012, FKBP:synthetic divalent FKBP ligands, FRB:rapamycin/FKBP, cyclophilin:cyclosporin, DHFR:methotrexate, TetR:tetracycline or doxycycline or other analogs or mimics thereof, progesterone receptor:RU486, ecodysone receptor:ecdysone or muristerone A or other analogs or mimics thereof, and DNA gyrase:coumermycin.

In some embodiments, an apoptosis inducing molecule can be selectively activated in response to an exogenous ligand, for example, by its chemically induced dimerization, (CID) (See for example, US20040040047A1, WO 95/02684, U.S. patent application Ser. No. 08/093,499 and Ser. No. 08/179,143.Stasi et al, N Engl J MEd, 2011). Accordingly, in some embodiments, the apoptosis inducing molecule is fused to an inducer ligand binding domain.

In some embodiments, the expression of the suicide gene can be regulated by an inducible promoter. In some embodiments, the nucleic acid encoding an apoptosis inducing molecule is operably linked to a nucleic acid sequence encoding a regulatory element (e.g., a promoter). Several examples of inducible promoters are well known in the art. Non limiting examples include cyclooxygenase promoter, a tumor necrosis factor promoter, an interleukin regulated promoter, alcohol-regulated promoter, steroid regulated promoter, dexamethasone regulated promoter, tetracycline regulated promoter, metal regulated promoter, light regulated promoter, and temperature regulated promoter.

### Enriching

In some embodiments, subject methods include (i) a step of enriching the host cell population for the cells that are in a desired phase(s) of the cell cycle, and/or (ii) a step of blocking the host cell at a desired phase in the cell cycle. The cell cycle is the series of events that take place in a cell leading to its division and duplication (replication) that produces two daughter cells. Two major phases of the cell cycle are the S phase (DNA synthesis phase), in which DNA duplication occurs, and the M phase (mitosis), in which the chromosomes segregation and cell division occurs. The eukaryotic cell cycle is traditionally divided into four sequential phases: G1, S, G2, and M. G1, S, and G2 together can collectively be referred to as “interphase”. Under certain conditions, cells can delay progress through G1 and can enter a specialized resting state known as G0 (G zero), in which they can remain for days, weeks, or even years before resuming proliferation. The period of transition from one state to another can be referred to using a hyphen, for example, G1/S, G2/M, etc. As is known in the art, various checkpoints exist throughout the cell cycle at which a cell can monitor conditions to determine whether cell cycle progression should occur. For example, the G2/M DNA damage checkpoint serves to prevent cells from entering mitosis (M-phase) with genomic DNA damage.

A step of enriching a population of eukaryotic cells for cells in a desired phase of the cell cycle (e.g., G1, S, G2, M, G1/S, G2/M, G0, etc., or any combination thereof), and can be performed using any convenient method (e.g., a cell separation method and/or a cell synchronization method).

In some cases, the method includes a step of enriching a population of the host cells for cells in the G0 phase of the cell cycle. For example, in some cases, a subject method includes: (a) enriching a population of eukaryotic cells for cells in the G0 phase of the cell cycle; and (b) contacting the GEMS construct and/or the donor nucleic acid sequences with a Cas9 targeting complex (e.g., via introducing into the host cell(s) at least one component of a Cas9 targeting complex) (e.g., contacting the GEMS construct and/or donor nucleic acid sequences with (i) a Cas9 protein; and (ii) a guide polynucleotide.

In some cases, the method includes a step of enriching a population of host cells for cells in the G1 phase of the cell cycle. For example, in some cases, the method includes: (a) enriching a population of the host cells for cells in the G1 phase of the cell cycle; and (b) contacting the GEMS construct and/or the donor nucleic acid sequences with a Cas9 targeting complex (e.g., via introducing into the host cell(s) at least one component of a Cas9 targeting complex) (e.g., contacting the GEMS construct and/or donor nucleic acid sequences with (i) a Cas9 protein; and (ii) a guide RNA.

In some cases, the method includes a step of enriching a population of the host cells for cells in the G2 phase of the cell cycle. For example, in some cases, the method includes: (a) enriching a population of the host cells for cells in the G2 phase of the cell cycle; and (b) contacting the GEMS construct and/or donor nucleic acid sequences with a Cas9 targeting complex (e.g., via introducing into the host cell(s) at least one component of a Cas9 targeting complex) (e.g., contacting the GEMS construct and/or donor nucleic acid sequences with (i) a Cas9 protein; and (ii) a guide RNA.

In some cases, the method includes a step of enriching a population of the host cells for cells in the S phase of the cell cycle. For example, in some cases, the method includes: (a) enriching a population of the host cells for cells in the S phase of the cell cycle; and (b) contacting the GEMS construct and/or donor nucleic acid sequences with a Cas9 targeting complex (e.g., via introducing into the host cell(s) at least one component of a Cas9 targeting complex) (e.g., contacting the GEMS construct and/or donor nucleic acid sequences with (i) a Cas9 protein; and (ii) a guide RNA.

In some cases, the method includes a step of enriching a population of the host cells for cells in the M phase of the cell cycle. For example, in some cases, the method includes: (a) enriching a population of the host cells for cells in the M phase of the cell cycle; and (b) contacting the GEMS construct and/or donor nucleic acid sequences with a Cas9 targeting complex (e.g., via introducing into the host cell(s) at least one component of a Cas9 targeting complex) (e.g., contacting the GEMS construct and/or donor nucleic acid sequences with (i) a Cas9 protein; and (ii) a guide RNA.

In some cases, the method includes a step of enriching a population of the host cells for cells in the G1/S transition of the cell cycle. For example, in some cases, the method includes: (a) enriching a population of the host cells for cells in the G1/S transition of the cell cycle; and (b) contacting the GEMS construct and/or donor nucleic acid sequences with a Cas9 targeting complex (e.g., via introducing into the host cell(s) at least one component of a Cas9 targeting complex) (e.g., contacting the GEMS construct and/or donor nucleic acid sequences with (i) a Cas9 protein; and (ii) a guide RNA.

In some cases, the method includes a step of enriching a population of the host cells for cells in the G2/M transition of the cell cycle. For example, in some cases, the method includes: (a) enriching a population of the host cells for cells in the G2/M transition of the cell cycle; and (b) contacting the GEMS construct and/or donor nucleic acid sequences with a Cas9 targeting complex (e.g., via introducing into the host cell(s) at least one component of a Cas9 targeting complex) (e.g., contacting the GEMS construct and/or donor nucleic acid sequences with (i) a Cas9 protein; and (ii) a guide RNA.

By “enrich” is meant increasing the fraction of desired cells in the resulting cell population. For example, in some cases, enriching includes selecting desirable cells (e.g., cells that are in the desired phase of the cell cycle) away from undesirable cells (e.g., cells that are not in the desired phase of the cell cycle), which can result in a smaller population of cells, but a greater fraction (i.e., higher percentage) of the cells of the resulting cell population will be desirable cells (e.g., cells that are in the desired phase of the cell cycle). Cell separation methods can be an example of this type of enrichment. In other cases, enriching includes converting undesirable cells (e.g., cells that are not in the desired phase of the cell cycle) into desirable cells (e.g., cells that are in the desired phase of the cell cycle), which can result in a similar size population of cells as the starting population, but a greater fraction of those cells can be desirable cells (e.g., cells that are in the desired phase of the cell cycle). Cell synchronization methods can be an example of this type of enrichment. In some cases, enrichment can both change the overall size of the resulting cell population (compared to the size of the starting population) and increase the fraction of desirable cells. For example, multiple methods/techniques can be combined (e.g., to improve enrichment, to enrich for cells a more than one desired phase of the cell cycle, etc.).

In some cases, enriching includes a cell separation method. Any convenient cell separation method can be used to enrich for cells that are at various phases of the cell cycle. Suitable cell separation techniques for enrichment of cells at particular phases of the cell cycle include, but are not limited to: (i) mitotic shake-off (M-phase; mechanical separation on the basis of cell adhesion properties, e.g., adherent cells in the mitotic phase detach from the surface upon gentle shaking, tapping, or rinsing); (ii) countercurrent centrifugal elutriation (CCE) (G1, S, G2/M, and intermediate states; physical separation on the basis of cell size and density); and (iii) flow cytometry and cell sorting (e.g., G0, G1, S, G2/M; physical separation based on specific intracellular, e.g., DNA, content) and cell surface and/or size properties).

Mitotic shake-off generally includes dislodgment of low adhesive, mitotic cells by agitation (see for example, Beyrouthy et. al., PLoS ONE 3, e3943 (2008); Schorl, C. & Sedivy, Methods 41, 143-150 (2007)). Countercurrent centrifugal elutriation (CCE) generally includes the separation of cells according to their sedimentation velocity in a gravitational field where the liquid containing the cells is made to flow against the centrifugal force with the sedimentation rate of cells being proportional to their size (see for example, Grosse et. al., Prep Biochem Biotechnol. 2012; 42(3):217-33; Banfalvi et. al., Nat. Protoc. 3, 663-673 (2008)). Flow cytometry methods generally include the characterization of cells according to antibody and/or ligand and/or dye-mediated fluorescence and scattered light in a hydrodynamically focused stream of liquid with subsequent electrostatic, mechanical or fluidic switching sorting (see for example, Coquelle et. al., Biochem. Pharmacol. 72, 1396-1404 (2006); Juan et. al., Cytometry 49, 170-175 (2002)). For more information related to cell separation techniques, refer to, for example, Rosner et al., Nat Protoc. 2013 March; 8(3):602-26.

In some cases, enriching includes a cell synchronization method (i.e., synchronizing the cells of a cell population). Cell synchronization is a process by which cells at different stages of the cell cycle within a cell population (i.e., a population of cells in which various individual cells are in different phases of the cycle) are brought into the same phase. Any convenient cell synchronization method can be used in the subject methods to enrich for cells that are at a desired phase(s) of the cell cycle. For example, cell synchronization can be achieved by blocking cells at a desired phase in the cell cycle, which allows the other cells to cycle until they reach the blocked phase. For example, suitable methods of cell synchronization include, but are not limited to: (i) inhibition of DNA replication, DNA synthesis, and/or mitotic spindle formation (e.g., sometimes referred to herein as contacting a cell with a cell cycle blocking composition); (ii) mitogen or growth factor withdrawal (G0, G1, G0/G1; growth restriction-induced quiescence via, e.g., serum starvation and/or amino acid starvation); and (iii) density arrest (G1; cell-cell contact-induced activation of specific transcriptional programs) (see for example, Rosner et al., Nat Protoc. 2013 March; 8(3):602-26), which is hereby incorporated by reference in its entirety, and see references cited therein).

Various methods for cell synchronization is known to one of ordinary skill in the art and any convenient method can be used. For additional methods for cell synchronization (e.g., synchronization of plant cells), see, for example, Sharma, Methods in Cell Science, 1999, Volume 21, Issue 2-3, pp 73-78 (“Synchronization in plant cells-an introduction”); Dolezel et al., Methods in Cell Science, 1999, Volume 21, Issue 2-3, pp 95-107 (“Cell cycle synchronization in plant root meristems”); Kumagai-Sano et al., Nat Protoc. 2006; 1(6):2621-7; and Cools et al., The Plant Journal (2010) 64, 705-714; and Rosner et al., Nat Protoc. 2013 March; 8(3):602-26; all of which are hereby incorporated by reference in their entirety.

### Checkpoint Inhibitors

In some embodiments, a cell (or cells of a cell population), is blocked at a desired phase of the cell cycle (e.g., by contacting the cell with a cycle blocking composition such as a checkpoint inhibitor). In some embodiments, cells of a cell population are synchronized (e.g., by contacting the cells with a cell cycle blocking composition). A cell cycle blocking composition (e.g., checkpoint inhibitors) can include one or more cell cycle blocking agents. The terms “cell cycle blocking agent” and “checkpoint inhibitor” refer to an agent that blocks (e.g., reversibly blocks (pauses), irreversibly blocks) a cell at a particular point in the cell cycle such that the cell cannot proceed further. Suitable cell cycle blocking agents include reversible cell cycle blocking agents. Reversible cell cycle blocking agents do not render the cell permanently blocked. In other words, when reversible cell cycle blocking agent is removed from the cell medium, the cell is free to proceed through the cell cycle. Cell cycle blocking agents are sometimes referred to in the art as cell synchronization agents because when such agents contact a cell population (e.g., a population having cells that are at different stages of the cell cycle), the cells of the population become blocked at the same phase of the cell cycle, thus synchronizing the population of cells relative to that particular phase of the cell cycle. When the cell cycle blocking agent used is reversible, the cells can then be “released” from cell cycle block.

Suitable cell cycle blocking agents include, but are not limited to: nocodazole (G2, M, G2/M; inhibition of microtubule polymerization), colchicine (G2, M, G2/M; inhibition of microtubule polymerization); demecolcine (colcemid) (G2, M, G2/M; inhibition of microtubule polymerization); hydroxyurea (G1, S, G1/S; inhibition of ribonucleotide reductase); aphidicolin (G1, S, G1/S; inhibition of DNA polymerase-alpha and DNA polymerase-delta); lovastatin (G1; inhibition of HMG-CoA reductase/cholesterol synthesis and the proteasome); mimosine (G1, S, G1/S; inhibition of thymidine, nucleotide biosynthesis, inhibition of Ctf4/chromatin binding); thymidine (G1, S, G1/S; excess thymidine-induced feedback inhibition of DNA replication); latrunculin A (M; delays anaphase onset, actin polymerization inhibitor, disrupts interpolar microtubule stability); and latrunculin B (M; actin polymerization inhibitor).

Suitable cell cycle blocking agents can include any agent that has the same or similar function as the agents above (e.g., an agent that inhibits microtubule polymerization, an agent that inhibits ribonucleotide reductase, an agent that inhibits DNA polymerase-alpha and/or DNA polymerase-delta, an agent that inhibits HMG-CoA reductase and/or cholesterol synthesis, an agent that inhibits nucleotide biosynthesis, an agent that inhibits DNA replication, i.e., inhibit DNA synthesis, an agent that inhibits initiation of DNA replication, an agent that inhibits deoxycytosine synthesis, an agent that induces excess thymidine-induced feedback inhibition of DNA replication, and agent that disrupts interpolar microtubule stability, an agent that inhibits actin polymerization, and the like). Suitable agents that block G1 can include: staurosporine, dimethyl sulfoxide (DMSO), glycocorticosteroids, and/or mevalonate synthesis inhibitors. Suitable agents that block G2 phase can include CDK1 inhibitors e.g., RO-3306. Suitable agents that block M can include cytochalasin D.

Non-limiting examples of suitable cell cycle blocking agents include cobtorin; dinitroaniline; benefin (benluralin); butralin; dinitramine; ethalfluralin; oryzalin; pendimethalin; trifluralin; amiprophos-methyl; butamiphos dithiopyr; thiazopyr propyzamider-pronamide-tebutam DCPA (chlorthal-dimethyl); anisomycin; alpha amanitin; jasmonic acid; abscisic acid; menadione; cryptogeine; hydrogen peroxide; sodium permanganate; indomethacin; epoxomycin; lactacystein; icrf 193; olomoucine; roscovitine; bohemine; K252a; okadaic acid; endothal; caffeine; MG132; and cycline dependent kinase inhibitors. For more information regarding cell cycle blocking agents, see Merrill G F, Methods Cell Biol. 1998; 57:229-49, which is hereby incorporated by reference in its entirety.

### Donor Nucleic Acid Sequences

The terms “donor nucleic acid sequence(s)”, “donor gene(s)” or “donor gene(s) of interest” or “exogenous polynucleotide” or “transgene” are used interchangeably herein and refer to the nucleic acid sequence(s) or gene(s) encoding a protein of interest to be inserted or is inserted into the host cell genome at the multiple gene editing site. Typically, the exogenous polynucleotide encoding a polypeptide of interest will be DNA. In some embodiments, the exogenous polynucleotide can further comprise ribonucleotides, nucleotide analogs, or combinations thereof. A nucleotide analog refers to a nucleotide having a modified purine or pyrimidine base, or a nucleotide comprising a modified ribose moiety. Nucleotide analogs also include dideoxy nucleotides, 2′-O-methyl nucleotides, locked nucleic acids (LNA), peptide nucleic acids (PNA), and morpholinos. The nucleotides may be linked by phosphodiester, phosphothioate, phosphoramidite, phosphorodiamidate bonds, or combinations thereof.

In some embodiments, an exogenous polynucleotide encodes a therapeutic protein. In some embodiments, the exogenous polynucleotide encodes a desired recombinant protein. A protein encoded by an exogenous polynucleotide can be any protein, including those useful in biotherapeutic (e.g., therapeutic polypeptide such as hormone, antibody, insulin and the like) and/or diagnostic application, as well as any recombinant protein useful in industrial applications. For example, the protein (e.g., a therapeutic polypeptide) encoded by an exogenous polynucleotide can be, without limit, an antibody, a fragment of an antibody, a monoclonal antibody, a humanized antibody, a humanized monoclonal antibody, a chimeric antibody, an IgG molecule, an IgG heavy chain, an IgG light chain, an Fc region, an IgA molecule, an IgD molecule, an IgE molecule, an IgM molecule, Fc fusion proteins, a vaccine, a growth factor, a cytokine, an interferon, an interleukin, a hormone, a clotting (or coagulation) factor, a blood component, an enzyme, a nutraceutical protein, a glycoprotein, a functional fragment or functional variant of any of the forgoing, or a fusion protein comprising any of the foregoing proteins and/or functional fragments or variants thereof. In exemplary embodiments, a protein encoded by an exogenous polynucleotide is a human or humanized protein.

In some embodiments, the exogenous polynucleotide can be linked to a nucleic acid sequence encoding an amplifiable selectable marker such as hypoxanthine-guanine phosphoribosyltransferase (HPRT), dihydrofolate reductase (DHFR), and/or glutamine synthase (GS).

In other embodiments, the exogenous polynucleotide can be linked to a nucleic acid sequence encoding a reporter protein such as a fluorescent protein, glutathione-S-transferase (GST), chitin binding protein (CBP), maltose binding protein, beta-galactosidase, thioredoxin (TRX), biotin carboxyl carrier protein (BCCP), or calmodulin, and/or a selectable marker polypeptide disclosed herein including for example, a modified selectable marker polypeptide.

In some embodiments, an exogenous polynucleotide can encode a mammalian protein, an artificial protein (e.g. a fusion protein or mutated protein), or a human protein. In some embodiments, exogenous polynucleotide can be of natural origin. Alternatively, the exogenous polynucleotide can be completely of synthetic origin, produced in vitro. Furthermore, exogenous polynucleotide can comprise any combination of isolated naturally occurring DNA molecules, or any combination of an isolated naturally occurring DNA molecule and a synthetic DNA molecule. The exogenous polynucleotide encoding a product of interest can be obtained by standard procedures known in the art from cloned DNA (e.g., a DNA “library”), by chemical synthesis, by cDNA cloning, or by the cloning of genomic DNA, or fragments thereof, purified from the desired cell, or by PCR amplification and cloning. See, for example, Sambrook et al., Molecular Cloning, A Laboratory Manual, 3d. ed., Cold Spring Harbor Laboratory Press, Cold Spring Harbor, N.Y. (2001); Glover, D. M. (ed.), DNA Cloning: A Practical Approach, 2d. ed., MRL Press, Ltd., Oxford, U.K. (1995).

In some embodiments, the exogenous polynucleotide comprises a first polynucleotide encoding a first functional polypeptide. In some embodiments, the exogenous polynucleotide further comprises a second polynucleotide encoding a second functional polypeptide. In some embodiments, the first functional polypeptide can be different from the second functional polypeptide. In some embodiments, at least one of said first and second functional polypeptides can be a protein, hormone, antibody, glycoprotein or derivative or fragment thereof. In some embodiments, the first polynucleotide and the second polynucleotide are linked by a linker polynucleotide. In some embodiments, the first functional polypeptide is a heavy chain of an antibody, a light chain of an antibody of a functional fragment thereof. In some embodiments, the second functional polypeptide is a heavy chain of an antibody, a light chain of an antibody of a functional fragment thereof. In some embodiments, the first functional polypeptide is a heavy chain of an antibody, or a functional fragment thereof, and the second functional polypeptide is a light chain of an antibody, or a functional fragment thereof. In some embodiments, the first functional polypeptide is a light chain of an antibody, or a functional fragment thereof, and the second functional polypeptide is a heavy chain of an antibody, or a functional fragment thereof. In some embodiments, the first polynucleotide encode a first domain of a protein, and the second polynucleotide encodes a second domain of the same protein. In some embodiments, the linker polynucleotide encodes a rigid linker, a flexible linker, a cleavable linker, a self-cleavable linker, a peptide linker, a linker cleavable via ribosome skipping, or any combination thereof. In some embodiments, the self-cleavable linker comprises a 2A sequence e.g., a P2A, T2A, E2A, or a F2A sequence, or a furin recognition sequence. In some embodiments, the exogenous polynucleotide encodes two or more polypeptides linked by a linker polypeptide. In some embodiments, the exogenous polynucleotide encodes a first functional polypeptide and a second functional polypeptide linked by a linker polypeptide. In some embodiments, the exogenous polynucleotide encodes a multicistronic mRNA. In some embodiments, a linker polynucleotide can be an open-reading frame encoding a fusion protein such that a first polypeptide sequence is covalently linked (“fused”) by an intervening amino acid sequence to a second polypeptide sequence. In certain cases, the linker polypeptide is a cleavage-susceptible linker. In some embodiments, the exogenous polynucleotide encodes two or more polypeptides of interest as fusion proteins linked by a cleavage-susceptible linker polypeptide. In some cases, a linker polypeptide can link a functional domains together (as in flexible and rigid linkers) or releasing free functional domain in vivo as in in vivo cleavable linkers.

In some embodiments, a linker polynucleotide can be a non-coding, linking polynucleotide sequence such as a promoter (e.g., CAG promoter or CMV promoter) or an IRES (Internal Ribosome Entry Site) which function to couple a first polynucleotide to a second polynucleotide. As used herein, an “internal ribosome entry site” or “IRES” refers to an element that promotes direct internal ribosome entry to the translation initiation codon (also known as start codon) of a cistron (a protein encoding region), thereby leading to the cap-independent translation of the gene. See, e.g., Jackson R J, Howe 11 M T, Kaminski A (1990) Trends Biochem Sci 15 (12): 477-83) and Jackson R J and Kaminski, A. (1995) RNA 1 (10): 985-1000. The present invention encompasses the use of any cap-independent translation initiation sequence, in particular any IRES element that is able to promote direct internal ribosome entry to the initiation codon of a cistron. “Under translational control of an IRES” as used herein means that translation is associated with the IRES and proceeds in a cap-independent manner. As used herein, the term “IRES” encompasses functional variations of IRES sequences as long as the variation is able to promote direct internal ribosome entry to the initiation codon of a cistron. As used herein, “cistron” refers to a segment of a polynucleotide sequence (DNA) that contains all the information for production of single polypeptide chain.

In some embodiments, a linker polynucleotide comprises a viral 2A sequence. A 2A sequence can be derived from a picornaviral 2A sequence. A picornaviral 2A sequence can be selected from the group consisting of the Enteroviral 2A sequences, Rhinoviral 2A sequences, Cardioviral 2A sequences, Aphthoviral 2A sequences, Hepatoviral 2A sequences, Erboviral 2A sequences, Kobuviral 2A sequences, Teschoviral 2A sequences, and the Parechoviral 2A sequences. Linker polynucleotide encoding 2A linker can be shorter than IRES, having from 5 to 100 base pairs. In some embodiments, a linker polynucleotide can have 5, 10, 15, 20, 25, 30, 35, 40, 45, 50, 55, 60, 65, 70, 75, 80, 85, 90, or 100 base pairs in length. 2A linked genes can be expressed in one single open reading frame and “self-cleavage” can occur co-translationally between the last two amino acids, GP, at the C-terminus of the 2A polypeptide, giving rise to equal amounts of co-expressed proteins. In some cases, a polypeptide comprising a 2A sequence may not give rise to equal amounts of protein post cleavage. In some cases, a first functional polypeptide can be 10%, 20%, 30%, 40%, 50%, 60%, 70%, 80%, 90%, or up to 100% greater in concentration when compared to a second functional polypeptide. In some cases, a second functional polypeptide can be 10%, 20%, 30%, 40%, 50%, 60%, 70%, 80%, 90%, or up to 100% greater in concentration when compared to a second functional polypeptide. In some cases, the first functional polypeptide and the second functional polypeptide are expressed in same concentration. In some embodiments, the linker polypeptide is a viral 2A sequence. In some embodiments, the 2A polypeptide can be about 20 amino acids. In some cases, a 2A polypeptide can contain a consensus motif Asp-Val/Ile-Glu-X-Asn-Pro-Gly-Pro. A consensus motif sequence can act co-translationally. For example, formation of a normal peptide bond between a glycine and proline residue can be prevented, which can result in ribosomal skipping and, thereby, “cleavage” of a nascent polypeptide.

This effect can produce multiple genes at equimolar levels. A 2A peptide can allow translation of multiple proteins in a single open reading frame into a polypeptide that can be subsequently “cleaved” into individual polypeptide through a ribosome-skipping mechanism (Funston, Kallioinen et al. 2008). In some embodiments, a “2A” sequence can include: p2a, GSG-p2a, T2A, E2A, F2A, and BmCPV2A, BmIFV2A, and any combination thereof. A polynucleotide linker can be a DNA double strand, single strand, or a combination thereof. In some cases, a linker can be RNA.

Linker polypeptide may improve biological activity, increase expression yield, and achieving desirable pharmacokinetic profiles. A linker can also comprise hydrazone, peptide, disulfide, or thioesther. In some cases, a linker polypeptide described herein can include a flexible linker. Flexible linkers can be applied when a joined domain requires a certain degree of movement or interaction. Flexible linkers can be composed of small, non-polar (e.g., Gly) or polar (e.g., Ser or Thr) amino acids. A flexible linker can have sequences consisting primarily of stretches of Gly and Ser residues (“GS” linker). An example of a flexible linker can have the sequence of (Gly-Gly-Gly-Gly-Ser)n. By adjusting the copy number “n”, the length of this exemplary GS linker can be optimized to achieve appropriate separation of functional domains, or to maintain necessary inter-domain interactions. Besides GS linkers, other flexible linkers can be utilized for recombinant fusion proteins. In some cases, flexible linkers can also be rich in small or polar amino acids such as Gly and Ser, but can contain additional amino acids such as Thr and Ala to maintain flexibility. In other cases, polar amino acids such as Lys and Glu can be used to improve solubility. Flexible linkers included in linker sequences described herein, can be rich in small or polar amino acids such as Gly and Ser to provide good flexibility and solubility. Flexible linkers can be suitable choices when certain movements or interactions are desired for fusion protein domains. In addition, although flexible linkers may not have rigid structures, they can serve as a passive linker to keep a distance between functional domains. The length of a flexible linkers can be adjusted to allow for proper folding or to achieve optimal biological activity of the fusion proteins.

A linker described herein can further include a rigid linker in some cases. A rigid linker may be utilized to maintain a fixed distance between domains of a polypeptide. Examples of rigid linkers can be: Alpha helix-forming linkers, Pro-rich sequence, (XP)n, X-Pro backbone, A(EAAAK)nA (n=2-5), to name a few. Rigid linkers can exhibit relatively stiff structures by adopting α-helical structures or by containing multiple Pro residues in some cases. A linker described herein can be a cleavable linker, in some cases. In other cases, a linker is not cleavable. Linkers that are not cleavable may covalently join functional domains together to act as one molecule throughout an in vivo processes or an ex vivo process. A linker can also be cleavable in vivo. A cleavable linker can be introduced to release free functional domains in vivo. A cleavable linker can be cleaved by the presence of reducing reagents, proteases, to name a few. For example, a reduction of a disulfide bond may be utilized to produce a cleavable linker. In the case of a disulfide linker, a cleavage event through disulfide exchange with a thiol, such as glutathione, could produce a cleavage. In other cases, an in vivo cleavage of a linker in a recombinant fusion protein may also be carried out by proteases that can be expressed in vivo under pathological conditions (e.g. cancer or inflammation), in specific cells or tissues, or constrained within certain cellular compartments. In some cases, a cleavable linker may allow for targeted cleavage. For example, the specificity of many proteases can offer slower cleavage of a linker in constrained compartments. A cleavable linker can also comprise hydrazone, peptides, disulfide, or thioesther. For example, a hydrazone can confer serum stability. In other cases, a hydrazone can allow for cleavage in an acidic compartment. An acidic compartment can have a pH up to 7. A linker can also include a thioether. A thioether can be nonreducible A thioether can be designed for intracellular proteolytic degradation. In some cases, a linker can be engineered. For example, a linker can be designed to comprise chemical characteristics such as hydrophobicity. In some cases, at least two linker sequences can produce the same protein. A linker can be an engineered linker. Methods of designing linkers can be computational. In some cases, computational methods can include graphic techniques. Computation methods can be used to search for suitable peptides from libraries of three-dimensional peptide structures derived from databases. For example, a Brookhaven Protein Data Bank (PDB) can be used to span the distance in space between selected amino acids of a linker.

Each nuclease recognition sequence in the plurality of nuclease recognition sequences of a GEMS sequence or each first recognition sequence in the plurality of first recognition sequence for a recombinase in a GEMS sequence can be a site where an exogenous polynucleotide can be inserted. Insertion of a donor nucleic acid sequence can be within a recognition sequence (e.g., a first recognition sequence for a recombinase, or a nuclease recognition sequence). Insertion of a donor nucleic acid sequence can replace a recognition sequence (e.g., a first recognition sequence for a recombinase, or a nuclease recognition sequence). In an embodiment, the donor nucleic acid sequences encode a chimeric gene of interest (e.g., CAR). In an embodiment, the donor nucleic acid sequences encode a reporter gene. In an embodiment, the donor nucleic acid sequences encode a transgene. In an embodiment, the donor nucleic acid sequences encode dopamine or other neurotransmitter. In an embodiment, the donor nucleic acid sequences encode insulin or a pro-form of insulin, or other hormones.

In some embodiments, once the host cell has the GEMS sequence integrated, the host cell can be competent to receive donor nucleic acid sequences to be further inserted into the genome at a target site in the GEMS sequence. Donor nucleic acid sequences can be in DNA or RNA form, with DNA being preferred. Donor nucleic acid sequences can be provided on an additional plasmid or other suitable vector that is inserted into the host cell. In one aspect, provided herein is a donor nucleic acid construct comprising the donor nucleic acid described above. In some embodiments, the donor nucleic acid construct further comprises a nucleic acid sequence encoding a selectable marker. Useful selectable markers include, for example, antibiotic-resistance genes, such as puromycin resistance gene (puro), neomycin resistance gene (neo) (SEQ ID NO: 13), blasticidin resistance gene (bla) (SEQ ID NO: 19), and ampicillin resistance gene and the like. In some embodiments, the donor nucleic acid construct further comprises a nucleic acid sequence encoding a promoter region. In some embodiments, the selectable marker polypeptide is a WT selectable marker polypeptide. In some embodiments, the selectable marker polypeptide is a modified selectable marker polypeptide. In some embodiments, nucleic acid sequence encoding a selectable marker polypeptide include, for example, antibiotic-resistance genes, such as puromycin resistance gene (puro (SEQ ID NO: 13), neomycin resistance gene (neo) (SEQ ID NO: 84 or SEQ ID NO: 14), blasticidin resistance gene (bla) (SEQ ID NO: 19), and ampicillin resistance gene and the like. In some embodiments, the nucleic acid sequence encoding a selectable marker polypeptide is a puromycin resistance gene sequence comprises a nucleotide sequence of SEQ ID NO: 13. In some embodiments, the nucleic acid sequence encoding a selectable marker polypeptide is a puromycin resistance gene sequence comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 13. In an embodiment, the nucleic acid sequence encoding a selectable marker polypeptide is a blasticidin resistance gene sequence comprises a nucleotide sequence of SEQ ID NO: 19. In some embodiments, the nucleic acid sequence encoding a selectable marker polypeptide is a the hygromycin resistance gene sequence comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 81. In some embodiments, the nucleic acid sequence encoding a selectable marker polypeptide is a the hygromycin resistance gene sequence comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 81. In some embodiments, nucleic acid sequence encoding a modified selectable marker protein comprises a nucleotide sequence of SEQ ID NO: 84. In some embodiments, nucleic acid sequence encoding a modified selectable marker protein comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 84.

Non limiting examples of promoter include, CMV promoter (SEQ ID NO:11), EF-1alpha promoter (SEQ ID NO: 18). In some embodiments, the donor nucleic acid construct can further comprise a first donor flanking sequence homologous to a genomic sequence upstream of said GEMS sequence (5′ homology arm), and a second donor flanking sequence homologous to a genomic sequence downstream of said GEMS sequence (3′ homology arm). In some embodiments, nuclease recognition sequence can be selected, for example, from SEQ ID NOs: 85, 89, 91, 93, 95, 97, 99, 101, 103, 105, 107, 109, 111, 113, 115, 117, 119, 121, or reverse complements thereof. In some embodiments, the target sequence of the nuclease recognition sequences can be heterologous to the genome. The target sequence can be from about 10 to about 30 nucleotides in length, from about 15 to about 25 nucleotides in length, and from about 17 to about 24 nucleotides in length. In some aspects, the target sequence is about 20 nucleotides in length. In some embodiments, the target sequence can be GC-rich, such that at least about 40% of the target sequence is made up of G or C nucleotides. The GC content of the target sequence can from about 40% to about 80%, though GC content of less than about 40% or greater than about 80% can be used. In some embodiments, the target sequence can be AT-rich, such that at least about 40% of the target sequence is made up of A or T nucleotides. The AT content of the target sequence can from about 40% to about 80%, though AT content of less than about 40% or greater than about 80% can be used. In some embodiments, the target site is a nucleotide sequence selected from SEQ ID NOs: 89, 91, 93, 95, 97, 99, 101, and 103. In some embodiments, the 5′ homology arm sequence comprises a nucleotide sequence of SEQ ID NO: 7, and the 3′ homology arm sequence comprises a nucleotide sequence of SEQ ID NO: 8. Transfection, lipofection, or temporary membrane disruption such as electroporation or deformation can be used to insert the vector comprising the donor nucleic acid sequence into the host cell. Viral or non-viral vectors can be used to deliver the donor nucleic acid sequence in some aspects. The vector or plasmid comprising a donor nucleic acid sequence can comprises endonuclease recognition sequences upstream and downstream of the donor nucleic acid sequence, such that the vector can be cleaved by the same endonuclease that cleaves the multiple gene editing site.

The donor nucleic acid sequences can be exogenous genes, or portions thereof, including engineered genes. The donor nucleic acid sequences can encode any protein or portion thereof that the user desires that the host cell express. The donor nucleic acid sequences (including genes) can further comprise a reporter gene, which can be used to confirm expression. The expression product of the reporter gene can be substantially inert such that its expression along with the donor gene of interest does not interfere with the intended activity of the donor gene expression product, or otherwise interfere with other natural processes in the cell, or otherwise cause deleterious effects in the cell.

The donor nucleic acid sequence can also comprise regulatory elements that permit controlled expression of the donor gene. For example, the donor nucleic acid sequence can comprise a repressor operon or inducible operon. The expression of the donor nucleic acid sequence can thus be under regulatory control such that the gene is only expressed under controlled conditions. In some aspects, the donor nucleic acid sequence includes no regulatory elements, such that the donor gene is effectively constitutively expressed.

In some embodiments, the donor nucleic acid sequence encoding is the green fluorescent protein (GFP) (SEQ ID NO: 12) under a tetracycline (Tet)-inducible promoter. In an embodiment, a reporter gene (e.g., GFP) and a regulatory element inserted into the multiple gene editing site. Upon integration of e.g., the GFP and Tet-regulatory elements into the multiple gene editing site in the cell, exposure of the cell to e.g., tetracycline can induce the expression of e.g., GFP such that the expression can be confirmed and measured.

The number of donor nucleic acid sequences that can be inserted into the multiple gene editing site can vary. The number of potential donor nucleic acid sequences can be limited, for example, by the number of nuclease recognition sites in the GEMS sequence and/or the number of donor nucleic acid sequences whose expression the cell is capable of tolerating.

The size of any given donor nucleic acid sequences that can be inserted into the multiple gene editing site can vary. The size can be limited by the number of donor nucleic acid sequences being inserted into the multiple gene editing site and/or the number or size of the donor nucleic acid sequences the cell is capable of tolerating.

In some embodiments, the donor nucleic acid sequence can be inserted into any one of the plurality of nuclease recognition sequences of the GEMS sequence. Insertion can be facilitated by the particular nuclease, which cleaves the nuclease recognition site in the GEMS sequence and also cleaves the nuclease recognition site in the vector. The latter cleavage frees the donor nucleic acid sequence for insertion into the cleaved GEMS sequence. Insertion of the donor nucleic acid sequence can proceed via homologous or NHEJ in the cell. Thus, the nuclease recognition sequences can be tailored to nucleases that produce compatible ends at the site of the double stranded breaks in the vector DNA and in the multiple gene editing site. In some embodiments, multiple donor nucleic acid sequences can be sequentially inserted into the GEMS sequence. In some embodiments, multiple donor nucleic acid sequence can be simultaneously inserted into the GEMS sequence.

The nuclease can be a ZFN, TALEN, or CRISPR associated nuclease such as Cas9 nuclease. In some aspects, the nuclease can be a CRISPR associated nuclease such that a CRISPR associated nuclease is used to insert each donor nucleic acid into GEMS sequence. Cleavage of the GEMS sequence via a CRISPR associated nuclease such as Cas9 nuclease occurs by way of a guide RNA (gRNA) or a guide polynucleotide that is specific to the target sequence and PAM sequence combination of a given secondary endonuclease recognition site in the multiple gene editing site. The gRNA or the guide polynucleotide comprises a protospacer element that is complementary to the target sequence, and a CRISPR RNA (crRNA) and a transactivation crRNA (tracrRNA) chimera. The gRNA or the guide polynucleotide recruits the Cas9 nuclease to form a complex, which complex recognizes the target sequence and PAM sequence at the multiple gene editing site, and thereafter, the nuclease cleaves the multiple gene editing site.

Following insertion of the donor nucleic acid sequence, the host cell can be further manipulated in order to express the protein encoded by the donor nucleic acid sequence, for example, cultured in the presence of inducers or repressors. The host cell can also be cultured and propagated. In aspects where the host cell is a stem cell, the cell can be differentiated following insertion of the donor nucleic acid sequences. The differentiated stem cell can be cultured and propagated.

### Chimeric Antigen Receptor (CAR)

In an embodiment, the donor nucleic acid sequence is a chimeric antigen receptor (CAR). A CAR is an engineered receptor or an engineered receptor construct which grafts an exogenous specificity onto an immune effector cell. In some instances, a CAR comprises an extracellular domain (ectodomain) that comprises a target-specific binding element otherwise referred to as an antigen binding moiety or an antigen binding domain, a stalk region, a transmembrane domain and an intracellular (endodomain) domain. In some embodiments, CAR does not actually recognize the entire antigen; instead it binds to only a portion of the antigen's surface, an area called the antigenic determinant or epitope. In some instances, the intracellular domain further comprises one or more intracellular signaling domains or cytoplasmic signaling domains. In some instances, the intracellular domain further comprises a zeta chain portion. In some instances, a CAR as described herein further comprises one or more costimulatory domains and a signaling domain for T-cell activation.

In some embodiments, a CAR described herein comprises a target-specific binding element otherwise referred to as an antigen-binding moiety, an antigen binding domain or a predetermined cell surface protein. In embodiments, a CAR described herein engineered to target a tumor antigen of interest by way of engineering a desired antigen-binding moiety that specifically binds to an antigen on a tumor cell. In the context of the present disclosure, “tumor antigen” or “hyperproliferative disorder antigen” or “antigen associated with a hyperproliferative disorder,” refers to antigens that are common to specific hyperproliferative disorders such as cancer.

In some embodiments, the antigen binding moiety of a CAR described herein is specific to or binds CD19. In embodiments, the antigen binding domain comprises a single chain antibody fragment (scFv) comprising a variable domain light chain (VL) and variable domain heavy chain (VH) of a target antigen specific monoclonal antibody. In embodiments, the scFv is humanized. In some embodiments, the antigen binding moiety can comprise VH and VL that are directionally linked, for example, from N to C terminus, VH-linker-VL or VL-linker-VH. In some instances, the antigen binding domain recognizes an epitope of the target. In some embodiments, described herein include a CAR or a CAR-T cell, in which the antigen binding domain comprises a F(ab′)2, Fab′, Fab, Fv, or scFv.

In some embodiments, CD19 scFv is encoded by a nucleotide sequence comprising SEQ ID NO: 20. In some embodiments, CD19 scFv is encoded by a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 20. In some embodiments, the CD19 CAR comprise a nucleotide sequence of SEQ ID NO: 20. In some embodiments, the CD19 CAR comprise a nucleotide sequence of SEQ ID NO: 21. In some embodiments, the CD19 CAR comprise a nucleotide sequence of SEQ ID NO: 22. In some embodiments, the CD19 CAR comprise a nucleotide sequence of SEQ ID NO: 23. In some embodiments, the CD19 CAR comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 20. In some embodiments, the CD19 CAR comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 21. In some embodiments, the CD19 CAR comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 22. In some embodiments, the CD19 CAR comprises a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 23.

In embodiments described herein, a CAR can comprise an extracellular antibody-derived single-chain variable domain (scFv) for target recognition, wherein the scFv can be connected by a flexible linker to a transmembrane domain and/or an intracellular signaling domain(s) that includes, for instance, CD3-ζ for T-cell activation. Normally when T cells are activated in vivo, they receive a primary antigen induced TCR signal with secondary costimulatory signaling from CD28 that induces the production of cytokines (e.g., IL-2 and IL-21), which then feed back into the signaling loop in an autocrine/paracrine fashion. With this in mind, a CAR can include a signaling domain, for instance, a CD28 cytoplasmic signaling domain or other costimulatory molecule signaling domains such as 4-1BB signaling domain. Chimeric CD28 co-stimulation improves T-cell persistence by up-regulation of anti-apoptotic molecules and production of IL-2, as well as expanding T cells derived from peripheral blood mononuclear cells (PBMC). In one embodiment, CARS are fusions of single-chain variable fragments (scFv) derived from monoclonal antibodies specific for hepatitis B virus antigen. In another embodiment, CARs are fused to transmembrane domain and CD3-ζ endodomain. Such molecules result in the transmission of a zeta signal in response to recognition by the scFv of its target.

In one embodiment of the CAR ectodomain, a signal peptide directs the nascent protein into the endoplasmic reticulum, for instance, if the receptor is to be glycosylated and anchored in the cell membrane. Any eukaryotic signal peptide sequence is envisaged to be functional. Generally, the signal peptide natively attached to the amino-terminal most component is used (e.g., in a scFv with orientation light chain—linker—heavy chain, the native signal of the light-chain is used). In embodiments, the signal peptide is GM-CSFRa or IgK. Other signal peptides that can be used include signal peptides from CD8a and CD28.

The antigen recognition domain can be a scFv. There can however be alternatives. An antigen recognition domain from native T-cell receptor (TCR) alpha and beta single chains are envisaged, as they have simple ectodomains (e.g., CD4 ectodomain to recognize HIV infected cells) and as well as other recognition components such as a linked e.g., cytokine (which leads to recognition of cells bearing the cytokine receptor). Almost anything that binds a given target, such as e.g., tumor associated antigen, with high affinity can be used as an antigen recognition region.

The transmembrane domain can be derived from either a natural or a synthetic source. Where the source is natural, the domain can be derived from any membrane-bound or transmembrane protein. Suitable transmembrane domains can include, but not limited to, the transmembrane region(s) of alpha, beta or zeta chain of the T-cell receptor; or a transmembrane region from CD28, CD3 epsilon, CD3-ζ, CD45, CD4, CD5, CD8alpha, CD9, CD16, CD22, CD33, CD37, CD64, CD80, CD86, CD134, CD137 or CD154. Alternatively the transmembrane domain can be synthetic and can comprise hydrophobic residues such as leucine and valine. In some embodiments, a triplet of phenylalanine, tryptophan and valine is found at one or both termini of a synthetic transmembrane domain. In some embodiments, the transmembrane domain comprises a CD8α transmembrane domain or a CD3-ζ transmembrane domain. In some embodiments, the transmembrane domain comprises a CD8α transmembrane domain. In other embodiments, the transmembrane domain comprises a CD3-ζ transmembrane domain. In some embodiments, CD8 hinge and transmembrane domain is encoded by a nucleotide sequence comprising SEQ ID NO: 21. In some embodiments, CD8 hinge and transmembrane domain is encoded by a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 21.

The intracellular signaling domain, also known as cytoplasmic domain, of the CAR of the present disclosure, is responsible for activation of at least one of the normal effector functions of the immune cell in which the CAR has been placed. The term “effector function” refers to a specialized function of a cell. Effector function of a T cell, for example, can be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal. In some embodiments, the intracellular domain further comprises a signaling domain for T-cell activation. In some instances, the signaling domain for T-cell activation comprises a domain derived from TCRζ, FcRγ, FcRβ, CD3γ, CD3δ, CD3ε, CD5, CD22, CD79α, CD79β or CD66δ. In some cases, the signaling domain for T-cell activation comprises a domain derived from CD3-ζ; In some cases, the intracellular domain can comprise one or more costimulatory domains.

The cytoplasmic domain, also known as the intracellular signaling domain of a CAR described herein, is responsible for activation of at least one of the normal effector functions of the immune cell in which the CAR has been placed. The term “effector function” refers to a specialized function of a cell. Effector function of a T cell, for example, can be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in a CAR described herein can include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Signals generated through the TCR alone are generally insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner can contain signaling motifs which are known as immunoreceptor tyrosine-based activation motifs or ITAMs. Examples of ITAM-containing primary cytoplasmic signaling sequences that are of particular use in the present disclosure include, but not limited to, those derived from TCR zeta, FcR gamma, FcR beta, CD3 gamma, CD3 delta, CD3 epsilon, CD5, CD22, CD79a, CD79b, and CD66d. In embodiments, the cytoplasmic signaling molecule in a CAR described herein comprises a cytoplasmic signaling sequence derived from CD3 zeta.

In embodiments, the cytoplasmic domain of the CAR can be designed to comprise the CD3-signaling domain by itself or combined with any other desired cytoplasmic domain(s) useful in the context of a CAR described herein. For example, the cytoplasmic domain of the CAR can comprise a CD3 chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. In embodiments, costimulatory molecules can be used together, e.g., CD28 and 4-IBB or CD28 and OX40. Thus, while the present disclosure in exemplified primarily with 4-IBBζ and CD8α as the co-stimulatory signaling element, other costimulatory elements are within the scope of the present disclosure. In some embodiments, 4-IBB endodomain is encoded by a nucleotide sequence comprising SEQ ID NO: 22. In some embodiments, 4-IBB endodomain is encoded by a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 22.

The cytoplasmic signaling sequences within the cytoplasmic signaling portion of a CAR described herein can be linked to each other in a random or specified order. In one embodiment, the cytoplasmic domain comprises the signaling domain of CD3-zeta and the signaling domain of CD28. In another embodiment, the cytoplasmic domain comprises the signaling domain of CD3-zeta and the signaling domain of 4-IBB. In yet another embodiment, the cytoplasmic domain is comprises the signaling domain of CD3-zeta and the signaling domains of CD28 and 4-IBB. In some embodiments, CD3 zeta domain is encoded by a nucleotide sequence comprising SEQ ID NO: 23. In some embodiments, 4CD3 zeta domain is encoded by a nucleotide sequence having at least 50%, 55%, 60%, 65%, 70%, 75%, 80%, 85%, 90%, 91%, 92%, 93%, 94%, 95%, 96%, 97%, 98%, 99%, 99.5% or 100% identity with the nucleotide sequence of SEQ ID NO: 23.

The costimulatory signaling region refers to a portion of the CAR comprising the intracellular signaling domain of a costimulatory molecule. Costimulatory molecules are cell surface molecules other than antigens receptors or their ligands that are required for an efficient response of lymphocytes to antigen. Exemplary costimulatory domains include, but are not limited to, CD8, CD27, CD28, 4-IBB (CD137), ICOS, DAP10, DAP12, OX40 (CD134), CD3-zeta or fragment or combination thereof. In some instances, a CAR described herein comprises one or more, or two or more of costimulatory domains selected from CD8, CD27, CD28, 4-1BB (CD137), ICOS, DAP10, DAP12, OX40 (CD134) or fragment or combination thereof. In some instances, a CAR described herein comprises one or more, or two or more of costimulatory domains selected from CD27, CD28, 4-1BB (CD137), ICOS, OX40 (CD134) or fragment or combination thereof. In some instances, a CAR described herein comprises one or more, or two or more of costimulatory domains selected from CD8, CD28, 4-1BB (CD137), DAP10, DAP12 or fragment or combination thereof. In some instances, a CAR described herein comprises one or more, or two or more of costimulatory domains selected from CD28, 4-1BB (CD137), or fragment or combination thereof. In some instances, a CAR described herein comprises costimulatory domains CD28 and 4-1BB (CD137) or their respective fragments thereof. In some instances, a CAR described herein comprises costimulatory domains CD28 and OX40 (CD134) or their respective fragments thereof. In some instances, a CAR described herein comprises costimulatory domains CD8 and CD28 or their respective fragments thereof. In some instances, a CAR described herein comprises costimulatory domains CD28 or a fragment thereof. In some instances, a CAR described herein comprises costimulatory domains 4-1BB (CD137) or a fragment thereof. In some instances, a CAR described herein comprises costimulatory domains OX40 (CD134) or a fragment thereof. In some instances, a CAR described herein comprises costimulatory domains CD8 or a fragment thereof. In some instances, a CAR described herein comprises at least one costimulatory domain DAP10 or a fragment thereof. In some instances, a CAR described herein comprises at least one costimulatory domain DAP12 or a fragment thereof.

In general, CARs exist in a dimerized form and are expressed as a fusion protein that links the extracellular scFv (VH linked to VL) region, a transmembrane domain, and intracellular signaling motifs. The endodomain of the first generation CAR induces T cell activation solely through CD3-ζ signaling. The second generation CAR provides activation signaling through CD3-ζ and CD28, or other endodomains such as 4-1BB or OX40. The 3rd generation CAR activates T cells via a CD3-ζ-containing combination of three signaling motifs such as CD28, 4-1BB, or OX40.

In embodiments, provided herein is an isolated nucleic acid encoding a chimeric antigen receptor (CAR), wherein the CAR comprises (a) a CD binding domain; (b) a transmembrane domain; (c) a costimulatory signaling domain comprising 4-1BB or CD28, or both; and (d) a CD3 zeta signaling domain.

In embodiments, the CAR comprises a transmembrane domain that is fused to the extracellular domain of the CAR. In one embodiment, the transmembrane domain that naturally is associated with one of the domains in the CAR is used. In embodiments, the transmembrane domain is a hydrophobic alpha helix that spans the membrane.

The transmembrane domain can be derived from either a natural or a synthetic source. Where the source is natural, the domain can be derived from any membrane-bound or transmembrane protein. In some instances, a CAR comprises a transmembrane domain selected from a CD8α transmembrane domain or a CD3 transmembrane domain; one or more costimulatory domains selected from CD27, CD28, 4-1BB (CD137), ICOS, DAP10, OX40 (CD134) or fragment or combination thereof and a signaling domain from CD3. Transmembrane regions of particular use in this disclosure can be derived from (e.g., comprise at least the transmembrane region(s) of) the alpha, beta or zeta chain of the T-cell receptor, CD28, CD3 epsilon, CD45, CD4, CD5, CD8alpha, CD9, CD16, CD22, CD33, CD37, CD64, CD80, CD86, CD134, CD137 or CD154. Alternatively the transmembrane domain can be synthetic, in which case it will comprise predominantly hydrophobic residues such as leucine and valine. In embodiments, a triplet of phenylalanine, tryptophan and valine will be found at each end of a synthetic transmembrane domain.

Included in the scope of the present disclosure are nucleic acid sequences that encode functional portions of the CAR described herein. Functional portions encompass, for example, those parts of a CAR that retain the ability to recognize target cells, or detect, treat, or prevent a disease, to a similar extent, the same extent, or to a higher extent, as the parent CAR.

In embodiments, the CAR described herein contains additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity of the CAR, as compared to the biological activity of the parent CAR.

In some embodiments, a CAR described herein include (including functional portions and functional variants thereof) glycosylated, amidated, carboxylated, phosphorylated, esterified, N-acylated, cyclized via, e.g., a disulfide bridge, or converted into an acid addition salt and/or optionally dimerized or polymerized, or conjugated.

The plurality of nuclease recognition sites in a GEMS sequence integrated in a host cell can enable production of genetically engineered cells containing a complex array of logic gate CARs. Such cells can be genetically engineered to respond to an antigenic pattern, as opposed to a single tumor associated antigen. Such logic gated CARs are described in Davies DM and Maher J. (Gated chimeric antigen receptor T-cells: the next logical step in reducing toxicity. Transl Cancer Res 2016; 5(S1):561-S65), which is hereby incorporated by reference in its entirety. Logic gate CARS can include, for example, a first generation CAR, a second generation CAR, a chimeric costimulatory receptor (CCR), an inducible promoter of CAR expression, or an iCAR. The first generation CAR can comprise an extracellular domain (ectodomain) that comprises a target-specific binding element otherwise referred to as an antigen binding moiety or an antigen binding domain and an intracellular (endodomain) signaling domain. The second generation CAR can comprise an extracellular domain (ectodomain) that comprises a target-specific binding element otherwise referred to as an antigen binding moiety or an antigen binding domain, an intracellular (endodomain) signaling domain, and a costimulatory signaling domain (e.g., that increases the stimulatory activity of the endodomain in cis). The chimeric costimulatory receptor is an antigen binding receptor that increases the stimulatory activity of a CAR endodomain in trans. The inducible promoter of CAR expression is a receptor that induces CAR expression upon binding to its target antigen. (e.g., synthetic NOTCH). The iCAR is an inhibitory receptor which inhibits CAR activation upon binding to its target antigen.

The cell of the present disclosure can comprise a donor nucleic acid encoding a first generation CAR and a donor nucleic acid encoding a CCR. The cell of the present disclosure can comprise a donor nucleic acid encoding a second generation CAR and a donor nucleic acid encoding an inducible promoter of said CAR expression. The cell of the present disclosure can comprise a donor nucleic acid encoding a second generation CAR and a donor nucleic acid encoding an inducible promoter of said CAR expression. The cell of the present disclosure can comprise a donor nucleic acid encoding a second generation CAR and a donor nucleic acid encoding an iCAR.

### Delivery System

The present disclosure also provides delivery systems, such as viral-based systems, in which a nucleic acid described herein is inserted. Representative viral expression vectors include, but are not limited to, adeno-associated viral vectors, adenovirus-based vectors (e.g., the adenovirus-based Per.C6 system available from Crucell, Inc. (Leiden, The Netherlands)), lentivirus-based vectors (e.g., the lentiviral-based pLPI from Life Technologies (Carlsbad, Calif.)), retroviral vectors (e.g., the pFB-ERV plus pCFB-EGSH), and herpes virus-based vectors. In an embodiment, the viral vector is a lentivirus vector. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In an additional embodiment, the viral vector is an adeno-associated viral vector. In a further embodiment, the viral vector is a retroviral vector. In general, and in embodiments, a suitable vector contains an origin of replication functional in at least one organism, a promoter sequence, convenient restriction endonuclease sites, and one or more selectable markers.

Certain aspects disclosed herein can utilize vectors. Any plasmids and vectors can be used as long as they are replicable and viable in a selected host. Vectors known in the art and those commercially available (and variants or derivatives thereof) can be engineered to include one or more recombination sites for use in the methods. Vectors that can be used include, but not limited to, bacterial expression vectors (such as pBs, pQE-9 (Qiagen), phagescript, PsiX174, pBluescript SK, pB5KS, pNH8a, pNH16a, pNH18a, pNH46a (Stratagene), pTrc99A, pKK223-3, pKK233-3, pDR540, pRIT5 (Pharmacia), and variants or derivatives thereof), eukaryotic expression vectors (such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, pYACneo (Clontech), pSVK3, pSVL, pMSG, pCH110, pKK232-8 (Pharmacia, Inc.), p3′SS, pXT1, pSG5, pPbac, pMbac, pMClneo, pOG44 (Stratagene, Inc.), pYES2, pAC360, pBlueBa-cHis A, B, and C, pVL1392, pBlueBac111, pCDM8, pcDNA1, pZeoSV, pcDNA3, pREP4, pCEP4, pEBVHis (Invitrogen, Corp.), pWLneo, pSv2cat, pOG44, pXT1, pSG (Stratagene) pSVK3, pBPv, pMSG, pSVL (Pharmiacia), and variants or derivatives thereof), and any other plasmids and vectors replicable and viable in the host cell.

Vectors known in the art and those commercially available (and variants or derivatives thereof) can in accordance with the present disclosure be engineered to include one or more recombination sites for use in the methods of the present disclosure. Such vectors can be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, PerkinElmer, Pharmingen, Research Genetics, and Transposagen Pharmaceutical. Other vectors include pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET9, pKK223-3, pKK233-3, pDR540, pRIT5 (Pharmacia), pSPORT1, pSPORT2, pCMVSPORT2.0 and pSY-SPORT1 (Invitrogen) and variants or derivatives thereof. Viral vectors can also be used, such as lentiviral vectors (see, for example, WO 03/059923; Tiscornia et al. PNAS 100:1844-1848 (2003)).

Additional vectors of interest include pTrxFus, pThioHis, pLEX, pTrcHis, pTrcHis2, pRSET, pBlueBacHis2, pcDNA3.1/His, pcDNA3.1 (−)/Myc-His, pSecTag, pEBVHi5, pPIC9K, pPIC3.5K, pAO81S, pPICZ, pPICZA, pPICZB, pPICZC, pGAPZA, pGAPZB, pGAPZC, pBlueBac4.5, pBlueBacHis2, pMelBac, pSinReps, pSinHis, p11D, pND(SP 1), pVgRXR, pcDNA2.1, pYES2, pZErO1.1, pZErO-2.1, pCR-Blunt, pSE280, pSE380, pSE420, pVL1392, pVL1393, pCDM8, pcDNA1.1, pcDNA 1.1/Amp, pcDNA3.1, pcDNA3.1/Zeo, pSe, SV2, pRc/CMV2, pRc/RSV, pREP4, pREP7, pREP8, pREP9, pREP 10, pCEP4, pEBVHis, pCR3.1, pCR2.1, pCR3.1-Uni, and pCRBac from Invitrogen; .lamda., ExCell, .lamda., gt11, pTrc99A, pKK223-3, pGEX-1 λ T, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-lb(+), pT7Blue(R), pT7Blue-2, pCITE-4abc(+), pOCUS-2, pTAg, pET32L1C, pET-30LIC, pBAC-2cp LIC, pBACgus-2cp LIC, pT7Blue-2 LIC, pT7Blue-2, lamda SCREEN-1, lamda BlueSTAR, pET-3abcd, pET-7abc, pET9abcd, pET11abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd(+), pET-25b(+), pET26b(+), pET-27b(+), pET-28abc(+), pET-29abc(+), pET-30abc(+), pET-3 lb(+), pET-32abc(+), pET-33b(+), pBAC-1, pBACgus-1, pBAC4x-1, pBACgus4x-1, pBAC-3cp, pBACgus-2cp, pBACsurf-1, plg, Signal plg, pYX, Selecta Vecta-Neo, Selecta VectaHyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2Basic, pSEAP2-Contral, pSEAP2-Promoter, pSEAP2-Enhancer, pβgal-Basic, pβ-galControl, pβgal-Promoter, pβgal-Enhancer, pCMV, pTet-Off, pTet-On, pTK-Hyg, pRetro-Off, pRetro-On, pIRESlneo, pIRESihyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, λgt10, λgt11, pWE15, and λTriplEx from Clontech; Lambda ZAP II, pBK-CMV, pBK-RSV, pBluescript II KS +/−, pBluescript II SK +/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS +/1−, pBC KS +/−, pBC SK +/−, Phagescript, pCAL-n-EK, pCAL-n, pCAL-c, pCAL-kc, pET-3abcd, pET-11abcd, pSPUTK, pESP-1, pCMVLacI, pOPRSVI/MCS, pOPI3 CAT, pXT1, pSG5, pPbac, pMbac, pMClneo, pMClneo Poly A, pOG44, pOG45, pFRTI3GAL, pNEOβGAL, pRS403, pRS404, pRS405, pRS406, pRS413, pRS414, pRS415, and pRS416 from Stratagene. Additional vectors include, for example, pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp and variants or derivatives thereof.

These vectors can be used to express a gene, e.g., a transgene, or portion of a gene of interest. A gene of portion or a gene can be inserted by using known methods, such as restriction enzyme-based techniques.

Additional suitable vectors include integrating expression vectors, which can randomly integrate into the host cell's DNA, or can include a recombination site to enable the specific recombination between the expression vector and the host cell's chromosome. Such integrating expression vectors can utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNATM5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFN10A (ACT) FLEXI™ from Promega (Madison, Wis.). Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

In some embodiments, the vectors comprise a hEF1a1 promoter to drive expression of transgenes, a bovine growth hormone polyA sequence to enhance transcription, a woodchuck hepatitis virus posttranscriptional regulatory element (WPRE), as well as LTR sequences derived from the pFUGW plasmid.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (Molecular Cloning: A Laboratory Manual, Cold Spring Harbor Laboratory, New York (2001)). In embodiments, a method for the introduction of a polynucleotide into a host cell is calcium phosphate transfection or polyethylenimine (PEI) Transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid can be associated with a lipid. The nucleic acid associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids can be obtained from Avanti Polar Lipids, Inc. (Birmingham, Ala.). Stock solutions of lipids in chloroform or chloroform/methanol can be stored at about −20o C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5: 505-10 (1991)). However, compositions that have different structures in solution than the normal vesicular structure are also encompassed. For example, the lipids can assume a micellar structure or merely exist as non-uniform aggregates of lipid molecules. Also contemplated are lipofectamine-nucleic acid complexes.

### Therapeutic Compositions

In some aspects, the donor nucleic acid sequence encodes a therapeutic protein such as an antibody, a cytokine, a neurotransmitter, or a hormone. Thus, for example, when the host cell expresses the therapeutic protein, the host cell can serve as a therapeutic effector cell, or can have enhanced immunotherapeutic potential. In an embodiment, a pluripotent stem cell comprising the construct receives a donor nucleic acid sequence encoding a cytotoxic protein (Y), and is differentiated to a cytotoxic cell lineage and expanded, then expresses the cytotoxic protein. In an embodiment, the host cells comprising the construct can be used in therapeutic modalities, and can be engineered according to donor nucleic acid sequences inserted into the multiple gene editing site of the construct.

In some aspects, the cell can secrete the protein encoded by the donor nucleic acid. Thus, the cell can have further use as an expression host cell, whereby the protein is secreted in the cell culture medium, and later harvested and purified.

The cells comprising a GEMS can be used to study the effects of the protein encoded by the donor gene on the cell, including the effects on signal pathway, or the capacity to differentiate and still express the donor gene protein. Clinically, the cells can be used to express therapeutic proteins or provide therapeutic support to immune cells.

In some aspects, one or more donor sequences can be removed from the GEMS. For example, where a donor sequence is positioned between nuclease recognition sites, such sites can be utilized to cleave the GEMS sequence.

In some aspects, the GEMS sequence itself can be removed. Removal of the GEMS sequence can also remove any donor nucleic acid sequences inserted therein. A meganuclease recognition site can utilized to cleave the outer regions of the GEMS sequence to facilitate its removal from the genome, including removal from the safe harbor site (e.g., Rosa26, AAVS1, CCR5).

In some embodiments, following insertion of the GEMS sequence into a host cell, the host cell can be differentiated into neural lineage. The host cell can be a primary isolate stem cell, or stem cell line. The differentiation can occur prior to or following insertion of donor nucleic acid sequences into the multiple gene editing site in the stem cell host. In some embodiments, the cell comprises at least 2, 3, 4, 5, 6, 7, 8, 9, 10, 11, 12, 13, 14, 15, 16, 17, 18, 19, 20, 25 or more donor nucleic acid sequences. In some embodiments, the cell comprises at least 2, 3, 4, 5, 6, 7, 8, 9, 10, 11, 12, 13, 14, 15, 16, 17, 18, 19, 20, 25 or more unique donor nucleic acid sequences.

In some embodiments, the donor nucleic acid sequence can encode a chimeric antigen receptor. Following insertion of the multiple gene editing site into a host cell, the host cell can be differentiated into a cytotoxic T cell lineage or natural killer (NK) cell lineage. The host cell can be a primary isolate stem cell, or stem cell line. The differentiation can occur prior to or following insertion of donor nucleic acid sequences into the multiple gene editing site in the stem cell host. The donor nucleic acid sequences can encode one or more tumor targeting chimeric antigen receptors (CARs). Accordingly, in some embodiments, the cell can express at least 2, 3, 4, 5, 6, 7, 8, 9, 10, 11, 12, 13, 14, 15, 16, 17, 18, 19, 20, 21, 22, 23, 24, 25, or more unique chimeric antigen receptors. In some embodiments, the cell expressing at least 2, 3, 4, 5, 6, 7, 8, 9, 10, 11, 12, 13, 14, 15, 16, 17, 18, 19, 20, 21, 22, 23, 24, 25, or more unique chimeric antigen receptors is a NK T cell. The differentiated cells expressing the CARs can then be administered to cancer patients whose tumor cells express the CAR target. Without intending to be limited to any particular theory or mechanism of action, it is believed that the interaction of the CARs-expressing cytotoxic cells with tumor cells expressing CAR targets can facilitate killing of the tumor cells. The stem cells can be first isolated from the cancer patient, then returned to the patient following modification, differentiation, and expansion. The stem cells can be first isolated from a healthy donor, then administered to a cancer patient following modification, differentiation, and expansion. The cells can be directed to any tumor based on the CAR target, with the donor sequence tailored to the particular CARS expressed by the tumor.

In some embodiments, the donor nucleic acid sequence can encode dopamine or other neurotransmitter. The donor nucleic acid sequence encoding dopamine or other neurotransmitter can be under a regulatory control element, that modulates the level of dopamine or neurotransmitter expression according to the intake of a small molecule that affects the regulatory control element, for example, tetracycline to the tetracycline operon. The differentiated cells expressing dopamine can then be administered to a patient having a condition mediated by a dysregulation of dopamine expression, such as Parkinson's disease. Without intending to be limited to any particular theory or mechanism of action, it is believed that the expression of dopamine can mitigate the dysregulation of dopamine expression or other deficiency of dopamine, thereby treating the condition. The stem cells can be first isolated from the patient (e.g., Parkinson's Disease patient), then returned to the patient following modification, differentiation, and expansion. The stem cells can be first isolated from a healthy donor, then administered to the patient (e.g., Parkinson's Disease patient) following modification, differentiation, and expansion.

In some embodiments, the donor nucleic acid sequence can encode insulin or a pro-form of insulin, or other hormones. The differentiated cells expressing insulin or the pro-form thereof can then be administered to a patient having diabetes (Type 1 or Type 2), or other condition mediated by insulin dysregulation. Without intending to be limited to any particular theory or mechanism of action, it is believed that the expression of insulin can treat diabetes or other deficiency of insulin, thereby treating the condition. The stem cells can be first isolated from the patient (e.g., diabetes patient), then returned to the patient following modification, differentiation, and expansion. The stem cells can be first isolated from a healthy donor, then administered to the patient (e.g., diabetes patient) following modification, differentiation, and expansion.

The disclosure is not limited to the embodiments described and exemplified above, but is capable of variation and modification within the scope of the appended claims.

### Kits:

The constructs, vectors, recombinases and recombinase-coding sequences, and genetically engineered cells of the present disclosure can be formulated into kits. Components of such kits can include, but are not limited to, containers, instructions, solutions, buffers, culture medium, disposables, and hardware. The kits disclosed herein can be useful for example, for performing the methods of the present disclosure. In some embodiments, the kits of the present disclosure can comprise a GEMS construct provided for example, in a solution or a lyophilized form. In some embodiments, kits of the present disclosure comprise a host cell or a cell line comprising a host cell, which is desired to be genetically engineered by inserting a GEMS sequence in its genome and/or further genetically engineered by site-specific insertion of an exogenous polynucleotide encoding a desired protein within or adjacent to the GEMS sequence. A host cell can be provided in a solution form, for example a predetermined amount of viable cells in an appropriate culture medium or in a lyophilized form.

In one aspect provided herein are kits for generating a genetically engineered cell comprising a GEMS sequence comprising a plurality of first recognition sequence for a site-specific recombinase. A kit, for example, useful for generating a genetically engineered cell comprising a GEMS sequence can comprise for example, a GEMS construct disclosed herein and/or a host cell or a cell line comprising the host cell for inserting the GEMS construct for generating a genetically engineered cell comprising a GEMS sequence, for example, by methods disclosed herein.

In some embodiments, the kits of the present disclosure comprise a genetically engineered cell comprising a GEMS sequence comprising a plurality of a first recognition sequence for a site-specific recombinase inserted in its genome or a cell line comprising the genetically engineered cell. The genetically engineered cell comprising a GEMS sequence can be provided for example, in a solution form or a lyophilized form.

In some embodiments, the kits of the present disclosure comprise a donor vector disclosed herein (e.g., a donor vector comprising an exogenous polynucleotide encoding a desired polypeptide, and a second recognition site for a site-specific recombinase). The donor vector can be provided for example, in a solution or a lyophilized form.

In some embodiments, the kits of the present disclosure comprise an appropriate site specific recombinase or a vector comprising a nucleic acid sequence encoding the site-specific recombinase. The recombinase or vector encoding recombinase can be provided in a solution or a lyophilized form.

In one aspect provided herein is kit useful, for example, for (i) site-specific integration of an exogenous polynucleotide encoding a desired recombinant protein (e.g., a therapeutic polypeptide) within a genome of a cell, (ii) generating a genetically engineered cell comprising one or more copies of an exogenous polynucleotide encoding a desired protein (e.g., a therapeutic polypeptide) inserted within or adjacent to a GEMS sequence, or (iii) expression of a protein of interest, such as a therapeutic polypeptide of interest, for example, using the methods disclosed herein. In some embodiments, such a kit can include, a genetically engineered cell or a cell line comprising a genetically engineered cell comprising a GEMS sequence comprising a plurality of a first recognition sequence for a site specific recombinase as described above, an appropriate site-specific recombinase corresponding to the first recognition sequence or a vector comprising nucleic acid sequence encoding the recombinase, and a donor vector comprising an exogenous polynucleotide (e.g., encoding a polypeptide of interest) and a second recognition sequence for the site-specific recombinase. In other embodiments, such a kit can include for example, a host cell or cell line comprising a host cell, a GEMS construct comprising a GEMS sequence comprising a plurality of a first recognition sequence for a site specific recombinase as described above, an appropriate site-specific recombinase corresponding to the first recognition sequence or a vector comprising nucleic acid sequence encoding the recombinase, and a donor vector comprising an exogenous polynucleotide (e.g., encoding the therapeutic polypeptide of interest) and a second recognition sequence for the site-specific recombinase. The kit also includes instructions for completing site-specific integration of an exogenous polynucleotide encoding the protein of interest (e.g., therapeutic polypeptide such as antibody or a fragment thereof and the like). In one embodiment, the donor vector further includes nucleic acid sequence encoding a selectable marker polypeptide, a reporter gene sequence, and/or a regulatory control element sequence. Thus, the kit provides materials and reagents useful in genetically engineered cells for expression and production of of proteins (e.g., therapeutic polypeptide) as discussed above.

In some aspects, the kits disclosed herein comprise a genetically engineered cell or a cell line comprising a genetically engineered cell comprising one or multiple copies of an exogenous polynucleotide encoding a desired protein of inserted within the GEMS sequence within its genome. The kits of the present disclosure can further comprise one or more additional reagents useful for practicing the methods disclosed herein. In some embodiments, the kits can further comprise an appropriate medium for culturing the cells (e.g., host cell or genetically engineered cell), a diluent, a reconstitution medium, a buffer and the like. A kit generally includes a package with one or more containers holding the reagents, as one or more separate compositions or, optionally, as admixture where the compatibility of the reagents will allow. The kit may also include other material(s), which may be desirable from a user standpoint, such as a buffer(s), a diluent(s), culture medium/media, standard(s), and/or any other material useful in processing or conducting any step of the method detailed above. The kits provided herein preferably include instructions for practicing method of the present disclosure. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

These examples are provided for illustrative purposes only and not to limit the scope of the claims provided herein.

### EXAMPLE 1. Design a GEMS Construct Comprising a GEMS Sequence with Plurality of Nuclease Recognition Sequences (GEMS_Cas9sites) for CHO Cell Genome Engineering

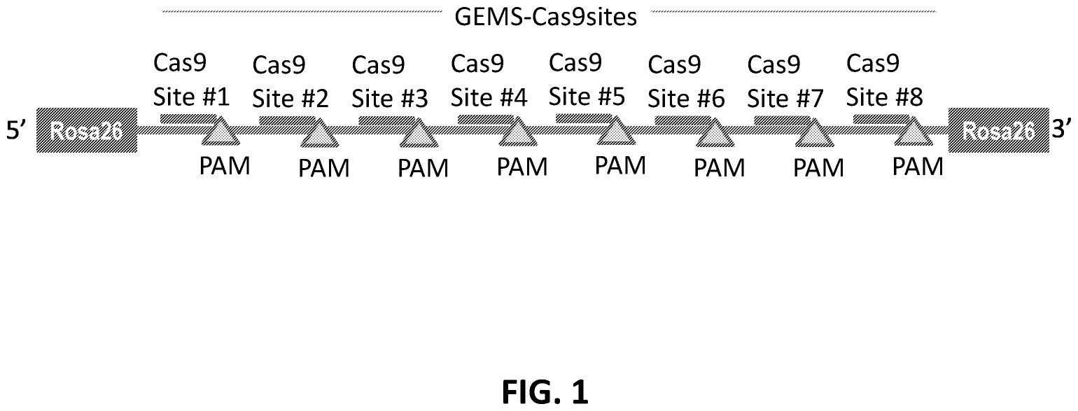

A CHO_Rosa26_GEMS_Cas9sites donor plasmid was generated that comprises a GEMS sequence with eight distinct nuclease recognition sequences. Each nuclease recognition sequence comprises a Cas9 targeting sequence and a PAM sequence (FIG. 1). These eight Cas9 targeting sequences were heterologous to the CHO genome and unique with no predicted high risk off-site targeting in CHO cell genome. These eight sites were flanked by 400 bp random sequences as spacers which serve as the 5′ and 3′ homology arms to facilitate homology recombination when transgene is engineered into any of these sites. The eight nuclease recognition sequence (each having a Cas9 targeting site and a PAM sequence), along with spacer sequences constitute the GEMS sequence which was heterologous to the CHO genome (SEQ ID NO: 1, or SEQ ID NO: 3). These eight Cas9 targeting sites serve as integration (insertion) sites to engineer as many as eight transgenes in the Rosa26 hot spot site by Cas9 nuclease-mediated homology recombination.

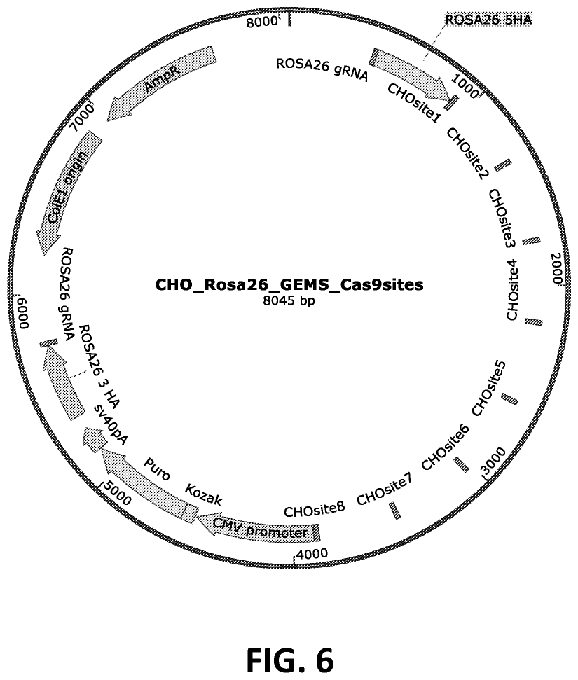

The GEMS donor plasmid CHO_Rosa26_GEMS_Cas9sites was constructed to facilitate site-specific engineering in the loci of Rosa26 of CHO cell genome. In this plasmid (FIG. 6), the GEMS sequence that has plurality of nuclease recognition sequences (GEMS_Cas9sites), and a selection cassette were flanked by ˜500 bp Rosa26 sequences surrounding the cutting site as the 5′ and 3′ homology arms (ROSA26 5HA and ROSA26 3HA) to facilitate homology recombination. The selection cassette was composed of puromycin selection marker (SEQ ID NO: 13). The expression of selection marker Puromycin was driven by CMV promoter (SEQ ID NO: 11). To facilitate homology recombination, Rosa26 targeting site sequences (SEQ ID NO: 9) were designed to flank the GEMS sequence to be inserted so the donor vector can also be cut during CRISPR-mediated cleavage.

### EXAMPLE 2. Engineering GEMS Construct Comprising a GEMS Sequence with Plurality of Nuclease Recognition Sequences (GEMS_Cas9sites) into the Rosa26 Genomic Loci of CHO-K1 Cells

A pCas9D10A_CHO_Rosa26gRNA single shot plasmid was constructed to express Cas9 with D10A mutation, and CHO cell Rosa26 targeting site sgRNA (SEQ ID NO: 10). In contrast to native Cas9 enzyme, D10A mutation leads Cas9 to nick DNA sequence in single strand without double strand cutting. CHO-kl cells were obtained from ATCC.

The GEMS sequence with multiple nuclease recognition sequences (CHO_Rosa26_GEMS_Cas9sites plasmid) was integrated into the Rosa26 site of CHO-kl cells by CRISPR/Cas9-mediated site specific integration. Equal amount of pCas9D10A_CHO_Rosa26gRNA single shot plasmid and CHO_Rosa26_GEMS_Cas9sites donor plasmid were transfected into 2×10⁶ CHO-K1 cells by electroporation using the 4D-Nucleofector™ System from Lonza. The transfected cells were cultured in media with puromycin to select puromycin resistant cells. Two weeks after transfection, puromycin resistant single cell colonies were formed and picked by cloning discs. The selected monoclonal cells were further propagated in the presence of puromycin selection.

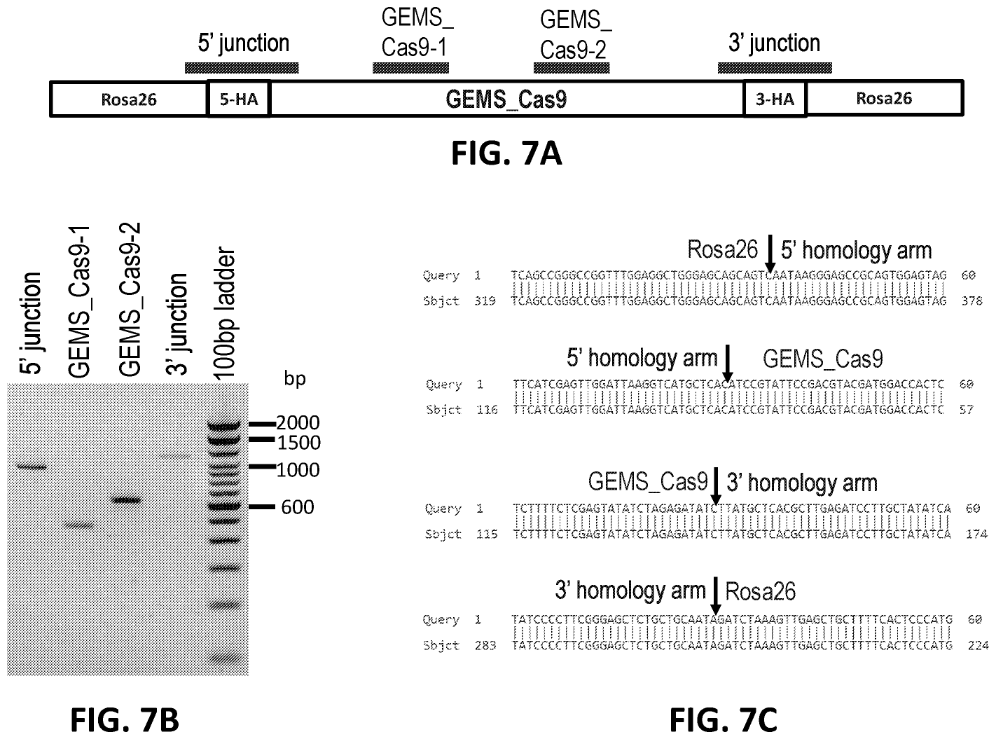

The genomic DNA from puromycin resistant monoclonal CHO-kl cells was prepared. The integration of GEMS sequence (GEMS_Cas9sites) into the cell genome was confirmed by PCR using primers specific to two distinct nuclease recognition sequences of the GEMS sequence (GEMS_Cas9-1 and GEMS_Cas9-2) followed by Sanger sequencing of the PCR products (FIG. 7A). Correct-sized GEMS_Cas9-1 and GEMS_Cas9-2 DNA fragments were amplified by PCR from the genomic DNA isolated from multiple cell clones (FIG. 7B), indicating the successful integration of GEMS sequence comprising plurality of nuclease recognition sequences (GEMS_Cas9sites) in the genome of CHO cells. The PCR products were further sequenced, and the identity of GEMS_Cas9sites sequence was confirmed.

The proper insertion of the GEMS sequence into the Rosa26 site was confirmed by analyzing the 5′ and 3′ junction sites between the Rosa26 site and the GEMS sequence by PCR using one primer specific to Rosa26 sequence and another primer specific to the GEMS sequence (FIG. 7A). The appropriate 5′ and 3′ junctions were confirmed by PCR with DNA bands with expected sizes (FIG. 7B). The PCR products were further sequenced by Sanger sequencing. Correct junctions between Rosa26 site and homology arm and between homology arm and GEMS sequence were confirmed for both 5′ and 3′ junction sites (FIG. 7C), indicating successful targeted integration of GEMS sequence (i.e., GEMS_Cas9sites sequence) in the Rosa26 site of CHO-K1 cells.

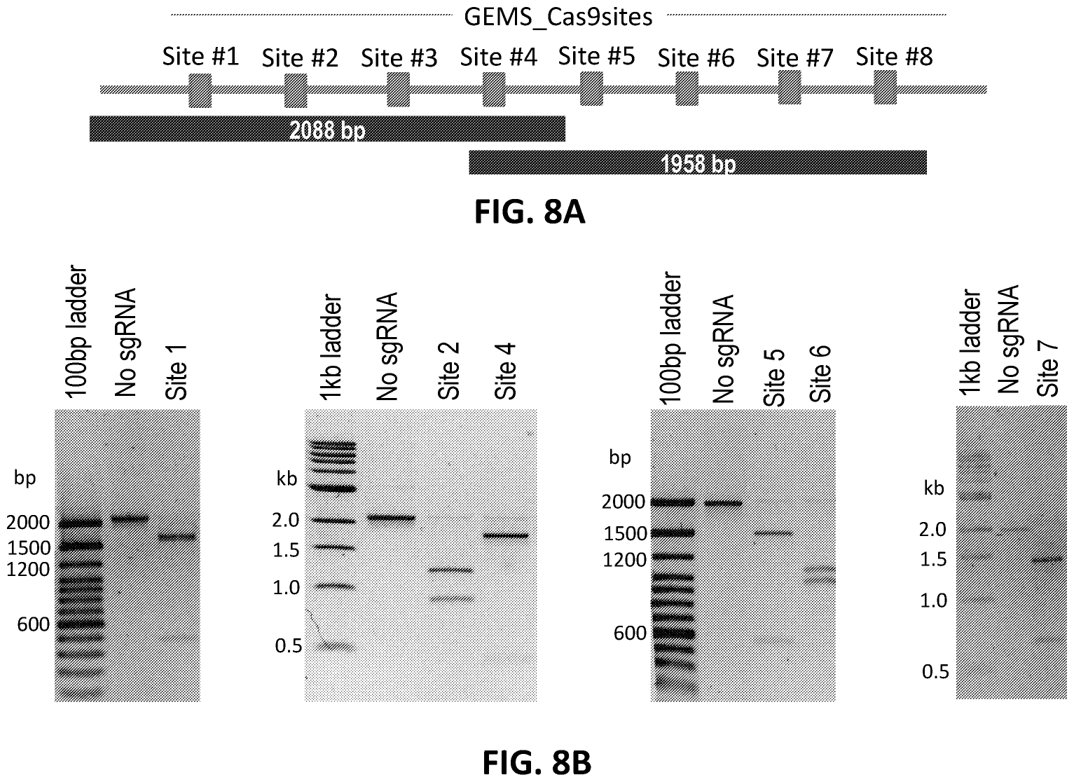

The resulting CHO-K1 cell lines with multiple Cas9 nuclease recognition sequences integrated in the Rosa26 loci can be employed for further engineering transgenes into the Cas9 nuclease recognition sequences by CRISPR/Cas9 nuclease mediated homology recombination. To demonstrate that Cas9-mediated CRISPR cuts the designed eight Cas9 nuclease recognition sequences of the GEMS sequence, an in vitro nuclease assay was performed. Briefly, two overlapping GEMS sequence template DNA; one spanning nuclease recognition sequences, Cas9 site 1 to site 4 with a fragment size of 2088 bp, and another one spanning the nuclease recognition sequences Cas9 site 4 to site 8 with a fragment size of 1958 bp, were PCR amplified and purified (FIG. 8A). sgRNA corresponding to target sequences of selected nuclease recognition sequences were synthesized. 10 pmol of Cas9 nuclease was pre-complexed with 10 pmol of each sgRNA corresponding to target sequences of select nuclease recognition sequences. This pre-complexed RNP was then added to 2 pmol of the appropriate template DNA covering the corresponding Cas9 sites, in a total reaction volume of 25 μl, and incubated at 37° C. for 1.5 hour followed by proteinaseK digestion for 10 min. The entire 25 μl reaction volume was then analyzed on agarose gel. Six sgRNA corresponding to Cas9 sites 1, 2, 4, 5, 6, and 7 of GEMS sequence (SEQ ID NO: 1) were tested in this in vitro nuclease assay for their ability to cut the target sequence of the select nuclease recognition sequences. It was observed that Cas9 nuclease could specifically and completely cut sites 1, 2, 4, 5, 6, and 7 of the GEMS (CHO GEMS_Cas9site) sequence in the presence of corresponding site-specific sgRNA (FIG. 8B).

### EXAMPLE 3. Engineering a Transgene Expressing CD19 CAR into Site 7 of GEMS Sequence Integrated in Rosa26 Genomic Loci of CHO-K1 Cells

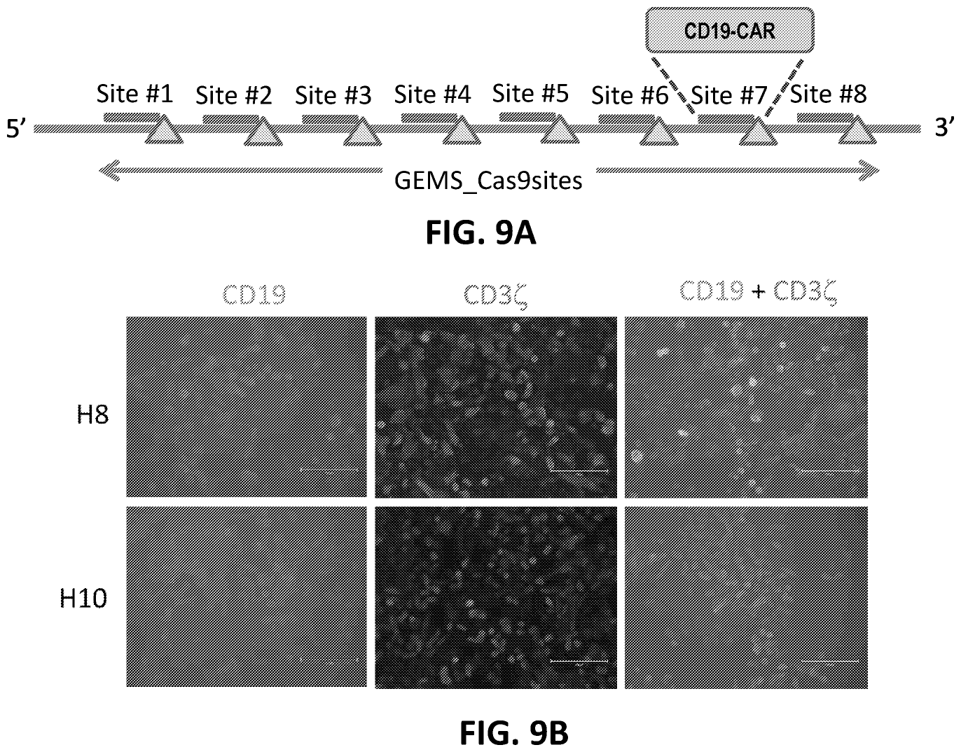

To engineer a transgene expressing CD19 CAR into site 7 of a GEMS sequence (GEMS-Cas9sites shown in FIG. 9A), a GEMSsite7sgRNA-pCas9D10A single shot plasmid was constructed to express GEMS site 7 sgRNA (SEQ ID NO: 102) and Cas9 with D10A mutation. In contrast to native Cas9 enzyme, D10A mutation leads Cas9 to nick DNA sequence in single strand without double strand cutting.

The GEMSsite7 donor plasmid with a transgene expressing CD19 CAR was constructed. The transgene expressed CD19 CAR composed of single chain Fv (scFv) (SEQ ID NO: 20) against CD19, a CD8 hinge and transmembrane domain (SEQ ID NO: 21) followed by 4-1BB costimulatory endodomain (SEQ ID NO: 22) and the CD3-zeta intracellular signaling domain (SEQ ID NO: 23), under the control of an exogenous promoter e.g., EF-1a promoter (SEQ ID NO: 18). In the donor plasmid, the CD19-CAR transgene, along with a hygromycin selection marker (SEQ ID NO: 81) under control of exogenous promoter e.g., CMV promoter (SEQ ID NO: 11), is flanked by GEMS sequence surrounding the selected site for integration of the transgene (i.e., site 7) as the 5′ and 3′ homology arms to facilitate homology recombination. To facilitate homology recombination, the target sequence of nuclease recognition site 7 (SEQ ID NO: 101) were designed to flank the CD19-CAR transgene to be inserted, so the donor vector can also be cut during CRISPR-mediated cleavage.

Equal amount of GEMSsite7sgRNA-pCas9D10A single shot plasmid and CD19 CAR GEMSsite7 donor plasmid were transfected into 2×10⁶ GEMS_Cas9sites modified CHO-K1 cells by electroporation using the 4D-Nucleofector™ System from Lonza. The transfected cells were cultured in media with hygromycin to select hygromycin resistant cells. About two weeks after transfection, hygromycin resistant single cell colonies formed and were picked by cloning discs. The selected monoclonal cells were further propagated in the presence of hygromycin selection.

The expression of CD19 CAR on the cell surface of monoclonal cell line with the transgene expressing CD19 CAR integrated in site 7 of the GEMS sequence was confirmed by immunostaining by an anti-CD3zeta antibody. Briefly, the cells were fixed on slides, permeabilized, and stained by an anti-CD3zeta mouse antibody or an isotype control antibody. The bound antibody was detected by a secondary goat anti-mouse antibody conjugated with Alexa594. The expression of CD19 CAR was detected by anti-CD3zeta antibody with Alexa594 signals along the cell surface (FIG. 9B) while no signal was detected using the isotype control antibody.

The expression of CD19 CAR on the cell surface of monoclonal cell line with the transgene expressing CD19 CAR integrated in site 7 of GEMS sequence was also confirmed by immunostaining by a CD19 Fc fusion protein. Briefly, the cells were fixed on slides, permeabilized, and stained by a CD19 Fc fusion protein. The bound CD19 protein by the CD19 scFv of CD19 CAR was detected by a secondary goat anti-human IgG antibody conjugated with Alexa488. The expression of CD19 CAR was detected by CD19 Fc fusion with Alexa488 signals along the cell surface (FIG. 9B). Besides, the immunostaining signals for CD3zeta colocalized with the immunostaining signals for CD19 in the cells (FIG. 9B).

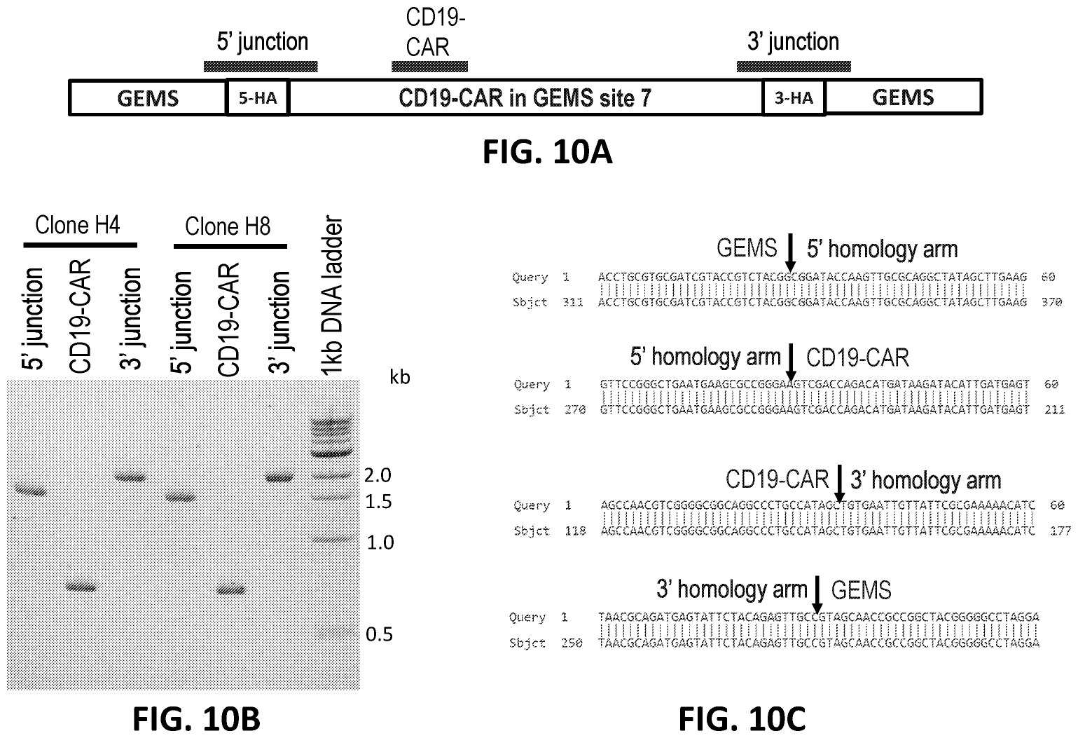

The genomic DNA from hygromycin resistant monoclonal cells were prepared. The targeted integration of a transgene expressing CD19 CAR in the genome of cloned cells was confirmed by PCR using primers specific to CD19-CAR transgene sequence (FIG. 10A). Amplified PCR bands with correct sizes were obtained confirming the site-specific integration of the transgene expressing CD19-CAR (FIG. 10B). Additionally, the correct site-specific insertion of CD19 CAR transgene into the site 7 of GEMS sequence was evaluated by analyzing the 5′ and 3′ junction sites between the GEMS sequence, and the inserted transgene by PCR. The PCR was done using one primer specific to GEMS sequence and another primer specific to the inserted transgene sequence (FIG. 10A). The appropriate 5′ and 3′ junction were confirmed by PCR which resulted in DNA bands with expected sizes (FIG. 10B). The PCR products were further sequenced by Sanger sequencing. Correct junction site sequences between the GEMS insertion site and homology arm and between homology arm and CD19 CAR transgene were confirmed for both 5′ and 3′ junction sites (FIG. 10C). The results indicate successful targeted integration of the transgene expressing CD19 CAR into the site 7 of the GEMS sequence of engineered CHO-K1 cells. The methods resulted in generation of a genetically engineered cell comprising a transgene expressing CD19-CAR inserted in site 7 of the GEMS sequence that is inserted within the genome of CHO-K1 cells. The genetically engineered cells generated expressed the CD-19 CAR on its cell surface.

### EXAMPLE 4. Engineering Transgene Expressing Green Fluorescent Protein (GFP) into Site 2, and Transgene Expressing CD19-CAR in Site 7 of a GEMS Sequence Integrated in Rosa26 Genomic Loci of CHO-K1 Cells

This Example demonstrates engineering multiple transgenes (e.g., transgene expressing CD19-CAR, and transgene expressing GFP) into different nuclease recognition sequences of a GEMS sequence that is integrated within a Rosa26 genomic loci of CHO-K1 cells. The transgene expressing CD19-CAR was engineered in the site 7 of the GEMS sequence as described in Example 3 above to generate genetically engineered cells expressing CD19-CAR.

To further engineer transgene expressing GFP in site 2 of the GEMS sequence of the generated genetically engineered cells, a GEMSsite2sgRNA-pCas9D10A single shot plasmid was constructed to express GEMS site 2 sgRNA (SEQ ID NO: 92) and Cas9 with D10A mutation. In contrast to native Cas9 enzyme, D10A mutation leads Cas9 to nick DNA sequence in single strand without double strand cutting.

The GFP_GEMSsite2 donor plasmid was constructed to express GFP (SEQ ID NO: 12) under the control of e.g., EF-1α promoter (SEQ ID NO: 18). The donor plasmid has the transgene expressing GFP, along with a blasticidin selection marker (SEQ ID NO: 19) under control of an exogenous promoter e.g., CMV promoter (SEQ ID NO: 11), is flanked by GEMS sequence surrounding the selected site for transgene integration (site 2) as the 5′ and 3′ homology arms to facilitate homology recombination. To facilitate homology recombination, the target sequence of GEMS site 2 (SEQ ID NO: 91) were designed to flank the transgene sequence to be inserted so the donor vector can also be cut during CRISPR-mediated cleavage.

Equal amount of GEMSsite2sgRNA-pCas9D10A single shot plasmid and GFP_GEMSsite2 donor plasmid were transfected into 2×10⁶ CHO-K1 cells with CD19-CAR engineered in site 7 by electroporation using the 4D-Nucleofector™ System from Lonza. The transfected cells were cultured in media with blasticidin to select blasticidin resistant cells. About two weeks after transfection, blasticidin resistant single cell colonies formed and were picked by cloning discs. The selected monoclonal cells were further propagated in the presence of blasticidin selection.

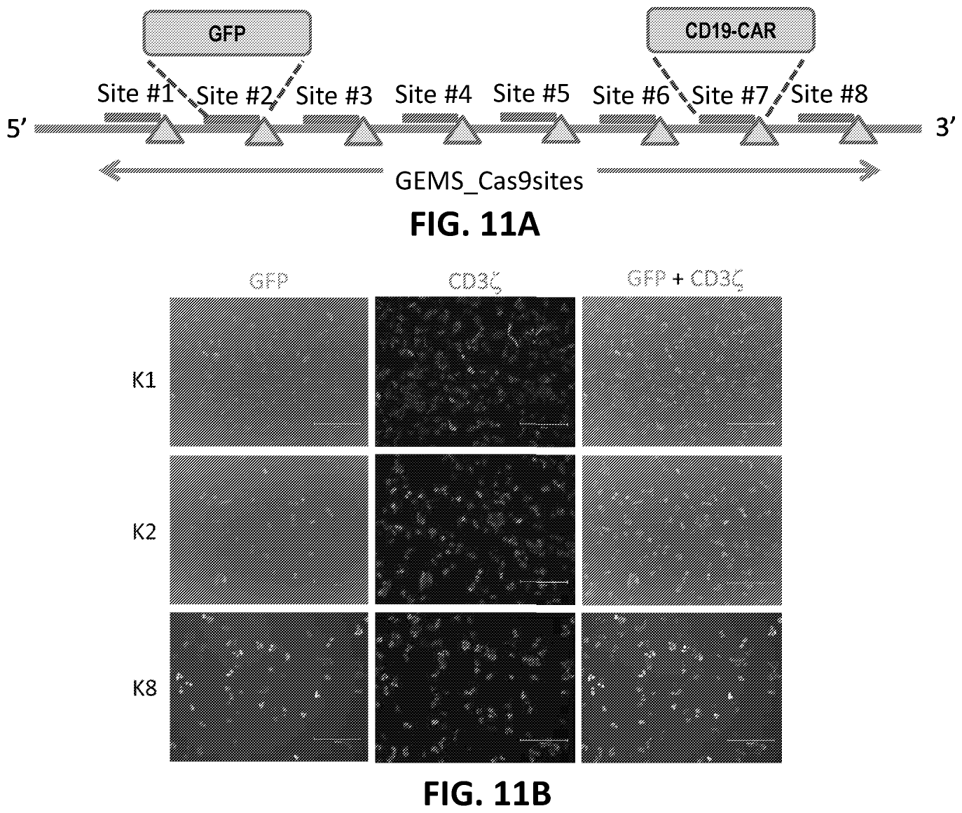

The expression of GFP in the monoclonal cell line was detected by fluorescent microscope (FIG. 11B). The expression of CD19 CAR on the cell surface of monoclonal cell line with CD19 CAR transgene integrated in site 7 of GEMS sequence was evaluated by immunostaining by an anti-CD3zeta antibody. Briefly, the cells were fixed on slides, permeabilized, and stained by an anti-CD3zeta mouse antibody. The bound antibody was detected by a secondary goat anti-mouse antibody conjugated with Alexa594. The expression of CD19 CAR was detected by anti-CD3zeta antibody with Alexa594 signals along the cell surface (FIG. 11B). The GFP signal colocalized with the immunostaining signals for CD3zeta in the cells (FIG. 11B).

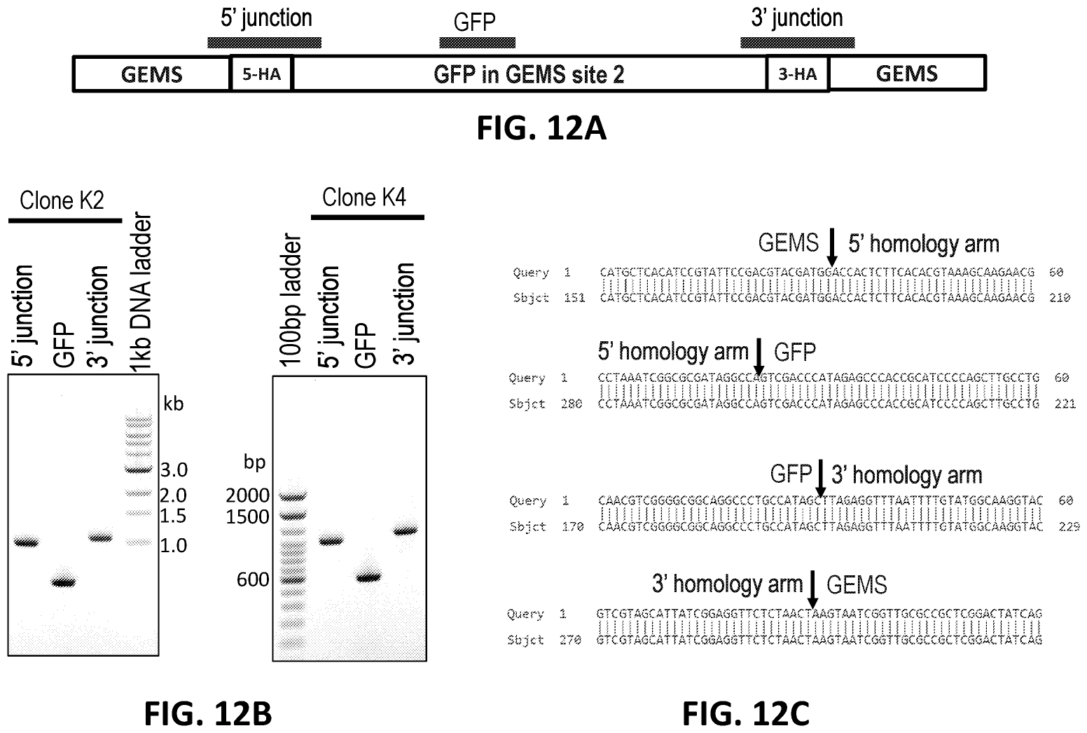

The genomic DNA from blasticidin resistant monoclonal cells were prepared. The presence of GFP sequence in the genome of cloned cells was confirmed by PCR using primers specific to GFP sequence (FIG. 12A) which resulted in amplified PCR bands with correct sizes (FIG. 12B). Additionally, the correct targeted insertion of GFP into the site 2 of GEMS sequence was evaluated by analyzing the 5′ and 3′ junction sites between the GEMS sequence and the inserted transgene by PCR. The PCR was done using one primer specific to the GEMS sequence and another primer specific to the inserted transgene (FIG. 12A). The appropriate 5′ and 3′ junction were confirmed by PCR resulting in DNA bands with expected sizes (FIG. 12B). The PCR products were further sequenced by Sanger sequencing. Correct junction site sequences between selected GEMS site and homology arm and between homology arm and GFP transgene were confirmed for both 5′ and 3′ junction sites (FIG. 12C). The results indicated successful targeted integration of transgene expressing GFP into the site 7 of GEMS sequence of CHO-K1 cells, in addition to the transgene expressing CD19-CAR already engineered in site 7 of GEMS Cas9sites.

The methods described in this example resulted in generation of genetically engineered cell comprising multiple transgene inserted within a GEMS sequence that is inserted within the genome of CHO-K1 cells, i.e., transgene expressing CD19-CAR inserted in site 7, and transgene expressing GFP inserted in site 2. The genetically engineered cells generated by methods described in this Example expressed CD19-CAR, and GFP.

### EXAMPLE 5. Engineering Transgene Expressing an Antibody (e.g., a PD-L1 Binding Antibody, or a VEGF Binding Antibody) into Site 4, Transgene Expressing GFP in Site 2, and Transgene Expressing CD19 CAR in Site 7 of a GEMS Sequence in the Rosa26 Genomic Loci of CHO-K1 Cells

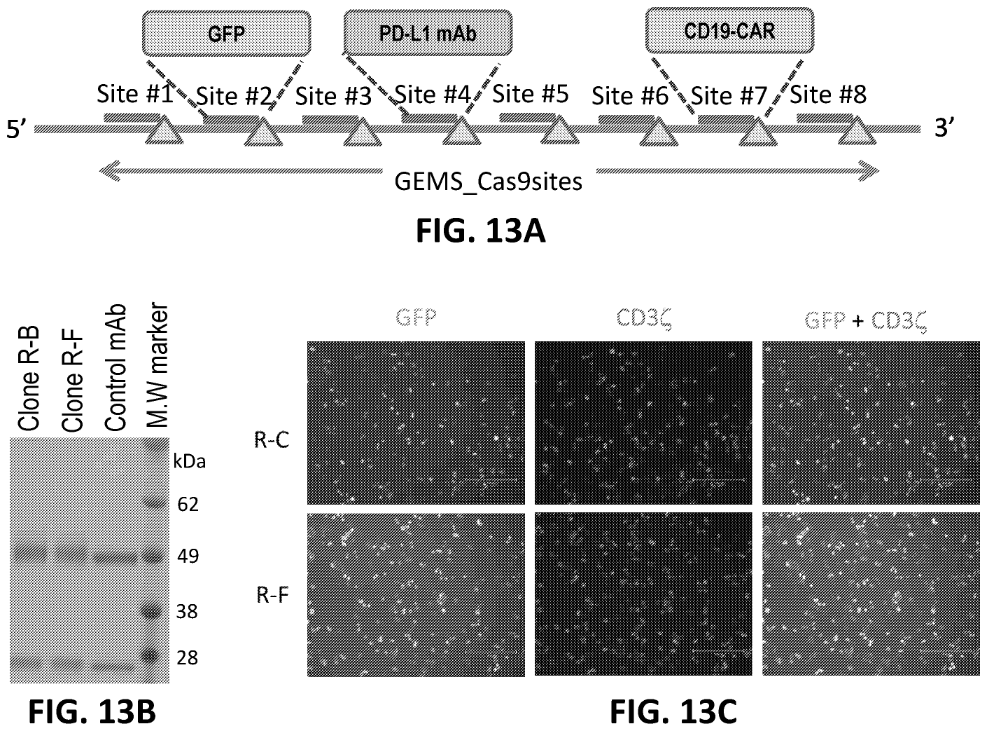

This Example demonstrates engineering multiple transgenes in different nuclease recognition sequences of a GEMS sequence that is integrated in the genome of a CHO-K1 cell. The transgene expressing CD19-CAR was engineered in the site 7 of the GEMS sequence as described in Example 3 above to generate genetically engineered cells expressing CD19-CAR. The transgene expressing GFP was engineered in the site 7 of the GEMS sequence as described in Example 4 above to generate genetically engineered cells expressing CD19-CAR, and GFP. To further demonstrate the capability to engineer a third transgene in the GEMS sequence, a transgene expressing an antibody, e, g., a PD-L1 binding antibody was engineered into site 4 of GEMS sequence, further to transgene expressing GFP engineered in site 2, and transgene expressing CD19 CAR engineered in site 7 of GEMS-Cas9sites (FIG. 13A). To do this, a GEMSsite4sgRNA-pCas9D10A single shot plasmid was constructed to express GEMS site 4 sgRNA (SEQ ID NO: 96) and Cas9 with D10A mutation. In contrast to native Cas9 enzyme, D10A mutation leads Cas9 to nick DNA sequence in single strand without double strand cutting.

The PDL1mAb_GEMSsite4 donor plasmid was constructed to express the light chain polypeptide (SEQ ID NO: 85), and the heavy chain polypeptide of the PD-L1 binding antibody (SEQ ID NO: 86). The transgene expressing the antibody (e.g., a PD-L1 binding antibody) had a nucleic acid sequence encoding the light chain and the nucleic acid sequence encoding the heavy chain linked by Thosea asigna virus 2A (T2A) self-cleavage peptide (SEQ ID NO: 87), and were under the control of an exogenous promoter e.g., EF-1a promoter (SEQ ID NO: 18). The transgene expressing the antibody, along with a neomycin selection marker under the control of an exogenous promoter e.g., CMV promoter (SEQ ID NO: 11), was flanked by GEMS sequence surrounding the selected target site for insertion of transgene (site 4) as the 5′ and 3′ homology arms to facilitate homology recombination. To facilitate homology recombination, target sequence of site 4 (SEQ ID NO: 95) were designed to flank the transgene expressing the antibody to be inserted so the donor vector can also be cut during CRISPR-mediated cleavage.

Equal amount of GEMSsite4sgRNA-pCas9D10A single shot plasmid and PDL1mAb_GEMSsite4 donor plasmid were transfected by electroporation using the 4D-Nucleofector™ System from Lonza into 2×10⁶ CHO-K1 cells that had a transgene expressing GFP engineered in site 2 and a transgene expressing CD19-CAR engineered in site 7. The transfected cells were cultured in media with geneticin to select geneticin resistant cells. About two weeks after transfection, geneticin resistant single cell colonies formed and were picked by cloning discs. The selected monoclonal cells were further propagated in the presence of geneticin selection.

The expression of the PD-L1 binding antibody from the monoclonal cell lines was confirmed by antibody purification followed by SDS-PAGE. Briefly, the culture media for the monoclonal cell lines was collected and the PD-L1 binding antibody secreted by the cells was purified using protein A agarose beads pull down. The purified antibody was subjected to SDS-PAGE analysis under reduced condition. The heavy chain and light chain of the PD-L1 binding antibody were detected from two representative cell lines (FIG. 13B). Further ELISA assays confirmed the binding to PD-L1 antigen by the purified PD-L1 binding antibody.

Besides PD-L1 binding antibody, the expression of GFP in the monoclonal cell line was detected by fluorescent microscope (FIG. 13C). Additionally, the expression of CD19 CAR on the cell surface of monoclonal cell line was confirmed by immunostaining by an anti-CD3zeta antibody. Briefly, the cells were fixed on slides, permeabilized, and stained by an anti-CD3zeta mouse antibody. The bound antibody was detected by a secondary goat anti-mouse antibody conjugated with Alexa594. The expression of CD19 CAR was detected by anti-CD3zeta antibody with Alexa594 signals along the cell surface (FIG. 13C). The GFP signal colocalized with the immunostaining signals for CD3zeta in the cells (FIG. 13C). These expression studies confirmed the expression of three transgens in the genetically engineered cells with the three transgenes engineered in three different nuclease recognition sequences of a GEMS sequence.

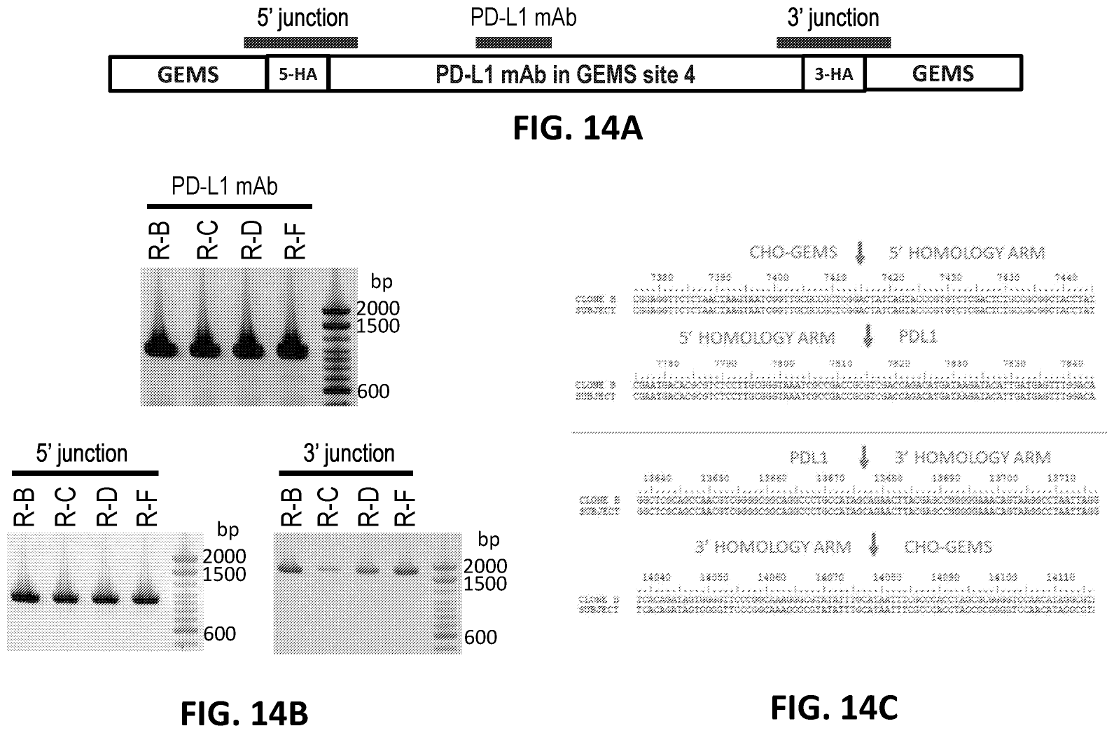

The genomic DNA from geneticin resistant monoclonal cells were prepared. The presence of the transgene expressing an antibody (PD-L1 binding antibody) in the genome of cloned cells was confirmed by PCR using primers specific to the transgene (FIG. 14A) which resulted in amplified PCR bands with correct sizes (FIG. 14B). The correct insertion of transgene expressing an antibody (e.g., a PD-L1 binding antibody) into the site 4 of GEMS sequence was confirmed by analyzing the 5′ and 3′ junction sites between the GEMS sequence and the inserted transgene by PCR using one primer specific to GEMS sequence and another primer specific to the inserted transgene (FIG. 14A). The appropriate 5′ and 3′ junction were confirmed by PCR with DNA bands with expected sizes (FIG. 14B). The PCR products were further sequenced by Sanger sequencing. Correct junction site sequences between GEMS site selected for insertion and homology arm and between homology arm and transgene expressing an antibody were confirmed for both 5′ and 3′ junction sites (FIG. 14C). The results indicated successful targeted integration of transgene expressing an antibody (e.g., a PD-L1 antibody) into the site 4 of GEMS sequence within genome of CHO-K1 cells, with GFP engineered in site 2 and CD19-CAR engineered in site 7. Further PCR and sequencing work confirmed the presence of GFP in site 2, and CD19-CAR in site 7 of GEMS sequence in the established monoclonal cell lines.

### EXAMPLE 6. Design a GEMS Construct with a GEMS Sequence that has Multiple Recombinase Recognition Sequences (GEMS_Bxb1sites) for CHO Cell Genome Engineering

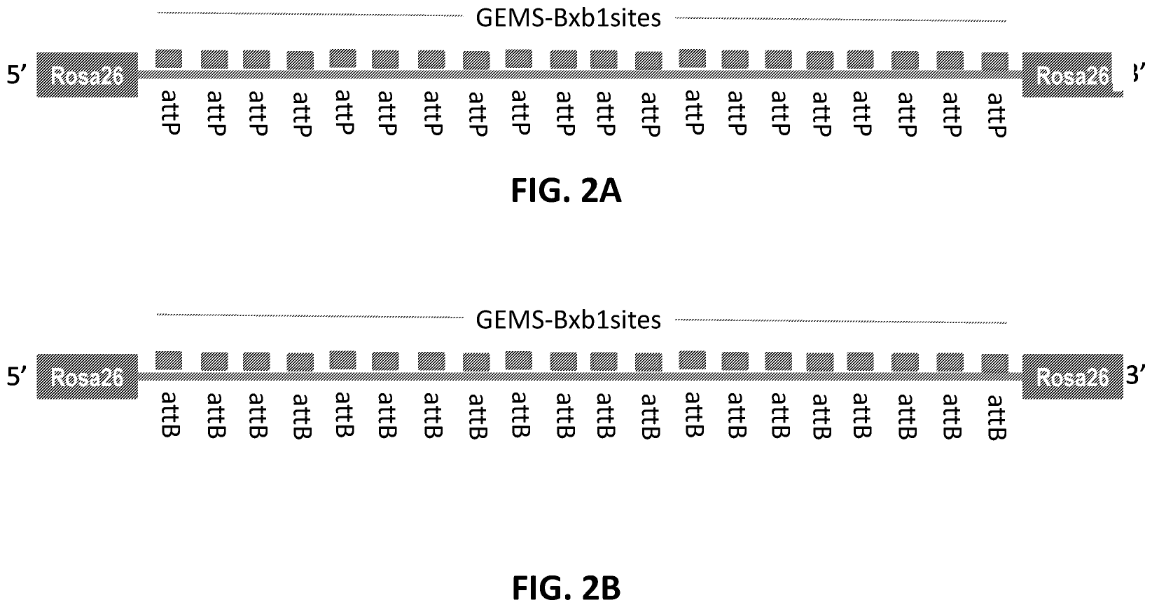

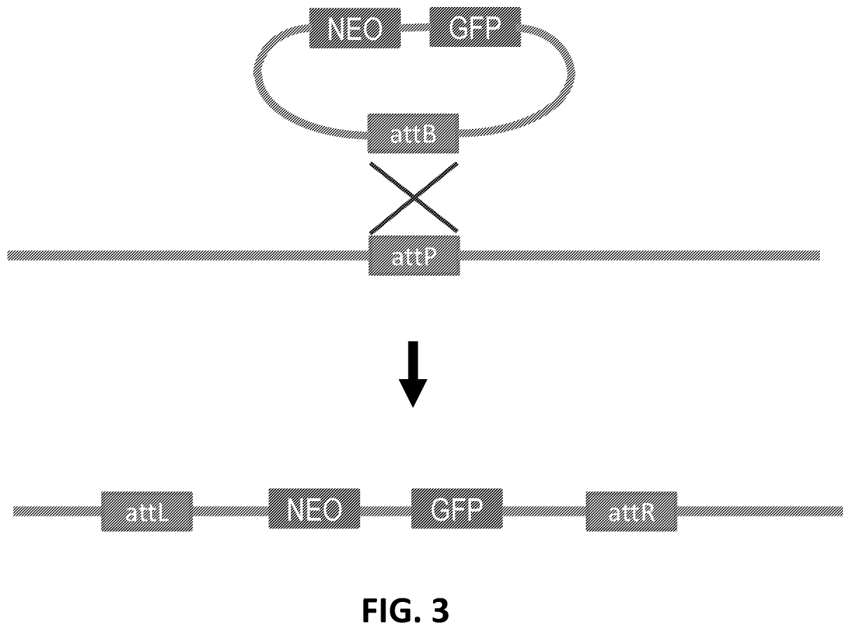

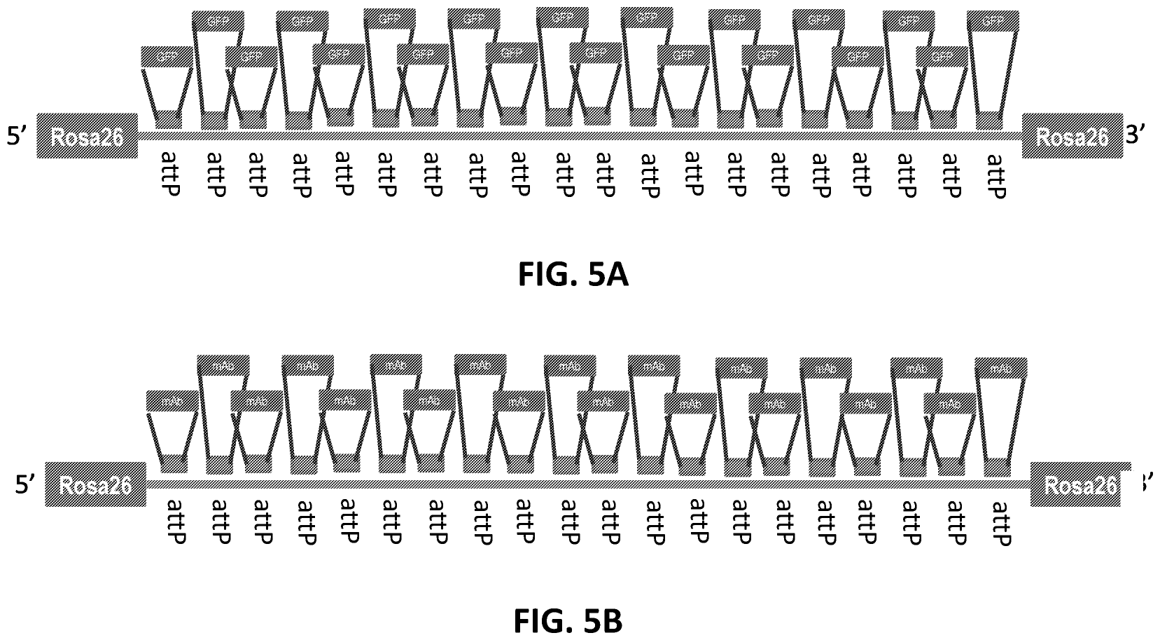

A CHO_Rosa26_GEMS_Bxb1sites plasmid was generated that comprises a GEMS sequence with twenty Bxb1 recombinase sequences, i.e., attP. (FIG. 2A). These attP sequences (SEQ ID NO: 106) were flanked by 50 bp random sequences as the spacer. These twenty attP sites serve as integration (insertion) sites to engineer as many as twenty transgenes in the Rosa26 safe harbor site by Bxb1 recombinase mediated cassette exchange (FIG. 3-4). For example, multiple copies of transgene expressing the heavy and light chains of an antibody (e.g., a PD-L1 binding antibody, or a VEGF binding antibody) are engineered into these attP sites to facilitate stable and high expression of therapeutic antibody molecules (FIG. 5B), as described below.

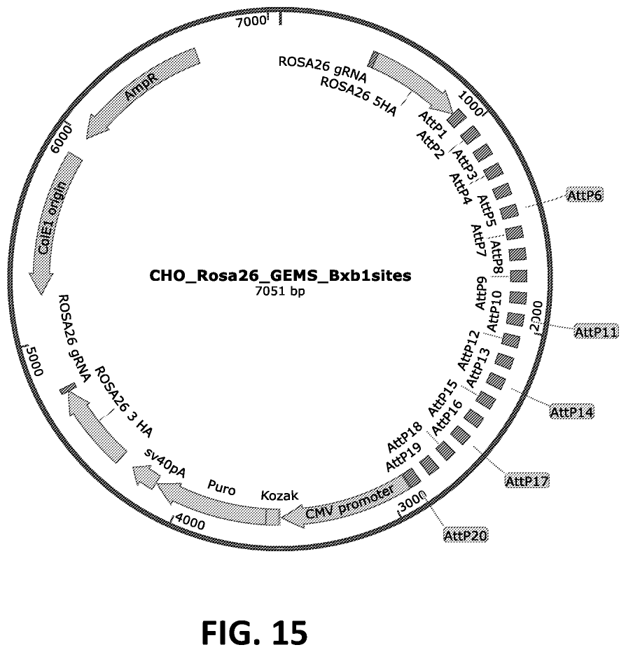

CHO_Rosa26_GEMS_Bxb1sites plasmid was constructed with arrays of distinct gene editing sites engineered in their GEMS sequences to facilitate site-specific engineering in the loci of Rosa26 of CHO cell genome. In this plasmid (FIG. 15), the GEMS sequence and a selection cassette were flanked by ˜500 bp Rosa26 sequences surrounding the cutting site as the 5′ and 3′ homology arms (ROSA26 5HA and ROSA26 3HA) to facilitate homology recombination. The selection cassette was composed of puromycin selection marker (SEQ ID NO: 13) driven by an exogenous promoter e.g., a CMV promoter (SEQ ID NO: 11). To facilitate homology recombination, Rosa26 targeting site sequence (SEQ ID NO: 9) were designed to flank the transgene sequence to be inserted so the donor vector can also be cut during CRISPR-mediated cleavage.

### EXAMPLE 7. Engineering a GEMS Construct with a GEMS Sequence that has Multiple Recombinase Recognition Sequences (GEMS_Bxb1sites) into the Rosa26 Genomic Loci of CHO-K1 Cells

GEMS_Bxb1sites were integrated into the Rosa26 site of CHO-kl cells by CRISPR/Cas9-mediated site specific integration. Equal amount of pCas9D10A_CHO_Rosa26gRNA single shot plasmid and CHO_Rosa26_GEMS_Bxb1sites donor plasmid were transfected into 2×10⁶ CHO-K1 cells by electroporation using the 4D-Nucleofector™ System from Lonza. The transfected cells were cultured in media with puromycin to select puromycin resistant cells. Two weeks after transfection, puromycin resistant single cell colonies were formed and picked by cloning discs. The selected monoclonal cells were further propagated in the presence of puromycin selection.

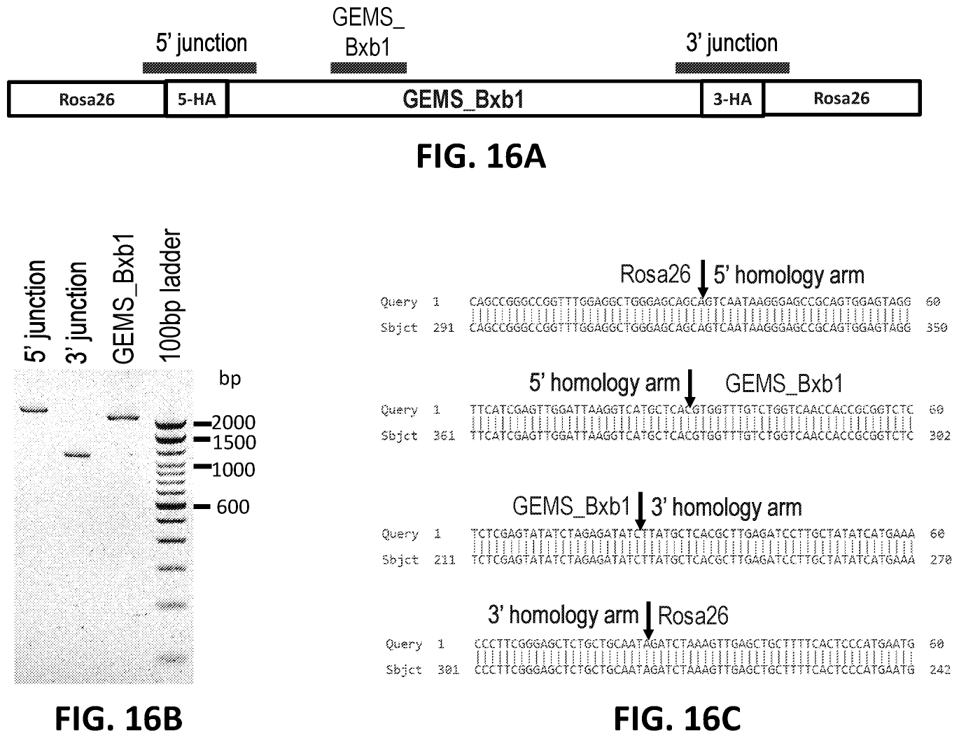

The genomic DNA from puromycin resistant monoclonal CHO-kl cells were prepared. The CHO-GEMS-Bxb1sites sequences integrated into the cell genome were evaluated by PCR using primers specific to corresponding GEMS sequences followed by Sanger sequencing of the PCR products (FIG. 16A). Correct-sized GEMS_Bxb1 fragment was amplified by PCR from genomic DNA isolated from multiple cell clones examined with an example illustrated in FIG. 16B, indicating the successful integration of a GEMS sequence that has multiple recombinase recognition sequences (GEMS_Bxb1sites) in cell genome. The PCR products were further sequenced and the identity of GEMS_Bxb1sites sequence was confirmed.

The proper insertion of GEMS sequences into the Rosa26 site was evaluated by analyzing the 5′ and 3′ junction sites between the Rosa26 site and the inserted GEMS sequence by PCR using one primer specific to Rosa26 sequence and another primer specific to the inserted GEMS sequence (FIG. 16A). The appropriate 5′ and 3′ junctions were confirmed by PCR which resulted in DNA bands with expected sizes (FIG. 16B). The PCR products were further sequenced by Sanger sequencing. Correct junctions between Rosa26 site and homology arm and between homology arm and GEMS_Bxb1 targeting cassette were confirmed for both 5′ and 3′ junction sites (FIG. 16C), indicating successful targeted integration of GEMS_Bxb1sites sequence in the Rosa26 site of CHO-K1 cells.

### EXAMPLE 8. Engineering Two Distinct GEMS Sequences into the H11 Site of CHO-K1 Cells

Besides Rosa26 site, the two distinct GEMS sequences GEMS_Bxb1sites, and GEMS_Cas9sites described in Examples above, are engineered in other safe harbor genomic sites of CHO cell genome, for example the Hipp11 (H11) locus. The Hipp11 (H11) locus, which is situated between the DRG1 and EIF4ENIF1 genes in mice, humans, and pigs, offers great potential for stable gene knock-in and expression at high levels. The robust and ubiquitous function of H11 has been demonstrated in mice, pigs, human embryonic stem (hES) cells, and induced pluripotent stem (iPS) cells. H11 is utilized as an alternative site for engineering the two distinct GEMS sequences, GEMS_Bxb1sites, and GEMS_Cas9sites described in Examples above.

Two candidate specific integration site sequences with predicted high efficacy score by CRISPRater are identified in H11 locus. The sequence of one integration site is: gaagtcctttcgagggcatgagg at coordination NW 003614682.1:81036-81058. The sequence of another site is: gtagcgttgtgacaggctttcgg at coordination NW 003614682.1:83297-83319. The efficiency of Cas9 mediated cleavage at these two sites is evaluated in surveyor nuclease assay. Then a similar strategy is employed to engineer the GEMS sequences in H11 locus as that to the Rosa26 locus. Specifically, a single shot plasmid expressing Cas9D10A and H11 targeting site sgRNA and a GEMS donor plasmid with the multiple nuclease recognition sequences i.e., GEMS_Cas9sites flanked by 5′ and 3′ homology arm sequences derived by sequences flanking the H11 locus, and another GEMS donor plasmid with multiple recombinase recognition sequences i.e., GEMS_Bxb1sites flanked by 5′ and 3′ homology arm sequences derived by sequences flanking the H11 locus are designed.

### EXAMPLE 9. Engineering Transgene Expressing GFP into the GEMS Sequence, GEMS_Bxb1sites Inserted in a Rosa26 Genomic Loci of CHO K1 Cells

An expression plasmid (pMAX-Bxb1) was constructed in which the expression of Bxb1 recombinase was driven by CMV promoter. In this construct, DNA sequence encoding SV40 nuclear localization signal was engineered in frame with the N-terminus of Bxb1 sequence (SEQ ID NO: 108). The nuclear localization signal facilitates the expression of Bxb1 recombinase in cell nuclei to promote Bxb1 mediated cassette exchange. Besides pMAX-Bxb1, an alternative expression plasmid (pcDNA3.4-Bxb1) was constructed in which the expression of Bxb1 recombinase without the SV40 muclear localization signal was driven by CMV promoter.

A donor plasmid comprising a transgene expressing GFP (pSP72-attB-CMV-HYG-EF1a-GFP) was constructed for engineering the transgene into the multiple recombination recognition sequences, e.g., Bxb1 recognition sites of the established CHO-K1 cell line that has GEMS sequence, GEMS_Bxb1sites engineered in a Rosa26 genomic loci. An attB sequence (SEQ ID NO: 107) was engineered on transgene expressing GFP to facilitate DNA recombination between the attB site of donor plasmid, pSP72-attB-CMV-HYG-EF1a-GFP, and the attP sites of GEMS sequence (GEMS-Bxb1sites) in the engineered CHO cell genome by Bxb1 mediated cassette exchange. The expression of GFP is driven by EF1-a promoter (SEQ ID NO: 18). In this construct, a hygromycin selection marker under CMV promoter was engineered.

Besides pSP72-attB-CMV-HYG-EF1a-GFP, other donor vector constructs were also generated with the same design as pSP72-attB-CMV-HYG-EF1a-GFP except the expression of GFP is driven by human CMV promoter (SEQ ID NO; 11), mouse CMV promoter (SEQ ID NO: 82), or Chinese Hamster EF1a promoter (SEQ ID NO: 83).

Instead of a wild-type hygromycin selection marker, a mutated neomycin-phosphotransferase (NPT) that has a D227V mutation and exhibits reduced enzyme activity due to the D227V mutation was also engineered as a selection marker. The mutated NPT is expressed under control of an exogenous promoter e.g., a CMV promoter. The use of mutant NPT in the donor plasmid comprising a transgene is to facilitate the selection of cells with multiple copies of transgene engineered into the cell genome. The reduced NPT activity is compensated by enhanced NPT expression due to high copy number of mutated NPT along with the transgene.

For targeted insertion of a transgene expressing GFP, equal amount of pcDNA3.4-Bxb1 plasmid expressing Bxb1 recombinase and pSP72-attB-CMV-HYG-EF1a-GFP donor expression plasmid were transfected into 2×10⁶ CHO-K1 cells with GEMS_Bxb1sites engineered in Rosa26 by electroporation using the 4D-Nucleofector™ System from Lonza. The transfected cells were cultured in media with hygromycin to select cells resistant to hygromycin. Hygromycin resistant single cell colonies were formed and picked by cloning discs.

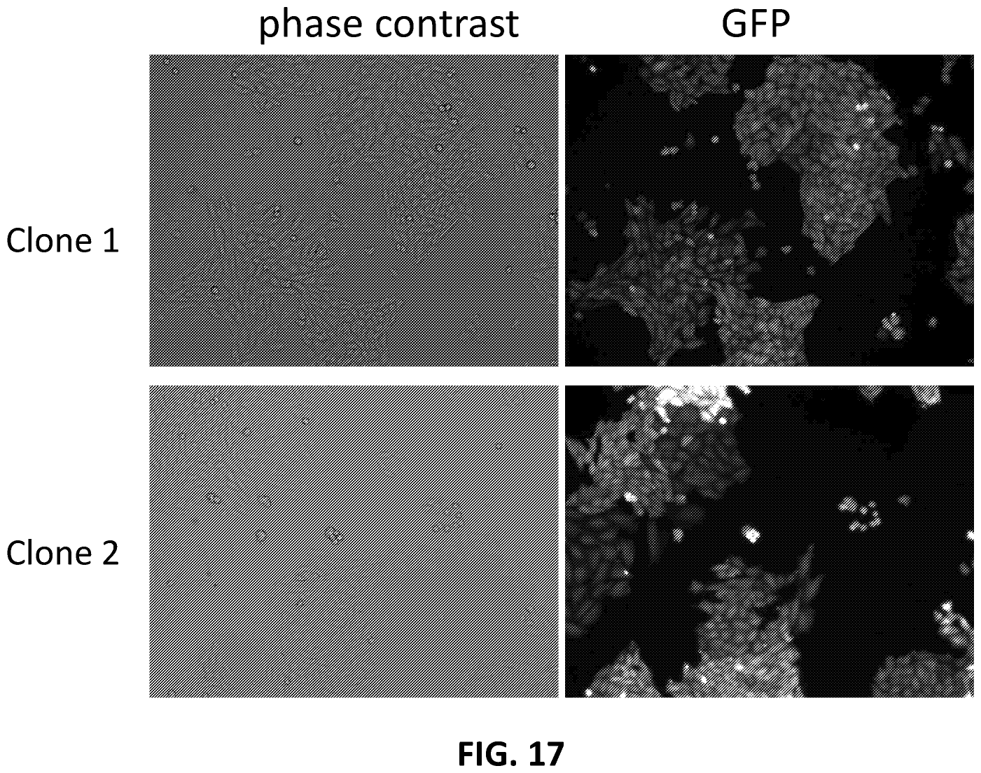

The expression of GFP in the selected monoclonal cell lines were observed under fluorescent microscope. As shown in FIG. 17 of two representative clones, the expression of GFP was observed in majority of the cells. The genomic DNA from hygromycin resistant monoclonal cells are prepared. The presence and insertion of GFP-expression cassette into the multiple attP sites of GEMS Bxb1sites are evaluated by PCR and sequencing. The copy number of transgene expressing GFP in these sites are deduced then the correlation between GFP expression level versus the copy number of transgene expressing GFP is established.

### EXAMPLE 10. Engineering Transgene Expressing an Antibody into GEMS_Bxb1sites that is Inserted in Rosa26 Genomic Loci in CHO K1 Cells

A donor plasmid with a transgene expressing an antibody, e.g., a PD-L1 binding antibody (pSP72-attB-CMV-HYG-EF1a-PDL1mAb) was constructed for the engineering the transgene expressing the antibody into the multiple Bxb1 sites of the established CHO-K1 cell line with GEMS_Bxb1sites inserted Rosa26. An attB sequence is engineered on this donor plasmid to facilitate DNA recombination between the attB site of pSP72-attB-CMV-HYG-EF1a-PDL1mAb, and the multiple attP sites of GEMS-Bxb1sites in the engineered CHO cell genome by Bxb1 mediated cassette exchange. The transgene expressing an antibody has a nucleic acid sequence that encodes a light chain polypeptide (SEQ ID NO: 85) and a nucleic acid sequence that encodes a heavy chain polypeptide (SEQ ID NO: 86) of the antibody that is separated by a Thosea asigna virus 2A (T2A) sequence (SEQ ID NO: 87). The expression of the transgene is driven by EF1a promoter (SEQ ID NO: 18). The self-cleavage of T2A peptide can facilitate the separation of the translated heavy and light chain polypeptides. In this construct, a hygromycin selection marker under CMV promoter is also constructed.

Besides pSP72-attB-CMV-HYG-EF1a-PDL1mAb, other donor vector constructs were also generated with the same design as pSP72-attB-CMV-HYG-EF1a-PDL1mAb except the expression of mAb is driven by human CMV promoter (SEQ ID NO; 11), mouse CMV promoter (SEQ ID NO: 82) or Chinese Hamster EF-1a promoter (SEQ ID NO: 83).

Instead of a wild-type hygromycin selection marker, a mutated neomycin selection marker that has a neomycin-phosphotransferase with a D227V mutation and that exhibits a reduced enzyme activity is engineered with the expression of mutant neomycin selection marker under CMV promoter. The use of mutant NPT in the donor plasmid comprising a transgene is to facilitate the selection of cells with multiple copies of transgene engineered into the cell genome. The reduced NPT activity is compensated by enhanced NPT expression due to high copy number of mutated NPT along with the transgene.

Equal amount of pcDNA3.4-Bxb1 plasmid expressing Bxb1 recombinase and pSP72-attB-CMV-HYG-EF1a-PDL1 mAb donor plasmid are transfected into 2×10⁶ CHO-K1 cells with GEMS_Bxb1sites engineered in Rosa26 by electroporation using the 4D-Nucleofector™ System from Lonza. The transfected cells are cultured in selection media with hygromycin to select cells resistant to hygromycin. Hygromycin resistant single cell colonies are formed and picked by cloning discs.

The expression of PD-L1 binding antibody in the selected monoclonal cell lines are quantitated by ELISA assay using anti-IgG antibody to detect antibody secreted in the supernatant. The genomic DNA from hygromycin resistant monoclonal cells is prepared. The presence and insertion of transgene expressing an antibody into the multiple attP sites of GEMS_Bxb1sites are evaluated by PCR and sequencing. The copy number of transgene expressing an antibody in these sites are deduced then the correlation between antibody expression level versus the copy number of transgene expressing antibody is established.

Once high expression cell clones are obtained, the culture of engineered CHO-K1 cells is adapted into suspension format. The titer of antibody production of the engineered cell line will be determined. Besides engineering CHO-K1 cells as a proof of concept study, the same set of constructs and methods as described in Examples herein can be applied in the engineering of other cell lines for the production of therapeutic antibodies and other biologics molecules. The methods of engineering cells (e.g., CHO cells) described herein can be applied in the manufacture of therapeutic biologics molecules with defined and high production.

### Sequences

Provided herein is a representative list of certain sequences included in embodiments provided herein.

TABLE 7
Sequences

| SEQ | | |
|---|---|---|
| ID | | |
| NO | Description | Sequence (5′ to 3′) |
| 1 | GEMS sequence with | atccgtattccgacgtacgatggaccactcttcacacgta |
| | plurality of nuclease | aagcaagaacgtcgagcagtcatgaaagtcttagtaccgc |
| | recognition sequences; | acgtgccatcttactgcgaatattgcctgaagctgtaccg |
| | GEMS_Cas9sites | ttattggggggcaaagatgaagttctcctcttttcataat |
| | | tgtactgacgacagccgtgttcccggtttcttcagaggtt |
| | | aaagaataagggcttattgtaggcagagggacgccctttt |
| | | agtggctggcgttaagtatcttcggacccccttgtctatc |
| | | cagattaatcgaattctctcatttaggaccctagtaagtc |
| | | atcattggtatttgaatgcgaccccgaagaaaccgcctaa |
| | | aaatgtcaatggttggtccactaaacttcatttaatcaac |
| | | tcctaaatcggcgcgataggccagtattcgagtaggcgtc |
| | | gatgggttagaggtttaattttgtatggcaaggtacttcc |
| | | gatcttaatgaatggccggaagaggtacggacgcgatatg |
| | | cgggggtgagagggcaaataggcaggttcgccttcgtcac |
| | | gctaggaggcaattctataagaatgcacattgcatggata |
| | | cataaaatgtctcgaccgcttgcgcaacttgtgaagtgtc |
| | | tactatccctaagcccatttcccgcataataacccctgat |
| | | tgtgtccgcatctgatgctacccgggttgagttagcgtcg |
| | | agctcgcggaacttattgcatgagtagagttgagtaagag |
| | | ctgttagatggctcgctgagctaatagttgcccacagaac |
| | | gtcaagattagagaacggtcgtagcattatcggaggttct |
| | | ctaactaagtaatcggttgcgccgctcggactatcagtac |
| | | ccgtgtctcgactctgccgcggctacctatcgcctgaaag |
| | | ccagttggtgttaaggggtgctctgtccaggacgccacgc |
| | | gtagtgagacttacatgttcgttgggttcacccgactcgg |
| | | acctgagtggaccaaggacgcactcgagctctgagcccta |
| | | ctgtcgagaaatatgtatctcgcccccgcagcttgccagc |
| | | tctttcagtatcatggagcccatggttgaatgactcctat |
| | | aacgaacttcgacatggcaaaatccccccctcgcgacttc |
| | | aagagaagaagagtactgacttgagcgctcccagcacttc |
| | | agccaaggaagttaccaatttcttgtttccgaatgacacg |
| | | cgtctccttgcgggtaaatcgccgaccgcagtaatcggtt |
| | | gcgacgctcgggagaacttacgagccaggggaaacagtaa |
| | | ggcctaattaggtaaagggagtaagtgctcgaacggttca |
| | | gttgtaaccatatacttacgctggatcttctccggcgaat |
| | | ttttaccgtcaccaactacgagatttgaggtaaaccaaat |
| | | gagcacatagtggcgctatccgactatttccaaattgtaa |
| | | catatcgttccatgaaggccagagttacttaccggccctt |
| | | tccatgcgcgcgccataccctcctagttccccggttatct |
| | | ctccgaggagagagtgagcgatcctccgttaacatattgt |
| | | taccaatgacgtagctatgtattttgcacaggtagccaac |
| | | gggtttcacatttcacagatagtggggttcccggcaaagg |
| | | gcgtatatttgcataatttcgcccacctagcgcggggtcc |
| | | aacataggcgtaaactacgatggcacctactcagacgcag |
| | | ctcgtgcggcgtaaataacgtactcatcccaactgattct |
| | | cggcaatctacggagcgacatgattatcaacagctgtcta |
| | | gcagttctaatcttttgccatggtcgtaaaagcctccaag |
| | | agattgatcatacctatcggcacagaagtgacacgacgcc |
| | | gatgggtagcggactttaggtcaaccacagttcggtaggg |
| | | gacaggccctgcggcgtacatcactttgtatgtgcaacgt |
| | | gcccaagtggcgccaggcaagactcagctggttcctgtgt |
| | | tagctcgaggctaggcatgacagctctttgaacatgggct |
| | | gggggcctcgaacggtcgagaagcccatagtacctgcgtg |
| | | cgatcgtaccgtctacggcggataccaagttgcgcaggct |
| | | atagcttgaagctgtactatttcagggggggagccctgat |
| | | ggtctcttcttctgatgactcaactcgctagggtcgtgaa |
| | | gtcgattccttcgatggttaaaaatcaaaggctcagagtg |
| | | cagactggagcgcccatctaacggttcgcatctcgaatgc |
| | | tcggtcgcctttcacattccgcgaaaattcataccgctca |
| | | ttcactaggttgcgaagtctacactgatatatgaatccga |
| | | gctagagcagggctcttaaaattcggagtcgttgatgctc |
| | | aatactccaatcggttttttcgtgcaccaccgcgagtggc |
| | | tgacaagggtttgacattgagtagcaaggcagttccgggc |
| | | tgaatgaagcgccgggaaccttcagcaacgtttcgcgtgg |
| | | gtgtgaattgttattcgcgaaaaacatccgtccccgtggg |
| | | ggatagtcaccgacgccgttttatagaagtgtaggggaac |
| | | aggttggtttaactagcttaagaaagtaaattctgggatt |
| | | atactgtagtaatcactaatttacggtgagggttttatgg |
| | | cggatctttacaaattcaagccaggtgatttcaacaaatt |
| | | ttgctgacgatttaggcgcactatcccctaaactacaaat |
| | | tagaaaatagcgttccttgacggctagaattacctaccgg |
| | | cctccaccataccttcgatattcgcgcccactctcccatt |
| | | aatccgcacaagtggatgtgatgcgattgcccgctaagat |
| | | attctaacgtgtaacgcagatgagtattctacagagttgc |
| | | cgtagcaaccgccggctacggggg |
| 2 | I-SceI meganuclease | TAGGGATAACAGGGTAAT |
| | recognition site | |
| 3 | GEMS sequence with | ATCCGTATTCCGACGTACGATGGACCACTCTTCACACGTA |
| | plurality of nuclease | AAGCAAGAACGTCGAGCAGTCATGAAAGTCTTAGTACCGC |
| | recognition sequence; | ACGTGCCATCTTACTGCGAATATTGCCTGAAGCTGTACCG |
| | GEMS2_Cas9sites | TTATTGGGGGGCAAAGATGAAGTTCTCCTCTTTTCATAAT |
| | | TGTACTGACGACAGCCGTGTTCCCGGTTTCTTCAGAGGTT |
| | | AAAGAATAAGGGCTTATTGTAGGCAGAGGGACGCCCTTTT |
| | | AGTGGCTGGCGTTAAGTATCTTCGGACCCCCTTGTCTATC |
| | | CAGATTAATCGAATTCTCTCATTTAGGACCCTAGTAAGTC |
| | | ATCATTGGTATTTGAATGCGACCCCGAAGAAACCGCCTAA |
| | | AAATGTCAATGGTTGGTCCACTAAACTTCATTTAATCAAC |
| | | TCCTAAATCGGCGCGATAGGCCAGTATTCGAGTAGGCGTC |
| | | GATGGGTTAGAGGTTTAATTTTGTATGGCAAGGTACTTCC |
| | | GATCTTAATGAATGGCCGGAAGAGGTACGGACGCGATATG |
| | | CGGGGGTGAGAGGGCAAATAGGCAGGTTCGCCTTCGTCAC |
| | | GCTAGGAGGCAATTCTATAAGAATGCACATTGCATGGATA |
| | | CATAAAATGTCTCGACCGCTTGCGCAACTTGTGAAGTGTC |
| | | TACTATCCCTAAGCCCATTTCCCGCATAATAACCCCTGAT |
| | | TGTGTCCGCATCTGATGCTACCCGGGTTGAGTTAGCGTCG |
| | | AGCTCGCGGAACTTATTGCATGAGTAGAGTTGAGTAAGAG |
| | | CTGTTAGATGGCTCGCTGAGCTAATAGTTGCCCACAGAAC |
| | | GTCAAGATTAGAGAACGGTCGTAGCATTATCGGAGGTTCT |
| | | CTAACTAAGTAATCGGTTGCGCCGCTCGGACTATCAGTAC |
| | | CCGTGTCTCGACTCTGCCGCGGCTACCTATCGCCTGAAAG |
| | | CCAGTTGGTGTTAAGGGGTGCTCTGTCCAGGACGCCACGC |
| | | GTAGTGAGACTTACATGTTCGTTGGGTTCACCCGACTCGG |
| | | ACCTGAGTGGACCAAGGACGCACTCGAGCTCTGAGCCCTA |
| | | CTGTCGAGAAATATGTATCTCGCCCCCGCAGCTTGCCAGC |
| | | TCTTTCAGTATCATGGAGCCCATGGTTGAATGACTCCTA |
| | | TAACGAACTTCGACATGGCAAAATCCCCCCCTCGCGACTT |
| | | CAAGAGAAGAAGAGTACTGACTTGAGCGCTCCCAGCACTT |
| | | CAGCCAAGGAAGTTACCAATTTCTTGTTTCCGAATGACAC |
| | | GCGTCTCCTTGCGGGTAAATCGCCGACCGCTAATCCTCGC |
| | | GGTAACCGGTAGGAGAACTTACGAGCCAGGGGAAACAGTA |
| | | AGGCCTAATTAGGTAAAGGGAGTAAGTGCTCGAACGGTTC |
| | | AGTTGTAACCATATACTTACGCTGGATCTTCTCCGGCGAA |
| | | TTTTTACCGTCACCAACTACGAGATTTGAGGTAAACCAAA |
| | | TGAGCACATAGTGGCGCTATCCGACTATTTCCAAATTGTA |
| | | ACATATCGTTCCATGAAGGCCAGAGTTACTTACCGGCCCT |
| | | TTCCATGCGCGCGCCATACCCTCCTAGTTCCCCGGTTATC |
| | | TCTCCGAGGAGAGAGTGAGCGATCCTCCGTTAACATATTG |
| | | TTACCAATGACGTAGCTATGTATTTTGCACAGGTAGCCAA |
| | | CGGGTTTCACATTTCACAGATAGTGGGGTTCCCGGCAAAG |
| | | GGCGTATATTTGCATGGGCTTCTCGACCGTTCGAGGGGTC |
| | | CAACATAGGCGTAAACTACGATGGCACCTACTCAGACGCA |
| | | GCTCGTGCGGCGTAAATAACGTACTCATCCCAACTGATTC |
| | | TCGGCAATCTACGGAGCGACATGATTATCAACAGCTGTCT |
| | | AGCAGTTCTAATCTTTTGCCATGGTCGTAAAAGCCTCCAA |
| | | GAGATTGATCATACCTATCGGCACAGAAGTGACACGACGC |
| | | CGATGGGTAGCGGACTTTAGGTCAACCACAGTTCGGTAGG |
| | | GGACAGGCCCTGCGGCGTACATCACTTTGTATGTGCAACG |
| | | TGCCCAAGTGGCGCCAGGCAAGACTCAGCTGGTTCCTGTG |
| | | TTAGCTCGAGGCTAGGCATGACAGCTCTTTGAACATGGGC |
| | | TGGGGGCCTCGAACGGTCGAGAAGCCCATAGTACCTGCGT |
| | | GCGATCGTACCGTCTACGGCGGATACCAAGTTGCGCAGGC |
| | | TATAGCTTGAAGCTGTACTATTTCAGGGGGGGAGCCCTGA |
| | | TGGTCTCTTCTTCTGATGACTCAACTCGCTAGGGTCGTGA |
| | | AGTCGATTCCTTCGATGGTTAAAAATCAAAGGCTCAGAGT |
| | | GCAGACTGGAGCGCCCATCTAACGGTTCGCATCTCGAATG |
| | | CTCGGTCGCCTTTCACATTCCGCGAAAATTCATACCGCTC |
| | | ATTCACTAGGTTGCGAAGTCTACACTGATATATGAATCCG |
| | | AGCTAGAGCAGGGCTCTTAAAATTCGGAGTCGTTGATGCT |
| | | CAATACTCCAATCGGTTTTTTCGTGCACCACCGCGAGTGG |
| | | CTGACAAGGGTTTGACATTGAGTAGCAAGGCAGTTCCGGG |
| | | CTGAATGAAGCGCCGGGAACCTTCAGCAACGTTTCGCGTG |
| | | GGTGTGAATTGTTATTCGCGAAAAACATCCGTCCCCGTGG |
| | | GGGATAGTCACCGACGCCGTTTTATAGAAGTGTAGGGGAA |
| | | CAGGTTGGTTTAACTAGCTTAAGAAAGTAAATTCTGGGAT |
| | | TATACTGTAGTAATCACTAATTTACGGTGAGGGTTTTATG |
| | | GCGGATCTTTACAAATTCAAGCCAGGTGATTTCAACAAAT |
| | | TTTGCTGACGATTTAGGCGCACTATCCCCTAAACTACAAA |
| | | TTAGAAAATAGCGTTCCTTGACGGCTAGAATTACCTACCG |
| | | GCCTCCACCATACCTTCGATATTCGCGCCCACTCTCCCAT |
| | | TAATCCGCACAAGTGGATGTGATGCGATTGCCCGCTAAGA |
| | | TATTCTAACGTGTAACGCAGATGAGTATTCTACAGAGTTG |
| | | CCTTTATATGGGACGCGTACGCCGG |
| 4 | 5′ junction site reverse | CCGATAAAACACATGCGTCA |
| | primer | |
| | (5′AAVS1 | |
| | targCheckR1) | |
| 5 | 3′ junction site forward | CACGCGGTCGTTATAGTTCA |
| | primer (3′AAVS1 | |
| | targCheckF1) | |
| 6 | 3′ junction site reverse | CGGAGGAATATGTCCCAGAT |
| | primer | |
| | (3′AAVS1 | |
| | targCheckR1) | |
| 7 | CHO_Rosa26 5′ | gtcaataagggagccgcagtggagtaggcggggagaaggc |
| | homology arm | cgcaccctactcggctgggggaggggagtgccgcaatacc |
| | | tttctgggagttctctgctgcctcctgtcttctaaagacc |
| | | gccccgggactggaaggatcccttccccctttcccctcgt |
| | | gatctgcaagtcgaggctttctgggagatgggcgggagtc |
| | | ttctgggcaggcttgagggctaacctggtgcgtgggcgtt |
| | | gtcctgcaggggaattgaactggtgtaaaattggaagggt |
| | | gagaattcccacggattttcgtttgtgtcgggaggtgatt |
| | | gtaataggggcaaaggagggaaatgggagactaggtgctc |
| | | gcctggggttttgtgcagcaaaactacaggttattattaa |
| | | taagccttggagtatttttcatcgagttggattaaggtca |
| | | tgctcac |
| 8 | CHO_Rosa26 3′ | ttatgctcacgcttgagatccttgctatatcatgaaatta |
| | homology arm | tagtgtcgcaagttagaatacataaacagaattttagtgt |
| | | tttctacagggccctgcacttcactctttccctcctgctc |
| | | cctctgcagccctaccaaaagatattttagcactctcatt |
| | | tgagtccccttttcatttgttagtactggctcacccaatc |
| | | cctagacagagcactggcattcttcccctcatgatcttag |
| | | aagcctgatgagtcatgaaaccagacagattagttacacc |
| | | acaaattgaggctgtagctggggccttaccctgcagttct |
| | | tttatgcctccttagtacattttgttgactgtttgccttg |
| | | attttcattttctatccccttcgggagctctgctgcaata |
| 9 | CHO_Rosa26 CRISPR | tcaagcgtgagcataaaactcgg |
| | targeting sequence | |
| 10 | CHO_Rosa26 CRISPR | UCAAGCGUGAGCAUAAAACUGUUUUAGAGCUAGAAAUAGC |
| | guide RNA | AAGUUAAAAUAAGGCUAGUCCGUUAUCAACUUGAAAAAGU |
| | | GGCACCGAGUCGGUGCUUUU |
| 11 | CMV promoter | ACATTGATTATTGACTAGTTATTAATAGTAATCAATTACG |
| | | GGGTCATTAGTTCATAGCCCATATATGGAGTTCCGCGTTA |
| | | CATAACTTACGGTAAATGGCCCGCCTGGCTGACCGCCCAA |
| | | CGACCCCCGCCCATTGACGTCAATAATGACGTATGTTCCC |
| | | ATAGTAACGCCAATAGGGACTTTCCATTGACGTCAATGGG |
| | | TGGACTATTTACGGTAAACTGCCCACTTGGCAGTACATCA |
| | | AGTGTATCATATGCCAAGTACGCCCCCTATTGACGTCAAT |
| | | GACGGTAAATGGCCCGCCTGGCATTATGCCCAGTACATGA |
| | | CCTTATGGGACTTTCCTACTTGGCAGTACATCTACGTATT |
| | | AGTCATCGCTATTACCATGGTGATGCGGTTTTGGCAGTAC |
| | | ATCAATGGGCGTGGATAGCGGTTTGACTCACGGGGATTTC |
| | | CAAGTCTCCACCCCATTGACGTCAATGGGAGTTTGTTTTG |
| | | GCACCAAAATCAACGGGACTTTCCAAAATGTCGTAACAAC |
| | | TCCGCCCCATTGACGCAAATGGGCGGTAGGCGTGTACGGT |
| | | GGGAGGTCTATATAAGCAGAGCTCTCTGGCTAACTAGAGA |
| | | ACCCACTGCTTACTGG |
| 12 | GFP | ATGGAGAGCGACGAGAGCGGCCTGCCCGCCATGGAGATCG |
| | | AGTGCCGCATCACCGGCACCCTGAACGGCGTGGAGTTCGA |
| | | GCTGGTGGGCGGCGGAGAGGGCACCCCCAAGCAGGGCCGC |
| | | ATGACCAACAAGATGAAGAGCACCAAAGGCGCCCTGACCT |
| | | TCAGCCCCTACCTGCTGAGCCACGTGATGGGCTACGGCTT |
| | | CTACCACTTCGGCACCTACCCCAGCGGCTACGAGAACCCC |
| | | TTCCTGCACGCCATCAACAACGGCGGCTACACCAACACCC |
| | | GCATCGAGAAGTACGAGGACGGCGGCGTGCTGCACGTGAG |
| | | CTTCAGCTACCGCTACGAGGCCGGCCGCGTGATCGGCGAC |
| | | TTCAAGGTGGTGGGCACCGGCTTCCCCGAGGACAGCGTGA |
| | | TCTTCACCGACAAGATCATCCGCAGCAACGCCACCGTGGA |
| | | GCACCTGCACCCCATGGGCGATAACGTGCTGGTGGGCAGC |
| | | TTCGCCCGCACCTTCAGCCTGCGCGACGGCGGCTACTACA |
| | | GCTTCGTGGTGGACAGCCACATGCACTTCAAGAGCGCCAT |
| | | CCACCCCAGCATCCTGCAGAACGGGGGCCCCATGTTCGCC |
| | | TTCCGCCGCGTGGAGGAGCTGCACAGCAACACCGAGCTGG |
| | | GCATCGTGGAGTACCAGCACGCCTTCAAGACCCCCATCGC |
| | | CTTCGCCAGATCCCGCGCTCAGTCGTCCAATTCTGCCGTG |
| | | GACGGCACCGCCGGACCCGGCTCCACCGGATCTCGC |
| 13 | puromycin | ATGACCGAGTACAAGCCCACGGTGCGCCTCGCCACCCGCG |
| | | ACGACGTCCCCAGGGCCGTCCGCACCCTCGCCGCCGCGTT |
| | | CGCCGACTACCCCGCCACGCGCCACACCGTCGATCCGGAC |
| | | CGCCACATCGAGCGGGTCACCGAGCTGCAAGAACTCTTCC |
| | | TCACGCGCGTCGGGCTCGACATCGGCAAGGTGTGGGTCGC |
| | | GGACGACGGCGCCGCGGTGGCGGTCTGGACCACGCCGGAG |
| | | AGCGTCGAAGCGGGGGCGGTGTTCGCCGAGATCGGCCCGC |
| | | GCATGGCCGAGTTGAGCGGTTCCCGGCTGGCCGCGCAGCA |
| | | ACAGATGGAAGGCCTCCTGGCGCCGCACCGGCCCAAGGAG |
| | | CCCGCGTGGTTCCTGGCCACCGTCGGCGTCTCGCCCGACC |
| | | ACCAGGGCAAGGGTCTGGGCAGCGCCGTCGTGCTCCCCGG |
| | | AGTGGAGGCGGCCGAGCGCGCCGGGGTGCCCGCCTTCCTG |
| | | GAGACCTCCGCGCCCCGCAACCTCCCCTTCTACGAGCGGC |
| | | TCGGCTTCACCGTCACCGCCGACGTCGAGGTGCCCGAAGG |
| | | ACCGCGCACCTGGTGCATGACCCGCAAGCCCGGTGCC |
| 14 | neomycin | ATGATTGAACAAGATGGATTGCACGCAGGTTCTCCGGCCG |
| | | CTTGGGTGGAGAGGCTATTCGGCTATGACTGGGCACAACA |
| | | GACAATCGGCTGCTCTGATGCCGCCGTGTTCCGGCTGTCA |
| | | GCGCAGGGGCGCCCGGTTCTTTTTGTCAAGACCGACCTGT |
| | | CCGGTGCCCTGAATGAACTGCAGGACGAGGCAGCGCGGCT |
| | | ATCGTGGCTGGCCACGACGGGCGTTCCTTGCGCAGCTGTG |
| | | CTCGACGTTGTCACTGAAGCGGGAAGGGACTGGCTGCTAT |
| | | TGGGCGAAGTGCCGGGGCAGGATCTCCTGTCATCTCACCT |
| | | TGCTCCTGCCGAGAAAGTATCCATCATGGCTGATGCAATG |
| | | CGGCGGCTGCATACGCTTGATCCGGCTACCTGCCCATTCG |
| | | ACCACCAAGCGAAACATCGCATCGAGCGAGCACGTACTCG |
| | | GATGGAAGCCGGTCTTGTCGATCAGGATGATCTGGACGAA |
| | | GAGCATCAGGGGCTCGCGCCAGCCGAACTGTTCGCCAGGC |
| | | TCAAGGCGCGAATGCCCGACGGCGAGGATCTCGTCGTGAC |
| | | CCATGGCGATGCCTGCTTGCCGAATATCATGGTGGAAAAT |
| | | GGCCGCTTTTCTGGATTCATCGACTGTGGCCGGCTGGGTG |
| | | TGGCGGACCGCTATCAGGACATAGCGTTGGCTACCCGTGA |
| | | TATTGCTGAAGAGCTTGGCGGCGAATGGGCTGACCGCTTC |
| | | CTCGTGCTTTACGGTATCGCCGCTCCCGATTCGCAGCGCA |
| | | TCGCCTTCTATCGCCTTCTTGACGAGTTCTTC |
| 15 | MUC1 scFv | GATATCGTGATGACCCAGAGCCCCAGCTCTTTAACCGTGA |
| | | CAGCCGGCGAGAAGGTGACCATGATCTGCAAGAGCTCCCA |
| | | GTCTTTACTGAACAGCGGCGACCAGAAGAACTATTTAACT |
| | | TGGTATCAACAGAAGCCCGGACAGCCCCCCAAGCTGCTCA |
| | | TCTTCTGGGCCTCCACCAGAGAGAGCGGCGTGCCCGATCG |
| | | TTTTACCGGCTCCGGCTCCGGCACCGACTTTACTTTAACC |
| | | ATCAGCAGCGTGCAAGCTGAAGATTTAGCCGTGTACTACT |
| | | GCCAGAACGACTACAGCTACCCTTTAACATTCGGCGCCGG |
| | | CACCAAGCTGGAACTGAAGGGCGGCGGCGGTTCTGGAGGC |
| | | GGAGGCAGCGGCGGCGGCGGCAGCCAAGTTCAGCTGCAGC |
| | | AGTCCGACGCTGAGCTGGTGAAGCCCGGCAGCTCCGTCAA |
| | | GATTAGCTGCAAAGCCTCCGGCTACACCTTCACAGACCAC |
| | | GCCATTCACTGGGTGAAGCAGAAGCCCGAACAAGGTTTAG |
| | | AGTGGATCGGCCACTTCTCCCCCGGCAACACCGACATCAA |
| | | GTACAACGACAAGTTCAAGGGCAAGGCCACTTTAACCGTG |
| | | GATAGAAGCAGCTCCACCGCCTACATGCAGCTGAACTCTT |
| | | TAACCAGCGAGGATAGCGCCGTGTACTTCTGCAAGACCAG |
| | | CACCTTCTTCTTCGACTATTGGGGCCAAGGCACTACTTTA |
| | | ACAGTGTCCAGC |
| 16 | CD22 scFv | GATATCCAGATGACCCAATCCCCTAGCTCTCTGAGCGCCA |
| | | GCGTGGGAGACAGAGTGACAATCACATGTAGAGCTTCCCA |
| | | GACCATCTGGAGCTACCTCAACTGGTATCAGCAGAGGCCC |
| | | GGCAAGGCCCCTAATCTGCTCATTTATGCTGCCAGCTCCC |
| | | TCCAGTCCGGAGTGCCTTCTAGGTTCTCCGGAAGAGGCTC |
| | | CGGCACCGACTTCACACTGACCATCAGCTCTCTGCAAGCC |
| | | GAGGACTTCGCCACCTACTACTGCCAGCAGAGCTACAGCA |
| | | TCCCCCAGACCTTCGGCCAAGGCACCAAGCTGGAGATTAA |
| | | GGGCGGCGGCGGAAGCGGAGGAGGAGGCAGCGGAGGCGGC |
| | | GGCAGCCAAGTGCAACTGCAACAGTCCGGCCCCGGACTGG |
| | | TGAAACCCTCCCAGACACTGTCTCTGACATGCGCTATCAG |
| | | CGGCGATAGCGTGTCCAGCAACTCCGCCGCTTGGAACTGG |
| | | ATTAGACAGTCCCCTAGCAGAGGACTGGAATGGCTGGGAA |
| | | GAACCTACTATAGATCCAAGTGGTACAACGACTATGCCGT |
| | | GAGCGTGAAGTCTAGGATCACCATCAACCCCGATACCTCC |
| | | AAGAACCAGTTCTCTCTGCAGCTCAATAGCGTGACCCCCG |
| | | AGGACACCGCCGTGTACTATTGTGCCAGAGAGGTGACCGG |
| | | CGATCTGGAGGATGCCTTCGATATCTGGGGACAAGGCACC |
| | | ATGGTGACCGTGTCCTCC |
| 17 | 2B4 | TGGAGGAGGAAGAGAAAGGAGAAGCAGAGCGAGACCAGCC |
| | | CCAAGGAGTTTTTAACTATCTACGAGGACGTGAAGGATTT |
| | | AAAGACTCGTAGGAACCACGAGCAAGAACAGACATTCCCC |
| | | GGTGGCGGTAGCACCATCTACAGCATGATCCAGAGCCAGA |
| | | GCAGCGCTCCCACCAGCCAAGAACCCGCTTACACTTTATA |
| | | CTCTTTAATCCAGCCCTCTCGTAAGAGCGGCAGCAGAAAG |
| | | AGGAACCACAGCCCCAGCTTCAACAGCACTATCTATGAGG |
| | | TGATCGGCAAGAGCCAGCCCAAGGCCCAGAACCCCGCTCG |
| | | TCTGTCTCGTAAGGAGCTGGAAAACTTCGACGTGTACAGC |
| 18 | Human EF-1alpha | CGTGAGGCTCCGGTGCCCGTCAGTGGGCAGAGCGCACATC |
| | promoter | GCCCACAGTCCCCGAGAAGTTGGGGGGAGGGGTCGGCAAT |
| | | TGAACCGGTGCCTAGAGAAGGTGGCGCGGGGTAAACTGGG |
| | | AAAGTGATGTCGTGTACTGGCTCCGCCTTTTTCCCGAGGG |
| | | TGGGGGAGAACCGTATATAAGTGCAGTAGTCGCCGTGAAC |
| | | GTTCTTTTTCGCAACGGGTTTGCCGCCAGAACACAGGTAA |
| | | GTGCCGTGTGTGGTTCCCGCGGGCCTGGCCTCTTTACGGG |
| | | TTATGGCCCTTGCGTGCCTTGAATTACTTCCACCTGGCTG |
| | | CAGTACGTGATTCTTGATCCCGAGCTTCGGGTTGGAAGTG |
| | | GGTGGGAGAGTTCGAGGCCTTGCGCTTAAGGAGCCCCTTC |
| | | GCCTCGTGCTTGAGTTGAGGCCTGGCCTGGGCGCTGGGGC |
| | | CGCCGCGTGCGAATCTGGTGGCACCTTCGCGCCTGTCTCG |
| | | CTGCTTTCGATAAGTCTCTAGCCATTTAAAATTTTTGATG |
| | | ACCTGCTGCGACGCTTTTTTTCTGGCAAGATAGTCTTGTA |
| | | AATGCGGGCCAAGATCTGCACACTGGTATTTCGGTTTTTG |
| | | GGGCCGCGGGCGGCGACGGGGCCCGTGCGTCCCAGCGCAC |
| | | ATGTTCGGCGAGGCGGGGCCTGCGAGCGCGGCCACCGAGA |
| | | ATCGGACGGGGGTAGTCTCAAGCTGGCCGGCCTGCTCTGG |
| | | TGCCTGGCCTCGCGCCGCCGTGTATCGCCCCGCCCTGGGC |
| | | GGCAAGGCTGGCCCGGTCGGCACCAGTTGCGTGAGCGGAA |
| | | AGATGGCCGCTTCCCGGCCCTGCTGCAGGGAGCTCAAAAT |
| | | GGAGGACGCGGCGCTCGGGAGAGCGGGCGGGTGAGTCACC |
| | | CACACAAAGGAAAAGGGCCTTTCCGTCCTCAGCCGTCGCT |
| | | TCATGTGACTCCACGGAGTACCGGGCGCCGTCCAGGCACC |
| | | TCGATTAGTTCTCGAGCTTTTGGAGTACGTCGTCTTTAGG |
| | | TTGGGGGGAGGGGTTTTATGCGATGGAGTTTCCCCACACT |
| | | GAGTGGGTGGAGACTGAAGTTAGGCCAGCTTGGCACTTGA |
| | | TGTAATTCTCCTTGGAATTTGCCCTTTTTGAGTTTGGATC |
| | | TTGGTTCATTCTCAAGCCTCAGACAGTGGTTCAAAGTTTT |
| | | TTTCTTCCATTTCAGGTGTCGTGA |
| 19 | blasticidin | ATGGCCAAGCCTTTGTCTCAAGAAGAATCCACCCTCATTG |
| | | AAAGAGCAACGGCTACAATCAACAGCATCCCCATCTCTGA |
| | | AGACTACAGCGTCGCCAGCGCAGCTCTCTCTAGCGACGGC |
| | | CGCATCTTCACTGGTGTCAATGTATATCATTTTACTGGGG |
| | | GACCTTGTGCAGAACTCGTGGTGCTGGGCACTGCTGCTGC |
| | | TGCGGCAGCTGGCAACCTGACTTGTATCGTCGCGATCGGA |
| | | AATGAGAACAGGGGCATCTTGAGCCCCTGCGGACGGTGCC |
| | | GACAGGTGCTTCTCGATCTGCATCCTGGGATCAAAGCCAT |
| | | AGTGAAGGACAGTGATGGACAGCCGACGGCAGTTGGGATT |
| | | CGTGAATTGCTGCCCTCTGGTTATGTGTGGGAGGGC |
| 20 | CD19 scFv | GAAATTGTGATGACCCAGTCACCCGCCACTCTTAGCCTTT |
| | | CACCCGGTGAGCGCGCAACCCTGTCTTGCAGAGCCTCCCA |
| | | AGACATCTCAAAATACCTTAATTGGTATCAACAGAAGCCC |
| | | GGACAGGCTCCTCGCCTTCTGATCTACCACACCAGCCGGC |
| | | TCCATTCTGGAATCCCTGCCAGGTTCAGCGGTAGCGGATC |
| | | TGGGACCGACTACACCCTCACTATCAGCTCACTGCAGCCA |
| | | GAGGACTTCGCTGTCTATTTCTGTCAGCAAGGGAACACCC |
| | | TGCCCTACACCTTTGGACAGGGCACCAAGCTCGAGATTAA |
| | | AGGTGGAGGTGGCAGCGGAGGAGGTGGGTCCGGCGGTGGA |
| | | GGAAGCCAGGTCCAACTCCAAGAAAGCGGACCGGGTCTTG |
| | | TGAAGCCATCAGAAACTCTTTCACTGACTTGTACTGTGAG |
| | | CGGAGTGTCTCTCCCCGATTACGGGGTGTCTTGGATCAGA |
| | | CAGCCACCGGGGAAGGGTCTGGAATGGATTGGAGTGATTT |
| | | GGGGCTCTGAGACTACTTACTACAACTCATCCCTCAAGTC |
| | | ACGCGTCACCATCTCAAAGGACAACTCTAAGAATCAGGTG |
| | | TCACTGAAACTGTCATCTGTGACCGCAGCCGACACCGCCG |
| | | TGTACTATTGCGCTAAGCATTACTATTATGGCGGGAGCTA |
| | | CGCAATGGATTACTGGGGACAGGGTACTCTGGTCACCGTG |
| | | TCCAGC |
| 21 | CD8 hinge and | ACCACTACCCCAGCACCGAGGCCACCCACCCCGGCTCCTA |
| | transmembrane domain | CCATCGCCTCCCAGCCTCTGTCCCTGCGTCCGGAGGCATG |
| | | TAGACCCGCAGCTGGTGGGGCCGTGCATACCCGGGGTCTT |
| | | GACTTCGCCTGCGATATCTACATTTGGGCCCCTCTGGCTG |
| | | GTACTTGCGGGGTCCTGCTGCTTTCACTCGTGATCACTCT |
| | | TTACTGT |
| 22 | 4-1BB endodomain | AAGCGCGGTCGGAAGAAGCTGCTGTACATCTTTAAGCAAC |
| | | CCTTCATGAGGCCTGTGCAGACTACTCAAGAGGAGGACGG |
| | | CTGTTCATGCCGGTTCCCAGAGGAGGAGGAAGGCGGCTGC |
| | | GAACTG |
| 23 | CD3 zeta domain | CGCGTGAAATTCAGCCGCAGCGCAGATGCTCCAGCCTACA |
| | | AGCAGGGGCAGAACCAGCTCTACAACGAACTCAATCTTGG |
| | | TCGGAGAGAGGAGTACGACGTGCTGGACAAGCGGAGAGGA |
| | | CGGGACCCAGAAATGGGCGGGAAGCCGCGCAGAAAGAATC |
| | | CCCAAGAGGGCCTGTACAACGAGCTCCAAAAGGATAAGAT |
| | | GGCAGAAGCCTATAGCGAGATTGGTATGAAAGGGGAACGC |
| | | AGAAGAGGCAAAGGCCACGACGGACTGTACCAGGGACTCA |
| | | GCACCGCCACCAAGGACACCTATGACGCTCTTCACATGCA |
| | | GGCCCTGCCGCCTCGG |
| 81 | hygromycin | ATGAAAAAGCCTGAACTCACCGCGACGTCTGTCGAGAAGT |
| | | TTCTGATCGAAAAGTTCGACAGCGTCTCCGACCTGATGCA |
| | | GCTCTCGGAGGGCGAAGAATCTCGTGCTTTCAGCTTCGAT |
| | | GTAGGAGGGCGTGGATATGTCCTGCGGGTAAATAGCTGCG |
| | | CCGATGGTTTCTACAAAGATCGTTATGTTTATCGGCACTT |
| | | TGCATCGGCCGCGCTCCCGATTCCGGAAGTGCTTGACATT |
| | | GGGGAATTCAGCGAGAGCCTGACCTATTGCATCTCCCGCC |
| | | GTGCACAGGGTGTCACGTTGCAAGACCTGCCTGAAACCGA |
| | | ACTGCCCGCTGTTCTGCAGCCGGTCGCGGAGGCCATGGAT |
| | | GCGATCGCTGCGGCCGATCTTAGCCAGACGAGCGGGTTCG |
| | | GCCCATTCGGACCGCAAGGAATCGGTCAATACACTACATG |
| | | GCGTGATTTCATATGCGCGATTGCTGATCCCCATGTGTAT |
| | | CACTGGCAAACTGTGATGGACGACACCGTCAGTGCGTCCG |
| | | TCGCGCAGGCTCTCGATGAGCTGATGCTTTGGGCCGAGGA |
| | | CTGCCCCGAAGTCCGGCACCTCGTGCACGCGGATTTCGGC |
| | | TCCAACAATGTCCTGACGGACAATGGCCGCATAACAGCGG |
| | | TCATTGACTGGAGCGAGGCGATGTTCGGGGATTCCCAATA |
| | | CGAGGTCGCCAACATCTTCTTCTGGAGGCCGTGGTTGGCT |
| | | TGTATGGAGCAGCAGACGCGCTACTTCGAGCGGAGGCATC |
| | | CGGAGCTTGCAGGATCGCCGCGGCTCCGGGCGTATATGCT |
| | | CCGCATTGGTCTTGACCAACTCTATCAGAGCTTGGTTGAC |
| | | GGCAATTTCGATGATGCAGCTTGGGCGCAGGGTCGATGCG |
| | | ACGCAATCGTCCGATCCGGAGCCGGGACTGTCGGGCGTAC |
| | | ACAAATCGCCCGCAGAAGCGCGGCCGTCTGGACCGATGGC |
| | | TGTGTAGAAGTACTCGCCGATAGTGGAAACCGACGCCCCA |
| | | GCACTCGTCCGAGGGCAAAGGAA |
| 82 | Mouse CMV promoter | CTACGGTGGTCAGACCGAAGACTGCGACGGTACCGACGCT |
| | | GGTCGCGCCTCTTATACCCACGTAGAACGCAGCTCAGCCA |
| | | ATAGAATGAGTGCCAATATGGAATTTCCAGGGGAAAACCG |
| | | GGGCGGTGTTACGTTTTGGCTGCCCTTTCACTTCCCATTG |
| | | ACGTGTATTGGCTCGAGAACGGTACTTTCCCATTAATCAG |
| | | CTATGGGAAAGTACCGTTTAAAGGTCACGTTGCATTAGTT |
| | | TCAATAGCCCATTGACGTCAATGGTGGGAAAGTACATGGC |
| | | GTTTTAATTAAATGGCTGGAAAAACCCAATGACTCACCCC |
| | | TATTGACCTTATGTACGTGCCAATAATGGGAAAAACCCAT |
| | | TGACTCACCCCCTATTGACCTTTTGTACTGGGCAAAACCC |
| | | AATGGAAAGTCCCTATTGACTCAGTGTACTTGGCTCCAAT |
| | | GGGACTTTCCTGTTGATTCACCCCTATTGACCTTATGTAC |
| | | TGGGCAAAACCCATTGGAAAGTCCCTAATGACTCAGTATA |
| | | TCT |
| 83 | Chinese Hamster EF-1a | ACGGAACTCCCAGGGCGTGAAGCGCGCTTCAGGCTTCCAG |
| | (CHEF1-a) promoter | AGAAGCAGCTGGCGCTGGATGGAATGAACCAAGAGGCCAG |
| | | CACAGGGGCAGATCCGTCGAGCTCTCGGCCACCGAGCTGA |
| | | GCCCTTAGGTTCTGGGGCTGGGAAGGGTCCCTAGGATTGT |
| | | GCACCTCTCCCGCGGGGGACAAGCAGGGGATGGCGGGGCT |
| | | GACGTCGGGAGGTGGCCTCCACGGGAAGGGACACCCGGAT |
| | | CTCGACACAGCCTTGGCAGTGGAGTCAGGAAGGGTAGGAC |
| | | AGATTCTGGACGCCCTCTTGGCCAGTCCTCACCGCCCCAC |
| | | CCCCGATGGAGCCGAGAGTAATTCATACAAAAGGAGGGAT |
| | | CGCCTTCGCCCCTGGGAATCCCAGGGACCGTCGCTAAATT |
| | | CTGGCCGGCCTCCCAGCCCGGAACCGCTGTGCCCGCCCAG |
| | | CGCGGCGGGAGGAGCCTGCGCCTAGGGCGGATCGCGGGTC |
| | | GGCGGGAGAGCACAAGCCCACAGTCCCCGGCGGTGGGGGA |
| | | GGGGCGCGCTGAGCGGGGGCCCGGGAGCCAGCGCGGGGCA |
| | | AACTGGGAAAGTGGTGTCGTGTGCTGGCTCCGCCCTCTTC |
| | | CCGAGGGTGGGGGAGAACGGTATAAAAGTGCGGTAGTCGC |
| | | GTTGGACGTTCTTTTTCGCAACGGGTTTGCCGTCAGAACG |
| | | CAGGTGAGTGGCGGGTGTGGCCTCCGCGGGCCCGGGCTCC |
| | | CTCCTTTGAGCGGGGTCGGACCGCCGTGCGGGTGTCGTCG |
| | | GCCGGGCTTCTCTGCGAGCGTTCCCGCCCTGGATGGCGGG |
| | | CTGTGCGGGAGGGCGAGGGGGGGAGGCCTGGCGGCGGCCC |
| | | CGGAGCCTCGCCTCGTGTCGGGCGTGAGGCCTAGCGTGGC |
| | | TTCCGCCCCGCCGCGTGCCACCGCGGCCGCGCTTTGCTGT |
| | | CTGCCCGGCTGCCCTCGATTGCCTGCCCGCGGCCCGGGCC |
| | | AACAAAGGGAGGGCGTGGAGCTGGCTGGTAGGGAGCCCCG |
| | | TAGTCCGCATGTCGGGCAGGGAGAGCGGCAGCAGTCGGGG |
| | | GGGGGACCGGGCCCGCCCGTCCCGCAGCACATGTCCGACG |
| | | CCGCCTGGACGGGTAGCGGCCTGTGTCCTGATAAGGCGGC |
| | | CGGGCGGTGGGTTTTAGATGCCGGGTTCAGGTGGCCCCGG |
| | | GTCCCGGCCCGGTCTGGCCAGTACCCCGTAGTGGCTTAGC |
| | | TCCGAGGAGGGCGAGCCCGCCCGCCCGGCACCAGTTGCGT |
| | | GCGCGGAAAGATGGCCGCTCCCGGGCCCTGTAGCAAGGAG |
| | | CTCAAAATGGAGGACGCGGCAGCCCGGCGGAGCGGGGGGG |
| | | GTGAGTCACCCACACAAAGGAAGAGGGCCTTGCCCCTCGC |
| | | CGGCCGCTGCTTCCTGTGACCCCGTGGTGTACCGGCCGCA |
| | | CTTCAGTCACCCCGGGCGCTCTTTCGGAGCACCGCTGGCC |
| | | TCCGCTGGGGGAGGGGATCTGTCTAATGGCGTTGGAGTTT |
| | | GCTCACATTTGGTGGGTGGAGACTGTAGCCAGGCCAGCCT |
| | | GGCCATGGAAGTAATTCTTGGAATTTGCCCATTTTGAGTT |
| | | TGGAGCGAAGCTGATTGACAAAGCTGCTTAGCCGTTCAAA |
| | | GGTATTCTTCGAACTTTTTTTTTAAGGTGTTGTGAAAACC |
| | | ACCGC |
| 84 | neomycin- | ATGATCGAACAAGATGGACTGCACGCTGGCAGCCCAGCTG |
| | phosphotransferase with | CTTGGGTCGAGAGACTCTTCGGATACGACTGGGCTCAGCA |
| | D227V mutation | GACTATCGGCTGTAGCGATGCTGCTGTGTTCAGACTCTCC |
| | | GCTCAAGGAAGGCCAGTGCTCTTCGTCAAGACAGATCTGA |
| | | GCGGCGCTCTCAATGAACTGCAAGATGAGGCTGCTAGACT |
| | | GAGCTGGCTGGCCACTACTGGAGTCCCATGTGCTGCTGTG |
| | | CTGGACGTGGTCACTGAAGCTGGCAGAGATTGGCTGCTGC |
| | | TGGGCGAAGTGCCCGGCCAAGATCTGCTGTCCTCCCATCT |
| | | GGCCCCAGCTGAGAAGGTCTCCATCATGGCCGACGCCATG |
| | | AGGAGGCTGCACACACTCGATCCAGCCACTTGCCCTTTCG |
| | | ATCACCAAGCCAAGCATAGGATCGAAAGGGCTAGGACTAG |
| | | AATGGAGGCCGGACTCGTGGACCAAGATGATCTGGATGAA |
| | | GAGCACCAAGGACTGGCTCCAGCCGAACTGTTCGCTAGAC |
| | | TGAAGGCTAGGATGCCAGACGGCGAAGATCTGGTGGTCAC |
| | | TCACGGCGATGCTTGTCTGCCTAACATCATGGTCGAGAAC |
| | | GGAAGGTTCAGCGGCTTTATTGATTGCGGAAGGCTCGGAG |
| | | TGGCCGATAGATACCAAGATATCGCTCTGGCCACTAGAGT |
| | | GATCGCCGAGGAGCTGGGAGGAGAATGGGCCGATAGGTTT |
| | | CTGGTGCTCTACGGCATCGCCGCCCCAGATAGCCAGAGGA |
| | | TTGCCTTCTACAGACTGCTGGACGAGTTCTTT |
| 85 | Nucleic acid sequence | ATGGAAACTCACAGCCAAGTGTTCGTCTACATGCTGCTGT |
| | encoding a light chain | GGCTCAGCGGAGTCGAGGGAGACATTCAGATGACTCAGAG |
| | polypeptide of a PD-L1 | CCCTAGCTCCCTCTCCGCTTCCGTGGGAGATAGGGTCACT |
| | binding antibody | ATCACATGTAGGGCTTCCCAAGATGTCAGCACTGCCGTCG |
| | | CTTGGTATCAGCAGAAGCCCGGCAAAGCCCCTAAACTGCT |
| | | CATCTACAGCGCCAGCTTCCTCTATAGCGGAGTCCCTAGC |
| | | AGATTCTCCGGCAGCGGCAGCGGCACTGACTTCACTCTGA |
| | | CTATCAGCAGCCTCCAGCCAGAGGACTTCGCCACTTATTA |
| | | CTGCCAGCAGTATCTGTACCATCCAGCCACATTCGGCCAA |
| | | GGCACTAAAGTCGAGATCAAGAGGACAGTGGCCGCCCCAA |
| | | GCGTGTTCATCTTCCCTCCTTCCGACGAACAGCTCAAGAG |
| | | CGGCACAGCTTCCGTCGTCTGTCTGCTGAATAACTTCTAC |
| | | CCAAGGGAGGCCAAGGTGCAATGGAAAGTCGATAACGCTC |
| | | TGCAGTCCGGCAACAGCCAAGAGAGCGTGACAGAGCAAGA |
| | | TAGCAAGGATTCCACATACTCTCTGAGCTCCACACTGACT |
| | | CTGTCCAAAGCCGACTACGAGAAGCACAAGGTCTACGCTT |
| | | GTGAAGTGACTCACCAAGGACTGTCCTCCCCAGTGACTAA |
| | | GAGCTTCAATAGGGGCGAGTGC |
| 86 | Nucleic acid sequence | ATGGCTTGGGTCTGGACACTGCTGTTTCTCATGGCCGCCG |
| | encoding a heavy chain | CCCAAAGCATTCAAGCCGAGGTGCAGCTGGTCGAGAGCGG |
| | polypeptide of a PD-L1 | AGGAGGACTCGTGCAGCCCGGCGGCTCTCTGAGGCTGAGC |
| | binding antibody | TGTGCTGCCTCCGGCTTTACATTCTCCGACAGCTGGATTC |
| | | ACTGGGTGAGGCAAGCCCCCGGCAAAGGACTGGAATGGGT |
| | | CGCTTGGATCAGCCCATATGGAGGCTCCACATACTACGCC |
| | | GACAGCGTGAAGGGAAGGTTTACTATCAGCGCCGATACAT |
| | | CCAAGAACACAGCCTATCTGCAGATGAACTCTCTGAGAGC |
| | | CGAAGACAC |
| | | AGCTGTCTACTATTGCGCCAGAAGGCACTGGCCCGGCGGC |
| | | TTTGATTACTGGGGCCAAGGCACACTGGTGACTGTCTCCT |
| | | CCGCCAGCACTAAGGGCCCTTCCGTCTTCCCTCTGGCCCC |
| | | TAGCAGCAAAAGCACATCCGGCGGAACTGCTGCTCTCGGA |
| | | TGTCTGGTGAAAGACTACTTCCCAGAGCCAGTGACAGTCA |
| | | GCTGGAACAGCGGCGCCCTCACAAGCGGCGTCCATACTTT |
| | | CCCAGCCGTGCTGCAGAGCTCCGGACTGTATTCTCTGAGC |
| | | AGCGTGGTCACAGTCCCAAGCTCCTCTCTGGGCACACAAA |
| | | CATACATCTGTAATGTCAACCATAAACCAAGCAACACTAA |
| | | GGTGGATAAGAAGGTGGAGCCTAAGAGCTGTGACAAGACA |
| | | CACACATGCCCTCCATGCCCAGCTCCAGAGCTGCTCGGCG |
| | | GACCTAGCGTCTTCCTCTTCCCTCCTAAGCCAAAGGACAC |
| | | ACTCATGATCAGCAGAACACCAGAGGTCACATGTGTGGTC |
| | | GTGGACGTCAGCCACGAAGATCCAGAGGTCAAGTTTAACT |
| | | GGTACGTGGATGGAGTGGAAGTCCACAACGCCAAGACTAA |
| | | GCCTAGGGAGGAGCAGTATGCCAGCACTTATAGGGTCGTG |
| | | TCCGTGCTGACTGTCCTCCATCAAGATTGGCTCAATGGCA |
| | | AGGAGTACAAATGCAAGGTCTCCAACAAGGCTCTGCCAGC |
| | | CCCAATCGAGAAGACAATCTCCAAGGCCAAAGGCCAGCCA |
| | | AGAGAGCCTCAAGTCTACACACTCCCTCCATCTAGGGAGG |
| | | AGATGACAAAAAACCAAGTGTCTCTGACATGTCTGGTGAA |
| | | AGGCTTCTATCCTAGCGACATCGCCGTCGAATGGGAGTCC |
| | | AATGGCCAGCCAGAGAACAACTACAAAACTACACCTCCAG |
| | | TGCTCGATAGCGATGGCAGCTTCTTCCTCTACAGCAAGCT |
| | | GACAGTGGATAAGTCTAGGTGGCAGCAAGGCAACGTCTTC |
| | | AGCTGTTCCGTCATGCACGAGGCTCTCCATAACCATTACA |
| | | CTCAGAAGTCCCTCTCCCTCTCCCCCGGCAAG |
| 87 | Thosea asigna virus 2A | GGAGAGGGAAGAGGATCTCTGCTGACATGTGGAGATGTCG |
| | self-cleavage peptide | AGGAGAACCCCGGCCCA |
| 88 | Furin recognition | AGGAGAAAGAGG |
| | sequence | |
| 89 | CHO_GEMS_Cas9 site | ATCCGTATTCCGACGTACGATGG |
| | 1 targeting sequence | |
| 90 | CHO_GEMS_Cas9 site | AUCCGUAUUCCGACGUACGAGUUUUAGAGCUAGAAAUAGC |
| | 1 guide RNA | AAGUUAAAAUAAGGCUAGUCCGUUAUCAACUUGAAAAAGU |
| | | GGCACCGAGUCGGUGCUUUU |
| 91 | CHO_GEMS_Cas9 site | GTATTCGAGTAGGCGTCGATGGG |
| | 2 targeting sequence | |
| 92 | CHO_GEMS_Cas9 site | GUAUUCGAGUAGGCGUCGAUGUUUUAGAGCUAGAAAUAGC |
| | 2 guide RNA | AAGUUAAAAUAAGGCUAGUCCGUUAUCAACUUGAAAAAGU |
| | | GGCACCGAGUCGGUGCUUUU |
| 93 | CHO_GEMS Cas9 site | AAGTAATCGGTTGCGCCGCTCGG |
| | 3 targeting sequence | |
| 94 | CHO_GEMS_Cas9 site | AAGUAAUCGGUUGCGCCGCUGUUUUAGAGCUAGAAAUAGC |
| | 3 guide RNA | AAGUUAAAAUAAGGCUAGUCCGUUAUCAACUUGAAAAAGU |
| | | GGCACCGAGUCGGUGCUUUU |
| 95 | CHO_GEMS_Cas9 site | AGTAATCGGTTGCGACGCTCGGG |
| | 4 targeting sequence | |
| 96 | CHO_GEMS_Cas9 site | AGUAAUCGGUUGCGACGCUCGUUUUAGAGCUAGAAAUAGC |
| | 4 guide RNA | AAGUUAAAAUAAGGCUAGUCCGUUAUCAACUUGAAAAAGU |
| | | GGCACCGAGUCGGUGCUUUU |
| 97 | CHO_GEMS_Cas9 site | ATAATTTCGCCCACCTAGCGCGG |
| | 5 targeting sequence | |
| 98 | CHO_GEMS_Cas9 site | AUAAUUUCGCCCACCUAGCGGUUUUAGAGCUAGAAAUAGC |
| | 5 guide RNA | AAGUUAAAAUAAGGCUAGUCCGUUAUCAACUUGAAAAAGU |
| | | GGCACCGAGUCGGUGCUUUU |
| 99 | CHO_GEMS_Cas9 site | GCGTGCGATCGTACCGTCTACGG |
| | 6 targeting sequence | |
| 100 | CHO_GEMS_Cas9 site | GCGUGCGAUCGUACCGUCUAGUUUUAGAGCUAGAAAUAGC |
| | 6 guide RNA | AAGUUAAAAUAAGGCUAGUCCGUUAUCAACUUGAAAAAGU |
| | | GGCACCGAGUCGGUGCUUUU |
| 101 | CHO_GEMS_Cas9 site | CCTTCAGCAACGTTTCGCGTGGG |
| | 7 targeting sequence | |
| 102 | CHO_GEMS_Cas9 site | CCUUCAGCAACGUUUCGCGUGUUUUAGAGCUAGAAAUAGC |
| | 7 guide RNA | AAGUUAAAAUAAGGCUAGUCCGUUAUCAACUUGAAAAAGU |
| | | GGCACCGAGUCGGUGCUUUU |
| 103 | CHO_GEMS_Cas9 site | GTAGCAACCGCCGGCTACGGGGG |
| | 8 targeting sequence | |
| 104 | CHO GEMS_Cas9 site | GUAGCAACCGCCGGCUACGGGUUUUAGAGCUAGAAAUAGC |
| | 8 guide RNA | AAGUUAAAAUAAGGCUAGUCCGUUAUCAACUUGAAAAAGU |
| | | GGCACCGAGUCGGUGCUUUU |
| 105 | GEMS_Bxblsites | GTGGTTTGTCTGGTCAACCACCGCGGTCTCAGTGGTGTAC |
| | | GGTACAAACCCAACCACTCTTCACACGTAAAGCAAGAACG |
| | | TCGAGCAGTCATGAAAGTCTTAGTGGTTTGTCTGGTCAAC |
| | | CACCGCGGTCTCAGTGGTGTACGGTACAAACCCAGTACCG |
| | | CACGTGCCATCTTACTGCGAATATTGCCTGAAGCTGTACC |
| | | GTTAGTGGTTTGTCTGGTCAACCACCGCGGTCTCAGTGGT |
| | | GTACGGTACAAACCCATTGGGGGGCAAAGATGAAGTTCTC |
| | | CTCTTTTCATAATTGTACTGACGACAGTGGTTTGTCTGGT |
| | | CAACCACCGCGGTCTCAGTGGTGTACGGTACAAACCCAGC |
| | | CGTGTTCCCGGTTTCTTCAGAGGTTAAAGAATAAGGGCTT |
| | | ATTGTAGGGTGGTTTGTCTGGTCAACCACCGCGGTCTCAG |
| | | TGGTGTACGGTACAAACCCACAGAGGGACGCCCTTTTAGT |
| | | GGCTGGCGTTAAGTATCTTCGGACCCCCTTGTGGTTTGTC |
| | | TGGTCAACCACCGCGGTCTCAGTGGTGTACGGTACAAACC |
| | | CAGTCTATCCAGATTAATCGAATTCTCTCATTTAGGACCC |
| | | TAGTAAGTCATCGTGGTTTGTCTGGTCAACCACCGCGGTC |
| | | TCAGTGGTGTACGGTACAAACCCAATTGGTATTTGAATGC |
| | | GACCCCGAAGAAACCGCCTAAAAATGTCAATGGTGTGGTT |
| | | TGTCTGGTCAACCACCGCGGTCTCAGTGGTGTACGGTACA |
| | | AACCCATGGTCCACTAAACTTCATTTAATCAACTCCTAAA |
| | | TCGGCGCGATAGGCCAGTGGTTTGTCTGGTCAACCACCGC |
| | | GGTCTCAGTGGTGTACGGTACAAACCCATTAGAGGTTTAA |
| | | TTTTGTATGGCAAGGTACTTCCGATCTTAATGAATGGCGT |
| | | GGTTTGTCTGGTCAACCACCGCGGTCTCAGTGGTGTACGG |
| | | TACAAACCCACGGAAGAGGTACGGACGCGATATGCGGGGG |
| | | TGAGAGGGCAAATAGGCAGGGTGGTTTGTCTGGTCAACCA |
| | | CCGCGGTCTCAGTGGTGTACGGTACAAACCCATTCGCCTT |
| | | CGTCACGCTAGGAGGCAATTCTATAAGAATGCACATTGCA |
| | | TGGTGGTTTGTCTGGTCAACCACCGCGGTCTCAGTGGTGT |
| | | ACGGTACAAACCCAGATACATAAAATGTCTCGACCGCTTG |
| | | CGCAACTTGTGAAGTGTCTACTATGTGGTTTGTCTGGTCA |
| | | ACCACCGCGGTCTCAGTGGTGTACGGTACAAACCCACCCT |
| | | AAGCCCATTTCCCGCATAATAACCCCTGATTGTGTCCGCA |
| | | TCTGATGTGGTTTGTCTGGTCAACCACCGCGGTCTCAGTG |
| | | GTGTACGGTACAAACCCAGCTACCCGGGTTGAGTTAGCGT |
| | | CGAGCTCGCGGAACTTATTGCATGAGTAGTGGTTTGTCTG |
| | | GTCAACCACCGCGGTCTCAGTGGTGTACGGTACAAACCCA |
| | | GAGTTGAGTAAGAGCTGTTAGATGGCTCGCTGAGCTAATA |
| | | GTTGCCCACAGTGGTTTGTCTGGTCAACCACCGCGGTCTC |
| | | AGTGGTGTACGGTACAAACCCAGAACGTCAAGATTAGAGA |
| | | ACGGTCGTAGCATTATCGGAGGTTCTCTAACTGTGGTTTG |
| | | TCTGGTCAACCACCGCGGTCTCAGTGGTGTACGGTACAAA |
| | | CCCAACTATCAGTACCCGTGTCTCGACTCTGCCGCGGCTA |
| | | CCTATCGCCTGAAAGTGGTTTGTCTGGTCAACCACCGCGG |
| | | TCTCAGTGGTGTACGGTACAAACCCAGCCAGTTGGTGTTA |
| | | AGGGGTGCTCTGTCCAGGACGCCACGCGTAGTGAGAGTGG |
| | | TTTGTCTGGTCAACCACCGCGGTCTCAGTGGTGTACGGTA |
| | | CAAACCCACTTACATGTTCGTTGGGTTCACCCGACTCGGA |
| | | CCTGAGTGGACCAAGGACGTGGTTTGTCTGGTCAACCACC |
| | | GCGGTCTCAGTGGTGTACGGTACAAACCCA |
| 106 | attP | GTGGTTTGTCTGGTCAACCACCGCGGTCTCAGTGGTGTAC |
| | | GGTACAAACCCA |
| 107 | attB | GGCCGGCTTGTCGACGACGGCGGTCTCCGTCGTCAGGATC |
| | | ATCCGG |
| 108 | BXB1 with N-terminal | ATGCCAAAGAAGAAAAGGAAGGTGGGCTCCAGAGCTCTGG |
| | nuclear localization | TGGTCATCAGACTGAGCAGAGTGACTGACGCCACAACTTC |
| | signal (NLS) | CCCAGAGAGGCAACTGGAAAGCTGTCAGCAGCTCTGTGCC |
| | | CAGAGGGGATGGGATGTCGTGGGCGTCGCCGAGGATCTGG |
| | | ATGTCAGCGGCGCCGTCGACCCTTTCGATAGAAAGAGGAG |
| | | GCCAAATCTGGCCAGATGGCTGGCCTTTGAAGAGCAGCCA |
| | | TTCGACGTCATCGTGGCCTATAGGGTGGATAGGCTGACAA |
| | | GGTCCATTAGGCATCTGCAGCAGCTCGTGCATTGGGCCGA |
| | | GGATCACAAAAAGCTGGTCGTGAGCGCCACAGAGGCCCAT |
| | | TTCGACACTACAACTCCATTCGCTGCCGTCGTCATCGCTC |
| | | TCATGGGCACAGTCGCCCAGATGGAACTGGAGGCCATCAA |
| | | GGAGAGGAACAGAAGCGCCGCCCATTTCAACATTAGGGCC |
| | | GGCAAGTATAGGGGATCCCTCCCTCCATGGGGATATCTGC |
| | | CAACAAGAGTCGATGGCGAATGGAGACTGGTCCCAGATCC |
| | | AGTGCAGAGGGAAAGGATTCTCGAGGTGTACCACAGAGTG |
| | | GTGGACAATCACGAGCCTCTGCACCTCGTGGCTCATGATC |
| | | TGAATAGGAGAGGCGTGCTGAGCCCAAAAGATTACTTCGC |
| | | CCAGCTGCAAGGCAGAGAACCTCAAGGCAGAGAGTGGAGC |
| | | GCTACTGCTCTGAAGAGGAGCATGATTAGCGAGGCTATGC |
| | | TCGGCTACGCCACACTCAACGGAAAGACAGTGAGAGACGA |
| | | CGACGGAGCCCCTCTCGTGAGAGCTGAGCCAATTCTGACA |
| | | AGGGAGCAGCTGGAAGCTCTGAGGGCTGAACTCGTGAAAA |
| | | CATCTAGGGCTAAGCCAGCTGTCAGCACACCTTCTCTGCT |
| | | GCTGAGGGTGCTCTTCTGTGCTGTCTGCGGAGAGCCAGCC |
| | | TACAAGTTCGCTGGCGGCGGAAGGAAGCATCCAAGGTATA |
| | | GGTGTAGGTCCATGGGCTTCCCAAAACACTGCGGAAATGG |
| | | CACTGTGGCCATGGCTGAATGGGACGCTTTCTGCGAAGAA |
| | | CAAGTGCTCGATCTGCTGGGAGACGCCGAAAGGCTGGAAA |
| | | AGGTCTGGGTGGCTGGATCCGATAGCGCTGTCGAGCTGGC |
| | | CGAGGTGAATGCCGAGCTCGTCGATCTGACATCCCTCATT |
| | | GGCAGCCCAGCTTATAGGGCTGGAAGCCCACAGAGGGAGG |
| | | CTCTGGATGCTAGAATCGCTGCTCTGGCCGCTAGGCAAGA |
| | | GGAGCTCGAGGGACTGGAGGCTAGACCTAGCGGCTGGGAG |
| | | TGGAGAGAGACTGGCCAGAGATTCGGCGATTGGTGGAGAG |
| | | AACAAGACACAGCCGCCAAGAACACTTGGCTGAGGAGCAT |
| | | GAACGTGAGGCTGACTTTCGATGTCAGAGGCGGCCTCACA |
| | | AGGACAATCGACTTCGGAGACCTCCAAGAGTACGAGCAGC |
| | | ATCTGAGGCTCGGCAGCGTGGTGGAAAGGCTGCACACTGG |
| | | CATGAGC |
| 109 | Nucleic acid sequence | GACATCCAGATGACTCAATCCCCTTCCTCTCTGTCCGCCA |
| | encoding a light chain | GCGTCGGCGATAGGGTCACAATCACATGTAGCGCCTCCCA |
| | polypeptide of a VEGF | AGATATCAGCAACTATCTCAATTGGTATCAGCAGAAACCC |
| | binding antibody, | GGCAAGGCTCCTAAGGTCCTCATTTACTTCACATCCTCCC |
| | Bevacizumab | TCCACAGCGGAGTGCCTTCCAGATTTTCCGGCTCCGGCAG |
| | | CGGAACTGATTTCACTCTGACTATTTCCTCTCTGCAGCCA |
| | | GAGGACTTCGCCACATACTACTGCCAGCAGTACAGCACTG |
| | | TGCCATGGACATTCGGACAAGGAACAAAGGTCGAGATTAA |
| | | GAGGACTGTGGCCGCCCCTAGCGTCTTCATTTTCCCACCT |
| | | AGCGATGAGCAGCTGAAAAGCGGCACAGCCTCCGTGGTGT |
| | | GTCTGCTGAACAACTTCTACCCAAGGGAGGCTAAGGTGCA |
| | | ATGGAAAGTGGATAACGCTCTGCAGTCCGGCAATAGCCAA |
| | | GAGAGCGTGACTGAGCAAGACAGCAAGGACAGCACATACT |
| | | CCCTCAGCAGCACACTGACTCTGAGCAAGGCCGATTACGA |
| | | GAAGCACAAGGTGTACGCTTGTGAGGTGACACACCAAGGA |
| | | CTCAGCAGCCCAGTGACTAAGTCCTTTAATAGGGGCGAAT |
| | | GT |
| 110 | Nucleic acid sequence | GAGGTGCAGCTCGTCGAAAGCGGAGGAGGACTGGTCCAGC |
| | encoding a heavy chain | CCGGCGGAAGCCTCAGACTGAGCTGTGCCGCCAGCGGATA |
| | polypeptide of a VEGF | CACATTCACTAACTACGGAATGAATTGGGTGAGGCAAGCC |
| | binding antibody, | CCCGGCAAGGGACTGGAGTGGGTCGGATGGATCAACACTT |
| | Bevacizumab | ACACTGGAGAGCCAACATACGCTGCCGACTTCAAAAGGAG |
| | | ATTTACTTTCTCTCTGGATACATCCAAGAGCACAGCTTAC |
| | | CTCCAAATGAACTCTCTGAGGGCCGAAGACACTGCCGTGT |
| | | ACTACTGCGCCAAGTACCCACACTACTACGGCTCCAGCCA |
| | | CTGGTACTTTGACGTCTGGGGCCAAGGCACACTGGTGACA |
| | | GTCTCCTCCGCTTCCACAAAGGGACCTAGCGTGTTCCCAC |
| | | TGGCCCCTAGCAGCAAGAGCACAAGCGGCGGCACTGCTGC |
| | | TCTGGGATGTCTGGTGAAGGACTACTTCCCAGAGCCAGTC |
| | | ACTGTCAGCTGGAACAGCGGAGCTCTCACAAGCGGCGTGC |
| | | ATACATTCCCAGCTGTGCTGCAAAGCAGCGGACTCTACTC |
| | | TCTGTCCTCCGTCGTCACTGTCCCTAGCTCCTCTCTGGGA |
| | | ACACAGACATACATTTGCAACGTGAACCACAAGCCTAGCA |
| | | ACACTAAGGTGGATAAGAAGGTGGAGCCAAAGAGCTGTGA |
| | | CAAGACACACACATGTCCTCCATGCCCAGCCCCAGAGCTC |
| | | CTCGGAGGCCCAAGCGTCTTTCTCTTCCCTCCTAAGCCAA |
| | | AGGACACACTCATGATCTCTAGGACACCAGAGGTGACATG |
| | | TGTGGTGGTCGATGTGAGCCACGAGGACCCAGAGGTGAAG |
| | | TTCAACTGGTACGTGGATGGCGTGGAGGTGCATAATGCTA |
| | | AGACAAAGCCTAGAGAGGAGCAGTACAACTCCACTTACAG |
| | | AGTGGTGAGCGTGCTGACTGTGCTGCACCAAGATTGGCTC |
| | | AACGGCAAGGAGTATAAGTGCAAGGTGAGCAACAAGGCTC |
| | | TGCCAGCCCCAATTGAGAAGACTATCTCCAAGGCTAAGGG |
| | | CCAGCCTAGAGAGCCACAAGTGTACACACTCCCTCCAAGC |
| | | AGAGAGGAGATGACAAAGAACCAAGTGAGCCTCACTTGTC |
| | | TGGTCAAGGGCTTTTACCCAAGCGATATCGCCGTCGAGTG |
| | | GGAATCCAACGGCCAACCAGAAAACAACTACAAGACAACA |
| | | CCACCAGTGCTGGATAGCGATGGCAGCTTCTTTCTGTACA |
| | | GCAAGCTGACTGTGGACAAGTCTAGGTGGCAGCAAGGCAA |
| | | TGTGTTCAGCTGTTCCGTGATGCATGAGGCTCTCCACAAC |
| | | CACTACACACAGAAGTCTCTGTCCCTCTCCCCCGGCAAA |

1-267. (canceled)

268. A gene editing multi-site (GEMS) polynucleotide construct for insertion into an insertion site in a genome of a mammalian cell, wherein said GEMS polynucleotide construct comprises:
a GEMS polynucleotide sequence that comprises:
a plurality of first recognition sequences for a site specific recombinase,
wherein each of the plurality of first recognition sequences can undergo a site specific recombination with a second recognition sequence of the site specific recombinase, when contacted with the site specific recombinase,
wherein the GEMS polynucleotide sequence is heterologous to the genome; and
wherein the GEMS polynucleotide sequence is non-coding.

269. The GEMS polynucleotide construct of claim 268, wherein the site specific recombinase is a serine recombinase, or a tyrosine recombinase.

270. The GEMS polynucleotide construct of claim 268, wherein at least two of the plurality of first recognition sequences is a bacterial genomic recombination site (attB) or a phage genomic recombination site (attP).

271. The GEMS polynucleotide construct of claim 268, further comprising:
a first flanking insertion sequence homologous to a first genome sequence upstream of the insertion site, wherein the first flanking insertion sequence is located upstream of the GEMS polynucleotide sequence; and
a second flanking insertion sequence homologous to a second genome sequence downstream of the insertion site, wherein second flanking insertion sequence is located downstream of the GEMS polynucleotide sequence.

272. The GEMS polynucleotide construct of claim 268, wherein at least 2 of the plurality of first recognition sequences comprises a sequence selected from the group consisting of sequences SEQ ID NOs: 106, SEQ ID NO: 107 and reverse complements thereof.

273. The GEMS polynucleotide construct of claim 268, wherein the GEMS polynucleotide sequence comprises a sequence that is least 80% identical to SEQ ID NO: 105, wherein a sequence identity of the GEMS polynucleotide sequence to SEQ ID NO: 105 is calculated by BLASTN.

274. A mammalian cell that comprises the GEMS polynucleotide construct of claim 268.

275. A method of producing a genetically engineered cell, said method comprising:
(a) providing a cell that comprises a gene editing multi-site (GEMS) polynucleotide sequence in said cell's genome, wherein said GEMS polynucleotide sequence comprises a plurality of first recognition sequences for a site specific recombinase, wherein the GEMS polynucleotide sequence is heterologous to the genome, and wherein the GEMS polynucleotide sequence is non-coding;
(b) introducing in said cell, a donor vector and a site specific recombinase or a nucleic acid sequence that encodes the site specific recombinase,
wherein the donor vector comprises:
(i) an exogenous polynucleotide that encodes a therapeutic polypeptide; and
(ii) a second recognition sequence for the site specific recombinase, and
(iii) a nucleic acid sequence that encodes a modified selectable marker polypeptide, wherein the modified selectable marker polypeptide exhibits a reduced activity relative to a corresponding WT selectable marker polypeptide; and
(c) culturing the cell from step (b) under conditions permissive for the site specific recombination between the at least one of the plurality of first recognition sequences and the second recognition sequence, when contacted with the site specific recombinase, wherein the site specific recombination results in site specific insertion of the exogenous polynucleotide within the at least one of the plurality of first recognition sequence, thereby generating the genetically engineered cell.

276. The method of claim 275, wherein the site specific recombinase is a serine recombinase, or a tyrosine recombinase.

277. The method of claim 275, wherein
(a) at least one of the plurality of first recognition sequences is a bacterial genomic recombination site (attB) and the second recognition sequence is a phage genomic recombination site (attP), or
(b) at least one of the plurality of first recognition sequences is a phage genomic recombination site (attP), and the second recognition sequence is a bacterial genomic recombination site (attB).

278. The method of claim 275, wherein the nucleic acid sequence that encodes the modified selectable marker polypeptide is an antibiotic resistance gene, and wherein the reduced activity comprises reduced resistance to an antibiotic relative to the corresponding wild type selectable marker polypeptide.

279. The method of claim 275, wherein the modified selectable marker polypeptide comprises an amino acid substitution relative to the corresponding wild type selectable marker polypeptide.

280. The method of claim 275, wherein the modified selectable marker polypeptide is a neomycin phosphotransferase.

281. The method of claim 280, wherein the neomycin phosphotransferase comprises a D227V amino acid substitution relative to the corresponding wild type neomycin phosphotransferase.

282. The method of claim 275, wherein the therapeutic polypeptide is an antibody or a fragment thereof, a chimeric antigen receptor (CAR), a T-cell receptor (TCR), a B-cell receptor (BCR), an αβ receptor, a γδ T-receptor, dopamine, insulin, proinsulin, or a portion thereof, or a combination thereof.

283. The method of claim 275, wherein the therapeutic polypeptide comprises a heavy chain of an antibody and a light chain of an antibody linked by a linker.

284. The method of claim 275, wherein said method results in simultaneous insertion of two or more copies of the exogenous polypeptide in the GEMS polynucleotide sequence.

285. A gene editing multi-site (GEMS) polynucleotide construct for insertion into an insertion site in a genome of a Chinese hamster ovary (CHO) cell, wherein said GEMS polynucleotide construct comprises a GEMS polynucleotide sequence that comprises:
a plurality of nuclease recognition sequences,
wherein each of nuclease recognition sequences of the plurality of nuclease recognition sequences comprises a target sequence and a protospacer adjacent motif (PAM) sequence or reverse complements thereof,
wherein each of the nuclease recognition sequences of the plurality of nuclease recognition sequences comprises a recognition sequence for a Cas protein or a Cpf1 protein,
wherein the GEMS polynucleotide sequence is heterologous to the genome of the CHO cell, and wherein the GEMS polynucleotide sequence is non-coding.

286. The GEMS polynucleotide construct of claim 285, wherein at least one of the plurality of nuclease recognition sequences is selected from the group consisting of sequences SEQ ID NOs: 89, 91, 93, 95, 97, 99, 101, 103 and reverse complements thereof.

287. The GEMS polynucleotide construct of claim 285, wherein the GEMS polynucleotide sequence comprises a sequence that is at least about 80%, identical to SEQ ID NO: 1 or SEQ ID NO: 3, wherein a sequence identity of the GEMS polynucleotide sequence to SEQ ID NO: 1 or SEQ ID NO: 3 is calculated by BLASTN.


SEQUENCE LISTING (158 sequences)

SEQ ID NO: 1 - DNA, 2984 bp, Artificial Sequence
  Description of Artificial Sequence Synthetic polynucleotide
atccgtattccgacgtacgatggaccactcttcacacgtaaagcaagaacgtcgagcagtcatgaaagtcttagtaccgcacgtgccatcttactgcgaatattgcctgaagctgtaccgttattggggggcaaagatgaagttctcctcttttcataattgtactgacgacagccgtgttcccggtttcttcagaggttaaagaataagggcttattgtaggcagagggacgcccttttagtggctggcgttaagtatcttcggacccccttgtctatccagattaatcgaattctctcatttaggaccctagtaagtcatcattggtatttgaatgcgaccccgaagaaaccgcctaaaaatgtcaatggttggtccactaaacttcatttaatcaactcctaaatcggcgcgataggccagtattcgagtaggcgtcgatgggttagaggtttaattttgtatggcaaggtacttccgatcttaatgaatggccggaagaggtacggacgcgatatgcgggggtgagagggcaaataggcaggttcgccttcgtcacgctaggaggcaattctataagaatgcacattgcatggatacataaaatgtctcgaccgcttgcgcaacttgtgaagtgtctactatccctaagcccatttcccgcataataacccctgattgtgtccgcatctgatgctacccgggttgagttagcgtcgagctcgcggaacttattgcatgagtagagttgagtaagagctgttagatggctcgctgagctaatagttgcccacagaacgtcaagattagagaacggtcgtagcattatcggaggttctctaactaagtaatcggttgcgccgctcggactatcagtacccgtgtctcgactctgccgcggctacctatcgcctgaaagccagttggtgttaaggggtgctctgtccaggacgccacgcgtagtgagacttacatgttcgttgggttcacccgactcggacctgagtggaccaaggacgcactcgagctctgagccctactgtcgagaaatatgtatctcgcccccgcagcttgccagctctttcagtatcatggagcccatggttgaatgactcctataacgaacttcgacatggcaaaatccccccctcgcgacttcaagagaagaagagtactgacttgagcgctcccagcacttcagccaaggaagttaccaatttcttgtttccgaatgacacgcgtctccttgcgggtaaatcgccgaccgcagtaatcggttgcgacgctcgggagaacttacgagccaggggaaacagtaaggcctaattaggtaaagggagtaagtgctcgaacggttcagttgtaaccatatacttacgctggatcttctccggcgaatttttaccgtcaccaactacgagatttgaggtaaaccaaatgagcacatagtggcgctatccgactatttccaaattgtaacatatcgttccatgaaggccagagttacttaccggccctttccatgcgcgcgccataccctcctagttccccggttatctctccgaggagagagtgagcgatcctccgttaacatattgttaccaatgacgtagctatgtattttgcacaggtagccaacgggtttcacatttcacagatagtggggttcccggcaaagggcgtatatttgcataatttcgcccacctagcgcggggtccaacataggcgtaaactacgatggcacctactcagacgcagctcgtgcggcgtaaataacgtactcatcccaactgattctcggcaatctacggagcgacatgattatcaacagctgtctagcagttctaatcttttgccatggtcgtaaaagcctccaagagattgatcatacctatcggcacagaagtgacacgacgccgatgggtagcggactttaggtcaaccacagttcggtaggggacaggccctgcggcgtacatcactttgtatgtgcaacgtgcccaagtggcgccaggcaagactcagctggttcctgtgttagctcgaggctaggcatgacagctctttgaacatgggctgggggcctcgaacggtcgagaagcccatagtacctgcgtgcgatcgtaccgtctacggcggataccaagttgcgcaggctatagcttgaagctgtactatttcagggggggagccctgatggtctcttcttctgatgactcaactcgctagggtcgtgaagtcgattccttcgatggttaaaaatcaaaggctcagagtgcagactggagcgcccatctaacggttcgcatctcgaatgctcggtcgcctttcacattccgcgaaaattcataccgctcattcactaggttgcgaagtctacactgatatatgaatccgagctagagcagggctcttaaaattcggagtcgttgatgctcaatactccaatcggttttttcgtgcaccaccgcgagtggctgacaagggtttgacattgagtagcaaggcagttccgggctgaatgaagcgccgggaaccttcagcaacgtttcgcgtgggtgtgaattgttattcgcgaaaaacatccgtccccgtgggggatagtcaccgacgccgttttatagaagtgtaggggaacaggttggtttaactagcttaagaaagtaaattctgggattatactgtagtaatcactaatttacggtgagggttttatggcggatctttacaaattcaagccaggtgatttcaacaaattttgctgacgatttaggcgcactatcccctaaactacaaattagaaaatagcgttccttgacggctagaattacctaccggcctccaccataccttcgatattcgcgcccactctcccattaatccgcacaagtggatgtgatgcgattgcccgctaagatattctaacgtgtaacgcagatgagtattctacagagttgccgtagcaaccgccggctacggggg

SEQ ID NO: 2 - DNA, 18 bp, Artificial Sequence
  Description of Artificial Sequence Synthetic oligonucleotide
tagggataacagggtaat

SEQ ID NO: 3 - DNA, 2984 bp, Artificial Sequence
  Description of Artificial Sequence Synthetic polynucleotide
atccgtattccgacgtacgatggaccactcttcacacgtaaagcaagaacgtcgagcagtcatgaaagtcttagtaccgcacgtgccatcttactgcgaatattgcctgaagctgtaccgttattggggggcaaagatgaagttctcctcttttcataattgtactgacgacagccgtgttcccggtttcttcagaggttaaagaataagggcttattgtaggcagagggacgcccttttagtggctggcgttaagtatcttcggacccccttgtctatccagattaatcgaattctctcatttaggaccctagtaagtcatcattggtatttgaatgcgaccccgaagaaaccgcctaaaaatgtcaatggttggtccactaaacttcatttaatcaactcctaaatcggcgcgataggccagtattcgagtaggcgtcgatgggttagaggtttaattttgtatggcaaggtacttccgatcttaatgaatggccggaagaggtacggacgcgatatgcgggggtgagagggcaaataggcaggttcgccttcgtcacgctaggaggcaattctataagaatgcacattgcatggatacataaaatgtctcgaccgcttgcgcaacttgtgaagtgtctactatccctaagcccatttcccgcataataacccctgattgtgtccgcatctgatgctacccgggttgagttagcgtcgagctcgcggaacttattgcatgagtagagttgagtaagagctgttagatggctcgctgagctaatagttgcccacagaacgtcaagattagagaacggtcgtagcattatcggaggttctctaactaagtaatcggttgcgccgctcggactatcagtacccgtgtctcgactctgccgcggctacctatcgcctgaaagccagttggtgttaaggggtgctctgtccaggacgccacgcgtagtgagacttacatgttcgttgggttcacccgactcggacctgagtggaccaaggacgcactcgagctctgagccctactgtcgagaaatatgtatctcgcccccgcagcttgccagctctttcagtatcatggagcccatggttgaatgactcctataacgaacttcgacatggcaaaatccccccctcgcgacttcaagagaagaagagtactgacttgagcgctcccagcacttcagccaaggaagttaccaatttcttgtttccgaatgacacgcgtctccttgcgggtaaatcgccgaccgctaatcctcgcggtaaccggtaggagaacttacgagccaggggaaacagtaaggcctaattaggtaaagggagtaagtgctcgaacggttcagttgtaaccatatacttacgctggatcttctccggcgaatttttaccgtcaccaactacgagatttgaggtaaaccaaatgagcacatagtggcgctatccgactatttccaaattgtaacatatcgttccatgaaggccagagttacttaccggccctttccatgcgcgcgccataccctcctagttccccggttatctctccgaggagagagtgagcgatcctccgttaacatattgttaccaatgacgtagctatgtattttgcacaggtagccaacgggtttcacatttcacagatagtggggttcccggcaaagggcgtatatttgcatgggcttctcgaccgttcgaggggtccaacataggcgtaaactacgatggcacctactcagacgcagctcgtgcggcgtaaataacgtactcatcccaactgattctcggcaatctacggagcgacatgattatcaacagctgtctagcagttctaatcttttgccatggtcgtaaaagcctccaagagattgatcatacctatcggcacagaagtgacacgacgccgatgggtagcggactttaggtcaaccacagttcggtaggggacaggccctgcggcgtacatcactttgtatgtgcaacgtgcccaagtggcgccaggcaagactcagctggttcctgtgttagctcgaggctaggcatgacagctctttgaacatgggctgggggcctcgaacggtcgagaagcccatagtacctgcgtgcgatcgtaccgtctacggcggataccaagttgcgcaggctatagcttgaagctgtactatttcagggggggagccctgatggtctcttcttctgatgactcaactcgctagggtcgtgaagtcgattccttcgatggttaaaaatcaaaggctcagagtgcagactggagcgcccatctaacggttcgcatctcgaatgctcggtcgcctttcacattccgcgaaaattcataccgctcattcactaggttgcgaagtctacactgatatatgaatccgagctagagcagggctcttaaaattcggagtcgttgatgctcaatactccaatcggttttttcgtgcaccaccgcgagtggctgacaagggtttgacattgagtagcaaggcagttccgggctgaatgaagcgccgggaaccttcagcaacgtttcgcgtgggtgtgaattgttattcgcgaaaaacatccgtccccgtgggggatagtcaccgacgccgttttatagaagtgtaggggaacaggttggtttaactagcttaagaaagtaaattctgggattatactgtagtaatcactaatttacggtgagggttttatggcggatctttacaaattcaagccaggtgatttcaacaaattttgctgacgatttaggcgcactatcccctaaactacaaattagaaaatagcgttccttgacggctagaattacctaccggcctccaccataccttcgatattcgcgcccactctcccattaatccgcacaagtggatgtgatgcgattgcccgctaagatattctaacgtgtaacgcagatgagtattctacagagttgcctttatatgggacgcgtacgccgg

SEQ ID NO: 4 - DNA, 20 bp, Artificial Sequence
  Description of Artificial Sequence Synthetic oligonucleotide
ccgataaaacacatgcgtca

SEQ ID NO: 5 - DNA, 20 bp, Artificial Sequence
  Description of Artificial Sequence Synthetic oligonucleotide
cacgcggtcgttatagttca

SEQ ID NO: 6 - DNA, 20 bp, Artificial Sequence
  Description of Artificial Sequence Synthetic oligonucleotide
cggaggaatatgtcccagat

SEQ ID NO: 7 - DNA, 447 bp, Artificial Sequence
  Description of Artificial Sequence Synthetic polynucleotide
gtcaataagggagccgcagtggagtaggcggggagaaggccgcaccctactcggctgggggaggggagtgccgcaatacctttctgggagttctctgctgcctcctgtcttctaaagaccgccccgggactggaaggatcccttccccctttcccctcgtgatctgcaagtcgaggctttctgggagatgggcgggagtcttctgggcaggcttgagggctaacctggtgcgtgggcgttgtcctgcaggggaattgaactggtgtaaaattggaagggtgagaattcccacggattttcgtttgtgtcgggaggtgattgtaataggggcaaaggagggaaatgggagactaggtgctcgcctggggttttgtgcagcaaaactacaggttattattaataagccttggagtatttttcatcgagttggattaaggtcatgctcac

SEQ ID NO: 8 - DNA, 400 bp, Artificial Sequence
  Description of Artificial Sequence Synthetic polynucleotide
ttatgctcacgcttgagatccttgctatatcatgaaattatagtgtcgcaagttagaatacataaacagaattttagtgttttctacagggccctgcacttcactctttccctcctgctccctctgcagccctaccaaaagatattttagcactctcatttgagtccccttttcatttgttagtactggctcacccaatccctagacagagcactggcattcttcccctcatgatcttagaagcctgatgagtcatgaaaccagacagattagttacaccacaaattgaggctgtagctggggccttaccctgcagttcttttatgcctccttagtacattttgttgactgtttgccttgattttcattttctatccccttcgggagctctgctgcaata

SEQ ID NO: 9 - DNA, 23 bp, Artificial Sequence
  Description of Artificial Sequence Synthetic oligonucleotide
tcaagcgtgagcataaaactcgg

SEQ ID NO: 10 - RNA, 100 nt, Artificial Sequence
  Description of Artificial Sequence Synthetic polynucleotide
ucaagcgugagcauaaaacuguuuuagagcuagaaauagcaaguuaaaauaaggcuaguccguuaucaacuugaaaaaguggcaccgagucggugcuuuu

SEQ ID NO: 11 - DNA, 616 bp, Artificial Sequence
  Description of Artificial Sequence Synthetic polynucleotide
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgg

SEQ ID NO: 12 - DNA, 756 bp, Artificial Sequence
  Description of Artificial Sequence Synthetic polynucleotide
atggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgc

SEQ ID NO: 13 - DNA, 597 bp, Artificial Sequence
  Description of Artificial Sequence Synthetic polynucleotide
atgaccgagtacaagcccacggtgcgcctcgccacccgcgacgacgtccccagggccgtccgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 14 - DNA, 792 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgaatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 15 - DNA, 732 bp, Artificial Sequence
  Description of Artificial Sequence Synthetic polynucleotide
gatatcgtgatgacccagagccccagctctttaaccgtgacagccggcgagaaggtgaccatgatctgcaagagctcccagtctttactgaacagcggcgaccagaagaactatttaacttggtatcaacagaagcccggacagccccccaagctgctcatcttctgggcctccaccagagagagcggcgtgcccgatcgttttaccggctccggctccggcaccgactttactttaaccatcagcagcgtgcaagctgaagatttagccgtgtactactgccagaacgactacagctaccctttaacattcggcgccggcaccaagctggaactgaagggcggcggcggttctggaggcggaggcagcggcggcggcggcagccaagttcagctgcagcagtccgacgctgagctggtgaagcccggcagctccgtcaagattagctgcaaagcctccggctacaccttcacagaccacgccattcactgggtgaagcagaagcccgaacaaggtttagagtggatcggccacttctcccccggcaacaccgacatcaagtacaacgacaagttcaagggcaaggccactttaaccgtggatagaagcagctccaccgcctacatgcagctgaactctttaaccagcgaggatagcgccgtgtacttctgcaagaccagcaccttcttcttcgactattggggccaaggcactactttaacagtgtccagc

SEQ ID NO: 16 - DNA, 738 bp, Artificial Sequence
  Description of Artificial Sequence Synthetic polynucleotide
gatatccagatgacccaatcccctagctctctgagcgccagcgtgggagacagagtgacaatcacatgtagagcttcccagaccatctggagctacctcaactggtatcagcagaggcccggcaaggcccctaatctgctcatttatgctgccagctccctccagtccggagtgccttctaggttctccggaagaggctccggcaccgacttcacactgaccatcagctctctgcaagccgaggacttcgccacctactactgccagcagagctacagcatcccccagaccttcggccaaggcaccaagctggagattaagggcggcggcggaagcggaggaggaggcagcggaggcggcggcagccaagtgcaactgcaacagtccggccccggactggtgaaaccctcccagacactgtctctgacatgcgctatcagcggcgatagcgtgtccagcaactccgccgcttggaactggattagacagtcccctagcagaggactggaatggctgggaagaacctactatagatccaagtggtacaacgactatgccgtgagcgtgaagtctaggatcaccatcaaccccgatacctccaagaaccagttctctctgcagctcaatagcgtgacccccgaggacaccgccgtgtactattgtgccagagaggtgaccggcgatctggaggatgccttcgatatctggggacaaggcaccatggtgaccgtgtcctcc

SEQ ID NO: 17 - DNA, 360 bp, Artificial Sequence
  Description of Artificial Sequence Synthetic polynucleotide
tggaggaggaagagaaaggagaagcagagcgagaccagccccaaggagtttttaactatctacgaggacgtgaaggatttaaagactcgtaggaaccacgagcaagaacagacattccccggtggcggtagcaccatctacagcatgatccagagccagagcagcgctcccaccagccaagaacccgcttacactttatactctttaatccagccctctcgtaagagcggcagcagaaagaggaaccacagccccagcttcaacagcactatctatgaggtgatcggcaagagccagcccaaggcccagaaccccgctcgtctgtctcgtaaggagctggaaaacttcgacgtgtacagc

SEQ ID NO: 18 - DNA, 1184 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 19 - DNA, 396 bp, Artificial Sequence
  Description of Artificial Sequence Synthetic polynucleotide
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggc

SEQ ID NO: 20 - DNA, 726 bp, Artificial Sequence
  Description of Artificial Sequence Synthetic polynucleotide
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagc

SEQ ID NO: 21 - DNA, 207 bp, Artificial Sequence
  Description of Artificial Sequence Synthetic polynucleotide
accactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgt

SEQ ID NO: 22 - DNA, 126 bp, Artificial Sequence
  Description of Artificial Sequence Synthetic polynucleotide
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 23 - DNA, 336 bp, Artificial Sequence
  Description of Artificial Sequence Synthetic polynucleotide
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 24 - DNA, 20 bp, Artificial Sequence
  Description of Artificial Sequence Synthetic oligonucleotide
tgcttgtgcatacataacaa

SEQ ID NO: 25 - DNA, 20 bp, Artificial Sequence
  Description of Artificial Sequence Synthetic oligonucleotide
cccgcaatagagagctttga

SEQ ID NO: 26 - DNA, 20 bp, Artificial Sequence
  Description of Artificial Sequence Synthetic oligonucleotide
ttgcagcgcgcagagcatct

SEQ ID NO: 27 - DNA, 20 bp, Artificial Sequence
  Description of Artificial Sequence Synthetic oligonucleotide
ttttgctacatcttgtaata

SEQ ID NO: 28 - DNA, 20 bp, Artificial Sequence
  Description of Artificial Sequence Synthetic oligonucleotide
atacagtacgcgtgtaacaa

SEQ ID NO: 29 - DNA, 20 bp, Artificial Sequence
  Description of Artificial Sequence Synthetic oligonucleotide
tacgatgagaaagcaatcga

SEQ ID NO: 30 - DNA, 20 bp, Artificial Sequence
  Description of Artificial Sequence Synthetic oligonucleotide
caatgacaatagcgataacg

SEQ ID NO: 31 - DNA, 20 bp, Artificial Sequence
  Description of Artificial Sequence Synthetic oligonucleotide
tgaattagatttgcgttact

SEQ ID NO: 32 - DNA, 20 bp, Artificial Sequence
  Description of Artificial Sequence Synthetic oligonucleotide
tgtgttagcgcgctgatctg

SEQ ID NO: 33 - RNA, 20 nt, Artificial Sequence
  Description of Artificial Sequence Synthetic oligonucleotide
ugaauuagauuugcguuacu

SEQ ID NO: 34 - RNA, 20 nt, Artificial Sequence
  Description of Artificial Sequence Synthetic oligonucleotide
ucacaaucacucaagaagca

SEQ ID NO: 35 - RNA, 20 nt, Artificial Sequence
  Description of Artificial Sequence Synthetic oligonucleotide
cuuuagacacaguaagacaa

SEQ ID NO: 36 - RNA, 20 nt, Artificial Sequence
  Description of Artificial Sequence Synthetic oligonucleotide
cccgcaauagagagcuuuga

SEQ ID NO: 37 - DNA, 20 bp, Artificial Sequence
  Description of Artificial Sequence Synthetic oligonucleotide
  Description of Combined DNA/RNA Molecule Synthetic oligonucleotide
gaacguatcugcaugucuag

SEQ ID NO: 38 - RNA, 20 nt, Artificial Sequence
  Description of Artificial Sequence Synthetic oligonucleotide
caugccuuuagaauucagua

SEQ ID NO: 39 - RNA, 20 nt, Artificial Sequence
  Description of Artificial Sequence Synthetic oligonucleotide
uguguuagcgcgcugaucug

SEQ ID NO: 40 - RNA, 20 nt, Artificial Sequence
  Description of Artificial Sequence Synthetic oligonucleotide
uacgaagucgagauaaaaug

SEQ ID NO: 41 - RNA, 20 nt, Artificial Sequence
  Description of Artificial Sequence Synthetic oligonucleotide
gcauaaccaguacgcaagau

SEQ ID NO: 42 - RNA, 20 nt, Artificial Sequence
  Description of Artificial Sequence Synthetic oligonucleotide
uuuugcuacaucuuguaaua

SEQ ID NO: 43 - RNA, 20 nt, Artificial Sequence
  Description of Artificial Sequence Synthetic oligonucleotide
auuauaauauucaguagaaa

SEQ ID NO: 44 - DNA, 20 bp, Artificial Sequence
  Description of Artificial Sequence Synthetic oligonucleotide
  Description of Combined DNA/RNA Molecule Synthetic oligonucleotide
cagctacgagucacgaugua

SEQ ID NO: 45 - RNA, 20 nt, Artificial Sequence
  Description of Artificial Sequence Synthetic oligonucleotide
caaugacaauagcgauaacg

SEQ ID NO: 46 - RNA, 20 nt, Artificial Sequence
  Description of Artificial Sequence Synthetic oligonucleotide
guuacguucgcgaagcguug

SEQ ID NO: 47 - RNA, 20 nt, Artificial Sequence
  Description of Artificial Sequence Synthetic oligonucleotide
gcguaacaacuucugaguug

SEQ ID NO: 48 - RNA, 20 nt, Artificial Sequence
  Description of Artificial Sequence Synthetic oligonucleotide
ugcuugugcauacauaacaa

SEQ ID NO: 49 - DNA, 20 bp, Artificial Sequence
  Description of Artificial Sequence Synthetic oligonucleotide
  Description of Combined DNA/RNA Molecule Synthetic oligonucleotide
gcgcuactcgaacgctacgu

SEQ ID NO: 50 - RNA, 20 nt, Artificial Sequence
  Description of Artificial Sequence Synthetic oligonucleotide
acgagacugugcuugugcga

SEQ ID NO: 51 - RNA, 20 nt, Artificial Sequence
  Description of Artificial Sequence Synthetic oligonucleotide
uugcagcgcgcagagcaucu

SEQ ID NO: 52 - RNA, 20 nt, Artificial Sequence
  Description of Artificial Sequence Synthetic oligonucleotide
gacgcguuaauaaauagcau

SEQ ID NO: 53 - RNA, 20 nt, Artificial Sequence
  Description of Artificial Sequence Synthetic oligonucleotide
uucgauugcuuuagaauuaa

SEQ ID NO: 54 - RNA, 20 nt, Artificial Sequence
  Description of Artificial Sequence Synthetic oligonucleotide
auacaguacgcguguaacaa

SEQ ID NO: 55 - RNA, 20 nt, Artificial Sequence
  Description of Artificial Sequence Synthetic oligonucleotide
gauuuagcauaacgcgucag

SEQ ID NO: 56 - RNA, 20 nt, Artificial Sequence
  Description of Artificial Sequence Synthetic oligonucleotide
cgcgucuacuugcaaugacg

SEQ ID NO: 57 - RNA, 20 nt, Artificial Sequence
  Description of Artificial Sequence Synthetic oligonucleotide
uacgaugagaaagcaaucga

SEQ ID NO: 58 - RNA, 20 nt, Artificial Sequence
  Description of Artificial Sequence Synthetic oligonucleotide
uuucuaaaucgcucgcagau

SEQ ID NO: 59 - RNA, 20 nt, Artificial Sequence
  Description of Artificial Sequence Synthetic oligonucleotide
cacuaucgagcugucacgcu

SEQ ID NO: 60 - RNA, 20 nt, Artificial Sequence
  Description of Artificial Sequence Synthetic oligonucleotide
ugagaaugcacugcgaucag

SEQ ID NO: 61 - RNA, 20 nt, Artificial Sequence
  Description of Artificial Sequence Synthetic oligonucleotide
caggaucuauguacgaucga

SEQ ID NO: 62 - RNA, 20 nt, Artificial Sequence
  Description of Artificial Sequence Synthetic oligonucleotide
uuauacagcugcgugcgcgu

SEQ ID NO: 63 - RNA, 20 nt, Artificial Sequence
  Description of Artificial Sequence Synthetic oligonucleotide
auccguauuccgacguacga

SEQ ID NO: 64 - RNA, 20 nt, Artificial Sequence
  Description of Artificial Sequence Synthetic oligonucleotide
cguacugugauacacgcgac

SEQ ID NO: 65 - RNA, 20 nt, Artificial Sequence
  Description of Artificial Sequence Synthetic oligonucleotide
ggcgcuccgauaaaucgcua

SEQ ID NO: 66 - RNA, 20 nt, Artificial Sequence
  Description of Artificial Sequence Synthetic oligonucleotide
auuaccgauacgauacgaac

SEQ ID NO: 67 - RNA, 20 nt, Artificial Sequence
  Description of Artificial Sequence Synthetic oligonucleotide
acggacgcgcaaccgucguc

SEQ ID NO: 68 - RNA, 20 nt, Artificial Sequence
  Description of Artificial Sequence Synthetic oligonucleotide
uaaucgguugcgccgcucgg

SEQ ID NO: 69 - RNA, 20 nt, Artificial Sequence
  Description of Artificial Sequence Synthetic oligonucleotide
uuauuuaccccgcgcgaggu

SEQ ID NO: 70 - RNA, 20 nt, Artificial Sequence
  Description of Artificial Sequence Synthetic oligonucleotide
guuguaucguacgucggucu

SEQ ID NO: 71 - RNA, 20 nt, Artificial Sequence
  Description of Artificial Sequence Synthetic oligonucleotide
aguauucgaguacgcgucga

SEQ ID NO: 72 - RNA, 20 nt, Artificial Sequence
  Description of Artificial Sequence Synthetic oligonucleotide
guauucgaguacgcgucgau

SEQ ID NO: 73 - RNA, 20 nt, Artificial Sequence
  Description of Artificial Sequence Synthetic oligonucleotide
gcgugcgaucguaccgugua

SEQ ID NO: 74 - RNA, 20 nt, Artificial Sequence
  Description of Artificial Sequence Synthetic oligonucleotide
cgcauggcaaucuacgcgcg

SEQ ID NO: 75 - RNA, 20 nt, Artificial Sequence
  Description of Artificial Sequence Synthetic oligonucleotide
gugaaccgacccggucgauc

SEQ ID NO: 76 - RNA, 20 nt, Artificial Sequence
  Description of Artificial Sequence Synthetic oligonucleotide
uucuucgauacgguacgaau

SEQ ID NO: 77 - RNA, 20 nt, Artificial Sequence
  Description of Artificial Sequence Synthetic oligonucleotide
uuuauaugggacgcguacgc

SEQ ID NO: 78 - RNA, 20 nt, Artificial Sequence
  Description of Artificial Sequence Synthetic oligonucleotide
agaguggccgcgauaaucga

SEQ ID NO: 79 - RNA, 20 nt, Artificial Sequence
  Description of Artificial Sequence Synthetic oligonucleotide
uaauccucgcgguaaccggu

SEQ ID NO: 80 - RNA, 20 nt, Artificial Sequence
  Description of Artificial Sequence Synthetic oligonucleotide
agagugggcgcgaauaucgu

SEQ ID NO: 81 - DNA, 1023 bp, Artificial Sequence
  Description of Artificial Sequence Synthetic polynucleotide
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaa

SEQ ID NO: 82 - DNA, 523 bp, Artificial Sequence
  Description of Artificial Sequence Synthetic polynucleotide
ctacggtggtcagaccgaagactgcgacggtaccgacgctggtcgcgcctcttatacccacgtagaacgcagctcagccaatagaatgagtgccaatatggaatttccaggggaaaaccggggcggtgttacgttttggctgccctttcacttcccattgacgtgtattggctcgagaacggtactttcccattaatcagctatgggaaagtaccgtttaaaggtcacgttgcattagtttcaatagcccattgacgtcaatggtgggaaagtacatggcgttttaattaaatggctggaaaaacccaatgactcacccctattgaccttatgtacgtgccaataatgggaaaaacccattgactcaccccctattgaccttttgtactgggcaaaacccaatggaaagtccctattgactcagtgtacttggctccaatgggactttcctgttgattcacccctattgaccttatgtactgggcaaaacccattggaaagtccctaatgactcagtatatct

SEQ ID NO: 83 - DNA, 1645 bp, Artificial Sequence
  Description of Artificial Sequence Synthetic polynucleotide
acggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcaggggatggcggggctgacgtcgggaggtggcctccacgggaagggacacccggatctcgacacagccttggcagtggagtcaggaagggtaggacagattctggacgccctcttggccagtcctcaccgccccacccccgatggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcggaccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgagcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaaggtgttgtgaaaaccaccgc

SEQ ID NO: 84 - DNA, 792 bp, Artificial Sequence
  Description of Artificial Sequence Synthetic polynucleotide
atgatcgaacaagatggactgcacgctggcagcccagctgcttgggtcgagagactcttcggatacgactgggctcagcagactatcggctgtagcgatgctgctgtgttcagactctccgctcaaggaaggccagtgctcttcgtcaagacagatctgagcggcgctctcaatgaactgcaagatgaggctgctagactgagctggctggccactactggagtcccatgtgctgctgtgctggacgtggtcactgaagctggcagagattggctgctgctgggcgaagtgcccggccaagatctgctgtcctcccatctggccccagctgagaaggtctccatcatggccgacgccatgaggaggctgcacacactcgatccagccacttgccctttcgatcaccaagccaagcataggatcgaaagggctaggactagaatggaggccggactcgtggaccaagatgatctggatgaagagcaccaaggactggctccagccgaactgttcgctagactgaaggctaggatgccagacggcgaagatctggtggtcactcacggcgatgcttgtctgcctaacatcatggtcgagaacggaaggttcagcggctttattgattgcggaaggctcggagtggccgatagataccaagatatcgctctggccactagagtgatcgccgaggagctgggaggagaatgggccgataggtttctggtgctctacggcatcgccgccccagatagccagaggattgccttctacagactgctggacgagttcttt

SEQ ID NO: 85 - DNA, 702 bp, Artificial Sequence
  Description of Artificial Sequence Synthetic polynucleotide
atggaaactcacagccaagtgttcgtctacatgctgctgtggctcagcggagtcgagggagacattcagatgactcagagccctagctccctctccgcttccgtgggagatagggtcactatcacatgtagggcttcccaagatgtcagcactgccgtcgcttggtatcagcagaagcccggcaaagcccctaaactgctcatctacagcgccagcttcctctatagcggagtccctagcagattctccggcagcggcagcggcactgacttcactctgactatcagcagcctccagccagaggacttcgccacttattactgccagcagtatctgtaccatccagccacattcggccaaggcactaaagtcgagatcaagaggacagtggccgccccaagcgtgttcatcttccctccttccgacgaacagctcaagagcggcacagcttccgtcgtctgtctgctgaataacttctacccaagggaggccaaggtgcaatggaaagtcgataacgctctgcagtccggcaacagccaagagagcgtgacagagcaagatagcaaggattccacatactctctgagctccacactgactctgtccaaagccgactacgagaagcacaaggtctacgcttgtgaagtgactcaccaaggactgtcctccccagtgactaagagcttcaataggggcgagtgc

SEQ ID NO: 86 - DNA, 1401 bp, Artificial Sequence
  Description of Artificial Sequence Synthetic polynucleotide
atggcttgggtctggacactgctgtttctcatggccgccgcccaaagcattcaagccgaggtgcagctggtcgagagcggaggaggactcgtgcagcccggcggctctctgaggctgagctgtgctgcctccggctttacattctccgacagctggattcactgggtgaggcaagcccccggcaaaggactggaatgggtcgcttggatcagcccatatggaggctccacatactacgccgacagcgtgaagggaaggtttactatcagcgccgatacatccaagaacacagcctatctgcagatgaactctctgagagccgaagacacagctgtctactattgcgccagaaggcactggcccggcggctttgattactggggccaaggcacactggtgactgtctcctccgccagcactaagggcccttccgtcttccctctggcccctagcagcaaaagcacatccggcggaactgctgctctcggatgtctggtgaaagactacttcccagagccagtgacagtcagctggaacagcggcgccctcacaagcggcgtccatactttcccagccgtgctgcagagctccggactgtattctctgagcagcgtggtcacagtcccaagctcctctctgggcacacaaacatacatctgtaatgtcaaccataaaccaagcaacactaaggtggataagaaggtggagcctaagagctgtgacaagacacacacatgccctccatgcccagctccagagctgctcggcggacctagcgtcttcctcttccctcctaagccaaaggacacactcatgatcagcagaacaccagaggtcacatgtgtggtcgtggacgtcagccacgaagatccagaggtcaagtttaactggtacgtggatggagtggaagtccacaacgccaagactaagcctagggaggagcagtatgccagcacttatagggtcgtgtccgtgctgactgtcctccatcaagattggctcaatggcaaggagtacaaatgcaaggtctccaacaaggctctgccagccccaatcgagaagacaatctccaaggccaaaggccagccaagagagcctcaagtctacacactccctccatctagggaggagatgacaaaaaaccaagtgtctctgacatgtctggtgaaaggcttctatcctagcgacatcgccgtcgaatgggagtccaatggccagccagagaacaactacaaaactacacctccagtgctcgatagcgatggcagcttcttcctctacagcaagctgacagtggataagtctaggtggcagcaaggcaacgtcttcagctgttccgtcatgcacgaggctctccataaccattacactcagaagtccctctccctctcccccggcaag

SEQ ID NO: 87 - DNA, 57 bp, Artificial Sequence
  Description of Artificial Sequence Synthetic oligonucleotide
ggagagggaagaggatctctgctgacatgtggagatgtcgaggagaaccccggccca

SEQ ID NO: 88 - DNA, 12 bp, Artificial Sequence
  Description of Artificial Sequence Synthetic oligonucleotide
aggagaaagagg

SEQ ID NO: 89 - DNA, 23 bp, Artificial Sequence
  Description of Artificial Sequence Synthetic oligonucleotide
atccgtattccgacgtacgatgg

SEQ ID NO: 90 - RNA, 100 nt, Artificial Sequence
  Description of Artificial Sequence Synthetic polynucleotide
auccguauuccgacguacgaguuuuagagcuagaaauagcaaguuaaaauaaggcuaguccguuaucaacuugaaaaaguggcaccgagucggugcuuuu

SEQ ID NO: 91 - DNA, 23 bp, Artificial Sequence
  Description of Artificial Sequence Synthetic oligonucleotide
gtattcgagtaggcgtcgatggg

SEQ ID NO: 92 - RNA, 100 nt, Artificial Sequence
  Description of Artificial Sequence Synthetic polynucleotide
guauucgaguaggcgucgauguuuuagagcuagaaauagcaaguuaaaauaaggcuaguccguuaucaacuugaaaaaguggcaccgagucggugcuuuu

SEQ ID NO: 93 - DNA, 23 bp, Artificial Sequence
  Description of Artificial Sequence Synthetic oligonucleotide
aagtaatcggttgcgccgctcgg

SEQ ID NO: 94 - RNA, 100 nt, Artificial Sequence
  Description of Artificial Sequence Synthetic polynucleotide
aaguaaucgguugcgccgcuguuuuagagcuagaaauagcaaguuaaaauaaggcuaguccguuaucaacuugaaaaaguggcaccgagucggugcuuuu

SEQ ID NO: 95 - DNA, 23 bp, Artificial Sequence
  Description of Artificial Sequence Synthetic oligonucleotide
agtaatcggttgcgacgctcggg

SEQ ID NO: 96 - RNA, 100 nt, Artificial Sequence
  Description of Artificial Sequence Synthetic polynucleotide
aguaaucgguugcgacgcucguuuuagagcuagaaauagcaaguuaaaauaaggcuaguccguuaucaacuugaaaaaguggcaccgagucggugcuuuu

SEQ ID NO: 97 - DNA, 23 bp, Artificial Sequence
  Description of Artificial Sequence Synthetic oligonucleotide
ataatttcgcccacctagcgcgg

SEQ ID NO: 98 - RNA, 100 nt, Artificial Sequence
  Description of Artificial Sequence Synthetic polynucleotide
auaauuucgcccaccuagcgguuuuagagcuagaaauagcaaguuaaaauaaggcuaguccguuaucaacuugaaaaaguggcaccgagucggugcuuuu

SEQ ID NO: 99 - DNA, 23 bp, Artificial Sequence
  Description of Artificial Sequence Synthetic oligonucleotide
gcgtgcgatcgtaccgtctacgg

SEQ ID NO: 100 - RNA, 100 nt, Artificial Sequence
  Description of Artificial Sequence Synthetic polynucleotide
gcgugcgaucguaccgucuaguuuuagagcuagaaauagcaaguuaaaauaaggcuaguccguuaucaacuugaaaaaguggcaccgagucggugcuuuu

SEQ ID NO: 101 - DNA, 23 bp, Artificial Sequence
  Description of Artificial Sequence Synthetic oligonucleotide
ccttcagcaacgtttcgcgtggg

SEQ ID NO: 102 - RNA, 100 nt, Artificial Sequence
  Description of Artificial Sequence Synthetic polynucleotide
ccuucagcaacguuucgcguguuuuagagcuagaaauagcaaguuaaaauaaggcuaguccguuaucaacuugaaaaaguggcaccgagucggugcuuuu

SEQ ID NO: 103 - DNA, 23 bp, Artificial Sequence
  Description of Artificial Sequence Synthetic oligonucleotide
gtagcaaccgccggctacggggg

SEQ ID NO: 104 - RNA, 100 nt, Artificial Sequence
  Description of Artificial Sequence Synthetic polynucleotide
guagcaaccgccggcuacggguuuuagagcuagaaauagcaaguuaaaauaaggcuaguccguuaucaacuugaaaaaguggcaccgagucggugcuuuu

SEQ ID NO: 105 - DNA, 1990 bp, Artificial Sequence
  Description of Artificial Sequence Synthetic polynucleotide
gtggtttgtctggtcaaccaccgcggtctcagtggtgtacggtacaaacccaaccactcttcacacgtaaagcaagaacgtcgagcagtcatgaaagtcttagtggtttgtctggtcaaccaccgcggtctcagtggtgtacggtacaaacccagtaccgcacgtgccatcttactgcgaatattgcctgaagctgtaccgttagtggtttgtctggtcaaccaccgcggtctcagtggtgtacggtacaaacccattggggggcaaagatgaagttctcctcttttcataattgtactgacgacagtggtttgtctggtcaaccaccgcggtctcagtggtgtacggtacaaacccagccgtgttcccggtttcttcagaggttaaagaataagggcttattgtagggtggtttgtctggtcaaccaccgcggtctcagtggtgtacggtacaaacccacagagggacgcccttttagtggctggcgttaagtatcttcggacccccttgtggtttgtctggtcaaccaccgcggtctcagtggtgtacggtacaaacccagtctatccagattaatcgaattctctcatttaggaccctagtaagtcatcgtggtttgtctggtcaaccaccgcggtctcagtggtgtacggtacaaacccaattggtatttgaatgcgaccccgaagaaaccgcctaaaaatgtcaatggtgtggtttgtctggtcaaccaccgcggtctcagtggtgtacggtacaaacccatggtccactaaacttcatttaatcaactcctaaatcggcgcgataggccagtggtttgtctggtcaaccaccgcggtctcagtggtgtacggtacaaacccattagaggtttaattttgtatggcaaggtacttccgatcttaatgaatggcgtggtttgtctggtcaaccaccgcggtctcagtggtgtacggtacaaacccacggaagaggtacggacgcgatatgcgggggtgagagggcaaataggcagggtggtttgtctggtcaaccaccgcggtctcagtggtgtacggtacaaacccattcgccttcgtcacgctaggaggcaattctataagaatgcacattgcatggtggtttgtctggtcaaccaccgcggtctcagtggtgtacggtacaaacccagatacataaaatgtctcgaccgcttgcgcaacttgtgaagtgtctactatgtggtttgtctggtcaaccaccgcggtctcagtggtgtacggtacaaacccaccctaagcccatttcccgcataataacccctgattgtgtccgcatctgatgtggtttgtctggtcaaccaccgcggtctcagtggtgtacggtacaaacccagctacccgggttgagttagcgtcgagctcgcggaacttattgcatgagtagtggtttgtctggtcaaccaccgcggtctcagtggtgtacggtacaaacccagagttgagtaagagctgttagatggctcgctgagctaatagttgcccacagtggtttgtctggtcaaccaccgcggtctcagtggtgtacggtacaaacccagaacgtcaagattagagaacggtcgtagcattatcggaggttctctaactgtggtttgtctggtcaaccaccgcggtctcagtggtgtacggtacaaacccaactatcagtacccgtgtctcgactctgccgcggctacctatcgcctgaaagtggtttgtctggtcaaccaccgcggtctcagtggtgtacggtacaaacccagccagttggtgttaaggggtgctctgtccaggacgccacgcgtagtgagagtggtttgtctggtcaaccaccgcggtctcagtggtgtacggtacaaacccacttacatgttcgttgggttcacccgactcggacctgagtggaccaaggacgtggtttgtctggtcaaccaccgcggtctcagtggtgtacggtacaaaccca

SEQ ID NO: 106 - DNA, 52 bp, Artificial Sequence
  Description of Artificial Sequence Synthetic oligonucleotide
gtggtttgtctggtcaaccaccgcggtctcagtggtgtacggtacaaaccca

SEQ ID NO: 107 - DNA, 46 bp, Artificial Sequence
  Description of Artificial Sequence Synthetic oligonucleotide
ggccggcttgtcgacgacggcggtctccgtcgtcaggatcatccgg

SEQ ID NO: 108 - DNA, 1527 bp, Artificial Sequence
  Description of Artificial Sequence Synthetic polynucleotide
atgccaaagaagaaaaggaaggtgggctccagagctctggtggtcatcagactgagcagagtgactgacgccacaacttccccagagaggcaactggaaagctgtcagcagctctgtgcccagaggggatgggatgtcgtgggcgtcgccgaggatctggatgtcagcggcgccgtcgaccctttcgatagaaagaggaggccaaatctggccagatggctggcctttgaagagcagccattcgacgtcatcgtggcctatagggtggataggctgacaaggtccattaggcatctgcagcagctcgtgcattgggccgaggatcacaaaaagctggtcgtgagcgccacagaggcccatttcgacactacaactccattcgctgccgtcgtcatcgctctcatgggcacagtcgcccagatggaactggaggccatcaaggagaggaacagaagcgccgcccatttcaacattagggccggcaagtataggggatccctccctccatggggatatctgccaacaagagtcgatggcgaatggagactggtcccagatccagtgcagagggaaaggattctcgaggtgtaccacagagtggtggacaatcacgagcctctgcacctcgtggctcatgatctgaataggagaggcgtgctgagcccaaaagattacttcgcccagctgcaaggcagagaacctcaaggcagagagtggagcgctactgctctgaagaggagcatgattagcgaggctatgctcggctacgccacactcaacggaaagacagtgagagacgacgacggagcccctctcgtgagagctgagccaattctgacaagggagcagctggaagctctgagggctgaactcgtgaaaacatctagggctaagccagctgtcagcacaccttctctgctgctgagggtgctcttctgtgctgtctgcggagagccagcctacaagttcgctggcggcggaaggaagcatccaaggtataggtgtaggtccatgggcttcccaaaacactgcggaaatggcactgtggccatggctgaatgggacgctttctgcgaagaacaagtgctcgatctgctgggagacgccgaaaggctggaaaaggtctgggtggctggatccgatagcgctgtcgagctggccgaggtgaatgccgagctcgtcgatctgacatccctcattggcagcccagcttatagggctggaagcccacagagggaggctctggatgctagaatcgctgctctggccgctaggcaagaggagctcgagggactggaggctagacctagcggctgggagtggagagagactggccagagattcggcgattggtggagagaacaagacacagccgccaagaacacttggctgaggagcatgaacgtgaggctgactttcgatgtcagaggcggcctcacaaggacaatcgacttcggagacctccaagagtacgagcagcatctgaggctcggcagcgtggtggaaaggctgcacactggcatgagc

SEQ ID NO: 109 - DNA, 642 bp, Artificial Sequence
  Description of Artificial Sequence Synthetic polynucleotide
gacatccagatgactcaatccccttcctctctgtccgccagcgtcggcgatagggtcacaatcacatgtagcgcctcccaagatatcagcaactatctcaattggtatcagcagaaacccggcaaggctcctaaggtcctcatttacttcacatcctccctccacagcggagtgccttccagattttccggctccggcagcggaactgatttcactctgactatttcctctctgcagccagaggacttcgccacatactactgccagcagtacagcactgtgccatggacattcggacaaggaacaaaggtcgagattaagaggactgtggccgcccctagcgtcttcattttcccacctagcgatgagcagctgaaaagcggcacagcctccgtggtgtgtctgctgaacaacttctacccaagggaggctaaggtgcaatggaaagtggataacgctctgcagtccggcaatagccaagagagcgtgactgagcaagacagcaaggacagcacatactccctcagcagcacactgactctgagcaaggccgattacgagaagcacaaggtgtacgcttgtgaggtgacacaccaaggactcagcagcccagtgactaagtcctttaataggggcgaatgt

SEQ ID NO: 110 - DNA, 1359 bp, Artificial Sequence
  Description of Artificial Sequence Synthetic polynucleotide
gaggtgcagctcgtcgaaagcggaggaggactggtccagcccggcggaagcctcagactgagctgtgccgccagcggatacacattcactaactacggaatgaattgggtgaggcaagcccccggcaagggactggagtgggtcggatggatcaacacttacactggagagccaacatacgctgccgacttcaaaaggagatttactttctctctggatacatccaagagcacagcttacctccaaatgaactctctgagggccgaagacactgccgtgtactactgcgccaagtacccacactactacggctccagccactggtactttgacgtctggggccaaggcacactggtgacagtctcctccgcttccacaaagggacctagcgtgttcccactggcccctagcagcaagagcacaagcggcggcactgctgctctgggatgtctggtgaaggactacttcccagagccagtcactgtcagctggaacagcggagctctcacaagcggcgtgcatacattcccagctgtgctgcaaagcagcggactctactctctgtcctccgtcgtcactgtccctagctcctctctgggaacacagacatacatttgcaacgtgaaccacaagcctagcaacactaaggtggataagaaggtggagccaaagagctgtgacaagacacacacatgtcctccatgcccagccccagagctcctcggaggcccaagcgtctttctcttccctcctaagccaaaggacacactcatgatctctaggacaccagaggtgacatgtgtggtggtcgatgtgagccacgaggacccagaggtgaagttcaactggtacgtggatggcgtggaggtgcataatgctaagacaaagcctagagaggagcagtacaactccacttacagagtggtgagcgtgctgactgtgctgcaccaagattggctcaacggcaaggagtataagtgcaaggtgagcaacaaggctctgccagccccaattgagaagactatctccaaggctaagggccagcctagagagccacaagtgtacacactccctccaagcagagaggagatgacaaagaaccaagtgagcctcacttgtctggtcaagggcttttacccaagcgatatcgccgtcgagtgggaatccaacggccaaccagaaaacaactacaagacaacaccaccagtgctggatagcgatggcagcttctttctgtacagcaagctgactgtggacaagtctaggtggcagcaaggcaatgtgttcagctgttccgtgatgcatgaggctctccacaaccactacacacagaagtctctgtccctctcccccggcaaa

SEQ ID NO: 111 - protein, 5 aa, Artificial Sequence
  Description of Artificial Sequence Synthetic peptide
FHHTT

SEQ ID NO: 112 - RNA, 20 nt, Artificial Sequence
  Description of Artificial Sequence Synthetic oligonucleotide
cuuacuacaugugcguguuc

SEQ ID NO: 113 - RNA, 20 nt, Artificial Sequence
  Description of Artificial Sequence Synthetic oligonucleotide
acaaucauacgacgaguaaa

SEQ ID NO: 114 - protein, 8 aa, Artificial Sequence
  Description of Artificial Sequence Synthetic peptide
  MOD_RES (2)..(2): Val or Ile
  MOD_RES (4)..(4): Any amino acid
DXEXNPGP

SEQ ID NO: 115 - protein, 5 aa, Artificial Sequence
  Description of Artificial Sequence Synthetic peptide
GGGGS

SEQ ID NO: 116 - protein, 27 aa, Artificial Sequence
  Description of Artificial Sequence Synthetic peptide
  SITE (2)..(26): This sequence may encompass 2-5 “Glu Ala Ala Ala Lys” repeating units
AEAAAKEAAAKEAAAKEAAAKEAAAKA

SEQ ID NO: 117 - DNA, 23 bp, Cricetulus griseus
gaagtcctttcgagggcatgagg

SEQ ID NO: 118 - DNA, 23 bp, Cricetulus griseus
gtagcgttgtgacaggctttcgg

SEQ ID NO: 119 - DNA, 60 bp, Artificial Sequence
  Description of Artificial Sequence Synthetic oligonucleotide
tcagccgggccggtttggaggctgggagcagcagtcaataagggagccgcagtggagtag

SEQ ID NO: 120 - DNA, 60 bp, Artificial Sequence; identical to SEQ ID NO: 119
  Description of Artificial Sequence Synthetic oligonucleotide

SEQ ID NO: 121 - DNA, 60 bp, Artificial Sequence
  Description of Artificial Sequence Synthetic oligonucleotide
ttcatcgagttggattaaggtcatgctcacatccgtattccgacgtacgatggaccactc

SEQ ID NO: 122 - DNA, 60 bp, Artificial Sequence; identical to SEQ ID NO: 121
  Description of Artificial Sequence Synthetic oligonucleotide

SEQ ID NO: 123 - DNA, 60 bp, Artificial Sequence
  Description of Artificial Sequence Synthetic oligonucleotide
tcttttctcgagtatatctagagatatcttatgctcacgcttgagatccttgctatatca

SEQ ID NO: 124 - DNA, 60 bp, Artificial Sequence; identical to SEQ ID NO: 123
  Description of Artificial Sequence Synthetic oligonucleotide

SEQ ID NO: 125 - DNA, 60 bp, Artificial Sequence
  Description of Artificial Sequence Synthetic oligonucleotide
tatccccttcgggagctctgctgcaatagatctaaagttgagctgcttttcactcccatg

SEQ ID NO: 126 - DNA, 60 bp, Artificial Sequence; identical to SEQ ID NO: 125
  Description of Artificial Sequence Synthetic oligonucleotide

SEQ ID NO: 127 - DNA, 60 bp, Artificial Sequence
  Description of Artificial Sequence Synthetic oligonucleotide
acctgcgtgcgatcgtaccgtctacggcggataccaagttgcgcaggctatagcttgaag

SEQ ID NO: 128 - DNA, 60 bp, Artificial Sequence; identical to SEQ ID NO: 127
  Description of Artificial Sequence Synthetic oligonucleotide

SEQ ID NO: 129 - DNA, 60 bp, Artificial Sequence
  Description of Artificial Sequence Synthetic oligonucleotide
gttccgggctgaatgaagcgccgggaagtcgaccagacatgataagatacattgatgagt

SEQ ID NO: 130 - DNA, 60 bp, Artificial Sequence; identical to SEQ ID NO: 129
  Description of Artificial Sequence Synthetic oligonucleotide

SEQ ID NO: 131 - DNA, 60 bp, Artificial Sequence
  Description of Artificial Sequence Synthetic oligonucleotide
agccaacgtcggggcggcaggccctgccatagctgtgaattgttattcgcgaaaaacatc

SEQ ID NO: 132 - DNA, 60 bp, Artificial Sequence; identical to SEQ ID NO: 131
  Description of Artificial Sequence Synthetic oligonucleotide

SEQ ID NO: 133 - DNA, 60 bp, Artificial Sequence
  Description of Artificial Sequence Synthetic oligonucleotide
taacgcagatgagtattctacagagttgccgtagcaaccgccggctacgggggcctagga

SEQ ID NO: 134 - DNA, 60 bp, Artificial Sequence; identical to SEQ ID NO: 133
  Description of Artificial Sequence Synthetic oligonucleotide

SEQ ID NO: 135 - DNA, 60 bp, Artificial Sequence
  Description of Artificial Sequence Synthetic oligonucleotide
catgctcacatccgtattccgacgtacgatggaccactcttcacacgtaaagcaagaacg

SEQ ID NO: 136 - DNA, 60 bp, Artificial Sequence; identical to SEQ ID NO: 135
  Description of Artificial Sequence Synthetic oligonucleotide

SEQ ID NO: 137 - DNA, 60 bp, Artificial Sequence
  Description of Artificial Sequence Synthetic oligonucleotide
cctaaatcggcgcgataggccagtcgacccatagagcccaccgcatccccagcttgcctg

SEQ ID NO: 138 - DNA, 60 bp, Artificial Sequence; identical to SEQ ID NO: 137
  Description of Artificial Sequence Synthetic oligonucleotide

SEQ ID NO: 139 - DNA, 60 bp, Artificial Sequence
  Description of Artificial Sequence Synthetic oligonucleotide
caacgtcggggcggcaggccctgccatagcttagaggtttaattttgtatggcaaggtac

SEQ ID NO: 140 - DNA, 60 bp, Artificial Sequence; identical to SEQ ID NO: 139
  Description of Artificial Sequence Synthetic oligonucleotide

SEQ ID NO: 141 - DNA, 60 bp, Artificial Sequence
  Description of Artificial Sequence Synthetic oligonucleotide
gtcgtagcattatcggaggttctctaactaagtaatcggttgcgccgctcggactatcag

SEQ ID NO: 142 - DNA, 60 bp, Artificial Sequence; identical to SEQ ID NO: 141
  Description of Artificial Sequence Synthetic oligonucleotide

SEQ ID NO: 143 - DNA, 80 bp, Artificial Sequence
  Description of Artificial Sequence Synthetic oligonucleotide
cggaggttctctaactaagtaatcggttgcgccgctcggactatcagtacccgtgtctcgactctgccgcggctacctat

SEQ ID NO: 144 - DNA, 80 bp, Artificial Sequence; identical to SEQ ID NO: 143
  Description of Artificial Sequence Synthetic oligonucleotide

SEQ ID NO: 145 - DNA, 80 bp, Artificial Sequence
  Description of Artificial Sequence Synthetic oligonucleotide
cgaatgacacgcgtctccttgcgggtaaatcgccgaccgcgtcgaccagacatgataagatacattgatgagtttggaca

SEQ ID NO: 146 - DNA, 80 bp, Artificial Sequence; identical to SEQ ID NO: 145
  Description of Artificial Sequence Synthetic oligonucleotide

SEQ ID NO: 147 - DNA, 80 bp, Artificial Sequence
  Description of Artificial Sequence Synthetic oligonucleotide
  modified_base (55)..(55): a, c, t, g, unknown or other
ggctcgcagccaacgtcggggcggcaggccctgccatagcagaacttacgagccnggggaaacagtaaggcctaattagg

SEQ ID NO: 148 - DNA, 80 bp, Artificial Sequence; identical to SEQ ID NO: 147
  Description of Artificial Sequence Synthetic oligonucleotide
  modified_base (55)..(55): a, c, t, g, unknown or other

SEQ ID NO: 149 - DNA, 80 bp, Artificial Sequence
  Description of Artificial Sequence Synthetic oligonucleotide
tcacagatagtggggttcccggcaaagggcgtatatttgcataatttcgcccacctagcgcggggtccaacataggcgta

SEQ ID NO: 150 - DNA, 80 bp, Artificial Sequence; identical to SEQ ID NO: 149
  Description of Artificial Sequence Synthetic oligonucleotide

SEQ ID NO: 151 - DNA, 60 bp, Artificial Sequence
  Description of Artificial Sequence Synthetic oligonucleotide
cagccgggccggtttggaggctgggagcagcagtcaataagggagccgcagtggagtagg

SEQ ID NO: 152 - DNA, 60 bp, Artificial Sequence; identical to SEQ ID NO: 151
  Description of Artificial Sequence Synthetic oligonucleotide

SEQ ID NO: 153 - DNA, 60 bp, Artificial Sequence
  Description of Artificial Sequence Synthetic oligonucleotide
ttcatcgagttggattaaggtcatgctcacgtggtttgtctggtcaaccaccgcggtctc

SEQ ID NO: 154 - DNA, 60 bp, Artificial Sequence; identical to SEQ ID NO: 153
  Description of Artificial Sequence Synthetic oligonucleotide

SEQ ID NO: 155 - DNA, 60 bp, Artificial Sequence
  Description of Artificial Sequence Synthetic oligonucleotide
tctcgagtatatctagagatatcttatgctcacgcttgagatccttgctatatcatgaaa

SEQ ID NO: 156 - DNA, 60 bp, Artificial Sequence; identical to SEQ ID NO: 155
  Description of Artificial Sequence Synthetic oligonucleotide

SEQ ID NO: 157 - DNA, 60 bp, Artificial Sequence
  Description of Artificial Sequence Synthetic oligonucleotide
cccttcgggagctctgctgcaatagatctaaagttgagctgcttttcactcccatgaatg

SEQ ID NO: 158 - DNA, 60 bp, Artificial Sequence; identical to SEQ ID NO: 157
  Description of Artificial Sequence Synthetic oligonucleotide